(19) United States
(12) Patent Application Publication
(10) Pub. No.: US 2017/0000784 A1
(43) Pub. Date: Jan. 5, 2017

(54) Autophagy Inhibitors

(71) Applicants: Van Andel Research Institute, Grand Rapids, MI (US); Translational Genomics Research Institute, Phoenix, AZ (US)

(72) Inventors: Jeffrey Paul MacKeigan, East Grand Rapids, MI (US); Katie R. Martin, Rockford, MI (US); Megan Lynn Goodall, Denver, CO (US); Stephen Gately, Scottsdale, AZ (US); Tong Wang, Scottsdale, AZ (US)

(21) Appl. No.: 15/102,431

(22) PCT Filed: Dec. 5, 2014
(86) PCT No.: PCT/US14/68886

§ 371 Date: Jun. 7, 2016

Related U.S. Application Data
(60) Provisional application No. 61/913,321, filed on Dec. 8, 2013.

(51) Int. Cl. A61K 31/496; A61K 9/00; A61K 45/06; A61K 31/473

(52) U.S. Cl. CPC A61K 31/496; A61K 31/473; A61K 9/0053; A61K 45/06

(57) ABSTRACT

The present invention relates to compounds of Formula III, Formula 111(a), Formula V, Formula V(a), Formula A, Formula A 1, Formula A2, Formula A 3, or a pharmaceutically acceptable salt thereof that are useful as pharmaceutical agents, individually and/or in a combination with a chemotherapeutic agent: PLX-4032 (vemurafenib), or the catalytic mTOR inhibitor AZD8055, to treat a cancer and/or a cancer metastasis, for example a cancer harboring a BRAF protein kinase mutation and/or a HRAS protein mutation. Also, a method of treating and/or preventing malaria in a subject, the method comprising administering a therapeutically effective amount of a compound of Formula A, Formula A 1, Formula A2, Formula A 3, or a pharmaceutically acceptable salt thereof to the subject in need.

### CROSS-REFERENCE TO RELATED APPLICATIONS

This application claims priority to U.S. Provisional Application Ser. No. 61/913,321, filed Dec. 8, 2013, the disclosure of which is incorporated herein by reference in its entirety.

### FIELD OF THE INVENTION

The present invention relates to compounds and combinations of compounds that are useful as pharmaceutical agents, particularly as autophagy inhibitors.

### BACKGROUND OF THE INVENTION

Macroautophagy (autophagy) is an important mechanism for targeting cellular components including proteins, protein aggregates, and organelles for degradation in lysosomes. This catabolic, cellular self-digestion process is induced in response to starvation or stress, causing the formation of double membrane vesicles called autophagosomes that engulf proteins and organelles. Autophagosomes then fuse with lysosomes where the autophagosome and their cargo are degraded. This lysosome-mediated cellular self-digestion serves to recycle intracellular nutrients to sustain cell metabolism during starvation and to eliminate damaged proteins and organelles that accumulate during stress. Although elimination of individual proteins occurs by the ubiquitin-mediated proteasome degradation pathway, the autophagy pathway can eliminate protein aggregates and organelles. Thus, autophagy complements and overlaps with proteasome function to prevent the accumulation of damaged cellular components during starvation and stress. Through these functions, autophagy is an essential cellular stress response that maintains protein and organelle quality control, protects the genome from damage, and sustains cell and mammalian viability.

Autophagy is controlled by ATG proteins, initially identified in yeast, for which there are mammalian homologues (Levine, B., and Kroemer, G. (2008), Autophagy in the pathogenesis of disease, Cell 132, 27-42). ATG proteins are comprised of kinases, proteases, and two ubiquitin-like conjugation systems that likely function in concert with a host of unknown cellular proteins to control autophagosome formation, cargo recognition, engulfment, and trafficking to lysosomes.

Autophagy dysfunction is a major contributor to diseases including, but not limited to, neurodegeneration, liver disease, and cancer. Many human neurodegenerative diseases are associated with aberrant mutant and/or polyubiquitinated protein accumulation and excessive neuronal cell death.

Autophagy is also induced by stress and starvation in tumor cells, where it predominantly provides a prosurvival function. Metabolic stress is common, and autophagy localizes to metabolically-stressed tumor regions. Autophagy has been identified as an important survival pathway in epithelial tumor cells that enables long-term survival to metabolic stress (Degenhardt, K., et al. (2006), Autophagy promotes tumor cell survival and restricts necrosis, inflammation, and tumorigenesis, Cancer Cell 10, 51-64; Jin, S., and White, E. (2007), Role of autophagy in cancer: management of metabolic stress. Autophagy 3, 28-31; Karantza-Wadsworth, V., et al., (2007), Autophagy mitigates metabolic stress and genome damage in mammary tumorigenesis, Genes Dev 21, 1621-1635; Mathew, R. et al., (2007a), Role of autophagy in cancer, Nat Rev Cancer 7, 961-967; Mathew, R., et al. (2007b), Autophagy suppresses tumor progression by limiting chromosomal instability, Genes Dev 21, 1367-1381). Tumor cells with defined defects in autophagy accumulate p62-containing protein aggregates, damage DNA, and die in response to stress, whereas those with intact autophagy can survive for weeks, utilizing the autophagy survival pathway. Thus, autophagy prevents tumor cell damage and maintains metabolism. Tumor cells exploit this survival function to remain dormant, only to reemerge under more favorable conditions.

Paradoxically, autophagy defects through allelic loss of the essential autophagy gene beclin1 or through constitutive activation of the autophagy-suppressing PI-3 kinase/mTOR pathway are common in human tumors. Roughly half of human cancers may have impaired autophagy, either due to constitutive activation of the PI-3 kinase pathway or allelic loss of the essential autophagy gene beclin1, rendering them particularly susceptible to metabolic stress and autophagy inhibition (Jin et al., 2007; Jin, S., and White, E. (2008).

The importance of autophagy in cellular garbage disposal is clear, since autophagy is the only identified mechanism for the turnover of large cellular structures, such as organelles and protein aggregates. How organelles are recognized and directed to autophagosomes for degradation may involve organelle-specific processes, such as mitophagy and ER-phagy, that may mitigate oxidative stress emanating from dysfunctional organelles. Damaged proteins that accumulate during stress can be refolded, ubiquitinated, and degraded by the proteasome pathway, or aggregated and degraded by autophagy. To direct damaged or unfolded proteins to the autophagy pathway, p62 binds to polyubiquitinated proteins, forming protein aggregates by oligomerization, and to Atg8/LC3 on the autophagosome membrane to target aggregates to autophagosomes for degradation. Protein aggregation may be a protective mechanism to limit cellular exposure to toxic proteins through sequestration, as well as an efficient packaging and delivery mechanism that collects and directs damaged proteins to autophagosomes. Thus, the inability to dispose of p62 aggregates through autophagy appears to be toxic to normal tissues.

The ATG6/BECN1-Vps34-ATG8/LC3 complex regulates autophagosome formation. LC3 cleavage, lipidation, and membrane translocation are frequently utilized to monitor autophagy induction. The mechanism by which starvation and stress activate autophagy is controlled in part through the PI-3 kinase pathway via the protein kinase mTOR. Growth factor and nutrient availability promote mTOR activation that suppresses autophagy, whereas starvation and mTOR inactivation stimulate autophagy (Klionsky (2007), Nat Rev Mol Cell Biol 8, 931-937). While there are other mechanisms to regulate autophagy, mTOR provides a link between nutrient and growth factor availability, growth control, autophagy, and metabolism.

Autophagy plays an essential role in maintaining protein quality control, while defective autophagy is involved in the development of diseases including, but not limited to, cancer, neurodegenerative disorders, autoimmune disorders, cardiovascular disorders, metabolic disorders, hamartoma syndrome, genetic muscle disorders, and myopathies.

B-type Raf (BRAF) is a member of the Raf kinase family of serine/threonine-specific protein kinases. This protein plays a role in regulating the MAP kinase/ERKs signaling pathway, which affects cell division, differentiation, and secretion. A number of mutations in BRAF are known. In particular, the V600E mutation is prominent. Other BRAF mutations which have been found include: R461I, I462S, G463E, G463V, G465A, G465E, G465V, G468A, G468E, N580S, E585K, D593V, F594L, G595R, L596V, T598I, V599D, V599E, V599K, V599R, K600E, and A727V, and most of these mutations are clustered to two regions: the glycine-rich P loop of the N lobe and the activation segment and flanking regions. It has been reported by Davies, H., et al., Nature (2002) 417:949-954 that BRAF somatic missense mutations occur in 66% of malignant melanomas as well as at lower frequencies in other cancers. The mutations are in the kinase domain and a single substitution (V599E, now corrected to V600E) accounts for 80% of these mutations. These mutations result in proteins that have increased kinase activity. As these mutations are associated with malignant melanoma, inhibitors of the BRAF kinase proteins resulting from the V600E mutation have been employed as chemotherapeutic agents. Among these is Plexxikon 4032 (PLX-4032), also known as RG7204 and as ZELBORAF®. In one study, this inhibitor produced a 70% response rate in metastatic melanoma for patients with the mutation, but generally does not produce durable responses.

Therefore, there exists a need for identification of inhibitors of the autophagy survival pathway in, for example, cancer cells and cancer cells with mutations in protein kinases associated with deregulated growth control and kinase overexpression. Such inhibitors of autophagy can be used in the prevention, palliation, and/or treatment of cancer.

### SUMMARY OF THE INVENTION

In one aspect, disclosed is a compound of Formula III:

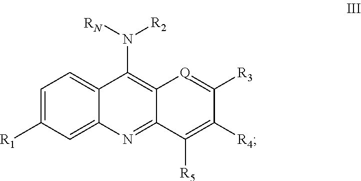

or a pharmaceutically acceptable salt thereof, wherein:
Q is CH or N;
R_{N} is —H, C₁-C₃ alkyl, or —CO—R_{N-1}, where R_{N-1} is C₁-C₃ alkyl or phenyl;
R₁ is —H, —F, —Cl, —Br, or —CF₃;
R₂ is —CH(R₂₋₁)ₙ₁—(CH₂)ₙ₂—Wₙ₃—X or —C*H—CH₂—CH₂—X₂—X₃—;
   n₁ is 0 or 1;
   R₂₋₁ is —H, C₁-C₃ alkyl, or C₃ cycloalkyl;
   n₂ is 0 through 3;
   n₃ is 0 or 1, with the provisos that (1) when n₁ or n₂ are other than 0, n₃ must be 0, (2) when n₃ is 1, n₁ and n₂ are both 0; (3) when n₁ is 1, X₁₋₂ and X₁₋₃ must be taken together with the attached nitrogen atom to form a monocyclic structure;
   W is a cyclic structure of three through seven atoms consisting of carbon, nitrogen, and sulfur, with the proviso that there not be more than one nitrogen or sulfur atom in the ring optionally containing 1 through 3 double bonds;
   X is —NX₁₋₂X₁₋₃, where X₁₋₂ and X₁₋₃ are the same or different and are C₁-C₄ substituted with one —OCH₃, —O—C₂H₅, alkoxy, haloalkoxy, haloalkyl, cyclopropyl, —CH₂-cyclopropyl, cyclobutyl, —SO₂—X₁₋₄ where X₁₋₄ is selected from —H and C₁-C₃ alkyl, —CO—X₁₋₄ where X₁₋₄ is as defined above, and where the X₁₋₂ and X₁₋₃ are taken together with the attached nitrogen atom to form a monocyclic structure consisting of four through seven atoms selected from the group consisting of carbon and nitrogen, with the proviso that the ring does not have more than two nitrogen atoms, —O—X₁₋₂ where X₁₋₂ is defined above;
   X₂ is —NX₁₋₂— or —O—, where X₁₋₂ is defined above;
   X₃ is —C*H—(CH₂)ₙ₄— or —(CH₂)ₙ₄—C*H— where n₄ is 0 through 2 and by convention * means the atoms marked with an asterisk (*) are bonded to each other resulting in the formation of a ring;
R₃ is —H, —F, —Cl, —Br, —CF₃, —OR₃₋₁ where R₃₋₁ is —H, C₁-C₆ alkyl or —CO—R₃₂ where R₃₋₂ is C₁-C₃ alkyl or phenyl, —N(R₃₋₁)₂ where the R₃₋₁ are the same or different and are as defined above, —SR₃₋₁ where R₃₋₁ is as defined above, —S(O)—R₃₋₁ where R₃₋₁ is as defined above, or —SO₂—R₃₋₁ where R₃₋₁ is as defined above;
R₄ is —H, —F, —Cl, —Br, —CF₃, —OR₄₋₁ where R₄₋₁ is —H, C₁-C₆ alkyl or —CO—R₄₋₂ where R₄₋₂ is C₁-C₃ alkyl or phenyl, —N(R₄₋₁)₂ where the R₄₋₁ are the same or different and are as defined above, —SR₄₋₁ where R₄₋₁ is as defined above, —S(O)—R₄₋₁ where R₄₋₁ is as defined above, or —SO₂—R₄₋₁ where R₄₋₁ is as defined above;
R₅ is —H, —F, —Cl, —Br, —CF₃, —OR₅₋₁ where R₅₋₁ is —H, C₁-C₆ alkyl or —CO—R₅₋₂ where R₅₋₂ is C₁-C₃ alkyl or phenyl, —N(R₅₋₁)₂ where the R₅₋₁ are the same or different and are as defined above, —SR₅₋₁ where R₅₋₁ is as defined above, —S(O)—R₅₋₁ where R₅₋₁ is as defined above, or —SO₂—R₅₋₁ where R₅₋₁ is as defined above;
   with the proviso that one of R₁, R₃, R₄ and R₅ must be other than —H.

In a further aspect, also disclosed is a compound of Formula V:

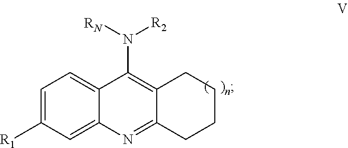

or a pharmaceutically acceptable salt thereof:
where n is 0 or 1;
where R_{N} is —H, C₁-C₃ alkyl, or —CO—R_{N-1}, where R_{N-1} is C₁-C₃ alkyl or phenyl;
where R₁ is —H, —F, —Cl, —Br, or —CF₃;
where R₂ is —CH(R₂₋₁)ₙ₂—(CH₂)ₙ₂—Wₙ₃—X
where n₁ is 0 or 1;
where R₂₋₁ is —H or C₁-C₃ alkyl;
where n₂ is 0 thru 3;
where n₃ is 0 or 1, with the provisos (1) that when n₁ or n₂ are other than 0, n₃ must be 0 and (2) that when n₃ is 1, n₁ and n₂ are both 0;
where W is a cyclic structure of three thru seven atoms consisting of carbon, nitrogen and sulfur with the proviso that there not be more than one nitrogen or sulfur atom in the ring optionally containing 1 thru 3 double bonds;
   where X is:
—NX₁₋₂X₁₋₃, where X₁₋₂ and X₁₋₃ are the same or different and are:
   —H,
   C₁-C₄ optionally substituted with one of —OH, —OCH₃, and —O—C₂H₅, cyclopropyl,
   CH₂-cyclopropyl,
   cyclobutyl,
   —CH₂—CH₂—N(X₁₋₄)(X₁₋₅) where X₁₋₄ and X₁₋₅ are the same or different and are —H and C₁-C₃ alkyl,
   —SO₂—X₁₋₄ where X₁₋₄ is as defined above,
   —CO—X₁₋₄ where X₁₋₄ is as defined above, and
   where the X₁₋₂ and X₁₋₃ are taken together with the attached nitrogen atom to form a monocyclic structure consisting of four thru seven atoms selected from the group consisting of carbon, nitrogen and oxygen with the provisos that the ring not have more than one oxygen atom and not more than two nitrogen atoms;
—O—X₁₋₂ where X₁₋₂ is as defined above;
—C*H—CH₂—CH₂—X₂—X₃— where
   X₂ is —NX₁₋₂— or —O—,
   X₃ is —C*H—(CH₂)ₘ₄— or —(CH₂)ₘ₄—C*H— where ₘ₄ is 0 thru 2 and by convention * means the atoms marked with an asterisk (*) are bonded to each other resulting in the formation of a ring, and pharmaceutically acceptable salts thereof.

In another aspect, also disclosed is a compound of Formula A:

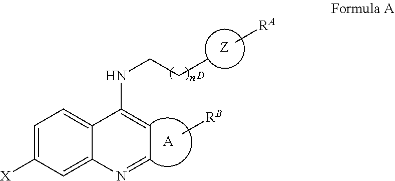

or a pharmaceutically acceptable salt thereof, wherein:

A is optionally substituted aryl or optionally substituted cycloalkyl;

Z is a 3 to 7 membered heterocycloalkyl;

X is H, halogen, or —CF₃;

n^{D} is 1 to 3;

R^{A} is optionally substituted C₁₋₆ alkyl; and

R^{B} is H, optionally substituted C₁₋₆ alkyl, or optionally substituted C₁₋₆ alkoxyl.

In one embodiment of Formula A, a compound or a pharmaceutically acceptable salt thereof has the structure of Formula A¹:

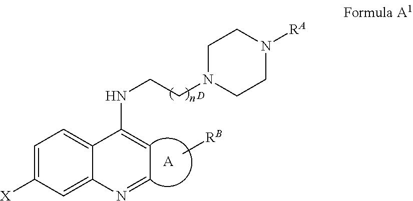

or a pharmaceutically acceptable salt thereof, wherein:

A is optionally substituted aryl or optionally substituted cycloalkyl;

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In one embodiment of Formula A, a compound has the structure of Formula A²:

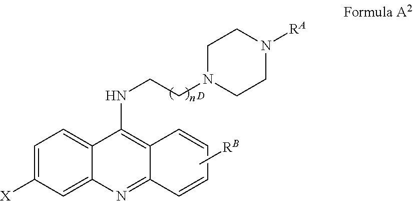

or a pharmaceutically acceptable salt thereof, wherein:

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In one embodiment of Formula A, the compound has the structure of Formula A³:

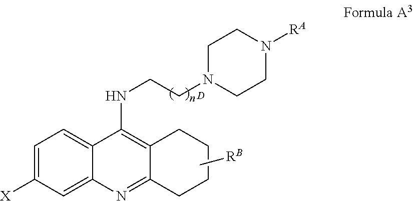

or a pharmaceutically acceptable salt thereof, wherein:

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

Also disclosed are pharmaceutical compositions containing compounds of Formulas III, III(a), V, or V(a).

Also disclosed are pharmaceutical compositions containing compounds of Formula A, Formula A¹, Formula A² or Formula A³.

Also disclosed are pharmaceutical compositions containing a combination of a compound of Formulas III, III(a), V, V(a), A, A¹, A² or A³.

Also disclosed are pharmaceutical compositions containing a compound of Formula III, Formula III(a), Formula V, Formula V(a), Formula A, Formula A¹, Formula A² or Formula A³, or a pharmaceutically acceptable salt thereof, in combination with a BRAF kinase inhibitor, or an mTOR inhibitor.

Also disclosed are processes for preparing compounds of Formulas III or V.

Also disclosed are methods of treating cancer, neurodegenerative disorders, autoimmune disorders, cardiovascular disorders, metabolic disorders, hamartoma syndrome, genetic muscle disorders, myopathies, and malaria, comprising administration to a patient or subject in need of such treatment a compound of Formula III, V, III(a), V(a), A, A¹, A² or A³ or a pharmaceutically salt thereof.

In one aspect, the present invention provides a method of treating a cancer harboring a B-type RAF kinase (BRAF-kinase) protein mutation in a subject in need thereof, the method comprising: administering to the subject, a pharmaceutical composition comprising a therapeutically effective amount of a compound of Formula A:

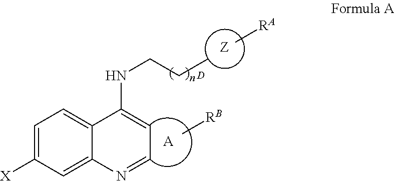

or a pharmaceutically acceptable salt thereof, wherein:

A is an optionally substituted aryl or optionally substituted cycloalkyl;

Z is a 3 to 7 membered heterocycloalkyl;

X is H, halogen, or —CF₃;

n^{D} is 1 to 3;

R^{A} is optionally substituted C₁₋₆ alkyl; and

R^{B} is H, optionally substituted C₁₋₆ alkyl, or optionally substituted C₁₋₆ alkoxyl.

In a further aspect, the present invention provides a method for the treatment of a cancer or a cancer metastasis in a subject, the method comprising: administering to the subject simultaneously or sequentially, a therapeutically effective amount of a combination of an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide (PLX-4032) and AZD-8055; and a compound of Formula III, Formula III(a), Formula V, Formula V(a), Formula A, Formula A¹, Formula A², Formula A³, or a pharmaceutically acceptable salt thereof.

Also disclosed are pharmaceutical compositions comprising a therapeutically effective amount of a compound of Formula III, Formula III(a), Formula V, Formula V(a), Formula A, Formula A¹, Formula A², Formula A³, or a pharmaceutically acceptable salt thereof, and an anti-cancer agent selected from PLX-4032 or AZD-8055 and a pharmaceutically acceptable excipient. In some embodiments, the pharmaceutical composition can comprise synergistically effective amounts of each component of a combination including a compound of Formula III, Formula III(a), Formula V, Formula V(a), Formula A, Formula A¹, Formula A² or Formula A³, or a pharmaceutically acceptable salt thereof, and an anti-cancer agent selected from PLX-4032 and AZD-8055.

Also disclosed are methods of sensitizing a cancer to the effects of a chemotherapeutic agent, the method includes administering to the subject with cancer, a pharmaceutical composition containing a compound of Formula III, Formula III(a), Formula V, Formula V(a), Formula A, Formula A¹, Formula A², Formula A³, or a pharmaceutically acceptable salt thereof prior to, concurrently, or subsequent to, administration of the chemotherapeutic.

In another aspect, the present invention provides a method for treating a cancer or a cancer metastasis in a subject, the method comprising administering to said subject, simultaneously or sequentially, a synergistically effective therapeutic amount of a combination of a compound of Formula III, Formula III(a), Formula V, Formula V(a), Formula A, Formula A¹, Formula A², Formula A³, or a pharmaceutically acceptable salt thereof, and an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide (PLX-4032) and AZD-8055.

In a further aspect, the present invention provides a method for the prevention and/or treatment of malaria in a subject in need of anti-malarial prevention or treatment, the method includes: administering to the subject, a therapeutically effective amount of a compound of Formula A:

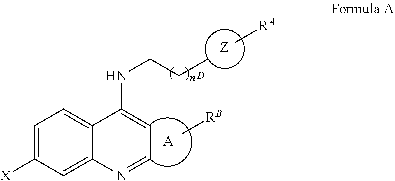

or a pharmaceutically acceptable salt thereof,
wherein:

A is an optionally substituted aryl or optionally substituted cycloalkyl;

Z is a 3 to 7 membered heterocycloalkyl;

X is H, halogen, or —CF₃;

n^{D} is 1 to 3;

R^{A} is optionally substituted C₁₋₆ alkyl; and

R^{B} is H, optionally substituted C₁₋₆ alkyl, or optionally substituted C₁₋₆ alkoxyl.

In a related embodiment, the method for the prevention and/or treatment of malaria in a subject in need of anti-malarial prevention or treatment includes administering to the subject in need thereof, a therapeutically effective amount of a compound of Formula A¹:

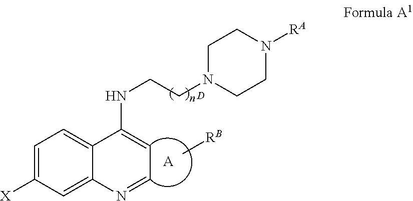

or a pharmaceutically acceptable salt thereof, wherein:

A is an optionally substituted aryl or optionally substituted cycloalkyl;

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In a further related embodiment, the method for the prevention and/or treatment of malaria in a subject in need of anti-malarial prevention or treatment includes administering to the subject in need thereof, a therapeutically effective amount of a compound of Formula A²:

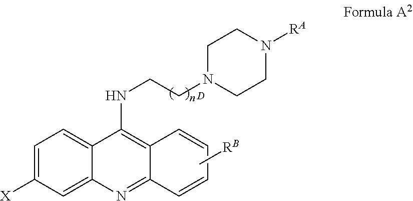

or a pharmaceutically acceptable salt thereof,

wherein:

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In a further related embodiment, the method for the prevention and/or treatment of malaria in a subject in need of anti-malarial prevention or treatment includes administering to the subject in need thereof, a therapeutically effective amount of a compound of Formula A³:

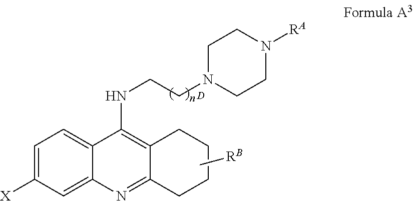

or a pharmaceutically acceptable salt thereof,
wherein:

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In various embodiments illustrated above, the compound of Formula A¹, A², or A³ or a pharmaceutically acceptable salt thereof can be formulated into a pharmaceutical composition in the form of a solution, a dispersion, a suspension, a powder, a capsule, a tablet, a pill, a time release capsule, a time release tablet, or a time release pill containing one or more doses of the compound of Formula A¹, A², or A³ or a pharmaceutically acceptable salt thereof. In various embodiments described herein, the pharmaceutical composition is administered to the subject intravenously, intramuscularly, subcutaneously, intraperitoneally, orally, or nasally. Therapeutically effective doses can include a dose amount of the compound of Formula A¹, A², or A³ or a pharmaceutically acceptable salt thereof, ranging from about 0.01 mg per kg body weight to about 100 mg per kg body weight.

In various embodiments, the subject in need of treatment and/or prevention may have or at risk of developing malaria, caused by a Plasmodium species selected from: Plasmodium falciparum, Plasmodium vivax, Plasmodium malariae, or Plasmodium ovale. In some of these embodiments, one or more of these Plasmodium species are chloroquine, mefloquine, sulfadoxine-pyrimethamine (SP), or artemisinin resistant.

### BRIEF DESCRIPTION OF THE FIGURES

FIG. 1A depicts tumor cell inhibition by Example 10 in cell lines H292, HCT116, and A375.

FIG. 1B depicts tumor cell inhibition by Example 10 in cell lines HCC1569, A498, and N87.

FIG. 2 depicts tumor cell inhibition by Example 10 in PLX-4032 resistant melanoma cell lines UACC1093 and UACC647.

FIG. 3A depicts tumor cell inhibition by Example 7 in cell line A375.

FIG. 3B depicts tumor cell inhibition by Example 26 in cell line A375.

FIG. 3C depicts tumor cell inhibition by Example 27 in cell line A375.

FIG. 4A depicts tumor cell inhibition by Example 10 in combination with PLX-4032 in cell line UACC1093.

FIG. 4B depicts tumor cell inhibition by Example 10 in combination with Temozolomide in cell line UACC1093.

FIG. 4C depicts tumor cell inhibition by Example 10 in combination with PLX-4032 in cell line UACC647.

FIG. 4D depicts tumor cell inhibition by Example 10 in combination with Temozolomide in cell line UACC647.

FIG. 5 depicts tumor cell weight in mice treated with Example 10.

FIG. 6A depicts a graph showing mean intensity quantification of red punctae on a dose response of Example 7 and Example 26 using image analysis software.

FIG. 6B depicts a graph showing percentage of cell viability after 48 hours of treatment with Example 7 and Example 26.

FIG. 7 depicts Western blot of U2OS cells treated with effective concentrations of piperaquine (100 μM), primaquine (100 μM), amodiaquine (50 μM), and artemisinin (50 μM) for three hours with and without rapamycin (100 nM) or bafilomycin A1 (100 nM). Cell lysates were probed by immunoblotting for endogenous LC3 (LC3-I: cytosolic; LC3-II: membrane-bound). Alpha-tubulin was included as a loading control. Quantification was performed using the Odyssey infrared imaging system.

FIG. 8A depicts a photomicrograph of U2OS cells expressing tandem fluorescent LC3 (tfLC3) were treated for 3 hours with chloroquine or quinacrine at the doses indicated, fixed, and imaged at 60× magnification. Green: GFP-LC3B; Red: RFP-LC3B, Blue: Hoechst (nuclei). Scale bars are 20 μm. Insets are at 2× magnification with scale bars set at 5 μm.

FIG. 8B depicts a line graph representing mean intensity of RFP-LC3B-positive puncta quantified using image analysis software on an average of 50 cells following treatment with chloroquine (CQ) (filled circles) or quinacrine (QN) (open circles) at the indicated doses. Error bars indicate standard deviation. Significant p-value of <0.05 (*), 0.01 (**), and 0.001 (***).

FIGS. 9A-9E depicts: (A) Images of U2OS-tfLC3 cells were first processed using a 2D blind deconvolution step within the image analysis software. Regions of Interest (ROIs) were then drawn around each cell. An intensity threshold was defined to include bright RFP-LC3-positive objects while minimizing background. The binary images created from thresholding is shown in red. Object data within the ROIs that is thresholded indicates an autophagosome. Data collected includes number of objects, which ROI object resides, mean intensity, and area. (B) Raw data of known autophagy inducers (rapamycin and AZD-8055) and inhibitor CQ showing puncta number, mean intensity, cell number, average intensity per cell, and puncta per cell values compared to a control. (C) Association plot of the mean intensity of RFP-LC3-positive puncta per cell against the puncta number per cell. The correlation coefficient (R²) was measured at 0.9899. (D) Relationship between number of puncta and puncta mean intensity (RFP-LC3) after VATG-027 treatment. The X-axis contains mean intensity bins while the Y-axis is the number of puncta. The light grey bars indicate the 0.3 μM concentration with the light grey area denoting distribution and the black bars indicate the 30 μM concentration with the dark grey area denoting distribution. The numbers above the bars are the total number of puncta. The higher concentration of VATG-027 shows a distribution of puncta with a higher mean intensity. (E) U2OS cells were treated with 30 μM of chloroquine, quinacrine, VATG-027, or VATG-032. Cells were stained with Hoechst (blue) and imaged in the red, green, and blue channels using the LUT settings for FIGS. 8 and 2. Representative images using VATG-027 in U2OS and tfLC3 cells at standard LUT settings and those set below cell auto-fluorescence are shown for comparison.

FIG. 10A depicts photomicrograph representing U2OS cells expressing tfLC3 were treated for 3 hours with chloroquine, VATG-027, or VATG-032 at the indicated doses, fixed, and imaged at 60× magnification. Green: GFP-LC3B; Red: RFP-LC3B, Blue: Hoechst (nuclei). Scale bars are 20 μm. Insets are at a 2.5× magnification with scale bars set at 8 μm.

FIG. 10B depicts line graph representing Mean pixel intensity of RFP-LC3 (red) puncta over a dose response with chloroquine (filled circles), VATG-027 (closed triangles, dashed line), and VATG-032 (open triangles, dashed line). Error bars indicate standard deviation. Significant p-value<0.05 (*), 0.01 (**), and 0.001 (***).

FIG. 10C depicts a FACS analysis of cleaved caspase-3 after treatment with 3 μM chloroquine, quinacrine, VATG-027, and VATG-032.

FIG. 10D depicts a line graph depicting percentage of cell viability compared to a DMSO control determined by CellTiter-Glo after 48 hours of treatment with chloroquine, quinacrine, VATG-027, or VATG-032. Error bars indicate standard deviation.

FIG. 11A depicts Western blots of U2OS cells treated with 1 μM, 3 μM, 10 μM and 30 μM of chloroquine, quinacrine, VATG-027, or VATG-032 for three hours. Cell lysates were probed by immunoblotting for endogenous LC3 (LC3-I: cytosolic; LC3-II: membrane-bound). Alpha-tubulin was included as a loading control.

FIG. 11B depicts a bar graph representing quantification of LC3-II western blot bands from FIG. 11A.

FIG. 12A depicts photomicrographs of U2OS cells treated for 3 hours with a vehicle control or 100 μM chloroquine, fixed, and analyzed by transmission electron microscopy (TEM). Accumulation in both size and number of electron dense and lucent vesicles, consistent with lysosomes and endosomes (black arrows), is observed following chloroquine treatment. Scale bar indicates 2 μm. Panels on the right are magnifications of the boxed regions (scale bars are 1.14 μm and 500 nm, respectively).

FIG. 12B depicts photomicrographs of U2OS cells treated for 3 hours with 3 μM of chloroquine, quinacrine, VATG-027, or VATG-032, fixed, and analyzed by TEM. Electron-dense and electron-lucent vesicles are indicated with black arrows. Scale bar indicates 2 μm in the images on the left. Panels on the right are magnified images of the boxed regions indicated by number (scale bars are 1.2 μm and 500 nm, respectively for panels 1 and 2).

FIG. 12C depicts Western blot of U2OS cells treated with 3 μM and 30 μM of chloroquine, quinacrine, VATG-027, and VATG-032 for 6 hours. Cell lysates were probed Using immunobloting for active cathepsin B. Alpha-tubulin was included as a loading control.

FIG. 13A depicts fluorescence microscopy of U2OS cells treated for 3 hours with vehicle control, or 3 μM autophagy inhibitor (chloroquine, quinacrine, VATG-027, or Example VATG-032) were stained with 100 nM LysoTracker Red for one hour prior to fixation, shown in red. Cells were stained by immunofluorescence with endogenous LAMP1 antibody and fluorescently conjugated secondary antibody (green), following cells were stained with Hoecsht (blue nuclei), and imaged at 60× magnification. Scale bars are 20 μm. Smaller insets are the red and green channels separated and magnified 1.5×.

FIG. 13B depicts 3D-graphical output representing intensity plots were generated using image analysis software and the intensities of red and green channels are displayed on the Z axis (peaks) of a 3D representation of the images in FIG. 13A.

FIG. 13C depicts bar graphs representing the quantification of co-localized LAMP1/LysoTracker Red as described in Example 8. Significant p-value<0.01 (**) and 0.001 (***) and Mander's co-localization coefficient (MCC).

FIG. 14 depicts fluorescence photomicrographs representing U2OS cells treated for 3 hours with vehicle control or 3 μM autophagy inhibitors (chloroquine, quinacrine, VATG-027, and VATG-032). Cells were stained with 100 nM LysoTracker Red for one hour prior to fixation and after fixation, were stained by immunofluorescence with endogenous LAMP1 antibody and fluorescently conjugated secondary antibody. Following, cells were stained with Hoecsht and imaged at 60× magnification. The ratio of LAMP1/LysoTracker Red was displayed on a colorimetric scale with red indicating only LAMP1 present, purple indicating only LysoTracker Red present, and green indicating both stains present. Images were then thresholded on the RGB scale to include only those puncta containing both LAMP1 and LysoTracker Red (green) and displayed in white. White puncta data were then exported and quantified.

FIG. 15A depicts Western blot of nine patient-derived melanoma cell lines were treated with 50 μM CQ for 0, 1, or 3 hours. Cell lysates were probed by immunoblotting for endogenous LC3 (LC3-I: cytosolic; LC3-II: membrane-bound). Alpha-tubulin was included as a loading control. Levels of LC3-II and tubulin were measured using quantitative western blotting machine.

FIG. 15B depicts a bar graph representing the levels of LC3-II and tubulin measured using quantitative western blotting and LC3-II normalized to α-tubulin. The fold change was determined by the change in LC3-II/α-tubulin from zero to three hours. Error bars indicate standard deviation. Significant p-value<0.05 (*) and 0.01 (**) compared to UACC2534 cells. Mutational status of BRAF and HRAS is indicated as mutant by (+) and wild-type by (−).

FIG. 16A depicts line graph representing melanoma A375 cell viability determined using the CellTiter-Glo luminescent assay after cells were treated for 48 hours with CQ, QN, Example 7, Example 27, and PLX-4032.

FIG. 16B depicts a photomicrograph of a Western blot of A375 cells treated with 0 μM, 10 nM, 100 nM, and 1 μM of PLX-4032 in the presence or absence of CQ (50 μM). Cell lysates and immunoblotting were used probe for total ERK1/2, phospho-ERK1/2, and LC3 (LC3-I: cytosolic; LC3-II: membrane-bound). Alpha-tubulin was included as a loading control.

FIG. 16C depicts a photomicrograph of a Western blot of U2OS cells were treated with 0 μM, 3 μM, and 30 μM chloroquine, quinacrine, VATG 027, or VATG 032 for three hours with and without PLX-4032 (400 nM). Cell lysates were probed by immunoblotting for endogenous LC3 (LC3-I: cytosolic; LC3-II: membrane-bound). Alpha-tubulin was included as a loading control

FIG. 17A depicts bar graphs representing soft agar assays using A375 cells treated every other day for three weeks in with the indicated doses of chloroquine, quinacrine, VATG-027, VATG-032, PLX-4032, or AZD-8055. Colonies were stained with crystal violet and quantified using image analysis software. Three independent experiments were averaged and error bars indicate standard deviation.

FIG. 17B depicts bar graphs representing soft agar colony formation assay using A375 cells that were treated every other day for three weeks with 3 μM of CQ, QN, VATG-032, and 1 μM VATG-027 in the presence or absence of PLX-4032 (400 nM). Colonies were stained with crystal violet and quantified using image analysis software. Three replicates were averaged and standard deviation is shown by error bars. p-value<0.05 (*), 0.01 (**), and 0.001 (***).

FIG. 17C depicts bar graphs representing soft agar assays were performed in 6-well plates using A375 cells that were treated every other day for three weeks at the IC₁₀ of AZD-8055 with and without the treatment at the IC₁₀ of chloroquine, quinacrine, VATG-027, or VATG-032. Colonies were stained with crystal violet and quantified using image analysis software. Three independent experiments were averaged. Error bars indicate standard. p-value<0.05 (*), 0.01 (**), and 0.001 (***).

FIG. 17D depicts a bar graph representing the percent change in total additivity of colony formation compared to the expected additive effect determined by the Bliss Independence model for each autophagy inhibitor in the presence or absence of AZD-8055.

FIG. 17E depicts a bar graph representing the survival of UACC91 cells treated every other day for three weeks at the IC₁₀ of PLX-4032 with and without the treatment at the IC₁₀ of quinacrine or VATG-032.

FIG. 18A depicts bar graphs representing soft agar assays were performed in six-welled plates using A375 cells that were treated every other day for three weeks at the IC₁₀ concentration of PLX-4032 (1.3 nM) in the presence or absence of the IC₁₀ concentration for chloroquine (274 nM), quinacrine (64 nM), VATG-027 (5 nM), or VATG-032 (2 nM). Colonies were stained with crystal violet and quantified using image analysis software. Three independent experiments were averaged and standard deviation is shown by error bars. Significant p-value<0.05 (*), 0.01 (**), and 0.001 (***).

FIG. 18B depicts a bar graph representing the total percent change in additivity above that of the expected additive effect determined by the Bliss Independence model for each autophagy inhibitor.

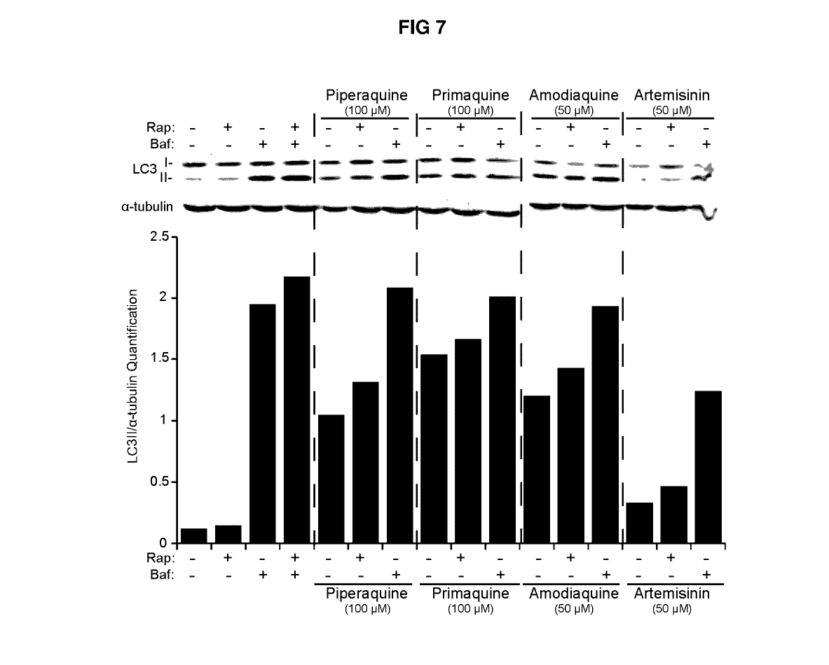

As used in the FIGS. 7-18B, and throughout the specification, VATG-027 refers to the compound of Example 7, and VATG-032 refers to the compound of Example 27 (See Table 1).

### DETAILED DESCRIPTION OF THE INVENTION

### Abbreviations and Definitions

The definitions and explanations below are for the terms as used throughout this entire document including both the specification and the claims.

| Abbreviation | Meaning |
|---|---|
| Ac | Acetyl |
| bALP | Bone-specific alkaline phosphatase |
| Br | Broad |
| ° C. | Degrees Celsius |
| c- | Cyclo |
| CBZ | CarboBenZoxy = benzyloxycarbonyl |
| CTx | Cross-linked C-terminal telopeptides of type-1 |
| | collagen |
| d | Doublet |
| dd | Doublet of doublet |
| dt | Doublet of triplet |
| DCM | Dichloromethane |
| DME | 1,2-dimethoxyethane |
| DMF | N,N-Dimethylformamide |
| DMSO | dimethyl sulfoxide |
| g | Gram(s) |
| h or hr | Hour(s) |
| HPLC | High pressure liquid chromatography |
| L | Liter(s) |
| M | Molar or molarity |
| m | Multiplet |
| mg | Milligram(s) |
| MHz | Megahertz (frequency) |
| Min | Minute(s) |
| mL | Milliliter(s) |
| μL | Microliter(s) |
| μM | Micromole(s) or micromolar |
| mM | Millimolar |
| Mmol | Millimole(s) |
| Mol | Mole(s) |
| MS | Mass spectral analysis |
| N | Normal or normality |
| nM | Nanomolar |
| NMR | Nuclear magnetic resonance spectroscopy |
| q | Quartet |
| RT | Room temperature |
| s | Singlet |
| t or tr | Triplet |
| TFA | Trifluoroacetic acid |
| THF | Tetrahydrofuran |

The symbol “-” means a single bond, “=” means a double bond, “≡” means a triple bond. The symbol “ [symbol] ” refers to a group on a double-bond as occupying either position on the terminus of a double bond to which the symbol is attached; that is, the geometry, E- or Z-, of the double bond is ambiguous. When a group is depicted removed from its parent Formula, the “˜,” symbol will be used at the end of the bond which was theoretically cleaved in order to separate the group from its parent structural Formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual Formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

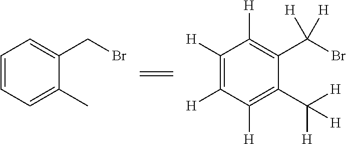

In this application, some ring structures are depicted generically and will be described textually. For example, in the schematic below, if in the structure on the left, ring A is used to describe a “spirocyclyl,” then if ring A is cyclopropyl, there are at most four hydrogens on ring A (when “R” can also be —H). In another example, as depicted on the right side of the schematic below, if ring B is used to describe a “phenylene” then there can be at most four hydrogens on ring B (assuming depicted cleaved bonds are not C—H bonds).

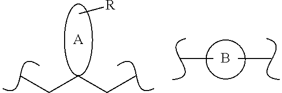

If a group “R” is depicted as “floating” on a ring system, as for example in the Formula:

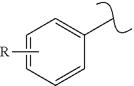

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused ring system, as for example in the formulae:

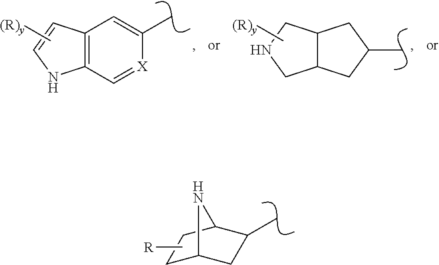

then, unless otherwise defined, a substituent “R” may reside on any atom of the fused ring system, assuming replacement of a depicted (for example the —NH— in the Formula above), implied (for example as in the Formula above, where the hydrogens are not shown but understood to be present), or expressly defined hydrogen (for example where in the Formula above, “X” equals ═CH—) from one of the ring atoms, so long as a stable structure is formed. In the example depicted, the “R” group may reside on either the 5-membered or the 6-membered ring of the fused ring system. In the Formula depicted above, when y is 2 for example, then the two “R's” may reside on any two atoms of the ring system, again assuming each replaces a depicted, implied, or expressly defined hydrogen on the ring.

When there are more than one such depicted “floating” groups, as for example in the formulae:

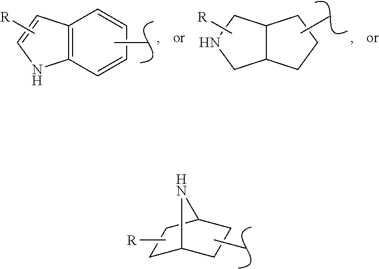

where there are two groups, namely, the “R” and the bond indicating attachment to a parent structure; then, unless otherwise defined, the “floating” groups may reside on any atoms of the ring system, again assuming each replaces a depicted, implied, or expressly defined hydrogen on the ring.

When a group “R” is depicted as existing on a ring system containing saturated carbons, as for example in the Formula:

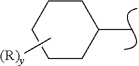

where, in this example, “y” can be more than one, assuming each replaces a currently depicted, implied, or expressly defined hydrogen on the ring; then, unless otherwise defined, where the resulting structure is stable, two “R's” may reside on the same carbon. A simple example is when R is a methyl group; there can exist a geminal dimethyl on a carbon of the depicted ring (an “annular” carbon). In another example, two R's on the same carbon, including that carbon, may form a ring, thus creating a spirocyclic ring (a “spirocyclyl” group) structure with the depicted ring as for example in the Formula:

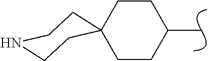

“Alkyl” is intended to include linear, branched, or cyclic hydrocarbon structures and combinations thereof, inclusively. For example, “C₈ alkyl” may refer to an n-octyl, iso-octyl, cyclohexylethyl, and the like. Lower alkyl refers to alkyl groups of from one to six carbon atoms. Examples of lower alkyl groups include methyl, ethyl, propyl, isopropyl, butyl, s-butyl, t-butyl, isobutyl, pentyl, hexyl and the like. Higher alkyl refers to alkyl groups containing more than eight carbon atoms. Exemplary alkyl groups are those of C₂₀ or below. Cycloalkyl is a subset of alkyl and includes cyclic hydrocarbon groups of from three to thirteen carbon atoms. Examples of cycloalkyl groups include c-propyl, c-butyl, c-pentyl, norbornyl, adamantyl and the like. In this application, alkyl refers to alkanyl, alkenyl, and alkynyl residues (and combinations thereof); it is intended to include cyclohexylmethyl, vinyl, allyl, isoprenyl, and the like. Thus when an alkyl residue having a specific number of carbons is named, all geometric isomers having that number of carbons are intended to be encompassed; thus, for example, either “butyl” or “C₄ alkyl” is meant to include n-butyl, sec-butyl, isobutyl, t-butyl, isobutenyl and but-2-yne radicals; and for example, “propyl” or “C₃ alkyl” each include n-propyl, propenyl, and isopropyl.

“Alkylene” refers to straight or branched chain divalent radical consisting solely of carbon and hydrogen atoms, containing no unsaturation and having from one to ten carbon atoms, for example, methylene, ethylene, propylene, n-butylene and the like. Alkylene is a subset of alkyl, referring to the same residues as alkyl, but having two points of attachment and, specifically, fully saturated. Examples of alkylene include ethylene (—CH₂CH₂—), propylene (—CH₂CH₂CH₂—), dimethylpropylene (—CH₂C(CH₃)₂CH₂—), and cyclohexylpropylene (—CH₂CH₂CH(C₆H₁₃).

“Alkoxy” or “alkoxyl” refers to the group —O-alkyl, for example including from one to eight carbon atoms of a straight, branched, cyclic configuration, unsaturated chains, and combinations thereof attached to the parent structure through an oxygen atom. Examples include methoxy, ethoxy, propoxy, isopropoxy, cyclopropyloxy, cyclohexyloxy and the like. Lower-alkoxy refers to groups containing one to six carbons.

“Amino” refers to the group —NH₂. “Substituted amino,” refers to the group —N(H)R or —N(R)R where each R is independently selected from the group: optionally substituted alkyl, optionally substituted alkoxy, optionally substituted aryl, optionally substituted heterocyclyl, acyl, carboxy, alkoxycarbonyl, sulfanyl, sulfinyl and sulfonyl, for example, diethylamino, methylsulfonylamino, furanyl-oxy-sulfonamino.

Aryl” refers to aromatic six- to fourteen-membered carbocyclic ring, for example, benzene, naphthalene, indane, tetralin, fluorene and the like, univalent radicals. As univalent radicals, the aforementioned ring examples are named, phenyl, naphthyl, indanyl, tetralinyl, and fluorenyl.

“Fused-polycyclic” or “fused ring system” refers to a polycyclic ring system that contains bridged or fused rings; that is, where two rings have more than one shared atom in their ring structures. In this application, fused-polycyclics and fused ring systems are not necessarily all aromatic ring systems. Typically, but not necessarily, fused-polycyclics share a vicinal set of atoms, for example naphthalene or 1,2,3,4-tetrahydro-naphthalene. A spiro ring system is not a fused-polycyclic by this definition, but fused polycyclic ring systems of the invention may themselves have spiro rings attached thereto via a single ring atom of the fused-polycyclic.

“Halogen” or “halo” refers to fluorine, chlorine, bromine or iodine. “Haloalkyl” and “haloaryl” refer generically to alkyl and aryl radicals that are substituted with one or more halogens, respectively. Thus, “dihaloaryl,” “dihaloalkyl,” “trihaloaryl” etc. refer to aryl and alkyl substituted with a plurality of halogens, but not necessarily a plurality of the same halogen; thus 4-chloro-3-fluorophenyl is within the scope of dihaloaryl.

“Heteroatom” refers to O, S, N, or P.

“Heterocyclyl” refers to a stable three- to fifteen-membered ring radical that consists of carbon atoms and from one to five heteroatoms selected from the group consisting of nitrogen, phosphorus, oxygen and sulfur. For purposes of this invention, the heterocyclyl radical may be a saturated, partially saturated, or unsaturated, monocyclic, bicyclic or tricyclic ring system, which may include fused or bridged ring systems as well as spirocyclic systems; and the nitrogen, phosphorus, carbon or sulfur atoms in the heterocyclyl radical may be optionally oxidized to various oxidation states. In a specific example, the group —S(O)₀₋₂—, refers to —S— (sulfide), —S(O)— (sulfoxide), and —SO₂— (sulfone). For convenience, nitrogens, particularly but not exclusively, those defined as annular aromatic nitrogens, are meant to include their corresponding N-oxide form, although not explicitly defined as such in a particular example. Thus, for a compound of the invention having, for example, a pyridyl ring; the corresponding pyridyl-N-oxide is meant to be included as another compound of the invention. In addition, annular nitrogen atoms may be optionally quaternized; and the ring radical may be partially or fully saturated or aromatic. Examples of heterocyclyl radicals include, but are not limited to, azetidinyl, acridinyl, benzodioxolyl, benzodioxanyl, benzofuranyl, carbazoyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, tetrahydrofuryl, tetrahydropyranyl, thienyl, benzothieliyl, thiamorpholinyl, thiamorpholinyl sulfoxide, thiamorpholinyl sulfone, dioxaphospholanyl, and oxadiazolyl.

“Heteroalicyclic” refers specifically to a non-aromatic heterocyclyl radical. A heteroalicyclic may contain unsaturation, but is not aromatic.

“Heterocycloalkyl” refers to a 3-10 membered mono- or bicyclic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety to form structures, such as tetrahydroisoquinoline, which would be categorized as heteroaryls.

“Heteroaryl” as used herein, refers to a monocyclic, bicyclic, or tricyclic ring system having 4 to 15 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl.

“Heterocyclylalkyl” refers to a residue in which a heterocyclyl is attached to a parent structure via one of an alkylene, alkylidene, or alkylidyne radical. Examples include (4-methylpiperazin-1-yl) methyl, (morpholin-4-yl) methyl, (pyridine-4-yl) methyl, 2-(oxazolin-2-yl) ethyl, 4-(4-methylpiperazin-1-yl)-2-butenyl, and the like. Both the heterocyclyl, and the corresponding alkylene, alkylidene, or alkylidyne radical portion of a heterocyclylalkyl group may be optionally substituted. “Lower heterocyclylalkyl” refers to a heterocyclylalkyl where the “alkyl” portion of the group has one to six carbons. “Heteroalicyclylalkyl” refers specifically to a heterocyclylalkyl where the heterocyclyl portion of the group is non-aromatic; and “heteroarylalkyl” refers specifically to a heterocyclylalkyl where the heterocyclyl portion of the group is aromatic Such terms may be described in more than one way, for example, “lower heterocyclylalkyl” and “heterocyclyl C₁₋₆ alkyl” are equivalent terms.

As used herein, “cyclic moiety” or “cyclic structure” includes cycloalkyl, heterocycloalkyl, cycloalkenyl, heterocycloalkenyl, aryl, or heteroaryl, each of which has been defined previously.

“Optional” or “optionally” means that the subsequently described event or circumstance may or may not occur, and that the description includes instances where said event or circumstance occurs and instances in which it does not. One of ordinary skill in the art would understand that, with respect to any molecule described as containing one or more optional substituents, that only sterically practical and/or synthetically feasible compounds are meant to be included. “Optionally substituted” refers to all subsequent modifiers in a term, for example in the term “optionally substituted arylC₁₋₈ alkyl,” optional substitution may occur on both the “C₁₋₈ alkyl” portion and the “aryl” portion of the molecule; and for example, optionally substituted alkyl includes optionally substituted cycloalkyl groups, which in turn are defined as including optionally substituted alkyl groups, potentially ad infinitum. A list of exemplary optional substitution are listed below in the definition of “substituted.”

“Substituted” alkyl, aryl, and heterocyclyl, refer respectively to alkyl, aryl, and heterocyclyl, wherein one or more (for example up to about five, in another example, up to about three) hydrogen atoms are replaced by a substituent independently selected from: optionally substituted alkyl (for example, fluoromethyl), optionally substituted aryl (for example, 4-hydroxyphenyl), optionally substituted arylalkyl (for example, 1-phenyl-ethyl), optionally substituted heterocyclylalkyl (for example, 1-pyridin-3-yl-ethyl), optionally substituted heterocyclyl (for example, 5-chloro-pyridin-3-yl or 1-methyl-piperidin-4-yl), optionally substituted alkoxy, alkylenedioxy (for example methylenedioxy), optionally substituted amino (for example, alkylamino and dialkylamino), optionally substituted amidino, optionally substituted aryloxy (for example, phenoxy), optionally substituted arylalkyloxy (for example, benzyloxy), carboxy (—CO2H), carboalkoxy (that is, acyloxy or —OC(═O)R), carboxyalkyl (that is, esters or —CO2R), carboxamido, benzyloxycarbonylamino (CBZ-amino), cyano, acyl, halogen, hydroxy, nitro, sulfanyl, sulfinyl, sulfonyl, thiol, halogen, hydroxy, oxo, carbamyl, acylamino, and sulfonamido.

“Sulfanyl” refers to the groups: —S-(optionally substituted alkyl), —S-(optionally substituted aryl), and —S-(optionally substituted heterocyclyl).

“Sulfinyl” refers to the groups: —S(O)—H, —S(O)-(optionally substituted alkyl), —S(O)-optionally substituted aryl), and —S(O)-(optionally substituted heterocyclyl).

“Sulfonyl” refers to the groups: —S(O₂)—H, —S(O₂)-(optionally substituted alkyl), —S(O₂)-optionally substituted aryl), —S(O₂)-(optionally substituted heterocyclyl), —S(O₂)-(optionally substituted alkoxy), —S(O₂)-optionally substituted aryloxy), and —S(O₂)-(optionally substituted heterocyclyloxy).

“Yield” for each of the reactions described herein is expressed as a percentage of the theoretical yield.

Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS).

The compounds of the invention, or their pharmaceutically acceptable salts, may have asymmetric carbon atoms, oxidized sulfur atoms or quaternized nitrogen atoms in their structure.

The compounds of the invention and their pharmaceutically acceptable salts may exist as single stereoisomers, racemates, and as mixtures of enantiomers and diastereomers. The compounds may also exist as geometric isomers. All such single stereoisomers, racemates and mixtures thereof, and geometric isomers are intended to be within the scope of this invention.

It is assumed that when considering generic descriptions of compounds of the invention for the purpose of constructing a compound, such construction results in the creation of a stable structure. That is, one of ordinary skill in the art would recognize that there can theoretically be some constructs which would not normally be considered as stable compounds (that is, sterically practical and/or synthetically feasible, supra).

When a particular group with its bonding structure is denoted as being bonded to two partners; that is, a divalent radical, for example, —OCH₂—, then it is understood that either of the two partners may be bound to the particular group at one end, and the other partner is necessarily bound to the other end of the particular group, unless stated explicitly otherwise.

Stated another way, divalent radicals are not to be construed as limited to the depicted orientation, for example “—OCH₂—” is meant to mean not only “—OCH₂—” as drawn, but also “—CH₂O—.”

With regard to various cyclic substituents, such as those within the scope of group W such as pyridinyl, when various positions of attachment are possible, such as for pyridine (i.e., pyridin-2-yl, pyridin-3-yl, and pyridin-4-yl), all are within the scope of the present invention.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. Enantiomers (R- and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents, or by converting on enantiomer to the other by asymmetric transformation. For a mixture of enantiomers, enriched in a particular enantiomer, the major component enantiomer may be further enriched (with concomitant loss in yield) by recrystallization.

The present invention includes all pharmaceutically acceptable isotopically-labelled compounds of Formula (I) wherein one or more atoms are replaced by atoms having the same atomic number, but an atomic mass or mass number different from the atomic mass or mass number usually found in nature. Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of the Formulas of the present invention can generally be prepared by conventional techniques known to those skilled in the art or by processes analogous to those described in the accompanying Examples and Preparations using an appropriate isotopically-labeled reagent in place of the non-labeled reagent previously employed.

“Patient” or “Subject” are used interchangeably and for the purposes of the present invention includes humans and other animals, particularly mammals, and other organisms. Thus the methods are applicable to both human therapy and veterinary applications. More specifically, the patient is a mammal, and in some embodiments, the patient or subject is human.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to his/her own knowledge and to this disclosure.

In more specific terms, the term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by Plasmodium or (ii) associated with Plasmodium activity, or (iii) characterized by activity (normal or abnormal) of Plasmodium or (2) reduce or inhibit the activity of Plasmodium; or (3) reduce or inhibit the growth of Plasmodium. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of Plasmodium; or at least partially reducing or inhibiting the growth of Plasmodium.

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) wherein the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8th Ed., Pergamon Press, Gilman et al. (eds), 1990 for a discussion of biotransformation). As used herein, the metabolite of a compound of the invention or its salt may be the biologically active form of the compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a compound of the present invention is known to one of skill in the art in light of the present disclosure.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

In addition, it is intended that the present invention cover compounds made either using standard organic synthetic techniques, including combinatorial chemistry or by biological methods, such as bacterial digestion, metabolism, enzymatic conversion, and the like.

“Treating” or “treatment” as used herein includes the treatment of a cancer in a human, which cancer is characterized by abnormal cellular proliferation, and invasion and includes at least one of: (i) preventing the disease-state from occurring in a human, in particular, when such human is predisposed to the disease-state but has not yet been diagnosed as having it; (ii) inhibiting the disease-state, i.e., arresting its development; and (iii) relieving the disease-state, i.e., causing regression of the disease-state. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

“Treating” or “treatment” as used herein also includes the treatment of malaria in a subject, or symptoms related thereto, as caused by a species of the malaria causing Plasmodium family of protozoans, including, Plasmodium falciparum, Plasmodium vivax, Plasmodium malariae, Plasmodium ovale, or Plasmodium berghei as illustrative examples of malaria causative organisms.

A “chemotherapeutic agent” is a biological (large molecule) or chemical (small molecule) compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, proteins, antibodies, photosensitizers, mTOR inhibitors, and kinase inhibitors. Chemotherapeutic agents include compounds used in “targeted therapy” and non-targeted conventional chemotherapy.

All temperatures are in degrees Celsius (° C.). 20-25° C. denotes room temperature.

Chromatography (column and flash chromatography) refers to purification/separation of compounds expressed as (support, eluent). It is understood that the appropriate fractions are pooled and concentrated to give the desired compound(s).

Saline refers to an aqueous saturated sodium chloride solution.

Alcohol refers to ethyl alcohol.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance, and bioavailability.

When solvent pairs are used, the ratios of solvents used are volume/volume (v/v).

When the solubility of a solid in a solvent is used the ratio of the solid to the solvent is weight/volume (wt/v).

The invention further encompasses aspects in which a protecting group is added to the compound. One skilled in the art would recognize that during the synthesis of complex molecules, one group on the disclosed compound may happen to interfere with an intended reaction that includes a second group on the compound. Temporarily masking or protecting the first group encourages the desired reaction. Protection involves introducing a protecting group to a group to be protected, carrying out the desired reaction, and removing the protecting group. Removal of the protecting group may be referred to as deprotection. Examples of compounds to be protected in some syntheses include hydroxy groups, amine groups, carbonyl groups, carboxyl groups, and thiols.

A protecting group may result from any chemical synthesis that selectively attaches a group that is resistant to certain reagents to the chemical group to be protected without significant effects on any other chemical groups in the molecule, remains stable throughout the synthesis, and is removed through conditions that do not adversely react with the protected group, nor any other chemical group in the molecule.

Protecting groups, reagents that add those groups, preparations of those reagents, protection and deprotection strategies under a variety of conditions, including complex syntheses with mutually complementary protecting groups, are all well known in the art. Examples of all of these may be found in Green et al, Protective Groups in Organic Chemistry 2nd Ed., (Wiley 1991), and Harrison et al, Compendium of Synthetic Organic Methods, Vols. 1-8 (Wiley, 1971-1996) both of which hereby incorporated by reference in its entirety.

Racemates, individual enantiomers, or diasteromers of the disclosed compound are prepared by specific synthesis or resolution through known methods. For example, the disclosed compound may be resolved into it enantiomers by the formation of diasteromeric pairs through salt formation using an optically active acid. Enantiomers are fractionally crystallized and the free base regenerated. In another example, enantiomers may be separated by chromatography. Such chromatography is any appropriate method that is appropriate to separate enantiomers such as HPLC on a chiral column as is known to those skilled in the art.

Cancer cells include any cells derived from a tumor, neoplasm, cancer, precancer, cell line, or any other source of cells that are ultimately capable of potentially unlimited expansion and growth. Cancer cells may be derived from naturally occurring sources or may be artificially created. Cancer cells may also be capable of invasion into other tissues and metastasis when placed into an animal host. Cancer cells further encompass any malignant cells that have invaded other tissues and/or metastasized. One or more cancer cells in the context of an organism may also be called a cancer, tumor, neoplasm, growth, metastasis, malignancy, or any other term used in the art to describe cells in a cancerous state.

Expansion of a cancer cell includes any process that results in an increase in the number of individual cells derived from a cancer cell. Expansion of a cancer cell may result from mitotic division, proliferation, or any other form of expansion of a cancer cell, whether in vitro or in vivo. Expansion of a cancer cell further encompasses invasion and metastasis. A cancer cell may be in physical proximity to cancer cells from the same clone or from different clones that may or may not be genetically identical to it. Such aggregations may take the form of a colony, tumor or metastasis, any of which may occur in vivo or in vitro. Slowing the expansion of the cancer cell may be brought about either by inhibiting cellular processes that promote expansion or by bringing about cellular processes that inhibit expansion. Processes that inhibit expansion include processes that slow mitotic division and processes that promote cell senescence or cell death. Examples of specific processes that inhibit expansion include capsase dependent and independent pathways, autophagy, necrosis, apoptosis, and mitochondrial dependent and independent processes.

Treatment is contemplated in living entities including but not limited to mammals (particularly humans) as well as other mammals include livestock (horses, cattle, sheep, pigs) and other animals generally bred for domesticated companion animals such as dogs and cats.

### Compounds

As indicated previously, in one aspect, the invention is directed to a compound of Formula III

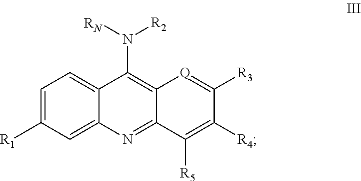

or a pharmaceutically acceptable salt thereof.

In some embodiments of a compound of Formula III, Q is CH.

In some embodiments, R₁ is —F, —Cl, or —Br. More particularly, R₁ is —Cl.

In some embodiments, R₃ is —OR₃₁. More particularly, R₃₋₁ is —Cl.

In some embodiments, R₄ and R₅ are —H.

In some embodiments RN is —H.

When X₁₋₂ and X₁₋₃ are taken together with the attached nitrogen atom to form a monocyclic structure consisting of four through seven atoms selected from the group consisting of carbon and nitrogen, the cyclic structure can be either saturated like piperazinyl or aromatic like pyridinyl.

Thus, in some embodiments, the monocyclic structure be selected from the group consisting of piperazin-1-yl optionally substituted in the 4-position with C₁-C₃ alkyl, —CO—(C₁-C₃ alkyl), —SO₂—H, or —SO₂—(C₁-C₃) alkyl; piperidin-1-yl and piperidin-4-yl both optionally substituted with one —F, —Cl, C₁-C₃ alkyl, —CO—(C₁-C₃ alkyl), —SO₂—H, or —SO₂—(C₁-C₃) alkyl; and pyrrolidin-1-yl, pyrrolinin-2-yl, and pyrrolidin-3-yl all optionally substituted with one —F, —Cl, C1-C₃ alkyl, —CO—(C₁-C₃ alkyl), —SO₂—H, or —SO2-(C₁-C₃) alkyl.

More particularly, X₁₋₂ and X₁₋₃ are cyclized to form pyrrolidin-1-yl, N-(1-methylpyrrolidin-3-yl), N-(4-methylpiperazin-1-yl), and N-(1ethylpiperadin-4-yl).

Also, when W is a cyclic structure of three through seven atoms consisting of carbon, nitrogen, and sulfur, the cyclic structure be selected from the group consisting of phenyl, thiazolyl, pyridinyl, and C₃-C₇ cycloalkyl.

In some embodiments, the compound of Formula III is a compound selected from Examples 5, 7, 10, and 16.

One embodiment of a compound of Formula III is a compound of Formula III(a):

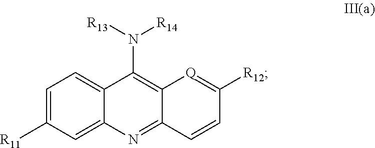

or a pharmaceutically acceptable salt thereof, wherein:
Q₁ is selected from the group consisting of CH and N;
R₁₁ is selected from the group consisting of H, F, Cl, Br, and C₁₋₃ haloalkyl;
R₁₂ is selected from the group consisting of H, F, Cl, Br, OH, C₁₋₃ alkyl, C₁₋₃ haloalkyl, and C₁₋₃ alkoxy;
R₁₃ is selected from the group consisting of H, C₁₋₃ alkyl, and C₁₋₃ haloalkyl;
R₁₄ is selected from the group consisting of optionally substituted 5- or 6-membered cycloalkyl or heterocycloalkyl, optionally substituted

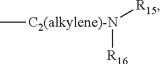

optionally substituted

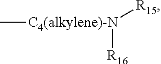

and optionally substituted

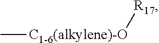

wherein the alkylene chains may be optionally substituted with up to 3 R₁₈;
R₁₅ and R₁₆ are each independently selected from the group consisting of H, alkyl, cycloalkyl, alkoxy, alkylamino, and sulfonyl;
or R₁₅ and R₁₆ may be joined together to form an optionally substituted 5- or 6-membered cycloalkyl or heterocycloalkyl;
R₁₇ is selected from the group consisting of H, alkyl, cycloalkyl, alkoxy, alkylamino, and sulfonyl; and
R₁₈ is selected from the group consisting of H, alkyl, cycloalkyl, alkoxy, alkylamino, and sulfonyl.

In one embodiment of the compound of Formula III(a), Q₁ is CH.

In another embodiment, Q₁ is N.

In one embodiment, R₁₁ is H, F, or Cl.

More particularly, R₁₁ is H.

In another embodiment, R₁₁ is F.

In yet another embodiment, R₁₁ is Cl.

In one embodiment, R₁₂ is H, F, Cl, OH, or C₁₋₃ alkoxy;

More particularly, R₁₂ is F or C₁₋₃ alkoxy.

More particularly, R₁₂ is F.

In another embodiment, R₁₂ is C₁₋₃ alkoxy.

More particularly, R₁₂ is methoxy.

In one embodiment, R₁₃ is H.

In another embodiment, R₁₃ is C₁₋₃ alkyl.

More particularly, R₁₃ is methyl.

In one embodiment, R₁₁ is C₁, and R₁₂ is methoxy.

In another embodiment, Q₁ is N, and R₁₂ is methoxy.

In another embodiment, Q₁ is N, and R₁₂ is H.

In another embodiment, R₁₁ is Br, and R₁₂ is methoxy.

In another embodiment, R₁₁ is F, and R₁₂ is methoxy.

In another embodiment, Q₁ is CH, and R₁₂ is Cl or F.

In one embodiment, R₁₃ is H, and Q₁ is CH.

In another embodiment, R₁₁ is Cl, and R₁₃ is H.

In any of the above embodiments of a compound of Formula III(a) provided above, R₁₄ is an optionally substituted 5- or 6-membered cycloalkyl or heterocycloalkyl,

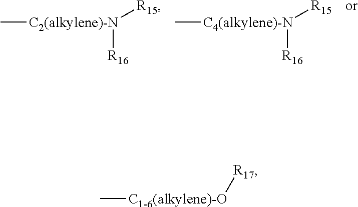

wherein the alkylene chains may be optionally substituted with up to 3 R₁₈.

In some embodiments, R₁₄ is an optionally substituted 5- or 6-membered heterocycloalkyl.

More particularly, R₁₄ is

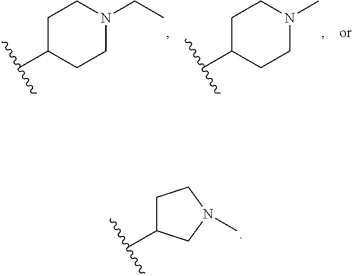

In some embodiments, R₁₄ is optionally substituted

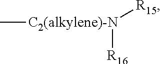

wherein the alkylene chain may be optionally substituted with up to 3 R₁₈.

More particularly, R₁₄ is

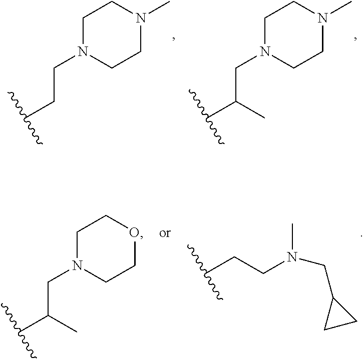

In some embodiments, R₁₄ is optionally substituted

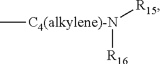

wherein the alkylene chain may be optionally substituted with up to 3 R₁₈.

More particularly, in some embodiments, R₁₄ is

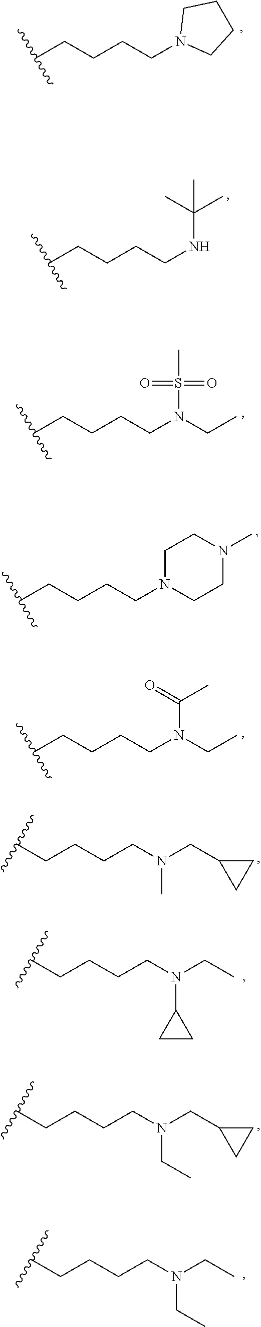

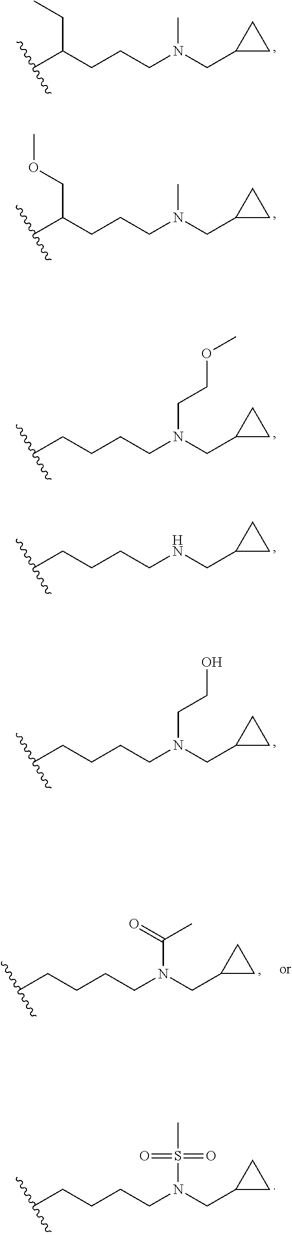

In some embodiments, R₁₄ is optionally substituted

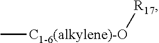

wherein the alkylene chain may be optionally substituted with up to 3 R₁₈.

More particularly, R₁₄ is

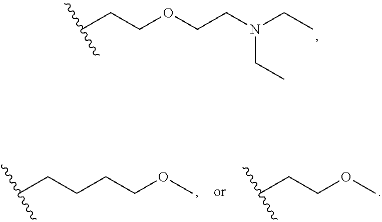

In another aspect, the invention is directed to a compound of Formula V

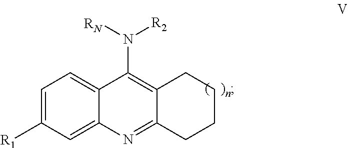

or a pharmaceutically acceptable salt thereof
where n is 0 or 1;
where R_{N} is —H, C₁-C₃ alkyl, or —CO—R_{N-1}, where R_{N-1} is C₁-C₃ alkyl or phenyl;
where R₁ is —H, —F, —Cl, —Br, or —CF₃;
where R₂ is —CH(R₂₋₁)ₙ₁—(CH₂)ₙ₂—Wₙ₃—X

where n₁ is 0 or 1;

where R₂₋₁ is —H or C₁-C₃ alkyl;

where n₂ is 0 thru 3;

where n₃ is 0 or 1, with the provisos (1) that when n₁ or n₂ are other than 0, n₃ must be 0 and (2) that when n₃ is 1, n₁ and n₂ are both 0;

where W is a cyclic structure of three thru seven atoms consisting of carbon, nitrogen and sulfur with the proviso that there not be more than one nitrogen or sulfur atom in the ring optionally containing 1 thru 3 double bonds;

where X is

—NX₁₋₂X₁₋₃, where X₁₋₂ and X₁₋₃ are the same or different and are:
—H,
C₁-C₄ optionally substituted with one of —OH, —OCH₃, and —O—C₂H₅, cyclopropyl,
CH₂-cyclopropyl,
cyclobutyl,
—CH₂—CH₂—N(X₁₋₄)(X₁₋₅) where X₁₋₄ and X₁₋₅ are the same or different and are —H and C₁-C₃ alkyl,
—SO₂—X₁₋₄ where X₁₋₄ is as defined above,
—CO—X₁₋₄ where X₁₋₄ is as defined above, and
where the X₁₋₂ and X₁₋₃ are taken together with the attached nitrogen atom to form a monocyclic structure consisting of four thru seven atoms selected from the group consisting of carbon, nitrogen and oxygen with the provisos that the ring not have more than one oxygen atom and not more than two nitrogen atoms;
—O—X₁₋₂ where X₁₋₂ is as defined above;
—C*H—CH₂—CH₂—X₂—X₃— where
   X₂ is —NX₁₋₂— or —O—,
   X₃ is —C*H—(CH₂)ₘ₄— or —(CH₂)ₘ₄—C*H— where ₘ₄ is 0 thru 2 and by convention means the atoms marked with an asterisk (*) are bonded to each other resulting in the formation of a ring, or a pharmaceutically acceptable salt thereof.

In some embodiments, R₁ is —F, —Cl, and —Br. More particularly, R₁ is —Cl.

In some embodiments, R_{N} is —H. In some embodiments, X₁₋₂ and X₁₋₃ are taken together with the attached nitrogen atom to form a monocyclic structure consisting of four through seven atoms selected from the group consisting of carbon, nitrogen and oxygen. In some embodiments, the monocyclic structure be selected from the group consisting of where X₁₋₂ and X₁₋₃ are cyclized to form a cyclic structure selected from the group consisting of piperazin-1-yl optionally substituted in the 4-position with C₁-C₃ alkyl, —CO—(C₁-C₃ alkyl), —SO₂—H, or —SO₂—(C₁-C₃) alkyl; piperidin-1-yl and piperidin-4-yl both optionally substituted with one —F, —Cl, C₁-C₃ alkyl, —CO—(C₁-C₃ alkyl), —SO₂—H, or —SO₂—(C₁-C₃) alkyl; morpholin-1-yl optionally substituted with one —F, —Cl, C₁-C₃ alkyl, —CO—(C₁-C₃ alkyl), —SO₂—H, or —SO₂—(C₁-C₃) alkyl; pyrrolidin-1-yl, pyrrolinin-2-yl, and pyrrolidin-3-yl all optionally substituted with one —F, —Cl, C₁-C₃ alkyl, —CO—(C₁-C₃ alkyl), —SO₂—H, or —SO₂—(C₁-C₃).

More particularly, X₁₋₂ and X₁₋₃ are cyclized to form pyrrolidin-1-yl, N-(1-methylpyrrolidin-3-yl), N-(4-methylpiperazin-1-yl), and N-(1ethylpiperadin-4-yl).

Also, when W is a cyclic structure of three through seven atoms consisting of carbon, nitrogen, and sulfur, that the cyclic structure be selected from the group consisting of phenyl, thiazolyl, pyridinyl, and C₃-C₇ cycloalkyl.

One embodiment of a compound of Formula V is a compound of Formula V(a):

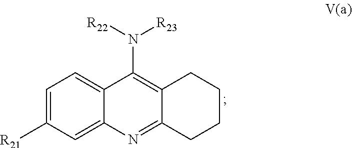

or a pharmaceutically acceptable salt thereof, wherein:
R₂₁ is selected from the group consisting of H, F, Cl, Br, and C₁₋₃ haloalkyl;
R₂₂ is selected from the group consisting of H, C₁₋₃ alkyl, and C₁₋₃ haloalkyl;
R₂₃ is selected from the group consisting of an optionally substituted 5- or 6-membered cycloalkyl or heterocycloalkyl, optionally substituted

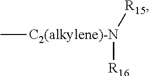

optionally substituted

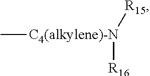

and optionally substituted

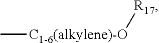

wherein the alkylene chains may be optionally substituted with up to 3 R₁₈;
R₁₅ and R₁₆ are each independently selected from the group consisting of H, alkyl, cycloalkyl, alkoxy, alkylamino, and sulfonyl;
or R₁₅ and R₁₆ may be joined together to form an optionally substituted 5- or 6-membered cycloalkyl or heterocycloalkyl;
R₁₇ is selected from the group consisting of H, alkyl, cycloalkyl, alkoxy, alkylamino, and sulfonyl; and
R₁₈ is selected from the group consisting of H, alkyl, cycloalkyl, alkoxy, alkylamino, and sulfonyl.

In one embodiment, R₂₁ is H, F, or Cl.

More particularly, R₂₁ is H.

In another embodiment, R₂₁ is F.

In yet another embodiment, R₂₁ is Cl.

In one embodiment, R₂₂ is H.

In another embodiment, R₂₂ is C₁₋₃ alkyl.

More particularly, R₂₂ is methyl.

In one embodiment, R₂₁ is Cl, and R₂₂ is H

In another embodiment, R₂₁ is Br, and R₂₂ is H.

In another embodiment, R₂₁ is F, and R₂₂ is methyl.

In any of the above embodiments of a compound of Formula V(a) provided above, R₂₃ is an optionally substituted 5- or 6-membered cycloalkyl or heterocycloalkyl,

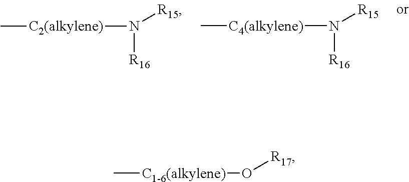

wherein the alkylene chains may be optionally substituted with up to 3 R₁₈.

In some embodiments, R₂₃ is optionally substituted 5- or 6-membered heterocycloalkyl.

More particularly, R₂₃ is

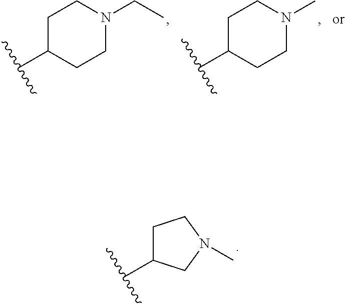

In some embodiments, R₂₃ is optionally substituted

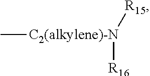

wherein the alkylene chain may be optionally substituted with up to 3 R₁₈.

More particularly, R₂₃ is

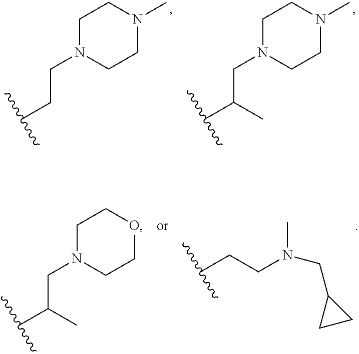

In some embodiments, R₂₃ is optionally substituted

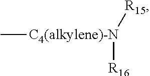

wherein the alkylene chain may be optionally substituted with up to 3 R₁₈.

More particularly, in some embodiments, R₂₃ is

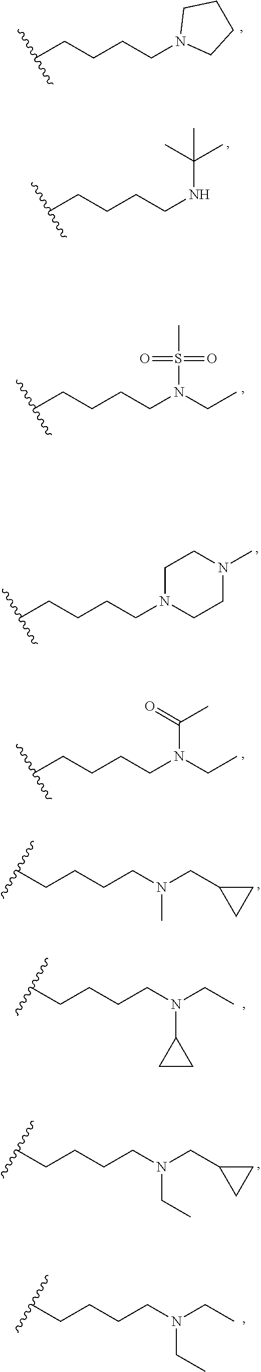

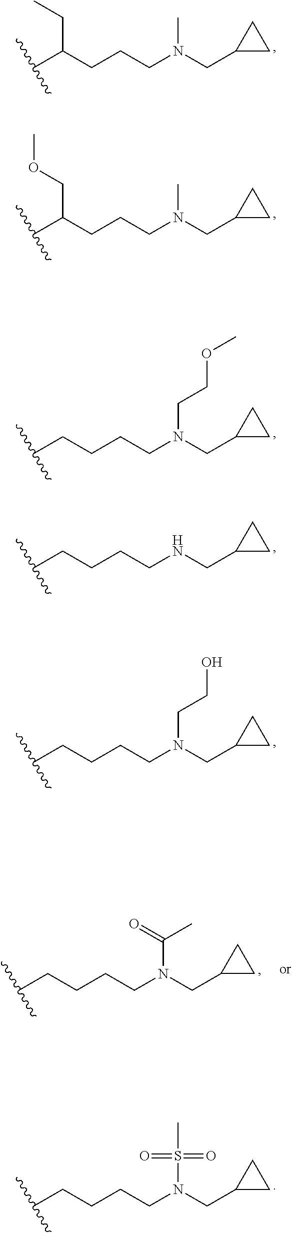

In some embodiments, R₂₃ is optionally substituted

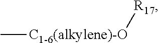

wherein the alkylene chain may be optionally substituted with up to 3 R₁₈.

More particularly, R₂₃ is

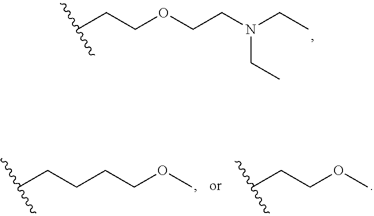

In another embodiment of a compound of Formula V, the compounds are of Formula V(b)

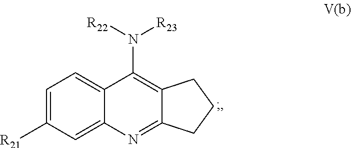

or a pharmaceutically acceptable salt thereof, wherein the variables and embodiments are as defined above for a compound of Formula V(a).

In another embodiment of a compound of the present invention, the compounds are of Formula A:

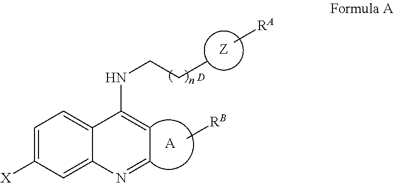

or a pharmaceutically acceptable salt thereof, wherein:

A is optionally substituted aryl or optionally substituted cycloalkyl;

Z is a 3 to 7 membered heterocycloalkyl;

X is H, halogen, or —CF₃;

n^{D} is 1 to 3;

R^{A} is optionally substituted C₁₋₆ alkyl; and

R^{B} is H, optionally substituted C₁₋₆ alkyl, or optionally substituted C₁₋₆ alkoxyl.

In one embodiment of Formula A, a compound or a pharmaceutically acceptable salt thereof has the structure of Formula A¹:

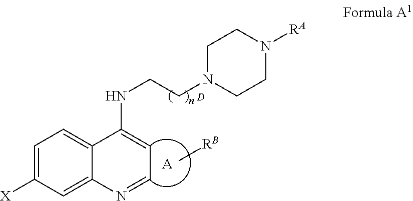

or a pharmaceutically acceptable salt thereof, wherein:

A is optionally substituted aryl or optionally substituted cycloalkyl;

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In one embodiment of Formula A, a compound or a pharmaceutically acceptable salt thereof has the structure of Formula A²:

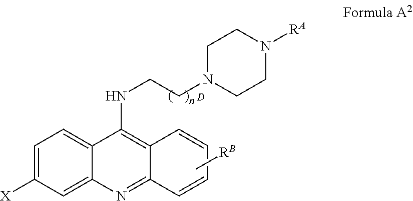

or a pharmaceutically acceptable salt thereof, wherein:

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In one embodiment of Formula A, a compound or a pharmaceutically acceptable salt thereof has the structure of Formula A³:

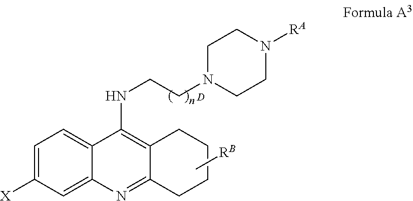

or a pharmaceutically acceptable salt thereof, wherein:

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In some embodiments of Formula A and Formula A¹, A is an optionally substituted aryl which, may be unsubstituted or substituted with one to three of hydroxyl, amino, C₁₋₆ alkoxyl, carboxy, cyano, and halogen, which is fused to the pyrimidine ring thereby forming a substituted tricyclic acridine core structure as shown in Formula A².

In some of these embodiments, A is optionally substituted phenyl. In some embodiments, A is phenyl unsubstituted or substituted with C₁₋₆ alkoxyl. In some embodiments, A is an optionally substituted 5-6 membered cycloalkyl which, may be unsubstituted or substituted with one to three of hydroxyl, amino, C₁₋₆ alkoxyl, carboxy, cyano, and halogen. In some of these embodiments, A is an optionally substituted cyclopentyl, or cyclohexyl. In some embodiments A is optionally substituted cyclohexyl, which is fused to the pyridine ring thereby forming a substituted tricyclic 1,2,3,4-tetrahydroacridine core as shown in Formula A³. In some embodiments, A is optionally substituted phenyl, or optionally substituted cyclohexyl. In some embodiments, A is phenyl. In some embodiments, A is phenyl substituted with methoxy. In some embodiments, A is unsubstituted cyclohexyl.

In some embodiments, Z is a 3-7 membered heterocycloalkyl, wherein 1-2 atoms in the ring structure are a heteroatom selected from the group consisting of N, S, or O. In some embodiments, 1-2 atoms on the ring structure are nitrogen. In some embodiments, the heterocycloalkyl contains one nitrogen atom. In some embodiments, the heterocycloalkyl contains two nitrogen atoms. In one embodiment, Z is piperazine.

In some embodiments, X is H, halogen, or —CF₃. In some embodiments, X is a halogen selected from F, Cl, or Br. In some embodiments, X is Cl.

In some embodiments of Formula A, R^{A} is optionally substituted alkyl. In some embodiments, R^{A} is optionally substituted C₁₋₆ alkyl selected from methyl, ethyl, propyl, butyl, pentyl or hexyl, each of which, may be unsubstituted or substituted with one to three of hydroxyl, amino, alkoxy, carboxy, cyano, and halogen. In some embodiments, R^{A} is optionally substituted methyl. In some embodiments, R^{A} is unsubstituted methyl.

In some embodiments of Formula A, R^{B} is H, optionally substituted C₁₋₆ alkyl, or optionally substituted C₁₋₆ alkoxyl. In some embodiments, R^{B} is optionally substituted C₁₋₆ alkyl, or optionally substituted C₁₋₆ alkoxyl, each of which, may be unsubstituted or substituted with one to three of hydroxyl, amino, alkoxy, carboxy, cyano, and halogen. In some embodiments, R^{B} is H or optionally substituted C₁₋₆ alkoxyl. In some of these embodiments, R^{B} is methoxy, ethoxy, propoxy, butoxy, pentoxy and hexyloxy each of which may be unsubstituted or substituted with one to three of hydroxyl, amino, C₁₋₆ alkoxy, carboxy, cyano, and halogen.

In some embodiments, R^{B} is H or methoxy.

In various embodiments, exemplary compounds of Formula A include:

Example 7

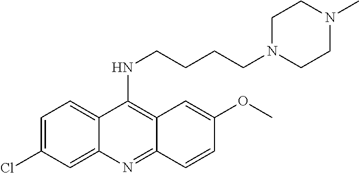 [SMILES: COc1ccc2nc3cc(Cl)ccc3c(NCCCCN3CCN(C)CC3)c2c1]

or a pharmaceutically acceptable salt thereof; and

Example 27

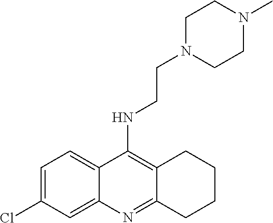 [SMILES: CN1CCN(CCNc2c3c(nc4cc(Cl)ccc24)CCCC3)CC1]

or a pharmaceutically acceptable salt thereof.

In one aspect, the compound of the invention is selected from the compounds provided in Table 1:

TABLE 1

| Example | Structure | Name |
|---|---|---|
| 1 | 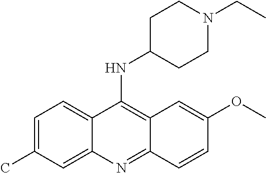 | 6-Chloro-N-(1-ethylpiperidin-4- yl)-2-methoxyacridin-9-amine |
| 2 | 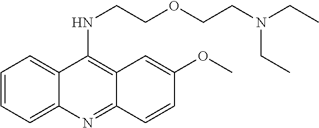 [SMILES: CCN(CC)CCOCCNc1c2ccccc2nc2ccc(OC)cc12] | 6-Chloro-N-(2-(2- (diethylamino)ethoxy)ethyl)-2- methoxyacridin-9-amine |
| 3 | 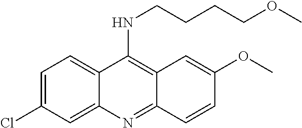 [SMILES: COCCCCNc1c2ccc(Cl)cc2nc2ccc(OC)cc12] | 6-Chloro-2-methoxy-N-(4- methoxybutyl)acridin-9-amine |
| 4 | 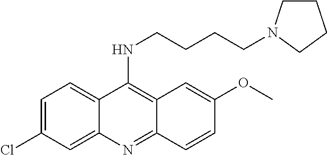 [SMILES: COc1ccc2nc3cc(Cl)ccc3c(NCCCCN3CCCC3)c2c1] | 6-Chloro-2-methoxy-N-(4- (pyrrolidin-1-yl)butyl)acridin-9- amine |
| 5 | 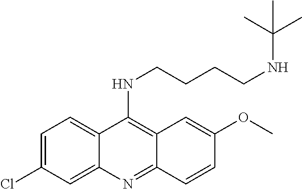 [SMILES: COc1ccc2nc3cc(Cl)ccc3c(NCCCCNC(C)(C)C)c2c1] | N¹-tert-butyl-N⁴-(6-chloro-2- methoxyacridin-9-yl)butane- 1,4-diamine |
| 6 | 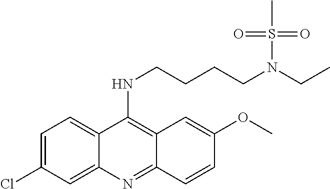 [SMILES: CCN(CCCCNc1c2ccc(Cl)cc2nc2ccc(OC)cc12)S(C)(=O)=O] | N-(4-(6-Chloro-2- methoxyacridin-9- ylamino)butyl)-N- ethylmethanesulfonamide |
| 7 | 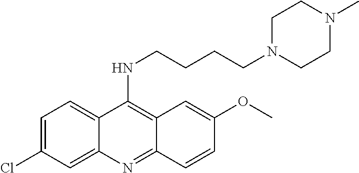 [SMILES: COc1ccc2nc3cc(Cl)ccc3c(NCCCCN3CCN(C)CC3)c2c1] | 6-Chloro-2-methoxy-N-(4-(4- methylpiperazin-1- yl)butyl)acridin-9-amine VATG-027 |
| 8 | 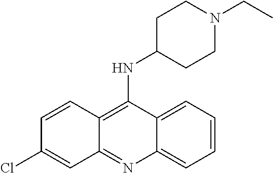 [SMILES: CCN1CCC(Nc2c3ccccc3nc3cc(Cl)ccc23)CC1] | 3-Chloro-N-(1-ethylpiperidin- 4-yl)acridin-9-amine |
| 9 | 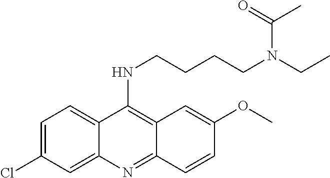 [SMILES: CCN(CCCCNc1c2ccc(Cl)cc2nc2ccc(OC)cc12)C(C)=O] | N-(4-(6-Chloro-2- methoxyacridin-9- ylamino)butyl)-N- ethylacetamide |
| 10 | 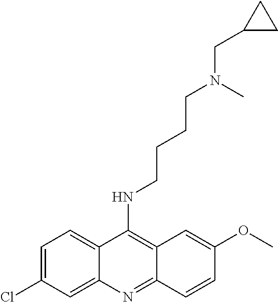 [SMILES: COc1ccc2nc3cc(Cl)ccc3c(NCCCCN(C)CC3CC3)c2c1] | N¹-(6-Chloro-2- methoxyacridin-9-yl)-N⁴- (cyclopropylmethyl)-N⁴- methylbutane-1,4-diamine |
| 11 | 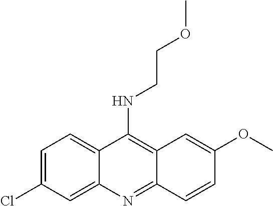 [SMILES: COCCNc1c2ccc(Cl)cc2nc2ccc(OC)cc12] | 6-Chloro-2-methoxy-N-(2- methoxyethyl)acridin-9-amine |
| 12 | 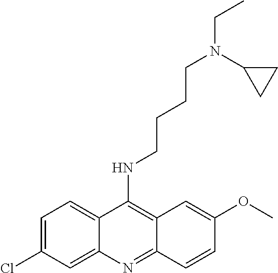 [SMILES: CCN(CCCCNc1c2ccc(Cl)cc2nc2ccc(OC)cc12)C1CC1] | N¹-(6-Chloro-2- methoxyacridin-9-yl)-N⁴- cyclopropyl-N⁴-ethylbutane- 1,4-diamine |
| 13 | 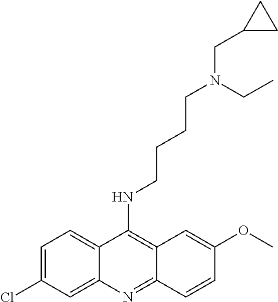 [SMILES: CCN(CCCCNc1c2ccc(Cl)cc2nc2ccc(OC)cc12)CC1CC1] | N¹-(6-Chloro-2- methoxyacridin-9-yl)-N⁴- (cycloproylmethyl)-N⁴- ethylbutane-1,4-diamine |
| 14 | 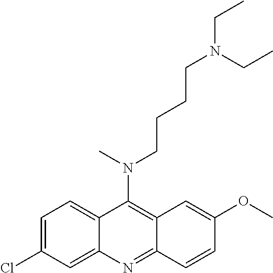 [SMILES: CCN(CC)CCCCN(C)c1c2ccc(Cl)cc2nc2ccc(OC)cc12] | N¹-(6-Chloro-2- methoxyacridin-9-yl)-N⁴,N⁴- diethyl-N¹-methylbutane-1,4- diamine |
| 15 | 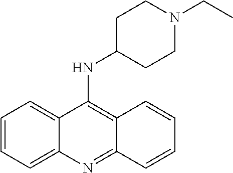 [SMILES: CCN1CCC(Nc2c3ccccc3nc3ccccc23)CC1] | N-(1-Ethylpiperidin-4- yl)acridin-9-amine |
| 16 | 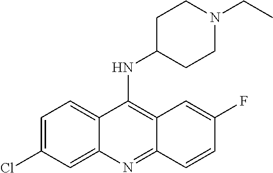 [SMILES: CCN1CCC(Nc2c3ccc(Cl)cc3nc3ccc(F)cc23)CC1] | 6-Chloro-N-(1-ethylpiperidin- 4-yl)-2-fluoroacridin-9-amine |
| 17 | 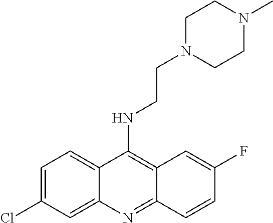 [SMILES: CN1CCN(CCNc2c3ccc(Cl)cc3nc3ccc(F)cc23)CC1] | 6-Chloro-2-fluoro-N-(2-(4- methylpiperazin-1- yl)ethyl)acridin-9-amine |
| 18 | 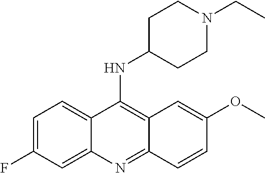 [SMILES: CCN1CCC(Nc2c3ccc(F)cc3nc3ccc(OC)cc23)CC1] | N-(1-Ethylpiperidin-4-yl)-6- fluoro-2-methoxyacridin-9- amine |
| 19 | 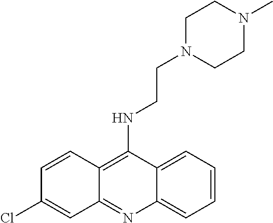 [SMILES: CN1CCN(CCNc2c3ccccc3nc3cc(Cl)ccc23)CC1] | 3-Chloro-N-(2-(4- methylpiperazin-1- yl)ethyl)acridin-9-amine |
| 20 | 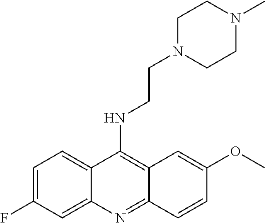 [SMILES: COc1ccc2nc3cc(F)ccc3c(NCCN3CCN(C)CC3)c2c1] | 6-Fluoro-2-methoxy-N-(2-(4- methylpiperazin-1- yl)ethyl)acridin-9-amine |
| 21 | 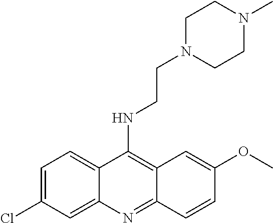 [SMILES: COc1ccc2nc3cc(Cl)ccc3c(NCCN3CCN(C)CC3)c2c1] | 6-Chloro-2-methoxy-N-(2-(4- methylpiperazin-1- yl)ethyl)acridin-9-amine |
| 22 | 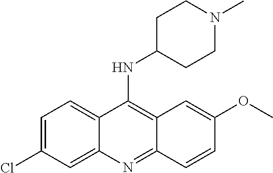 [SMILES: COc1ccc2nc3cc(Cl)ccc3c(NC3CCN(C)CC3)c2c1] | 6-Chloro-2-methoxy-N-(1- methylpiperidin-4-yl)acridin- 9-amine |
| 23 | 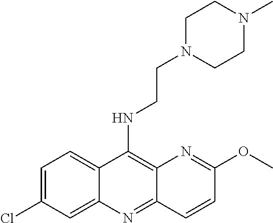 [SMILES: COc1ccc2nc3cc(Cl)ccc3c(NCCN3CCN(C)CC3)c2n1] | 7-Chloro-2-methoxy-N-(2-(4- methylpiperazin-1- yl)ethyl)benzo[b][1,5] naphthyridin-10-amine |
| 24 | 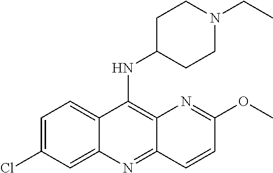 [SMILES: CCN1CCC(Nc2c3ccc(Cl)cc3nc3ccc(OC)nc23)CC1] | 7-Chloro-N-(1-ethylpiperidin- 4-yl)-2- methoxybenzo[b][1,5] naphthyridin-10-amine |
| 25 | 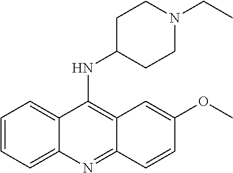 [SMILES: CCN1CCC(Nc2c3ccccc3nc3ccc(OC)cc23)CC1] | N-(1-Ethylpiperidin-4-yl)-2- methoxyacridin-9-amine |
| 26 | 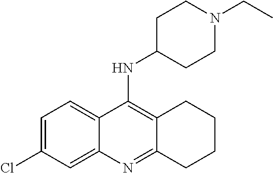 [SMILES: CCN1CCC(Nc2c3c(nc4cc(Cl)ccc24)CCCC3)CC1] | 6-Chloro-N-(1-ethylpiperidin- 4-yl)-1,2,3,4- tetrahydroacridin-9-amine |
| 27 | 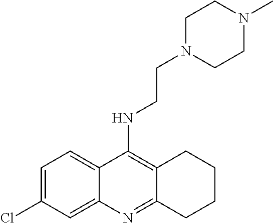 [SMILES: CN1CCN(CCNc2c3c(nc4cc(Cl)ccc24)CCCC3)CC1] | 6-Chloro-N-(2-(4- methylpiperazin-1-yl)ethyl)- 1,2,3,4-tetrahydroacridin-9- amine VATG-032 |
| 28 | 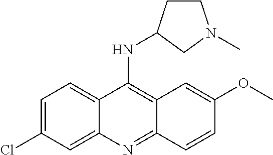 [SMILES: COc1ccc2nc3cc(Cl)ccc3c(NC3CCN(C)C3)c2c1] | 6-Chloro-2-methoxy-N-(1- methylpyrrolidin-3-yl)acridin- 9-amine |
| 29 | 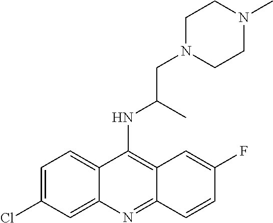 [SMILES: CC(CN1CCN(C)CC1)Nc1c2ccc(Cl)cc2nc2ccc(F)cc12] | 6-Chloro-2-fluoro-N-(1-(4- methylpiperazin-1-yl)propan- 2-yl)acridin-9-amine |
| 30 | 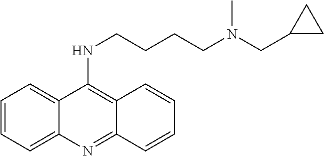 [SMILES: CN(CCCCNc1c2ccccc2nc2ccccc12)CC1CC1] | N¹-(acridin-9-yl)-N⁴- (cyclopropylmethyl)-N⁴- methylbutane-1,4-diamine |
| 31 | 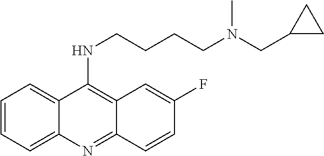 [SMILES: CN(CCCCNc1c2ccccc2nc2ccc(F)cc12)CC1CC1] | N¹-(cyclopropylmethyl)-N⁴-(2- fluoroacridin-9-yl)-N¹- methylbutane-1,4-diamine |
| 32 | 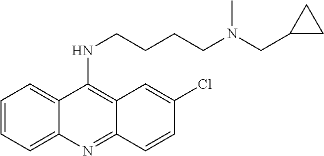 [SMILES: CN(CCCCNc1c2ccccc2nc2ccc(Cl)cc12)CC1CC1] | N¹-(2-chloroacridin-9-yl)-N⁴- (cyclopropylmethyl)-N⁴- methylbutane-1,4-diamine |
| 33 | 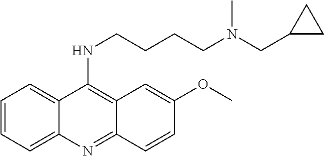 [SMILES: COc1ccc2nc3ccccc3c(NCCCCN(C)CC3CC3)c2c1] | N¹-(cyclopropylmethyl)-N⁴-(2- methoxyacridin-9-yl)-N¹- methylbutane-1,4-diamine |
| 34 | 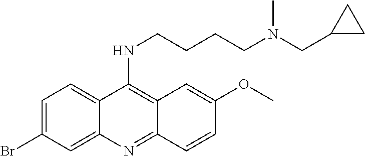 [SMILES: COc1ccc2nc3cc(Br)ccc3c(NCCCCN(C)CC3CC3)c2c1] | N¹-(6-bromo-2- methoxyacridin-9-yl)-N⁴- (cyclopropylmethyl)-N⁴- methylbutane-1,4-diamine |
| 35 | 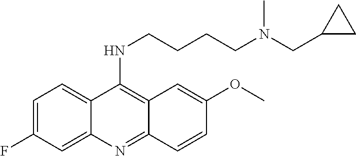 [SMILES: COc1ccc2nc3cc(F)ccc3c(NCCCCN(C)CC3CC3)c2c1] | N¹-(cyclopropyl-methyl)-N⁴- (6-fluoro-2-methoxyacridin-9- yl)-N¹-methylbutane-1,4- diamine |
| 36 | 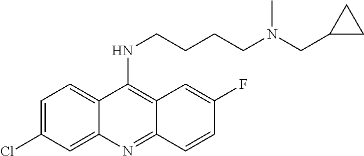 [SMILES: CN(CCCCNc1c2ccc(Cl)cc2nc2ccc(F)cc12)CC1CC1] | N¹-(6-chloro-2-fluoroacridin- 9-yl)-N⁴-(cyclopropylmethyl)- N⁴-methylbutane-1,4-diamine |
| 37 | 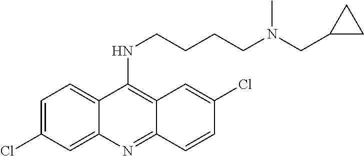 [SMILES: CN(CCCCNc1c2ccc(Cl)cc2nc2ccc(Cl)cc12)CC1CC1] | N¹-(cyclopropylmethyl)-N⁴- (2,6-dichloroacridin-9-yl)-N¹- methylbutane-1,4-diamine |
| 38 | 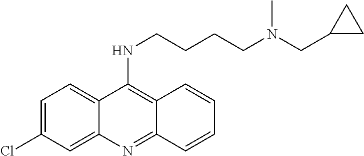 [SMILES: CN(CCCCNc1c2ccccc2nc2cc(Cl)ccc12)CC1CC1] | N¹-(3-chloroacridin-9-yl)-N⁴- (cyclopropylmethyl)-N⁴- methylbutane-1,4-diamine |
| 39 | 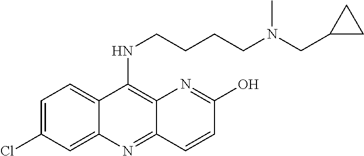 [SMILES: CN(CCCCNc1c2ccc(Cl)cc2nc2ccc(O)nc12)CC1CC1] | 7-chloro-10-(4-((cyclopropyl- methyl)(methyl)amino)butyl amino)benzo[b][1,5] naphthyridin-2-ol |
| 40 | 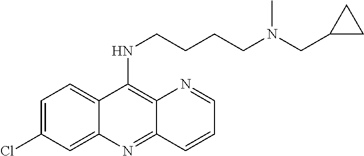 [SMILES: CN(CCCCNc1c2ccc(Cl)cc2nc2cccnc12)CC1CC1] | N¹-(7- chlorobenzo[b][1,5] naphthyridin-10-yl)-N⁴- (cyclopropylmethyl)-N⁴- methylbutane-1,4-diamine |
| 41 | 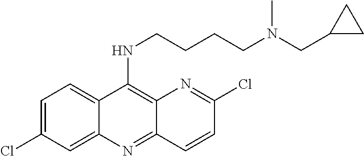 [SMILES: CN(CCCCNc1c2ccc(Cl)cc2nc2ccc(Cl)nc12)CC1CC1] | N¹-(cyclopropylmethyl)-N⁴- (2,7- dichlorobenzo[b][1,5] naphthyridin-10-yl)-N¹-methylbutane- 1,4-diamine |
| 42 | 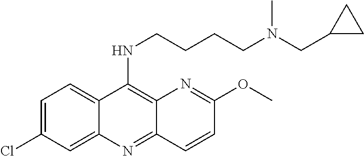 [SMILES: COc1ccc2nc3cc(Cl)ccc3c(NCCCCN(C)CC3CC3)c2n1] | N¹-(7-chloro-2-methoxy benzo[b][1,5]naphthyridin-10- yl)-N⁴-(cyclopropylmethyl)-N⁴- methylbutane-1,4-diamine |
| 43 | 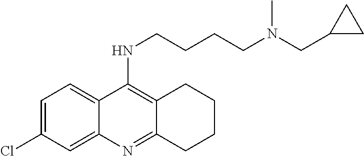 [SMILES: CN(CCCCNc1c2c(nc3cc(Cl)ccc13)CCCC2)CC1CC1] | N¹-(6-chloro-1,2,3,4- tetrahydroacridin-9-yl)-N⁴- (cyclopropylmethyl)-N⁴- methylbutane-1,4-diamine |
| 44 | 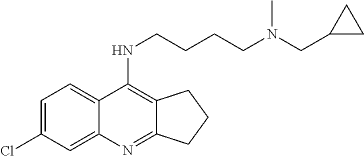 [SMILES: CN(CCCCNc1c2c(nc3cc(Cl)ccc13)CCC2)CC1CC1] | N¹-(6-chloro-2,3-dihydro-1H- cyclopenta[b]quinolin-9-yl)- N⁴-(cyclopropylmethyl)-N⁴- methylbutane-1,4-diamine |
| 45 | 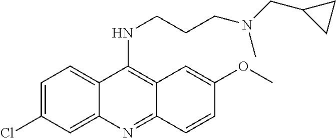 [SMILES: COc1ccc2nc3cc(Cl)ccc3c(NCCCN(C)CC3CC3)c2c1] | N¹-(6-chloro-2- methoxyacridin-9-yl)-N³- (cyclopropylmethyl-N³- methylpropane-1,3-diamine |
| 46 | 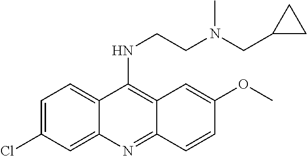 [SMILES: COc1ccc2nc3cc(Cl)ccc3c(NCCN(C)CC3CC3)c2c1] | N¹-(6-chloro-2- methoxyacridin-9-yl)-N²- (cyclopropylmethyl)-N²- methylethane-1,2-diamine |
| 47 | 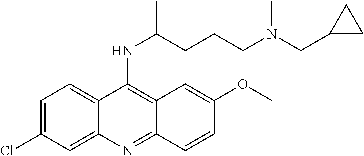 [SMILES: COc1ccc2nc3cc(Cl)ccc3c(NC(C)CCCN(C)CC3CC3)c2c1] | N⁴-(6-chloro-2- methoxyacridin-9-yl)-N¹- (cyclopropylmethyl)-N¹- methylpentane-1,4-diamine |
| 48 | 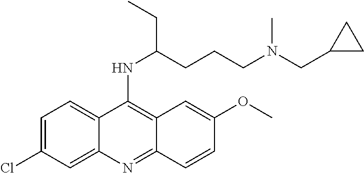 [SMILES: CCC(CCCN(C)CC1CC1)Nc1c2ccc(Cl)cc2nc2ccc(OC)cc12] | N⁴-(6-chloro-2- methoxyacridin-9-yl)-N¹- (cyclopropylmethyl)-N¹- methylhexane-1,4-diamine |
| 49 | 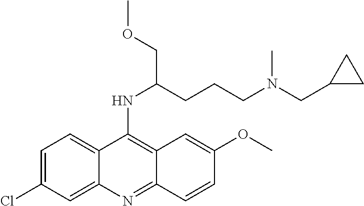 [SMILES: COCC(CCCN(C)CC1CC1)Nc1c2ccc(Cl)cc2nc2ccc(OC)cc12] | N⁴-(6-chloro-2- methoxyacridin-9-yl)-N¹- (cyclopropylmethyl)-5- methoxy-N¹-methylpentane- 1,4-diamine |
| 50 | 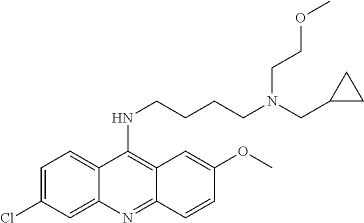 [SMILES: COCCN(CCCCNc1c2ccc(Cl)cc2nc2ccc(OC)cc12)CC1CC1] | N¹-(6-chloro-2- methoxyacridin-9-yl)-N⁴- (cyclopropylmethyl)-N⁴-(2- methoxyethyl)butane-1,4- diamine |
| 51 | 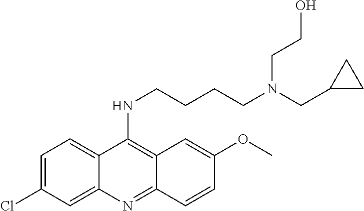 [SMILES: COc1ccc2nc3cc(Cl)ccc3c(NCCCCN(CCO)CC3CC3)c2c1] | 2-((4-(6-chloro-2- methoxyacridin-9- ylamino)butyl)(cyclopropylmethyl) amino)ethanol |
| 52 | 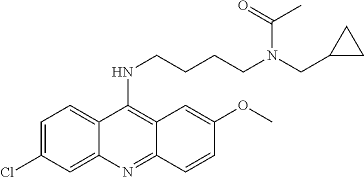 [SMILES: COc1ccc2nc3cc(Cl)ccc3c(NCCCCN(CC3CC3)C(C)=O)c2c1] | N-(4-(6-chloro-2- methoxyacridin-9- ylamino)butyl)-N- (cyclopropylmethyl)acetamide |
| 53 | 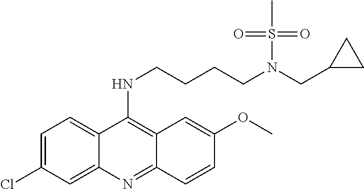 [SMILES: COc1ccc2nc3cc(Cl)ccc3c(NCCCCN(CC3CC3)S(C)(=O)=O)c2c1] | N-(4-(6-chloro-2- methoxyacridin-9- ylamino)butyl)-N- (cyclopropylmethyl) methanesulfonamide |
| 54 | 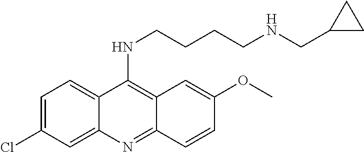 [SMILES: COc1ccc2nc3cc(Cl)ccc3c(NCCCCNCC3CC3)c2c1] | N¹-(6-chloro-2- methoxyacridin-9-yl)-N⁴- (cyclopropylmethyl)butane- 1,4-diamine |
| 55 | 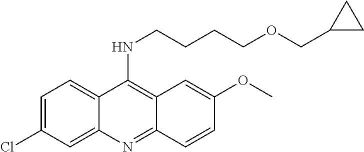 [SMILES: COc1ccc2nc3cc(Cl)ccc3c(NCCCCOCC3CC3)c2c1] | 6-chloro-N-(4- (cyclopropylmethoxy)butyl)- 2-methoxyacridin-9-amine |
| 56 | 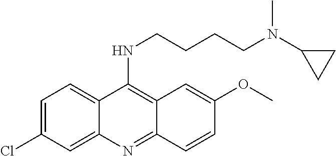 [SMILES: COc1ccc2nc3cc(Cl)ccc3c(NCCCCN(C)C3CC3)c2c1] | N¹-(6-chloro-2- methoxyacridin-9-yl)-N⁴- cyclopropyl-N⁴-methylbutane- 1,4-diamine |
| 57 | 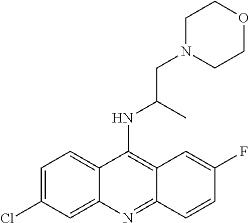 [SMILES: CC(CN1CCOCC1)Nc1c2ccc(Cl)cc2nc2ccc(F)cc12] | 6-chloro-2-fluoro-N-(1- morpholinopropan-2- yl)acridin-9-amine |
| 58 | 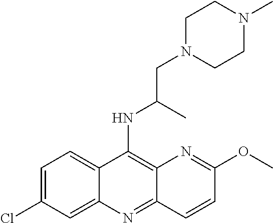 [SMILES: COc1ccc2nc3cc(Cl)ccc3c(NC(C)CN3CCN(C)CC3)c2n1] | 7-Chloro-2-methoxy-N-(1-(4- methylpiperazin-1-yl)propan- 2- yl)benzo[b][1,5]naphthyridin- 10-amine |
| 59 | 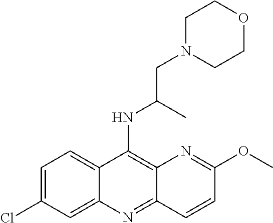 [SMILES: COc1ccc2nc3cc(Cl)ccc3c(NC(C)CN3CCOCC3)c2n1] | 7-Chloro-2-methoxy-N-(1- morpholinpropan-2- yl)benzo[b][1,5]naphthyridin- 10-amine |
| 60 | 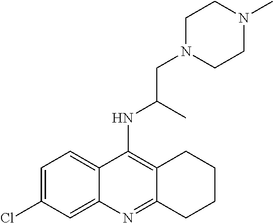 [SMILES: CC(CN1CCN(C)CC1)Nc1c2c(nc3cc(Cl)ccc13)CCCC2] | 6-Chloro-N-(1-(4- methylpiperazin-1-yl)propan- y-yl)-1,2,3,4- tetrahydroacridin-9-amine |
| 61 | 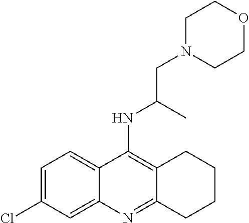 [SMILES: CC(CN1CCOCC1)Nc1c2c(nc3cc(Cl)ccc13)CCCC2] | 6-Chloro-N-(1- morpholinopropan-2-yl)- 1,2,3,4-tetrahydroacridin-9- amine |
| 62 | 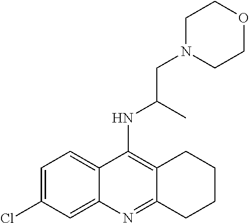 [SMILES: CC(CN1CCOCC1)Nc1c2c(nc3cc(Cl)ccc13)CCCC2] | 6-Chloro-N-(4-(4- methylpiperazin-1-yl)butan-2- yl)-1,2,3,4-tetrahydroacridin- 9-amine |
| 63 | 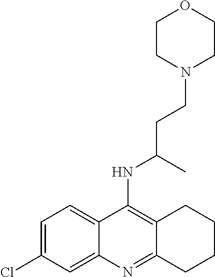 [SMILES: CC(CCN1CCOCC1)Nc1c2c(nc3cc(Cl)ccc13)CCCC2] | 6-Chloro-N-(4- morpholinobutan-2-yl)- 1,2,3,4-tetrahydroacridin-9- amine |
| 64 | 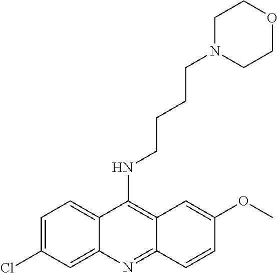 [SMILES: COc1ccc2nc3cc(Cl)ccc3c(NCCCCN3CCOCC3)c2c1] | 6-chloro-2-methoxy-N-(4- morpholinobutyl)acridin-9- amine, |

or a pharmaceutically acceptable salt thereof.

The compounds of Formulas III, V, and A are amines and, as such, form salts when reacted with acids. Thus, pharmaceutically acceptable salts of compounds of Formulas III V, and A are included within the scope of this invention. Pharmaceutically acceptable salts include salts of both inorganic and organic acids. The pharmaceutically acceptable salts are preferred over the corresponding free amines since they produce compounds that are more water soluble and more crystalline. Pharmaceutically acceptable salts are any salt which retains the activity of the parent compound and does not impart any deleterious or undesirable effect on the subject to whom it is administered and in the context in which it is administered. The preferred pharmaceutically acceptable salts include salts of the following acids acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, succinic, sulfamic, sulfanilic, sulfonic, sulfuric, tannic, tartaric, teoclic and toluenesulfonic. For other acceptable salts, see Int. J. Pharm., 33, 201-217 (1986) and J. Pharm. Sci., 66(1), 1, (1977).

In some aspects of the invention the disclosed compound, is in the form of a pharmaceutically acceptable salt. Pharmaceutically acceptable salts include any salt derived from an organic or inorganic acid. Examples of such salts include but are not limited to the following: salts of hydrobromic acid, hydrochloric acid, nitric acid, phosphoric acid, and sulphuric acid. Organic acid addition salts include, for example, salts of acetic acid, benzenesulphonic acid, benzoic acid, camphorsulphonic acid, citric acid, 2-(4-chlorophenoxy)-2-methylpropionic acid, 1, 2-ethanedisulphonic acid, ethanesulphonic acid, ethylenediaminetetraacetic acid (EDTA), fumaric acid, glucoheptonic acid, gluconic acid, glutamic acid, N-glycolylarsanilic acid, 4-hexylresorcinol, hippuric acid, 2-(4-hydroxybenzoyl) benzoicacid, 1-hydroxy-2-naphthoicacid, 3-hydroxy-2-naphthoic acid, 2-hydroxyethanesulphonic acid, lactobionic acid, n-dodecyl sulphuric acid, maleic acid, malic acid, mandelic acid, methanesulphonic acid, methyl sulpuric acid, mucic acid, 2-naphthalenesulphonic acid, pamoic acid, pantothenic acid, phosphanilic acid ((4-aminophenyl) phosphonic acid), picric acid, salicylic acid, stearic acid, succinic acid, tannic acid, tartaric acid, terephthalic acid, p-toluenesulphonic acid, 10-undecenoic acid, or any other such acid now known or yet to be disclosed. It will be appreciated by one skilled in the art that such pharmaceutically acceptable salts may be used in the formulation of a pharmacological composition. Such salts may be prepared by reacting the disclosed compounds with a suitable acid in a manner known by those skilled in the art.

Pharmaceutically acceptable anion salts include, but are not limited to, salts of the following acids: methanesulfonic, hydrochloric, hydrobromic, sulfuric, phosphoric, nitric, benzoic, citric, tartaric, fumaric, maleic, CH₃—(CH₂)ₙ—COOH where n is 0 through 4, and HOOC—(CH₂)N—COOH where n is as defined above.

### Processes for Making Compounds of Formula III or V

The compounds of Formula III, V, A or A¹ or a pharmaceutically acceptable salt thereof are prepared from known compounds by methods known to those skilled in the art. Thus a compound of Formula III is prepared from the corresponding compound of Formula I by coupling with an amine of Formula II, as depicted in Scheme 1.

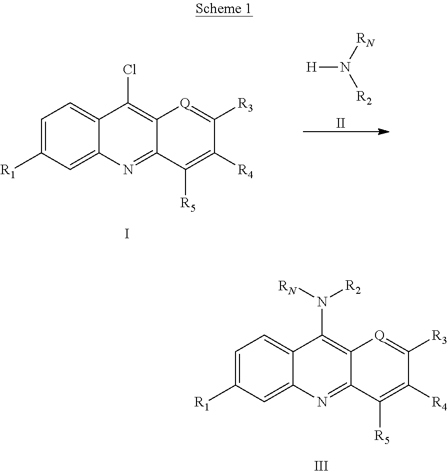

Similarly, the compound of Formula V is prepared by from the corresponding compound of Formula IV by coupling with an amine of Formula II, as depicted in Scheme 2:

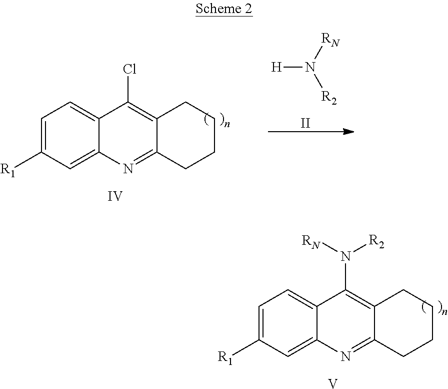

To the extent that some of the halides of Formula I, the amines of Formula II, and the halides of Formula IV are not known compounds, they can be readily prepared from known compounds by methods known to those skilled in the art.

More specifically, the halides of formulas I and IV are heated to about 100° C. in a solvent like phenol. To this mixture, the desired amine (II) is added, and the mixture is kept at about 100° C. for about 5 hours. The mixture is cooled, diluted with a solvent such as dichloromethane, and is worked up as is known to those skilled in the art. Example 1 illustrates the process.

### Pharmaceutical Compositions and Formulations

In another aspect, the invention further provides pharmaceutical compositions that include the compounds of Formula III, Formula III(a), Formula V, Formula V(a), Formula A, Formula A¹, Formula A², Formula A³, or a pharmaceutically acceptable salt thereof as the active pharmaceutical ingredient(s) for the treatment of any of the diseases described herein, including, cancer and the treatment and/or prevention of malaria. Such pharmaceutical compositions may take any physical form necessary depending on a number of factors including the desired method of administration and the physicochemical and stereochemical form taken by the compound or pharmaceutically acceptable salts of the compound. The concept of a pharmaceutical composition including one or more compounds of Formula III, Formula III(a), Formula V, Formula V(a), Formula A, Formula A¹, Formula A², Formula A³, or a pharmaceutically acceptable salt thereof, also encompasses the compounds or a pharmaceutically acceptable salt thereof without any other additive. The physical form of the pharmaceutical composition may affect the route of administration, and one skilled in the art would know to choose a route of administration that takes into consideration both the physical form of the compound and the disorder to be treated. Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, intraperitoneally, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, specifically in unit dosage forms suitable for simple administration of precise dosages. In some embodiments, pharmaceutical compositions comprising one or more doses of the compounds of the present invention can be formulated into a solution, a dispersion, a suspension, a powder, a capsule, a tablet, a pill, a time release capsule, a time release tablet, or a time release pill.

Pharmaceutical compositions including the compounds of Formula III, Formula III(a), Formula V, Formula V(a), Formula A, Formula A¹, Formula A², Formula A³, or a pharmaceutically acceptable salt thereof include materials capable of modifying the physical form of a dosage unit. In one example, the composition may include a material that forms a coating that surrounds and/or contains the pharmaceutical composition. Materials that may be used in such a coating, include, for example, sugar, shellac, gelatin, or any other inert coating agent.

In some embodiments, the compositions will include a conventional pharmaceutical carrier, excipient and/or diluent and a compound of the invention as the/an active agent, and, in addition, may include carriers and adjuvants, etc. Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The choice of formulation depends on various factors such as the mode of drug administration (e.g., for oral administration, formulations in the form of tablets, powders, pills or capsules) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions whether pharmaceutical or not, can be made suitable for parenteral injection and may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One specific route of administration is oral, using a convenient daily dosage regimen that can be adjusted according to the degree of severity of the disease or disorder to be treated, for example, the treatment of cancer, or the treatment and/or prevention of malaria.

In some embodiments, solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

In some embodiments, liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Pharmaceutical compositions of the compounds of Formula III, Formula III(a), Formula V, Formula V(a), Formula A, Formula A¹, Formula A², Formula A³, or a pharmaceutically acceptable salt thereof can be prepared as a gas or aerosol. Aerosols encompass a variety of systems including colloids and pressurized packages. Delivery of a composition in this form may include propulsion of a pharmaceutical composition including the disclosed compound through use of liquefied gas or other compressed gas or by a suitable pump system. Aerosols may be delivered in single phase, bi-phasic, or tri-phasic systems.

Compressed gases may be used to disperse a compound of this invention in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts or solvates, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 mg to about 1,000 mg per day. For a normal human adult having a body weight of about 70 kilograms, a dosage in the range of about 0.01 to about 100 mg per kilogram of body weight per day is an example. In an exemplary embodiment, the pharmaceutical composition of the present invention for the treatment of cancer or the treatment and/or prevention of malaria, contains a therapeutically effective dose amount of a compound of Formula A, A¹, A², or A³, Example 7, Example 27, or a pharmaceutically acceptable salt thereof, ranging from about 0.01 mg per kg body weight to about 100 mg per kg body weight.

In another exemplary embodiment, the pharmaceutical composition of the present invention for the treatment of cancer or the treatment and/or prevention of malaria, contains a therapeutically effective dose amount of the compound of Formula A, A¹, A², or A³, Example 7, Example 27, or a pharmaceutically acceptable salt thereof, ranging from about 1 mg per kg body weight to about 50 mg per kg body weight. In another exemplary embodiment, the pharmaceutical composition of the present invention for the treatment of cancer, or the treatment and/or prevention of malaria, contains a therapeutically effective dose amount of the compound of Formula A, A¹, A², or A³, Example 7, Example 27, or a pharmaceutically acceptable salt thereof, ranging from about 10 mg per kg body weight to about 50 mg per kg body weight.

In another embodiment, determination of an effective amount of the disclosed compounds is within the capability of those skilled in the art, especially in light of the detailed disclosure provided herein. The effective amount of a pharmaceutical composition used to effect a particular purpose, as well as its toxicity, excretion, and overall tolerance is determined in cell cultures, or animals by pharmaceutical and toxicological procedures known to those skilled in the art. For example, in some embodiments, the compounds of Formula III, Formula III(a), Formula V, Formula V(a), Formula A, Formula A¹, Formula A², Formula A³, or a pharmaceutically acceptable salt thereof will normally be administered 1-4 times daily; orally, rectally, parenterally, or other route of administration in an appropriate pharmaceutical compositions containing the active ingredient either as a free base or as a pharmaceutically acceptable acid addition salt in association with one or more pharmaceutically acceptable carriers. In some embodiments, suitable daily doses of the compounds of Formula III, Formula III(a), Formula V, Formula V(a), Formula A, Formula A¹, Formula A², Formula A³, or a pharmaceutically acceptable salt thereof for the treatment of the various diseases and disorders described herein, for example, the treatment of cancer, or the treatment and/or prevention of malaria can range from about 0.01 mg/kg to about 100 mg/kg for oral administration, preferably from about 0.01 mg/kg to about 50 mg/kg, and from about 0.05 mg/kg to about 50 mg/kg for parenteral administration, preferably from about 0.03 to about 3 mg/kg. The use and administration to a patient to be treated in the clinic would be readily apparent to a person of ordinary skill in the art.

The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

If a combination of two active agents including a compound of the present invention and a second active agent is formulated as a fixed dose, such combination products employ the compounds of this invention within the dosage range described above and the other pharmaceutically active agent(s) within its approved dosage range. Compounds of the instant invention may alternatively be used sequentially with known pharmaceutically acceptable agent(s) when a combination formulation is inappropriate. In one embodiment for the treatment of cancer, or the treatment and/or prevention of malaria, each dose of the compound of Formula A, A¹, A², A³, Example 7, Example 27, or a pharmaceutically acceptable salt thereof administered to the subject ranges from about 0.01 mg per kg body weight to about 100 mg per kg body weight, and one or more doses are administered one or more times per day, or one or more times per week. In one embodiment, an indicated daily dosage in the larger subject, e.g. humans, is in the range from about 0.5 mg to about 500 mg, conveniently administered, e.g. in divided doses up to four times a day or in retard form. Suitable unit dosage forms for oral administration comprise from ca. 1 to 500 mg of the compounds of the present invention, or a pharmaceutically acceptable salt thereof. In one embodiment, solid oral dosage forms can be manufactured as unit doses, wherein each unit dose comprises a compound or a pharmaceutically acceptable salt thereof formulated with one or more pharmaceutically acceptable excipients, wherein each tablet, pill, capsule or sachet of powder contains: 10 mg, or 20 mg, or 30 mg, or 40 mg, or 50 mg, or 60 mg, or 70 mg, or 80 mg, or 90 mg, or 100 mg, or 110 mg, or 120 mg, or 130 mg, or 140 mg, or 150 mg, or 160 mg, or 170 mg, or 180 mg, or 190 mg, or 200 mg, or 210 mg, or 220 mg, or 230 mg, or 240 mg, or 250 mg, or 260 mg, or 270 mg, or 280 mg, or 290 mg, or 300 mg, or 310 mg, or 320 mg, or 330 mg, or 340 mg, or 350 mg, or 360 mg, or 370 mg, or 380 mg, or 390 mg, or 400 mg, or 410 mg, or 440 mg, or 450 mg, or 460 mg, or 470 mg, or 480 mg, or 490 mg, or 500 mg of a compound of Formula III, Formula III(a), Formula V, Formula V(a), Formula A, A¹, A², A³, Example 7, Example 27, or a pharmaceutically acceptable salt thereof. Similarly, liquid dosages per one milliliter, or per 5 milliliters, or per 10 milliliters can contain: 10 mg, or 20 mg, or 30 mg, or 40 mg, or 50 mg, or 60 mg, or 70 mg, or 80 mg, or 90 mg, or 100 mg, or 110 mg, or 120 mg, or 130 mg, or 140 mg, or 150 mg, or 160 mg, or 170 mg, or 180 mg, or 190 mg, or 200 mg, or 210 mg, or 220 mg, or 230 mg, or 240 mg, or 250 mg, or 260 mg, or 270 mg, or 280 mg, or 290 mg, or 300 mg, or 310 mg, or 320 mg, or 330 mg, or 340 mg, or 350 mg, or 360 mg, or 370 mg, or 380 mg, or 390 mg, or 400 mg, or 410 mg, or 440 mg, or 450 mg, or 460 mg, or 470 mg, or 480 mg, or 490 mg, or 500 mg of a compound of Formula III, Formula III(a), Formula V, Formula V(a), Formula A, A¹, A², A³, Example 7, Example 27, or a pharmaceutically acceptable salt thereof. The determination of a therapeutically effective dose of one or more compounds of the present invention can be calculated or determined using a screening method as described in the examples sections below, or can be derived through controlled clinical trials using standard pharmacological procedures approved by governing drug regulatory bodies, such as the U.S. Food and Drug Administration (FDA).

In various examples, a pharmaceutical composition of the present invention for the treatment and/or prevention of one or more diseases described herein, may include a second effective compound of a distinct chemical Formula from the compounds of Formula III, Formula III(a), Formula V, Formula V(a), Formula A, Formula A¹, Formula A², Formula A³, or a pharmaceutically acceptable salt thereof. In one embodiment, the second effective compound may have the same or a similar molecular target or it may act upstream or downstream of the molecular target of the compounds of Formula III, Formula III(a), Formula V, Formula V(a), Formula A, Formula A¹, Formula A², Formula A³, or a pharmaceutically acceptable salt thereof with regard to one or more biochemical pathways. In some embodiments, specifically for the treatment of cancer, one or more compounds of Formula III, Formula III(a), Formula V, Formula V(a), Formula A, Formula A¹, Formula A², Formula A³, or a pharmaceutically acceptable salt thereof may be used in combination with additional agents. More particularly, the additional agent may be temozolomide, PLX-4032 or AZD-8055.

Examples of pharmaceutical compositions that may be used in combination with the compounds of Formula III, Formula III(a), Formula V, Formula V(a), Formula A, Formula A¹, Formula A², or Formula A³ include nucleic acid binding compositions such as cis-diamminedichloro platinum (II) (cisplatin), doxorubicin, 5-fluorouracil, taxol, and topoisomerase inhibitors such as etoposide, teniposide, irinotecan and topotecan. Still other pharmaceutical compositions include antiemetic compositions such as metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine and tropisetron.

Still other examples of pharmaceutical compositions that can be used in combination with a pharmaceutical composition of the compounds of Formula III, Formula III(a), Formula V, Formula V(a), Formula A, Formula A¹, Formula A², Formula A³, or a pharmaceutically acceptable salt thereof are hematopoietic colony stimulating factors. Examples of hematopoietic colony stimulating factors include, but are not limited to, filgrastim, sargramostim, molgramostim and epoietin alfa. Alternatively, the pharmaceutical composition of the compounds of Formula III, Formula III(a), Formula V, Formula V(a), Formula A, Formula A¹, Formula A², Formula A³, or a pharmaceutically acceptable salt thereof can be used in combination with an anxiolytic agent. Examples of anxiolytic agents include, but are not limited to, buspirone, and benzodiazepines such as diazepam, lorazepam, oxazapam, chlorazepate, clonazepam, chlordiazepoxide and alprazolam.

Pharmaceutical compositions that may be used in combination with pharmaceutical compositions that include the compounds of Formula III, Formula III(a), Formula V, Formula V(a), Formula A, Formula A¹, Formula A², or Formula A³ can include analgesic agents. Such agents may be opioid or non-opioid analgesic. Non-limiting examples of opioid analgesics include morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam, sulindac or any other analgesic.

In other aspects of the invention, pharmaceutical compositions of the compounds of Formula III, Formula III(a), Formula V, Formula V(a), Formula A, Formula A¹, Formula A², Formula A³, or a pharmaceutically acceptable salt thereof can be used in combination with a method that involves treatment of cancer ex vivo. One example of such a treatment is an autologous stem cell transplant. In this method, a diseased entity's autologous hematopoietic stem cells are harvested and purged of all cancer cells. A therapeutic amount of a pharmaceutical composition including the compounds of Formula III, Formula III(a), Formula V, Formula V(a), Formula A, Formula A¹, Formula A², Formula A³, or a pharmaceutically acceptable salt thereof can then be administered to the patient prior to restoring the entity's bone marrow by addition of either the patient's own or donor stem cells.

### Methods

Another aspect is a method treating a condition or disease, comprising administering to a subject in need of such treatment a compound or pharmaceutical composition of a compound of Formula III, Formula III(a), Formula V, Formula V(a), Formula A, Formula A¹, Formula A², Formula A³, or a pharmaceutically acceptable salt thereof.

In some embodiments, the disorder or disease is cancer, neurodegenerative disorders, autoimmune disorders, cardiovascular disorders, metabolic disorders, hamartoma syndrome, genetic muscle disorders, and myopathies.

In another aspect, the invention provides a method of treating cancer, comprising administering to a patient in need of such treatment (e.g., a human patient) a compound of Formula III, Formula III(a), Formula V, Formula V(a), Formula A, Formula A¹, Formula A², Formula A³, or a pharmaceutically acceptable salt thereof, or a pharmaceutical composition comprising a compound of Formula III, Formula III(a), Formula V, Formula V(a), Formula A, Formula A¹, Formula A², Formula A³, or a pharmaceutically acceptable salt thereof.

Cancers that may be treated by pharmaceutical compositions including the compounds of Formula III, Formula III(a), Formula V, Formula V(a), Formula A, Formula A¹, Formula A², Formula A³, or a pharmaceutically acceptable salt thereof, either alone or in combination with another treatment modality include solid tumors such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, including malignant melanoma, neuroblastoma, non-Hodgkin lymphoma, papillary thyroid carcinoma, non-small cell lung carcinoma, and adenocarcinoma of lung and retinoblastoma. In some embodiments, cancers that may be treated using the compositions and methods disclosed herein include: non-Hodgkin lymphoma, colorectal cancer, malignant melanoma, papillary thyroid carcinoma, non-small cell lung carcinoma, and adenocarcinoma of lung. In various embodiments, at least a portion of the cancer cells derived from the cancers and metastases of the cancers described above may also harbor a mutation in the gene encoding a B-type Raf (BRAF) protein kinase, for example a mutation selected from V600E, V600K, V600R, V600D or combinations thereof.

Addition of a pharmaceutical composition of the present invention to cancer cells includes all actions by which an effect of the pharmaceutical composition on the cancer cell is realized. The type of addition chosen will depend upon whether the cancer cells are in vivo, ex vivo, or in vitro, the physical or chemical properties of the pharmaceutical composition, and the effect the composition is to have on the cancer cell. Non-limiting examples of addition include addition of a solution including the pharmaceutical composition to tissue culture media in which in vitro cancer cells are growing; any method by which a pharmaceutical composition may be administered to an animal including intravenous, per os, parenteral, or any other of the methods of administration; or the activation or inhibition of cells that in turn have effects on the cancer cells such as immune cells (e.g. macrophages and CD8+ T cells) or endothelial cells that may differentiate into blood vessel structures in the process of angiogenesis or vasculogenesis.

Determination of an effective amount of the compounds of Formula III, Formula III(a), Formula V, Formula V(a), Formula A, Formula A¹, Formula A², Formula A³, or a pharmaceutically acceptable salt thereof is within the capability of those skilled in the art, especially in light of the detailed disclosure provided herein. The effective amount of a pharmaceutical composition used to effect a particular purpose as well as its toxicity, excretion, and overall tolerance is determined in cell cultures or animals by pharmaceutical and toxicological procedures. One example is the determination of the IC₅₀ (half maximal inhibitory concentration) of the pharmaceutical composition in vitro in cell lines or target molecules. Another example is the determination of the LD₅₀ (lethal dose causing death in 50% of the tested animals) of the pharmaceutical composition in experimental animals. The exact techniques used in determining an effective amount will depend on factors such as the type and physical/chemical properties of the pharmaceutical composition, the property being tested, and whether the test is to be performed in vitro or in vivo. The determination of an effective amount of a pharmaceutical composition is well known to one of skill in the art who will use data obtained from any tests in making that determination. Determination of an effective amount of the compounds of Formula III, Formula III(a), Formula V, Formula V(a), Formula A, Formula A¹, Formula A², Formula A³, or a pharmaceutically acceptable salt thereof for addition to a cancer cell also includes the determination of an effective therapeutic amount, including the formulation of an effective dose range for use in vivo, including in humans.

The toxicity and therapeutic efficacy of a pharmaceutical composition may be determined by standard pharmaceutical procedures in cell cultures or animals. Examples include the determination of the IC₅₀ (the half maximal inhibitory concentration) and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized.

The effective amount of compound of Formula III, Formula III(a), Formula V, Formula V(a), Formula A, Formula A¹, Formula A², Formula A³, or a pharmaceutically acceptable salt thereof to result in the slowing of expansion of the cancer cells would preferably result in a concentration at or near the target tissue that is effective in slowing cellular expansion in neoplastic cells, but have minimal effects on non-neoplastic cells, including non-neoplastic cells exposed to radiation or recognized chemotherapeutic chemical agents. Concentrations that produce these effects can be determined using, for example, apoptosis markers such as the apoptotic index and/or capsase activities either in vitro or in vivo.

The addition of a therapeutically effective amount of the compounds of Formula III, Formula III(a), Formula V, Formula V(a), Formula A, Formula A¹, Formula A², Formula A³, or a pharmaceutically acceptable salt thereof encompasses any method of dosing of a compound. Dosing of the disclosed compound may include single or multiple administrations of any of a number of pharmaceutical compositions that include the disclosed compound as an active ingredient. Examples include a single administration of a slow release composition, a course of treatment involving several treatments on a regular or irregular basis, multiple administrations for a period of time until a diminution of the disease state is achieved, preventative treatments applied prior to the instigation of symptoms, or any other dosing regimen known in the art or yet to be disclosed that one skilled in the art would recognize as a potentially effective regimen. A final dosing regimen including the regularity of and mode of administration will be dependent on any of a number of factors including but not limited to the subject being treated; the severity of the affliction; the manner of administration, the stage of disease development, the presence of one or more other conditions such as pregnancy, infancy, or the presence of one or more additional diseases that affects the choice of the mode of administration, the dose to be administered and the time period over which the dose is administered.

Pharmaceutical compositions that include the compounds of Formula III, Formula III(a), Formula V, Formula V(a), Formula A, Formula A¹, Formula A², Formula A³, or a pharmaceutically acceptable salt thereof may be administered prior to, concurrently with, or after administration of a second pharmaceutical composition that may or may not include the compound. If the compositions are administered concurrently, they are administered within one minute of each other. If not administered concurrently, the second pharmaceutical composition may be administered a period of one or more minutes, hours, days, weeks, or months before or after the pharmaceutical composition that includes the compound.

Alternatively, a combination of pharmaceutical compositions may be cyclically administered. Cycling therapy involves the administration of one or more pharmaceutical compositions for a period of time, followed by the administration of one or more different pharmaceutical compositions for a period of time and repeating this sequential administration, in order to reduce the development of resistance to one or more of the compositions, to avoid or reduce the side effects of one or more of the compositions, and/or to improve the efficacy of the treatment.

The invention further encompasses kits that facilitate the administration of the disclosed compound to a subject having a disease described herein, and is in need of treatment. An example of such a kit includes one or more unit dosages of the compounds of Formula III, Formula III(a), Formula V, Formula V(a), Formula A, Formula A¹, Formula A², Formula A³, or a pharmaceutically acceptable salt thereof. The unit dosage would be enclosed in a preferably sterile container and would be comprised of the compound(s) of Formula III, Formula III(a), Formula V, Formula V(a), Formula A, Formula A¹, Formula A², Formula A³, or a pharmaceutically acceptable salt thereof and a pharmaceutically acceptable carrier. In another aspect, the unit dosage would comprise one or more lyophilates of the compound. In this aspect of the invention, the kit may include another preferably sterile container enclosing a solution capable of dissolving the lyophilate. However, such a solution need not be included in the kit and may be obtained separately from the lyophilate. In another aspect, the kit may include one or more devices used in administrating the unit dosages or a pharmaceutical composition to be used in combination with the compound. Examples of such devices include, but are not limited to, a syringe, a drip bag, a patch or an enema. In some aspects of the invention, the device comprises the container that encloses the unit dosage.

Pharmaceutical compositions of the compounds of Formula III, Formula III(a), Formula V, Formula V(a), Formula A, Formula A¹, Formula A², Formula A³, or a pharmaceutically acceptable salt thereof are used in methods of treating cancer or malaria. Such methods involve the administration of a therapeutic amount of a pharmaceutical composition of the compound of Formula III, Formula III(a), Formula V, Formula V(a), Formula A, Formula A¹, Formula A², Formula A³, or a pharmaceutically acceptable salt thereof, to a mammal in which a cancer has been diagnosed. In some embodiments, methods of the present invention, for example, methods for the treatment of cancer, and/or a cancer metastases, comprise administering to a subject in need thereof, a therapeutically effective amount of a compound of Formula III, Formula III(a), Formula V, Formula V(a), Formula A, Formula A¹, Formula A², Formula A³, or a pharmaceutically acceptable salt thereof. In some of these embodiments, the cancer to be treated with the compounds of the present invention, include treatment of BRAF and/or HRAS mutated cancers. In some embodiments, methods of treating cancer can include administering to the subject in need thereof, a therapeutically effective amount of a combination comprising a compound of Formula III, Formula III(a), Formula V, Formula V(a), Formula A, Formula A¹, Formula A², Formula A³, or a pharmaceutically acceptable salt thereof with an anti-cancer therapeutic such as 5-[2-chloro-2-methylphenyl)-1-hydroxy-3-oxo-2,3-dihydro-1H-isoindol-1-yl-]-1H-benzimidazol-2-yl)carbamate or AZD-8055 are disclosed infra. In some of these embodiments, the cancer is a BRAF mutated cancer. In some embodiments, the compound of the present invention is a compound of Formula A, Formula A¹, Formula A², Formula A³, or a pharmaceutically acceptable salt thereof and it is combined with a therapeutically effective amount of 5-[2-chloro-2-methylphenyl)-1-hydroxy-3-oxo-2,3-dihydro-1H-isoindol-1-yl-]-1H-benzimidazol-2-yl)carbamate or AZD-8055 and administered as a combination treatment to a patient with cancer, for example, a patient with a BRAF mutated cancer.

In some embodiments, the present invention provides a method of treating a cancer, or a cancer metastasis in a subject in need thereof, the method comprising: administering to the subject, a therapeutically effective amount of a compound of Formula A:

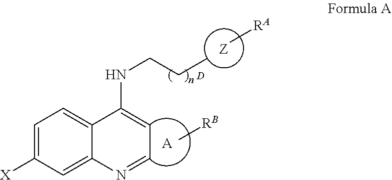

or a pharmaceutically acceptable salt thereof,
wherein:

A is an optionally substituted aryl or optionally substituted cycloalkyl;

Z is a 3 to 7 membered heterocycloalkyl;

X is H, halogen, or —CF₃;

n^{D} is 1 to 3;

R^{A} is optionally substituted C₁₋₆ alkyl; and

R^{B} is H, optionally substituted C₁₋₆ alkyl, or optionally substituted C₁₋₆ alkoxyl.

As discussed below, some of the cancers and/or metastases treatable with the compounds of the present invention may have a plurality of cancer cells forming the cancer tissues or cancer mass, some of which, i.e. at least a portion of these cells, having a BRAF protein kinase mutation or a mutation in the HRAS GTPase. In some embodiments, methods for the treatment of cancer can include treating a cancer and/or a cancer metastasis, wherein the cancer or cancer metastasis contain at least a portion of cancer cells that harbor a B-type RAF kinase (BRAF kinase) protein mutation and/or a mutation in HRAS protein. In these embodiments, the compounds of the present invention, including a compound of Formula III, Formula III(a), Formula V, Formula V(a), Formula A, Formula A¹, Formula A², Formula A³, or a pharmaceutically acceptable salt thereof when administered in therapeutically effective amounts to a subject in need thereof, can be used to treat such BRAF mutation and/or HRAS protein mutation containing cancers and/or metastases.

A therapeutic amount further includes the prevention of progression of the cancer to a neoplastic, malignant or metastatic state. Such preventative use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79). Hyperplasia is a form of controlled cell proliferation involving an increase in cell number in a tissue or organ, without significant alteration in structure or activity. For example, endometrial hyperplasia often precedes endometrial cancer and precancerous colon polyps often transform into cancerous lesions. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

Alternatively or in addition to the presence of abnormal cell growth characterized as hyperplasia, metaplasia, or dysplasia, the presence of one or more characteristics of a transformed phenotype or of a malignant phenotype, displayed in vivo or displayed in vitro by a cell sample derived from a patient can indicate the desirability of prophylactic/therapeutic administration of the pharmaceutical composition that includes the compound. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also id., at pp. 84-90 for characteristics associated with a transformed or malignant phenotype). Further examples include leukoplakia, in which a benign-appearing hyperplastic or dysplastic lesion of the epithelium, or Bowen's disease, a carcinoma in situ, are pre-neoplastic lesions indicative of the desirability of prophylactic intervention. In another example, fibrocystic disease including cystic hyperplasia, mammary dysplasia, adenosis, or benign epithelial hyperplasia is indicates desirability of prophylactic intervention.

In some aspects of the invention, use of the disclosed compounds may be determined by one or more physical factors such as tumor size and grade or one or more molecular markers and/or expression signatures that indicate prognosis and the likely response to treatment with the compound. For example, determination of estrogen (ER) and progesterone (PR) steroid hormone receptor status has become a routine procedure in assessment of breast cancer patients. See, for example, Fitzgibbons et al, Arch. Pathol. Lab. Med. 124:966-78, 2000. Tumors that are hormone receptor positive are more likely to respond to hormone therapy and also typically grow less aggressively, thereby resulting in a better prognosis for patients with ER+/PR+ tumors. In a further example, overexpression of human epidermal growth factor receptor 2 (HER-2/neu), a transmembrane tyrosine kinase receptor protein, has been correlated with poor breast cancer prognosis (see, e.g., Ross et al, The Oncologist 8:307-25, 2003), and Her-2 expression levels in breast tumors are used to predict response to the anti-Her-2 monoclonal antibody therapeutic trastuzumab (Herceptin®, Genentech, South San Francisco, Calif.).

In another aspect of the invention, the diseased entity exhibits one or more predisposing factors for malignancy that may be treated by administration of a pharmaceutical composition including the compound. Such predisposing factors include but are not limited to chromosomal translocations associated with a malignancy such as the Philadelphia chromosome for chronic myelogenous leukemia and t (14; 18) for follicular lymphoma; an incidence of polyposis or Gardner's syndrome that are indicative of colon cancer; benign monoclonal gammopathy which is indicative of multiple myeloma, kinship with persons who have had or currently have a cancer or precancerous disease, exposure to carcinogens, or any other predisposing factor that indicates in increased incidence of cancer now known or yet to be disclosed.

The invention further encompasses methods of treating cancer that comprise combination therapies that comprise the administration of a pharmaceutical composition including the disclosed compound and another treatment modality. Such treatment modalities include but are not limited to, radiotherapy, chemotherapy, surgery, immunotherapy, cancer vaccines, radioimmunotherapy, treatment with pharmaceutical compositions other than those which include the compounds of Formula III, Formula III(a), Formula V, Formula V(a), Formula A, Formula A¹, Formula A², or Formula A³, or any other method that effectively treats cancer in combination with the compounds of Formula III, Formula III(a), Formula V, Formula V(a), Formula A, Formula A¹, Formula A², or Formula A³. Combination therapies may act synergistically. That is, the combination of the two therapies is more effective than either therapy administered alone. This results in a situation in which lower dosages of both treatment modality may be used effectively. This in turn reduces the toxicity and side effects, if any, associated with the administration either modality without a reduction in efficacy.

In another aspect of the invention, the pharmaceutical composition including the compounds of Formula III, Formula III(a), Formula V, Formula V(a), Formula A, Formula A¹, Formula A², Formula A³, or a pharmaceutically acceptable salt thereof is administered in combination with a therapeutically effective amount of radiotherapy. The radiotherapy may be administered concurrently with, prior to, or following the administration of the pharmaceutical composition including the compound. The radiotherapy may act additively or synergistically with the pharmaceutical composition including the compound. This particular aspect of the invention would be most effective in cancers known to be responsive to radiotherapy. Cancers known to be responsive to radiotherapy include, but are not limited to, Non-Hodgkin's lymphoma, Hodgkin's disease, Ewing's sarcoma, testicular cancer, prostate cancer, ovarian cancer, bladder cancer, larynx cancer, cervical cancer, nasopharynx cancer, breast cancer, colon cancer, pancreatic cancer, head and neck cancer, esophogeal cancer, rectal cancer, small-cell lung cancer, non-small cell lung cancer, brain tumors, other CNS neoplasms, or any other such tumor.

Additional cancers that can be treated by pharmaceutical compositions of the compounds of Formula III, III(a), V, V(a), A, A¹, A², or A³ include blood borne cancers such as acute lymphoblastic leukemia (“ALL,”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), hairy cell leukemia, multiple myeloma, lymphoblastic leukemia, myelogenous leukemia, lymphocytic leukemia, myelocytic leukemia, Hodgkin's disease, non-Hodgkin's Lymphoma, Waldenstrom's macroglobulinemia, Heavy chain disease, and Polycythemia vera.

The compounds of Formula III, Formula III(a), Formula V, Formula V(a), Formula A, Formula A¹, Formula A², Formula A³, or a pharmaceutically acceptable salt thereof can be used to treat cancer and to treat neurodegenerative disorders, auto-immune disorders, cardiovascular disorders, metabolic disorders, hamartoma syndrome, malaria, genetic muscle disorders, and myopathy. It is to be understood that each of the compounds of Formulas III, III(a), V, V(a), A, A¹, A², and A³ as recited herein are useful for a number of the above conditions. It is well within the ability of those skilled in the art to easily determine which particular compound of Formula III, Formula III(a), Formula V, Formula V(a), Formula A, Formula A¹, Formula A², Formula A³, or a pharmaceutically acceptable salt thereof is useful for each particular condition without undue experimentation.

Further, compounds of Formula III, Formula III(a), Formula V, Formula V(a), Formula A, Formula A¹, Formula A², Formula A³, or a pharmaceutically acceptable salt thereof, can be used as cytostatic adjuvants to most small molecule/chemotherapy regimens, but the compounds also can be used as single agents. The compounds of Formulas III, III(a), V, V(a), A, A¹, A², and A³ can thus be used in combination with other drugs.

The exact dosage and frequency of administration depends on the particular compound of Formula III, Formula III(a), Formula V, Formula V(a), Formula A, Formula A¹, Formula A², Formula A³, or a pharmaceutically acceptable salt thereof used, the particular condition being treated, the severity of the condition being treated, the age, weight, general physical condition of the particular patient, other medication the individual may be taking as is well known to those skilled in the art and can be more accurately determined by measuring the blood level or concentration of the compound of Formula III, III(a), V, V(a), A, A¹, A², or A³ in the patient's blood and/or the patients response to the particular condition being treated.

Methods for Treating a BRAF Mutated and/or a HRAS Mutated Cancer

In some embodiments, methods are provided for the treatment of a cancer or a cancer metastasis. In one aspect, the method comprises treating a cancer or metastasis of the cancer, wherein the cancer and the metastasis harbors a B-type RAF protein kinase (BRAF-kinase) mutation and/or a HRAS protein mutation in a subject in need thereof.

An illustrative method for the treatment of a cancer or a cancer metastasis in a subject, wherein the cancer or cancer metastasis bears a BRAF protein kinase mutation, and/or a HRAS protein mutation, comprises administering to the subject, a pharmaceutical composition comprising a therapeutically effective amount of a compound of Formula III, Formula III(a), Formula V, Formula V(a), Formula A, Formula A¹, Formula A², Formula A³, or a pharmaceutically acceptable salt thereof.

In one illustrative method for the treatment of a cancer or a cancer metastasis in a subject, wherein the cancer or cancer metastasis bears a BRAF protein kinase mutation and/or a HRAS protein mutation, the method comprises administering to the subject, a pharmaceutical composition comprising a therapeutically effective amount of a compound of Formula A:

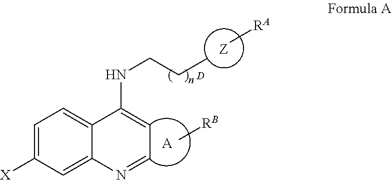

or a pharmaceutically acceptable salt thereof,
wherein:

A is an optionally substituted aryl or an optionally substituted cycloalkyl;

Z is a 3 to 7 membered heterocycloalkyl;

X is H, halogen, or —CF₃;

n^{D} is 1 to 3;

R^{A} is optionally substituted C₁₋₆ alkyl; and

R^{B} is H, optionally substituted C₁₋₆ alkyl, or optionally substituted C₁₋₆ alkoxyl.

In some embodiments, the compound of Formula A is a compound of Formula A¹:

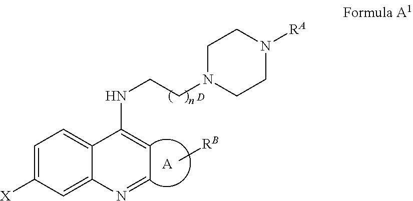

or a pharmaceutically acceptable salt thereof, wherein:
A is optionally substituted aryl or optionally substituted cycloalkyl;
X is H, halogen, or —CF₃;
n^{D} is 1 or 3;
R^{A} is optionally substituted C₁₋₆ alkyl; and
R^{B} is H, optionally substituted C₁₋₆ alkyl, or optionally substituted C₁₋₆ alkoxyl.

In some embodiments, the cancer or cancer metastasis cells bear a BRAF protein kinase mutation. In some embodiments, the cancer or the cancer metastasis harbors a HRAS protein mutation. In some embodiments, the HRAS protein mutation of the cancer is the mutation G13V.

In some embodiments, methods of treating cancers, tumors or metastases thereof include cancers, tumors or metastases thereof that bear a protein mutation in a BRAF protein and/or a protein mutation in a HRAS protein. In some of these embodiments, the cancers, tumors or metastases thereof bearing a protein mutation in a BRAF protein and/or a protein mutation in a HRAS protein are selected from: acute myeloid leukemia, melanoma, gliomas, sarcomas, histiocytic lymphoma, non-Hodgkin's lymphoma, thyroid cancer, papillary thyroid carcinoma, head and neck cancer, liver cancer, colorectal cancer, pancreatic cancer, breast cancer, ovarian cancer, lung cancer, and non-small cell lung carcinoma.

In some embodiments, the cancers, tumors or metastases thereof amenable to the treatment with a compound of the present invention is a melanoma cancer or a metastatic melanoma having a mutation in the cancer's BRAF protein kinase, and/or a mutation in the cancer's HRAS protein.

In another illustrative method for the treatment of a cancer or a cancer metastasis in a subject, wherein the cancer or cancer metastasis (or at least a portion of cancer cells therein) bears a BRAF protein kinase mutation, and/or a HRAS protein mutation, the method comprises administering to the subject, a pharmaceutical composition comprising a therapeutically effective amount of a compound of Formula A¹:

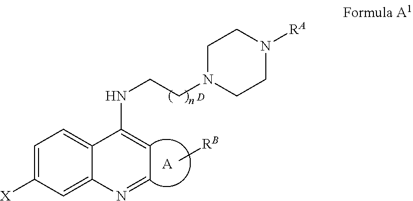

or a pharmaceutically acceptable salt thereof, wherein:

A is an optionally substituted aryl or optionally substituted cycloalkyl;

X is H, halogen, or —CF₃;

nD is 1 or 3;
RA is optionally substituted C₁₋₆ alkyl; and
RB is H, optionally substituted C₁₋₆ alkyl, or optionally substituted C₁₋₆ alkoxyl.

In another illustrative method for the treatment of a cancer or a cancer metastasis in a subject, wherein the cancer or cancer metastasis (or at least a portion of cancer cells therein) bears a BRAF protein kinase mutation, and/or a HRAS protein mutation, the method comprises administering to the subject, a pharmaceutical composition comprising a therapeutically effective amount of a compound of Formula A²:

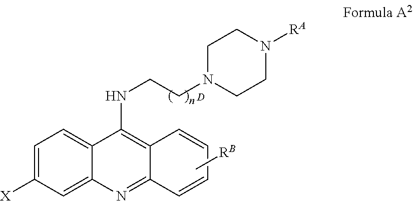

or a pharmaceutically acceptable salt thereof, wherein

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In another illustrative method for the treatment of a cancer or a cancer metastasis in a subject, wherein the cancer or cancer metastasis (or at least a portion of cancer cells therein) bears a BRAF protein kinase mutation, and/or a HRAS protein mutation, the method comprises administering to the subject, a pharmaceutical composition comprising a therapeutically effective amount of a compound of Formula A³:

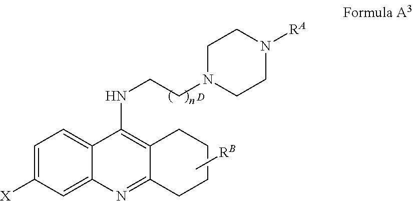

or a pharmaceutically acceptable salt thereof, wherein:

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In various embodiments, the methods for treatment of a cancer or a cancer metastasis harboring a BRAF protein kinase mutation, and/or a HRAS protein mutation comprises administering a therapeutically effective amount of:

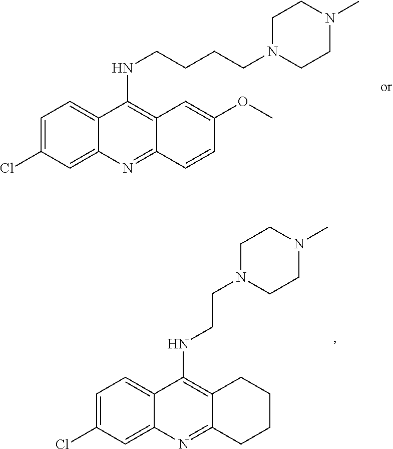 [SMILES: COc1ccc2nc3cc(Cl)ccc3c(NCCCCN3CCN(C)CC3)c2c1 ; CN1CCN(CCNc2c3c(nc4cc(Cl)ccc24)CCCC3)CC1]

or a pharmaceutically acceptable salt thereof.

In some embodiments, treatment of the subject's cancer and/or cancer metastasis results in a decrease in the size, and/or volume of the cancer, and/or a decrease in the number of cancer cells after exposing the subject's cancer and/or cancer metastasis to the pharmaceutical compositions comprising a compound of Formula A or a pharmaceutically acceptable salt thereof.

In some embodiments, the cancer or cancer metastasis or at least a portion of cells comprising the cancer or cancer metastasis will have a mutation in a BRAF protein kinase and/or a HRAS protein. Illustrative BRAF protein kinase mutations are known to those of skill in the art. In some embodiments, BRAF protein kinase mutations that may be sensitive to the activity of a compound of Formula A, can include: V600E, V600K, V600R, V600D or combinations thereof. Other BRAF protein kinase mutations are known in the oncological arts, and are also contemplated herein. In some embodiments, a cancer or cancer metastases harboring a BRAF mutation and/or a HRAS protein mutation can include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, including malignant melanoma, neuroblastoma, non-Hodgkin lymphoma, papillary thyroid carcinoma, non-small cell lung carcinoma, adenocarcinoma of lung and retinoblastoma. In some embodiments, the cancer that may be treated using the compositions, combinations and preparations disclosed herein can include: acute myeloid leukemia, melanoma, gliomas, sarcomas, histiocytic lymphoma, non-Hodgkin's lymphoma, thyroid cancer (for example, papillary thyroid carcinoma), head and neck cancer, liver cancer, colorectal cancer, pancreatic cancer, breast cancer, ovarian cancer, and lung cancer, for example, non-small cell lung carcinoma.

In some embodiments, the cancer or cancer metastases harboring a BRAF mutation is melanoma cancer or a metastatic melanoma. In some embodiments, the cancer or cancer metastases harboring a HRAS protein mutation is melanoma cancer or a metastatic melanoma.

The illustrative methods for treatment of the present disclosure provides a method of suppressing the growth of cancers, tumors, and/or neoplasms, or inhibiting the metastasis of a BRAF protein kinase mutated cancer, tumor and/or neoplasm in a subject in need thereof, for example a mammalian subject, including human subjects.

Methods for screening biopsied cancer or tumor samples, for example, melanoma tissue samples are known in the oncological arts. For example, determining the presence of a BRAF mutation, for example, a V600E, V600K, V600R, V600D or combinations thereof in a cancer sample can be accomplished using DNA amplification and sequencing the region of the BRAF gene containing nucleotide 1796, amplifying and digesting with a restriction endonuclease whose recognition or cleavage site is destroyed by the mutation (such as TspRI), primer extension methods, and hybridization to allele specific oligonucleotides operable to identify the transversion mutation at the nucleotide position 1799 (T1799A mutation) leading to the translation of the BRAF mutation V600E. Any method known in the art for detecting point mutations can be used. Genomic DNA, mRNA, or protein encoded by the BRAF gene may be assayed to determine the transversion mutation.

In another embodiment, a HRAS protein mutation, that may be sensitive to the activity of a compound of Formula III, Formula III(a), Formula V, Formula V(a), Formula A, Formula A¹, Formula A², Formula A³, or a pharmaceutically acceptable salt thereof, can include a mutation in the HRAS kinase protein. In some embodiments, the HRAS protein mutation includes G13V. Other mutations in the HRAS gene are known. GTPase HRAS also known as transforming protein p21 is an enzyme that in humans is encoded by the HRAS gene. The HRAS gene is located on the short (p) arm of chromosome 11 at position 15.5, from base pair 522,241 to base pair 525,549 (GenBank: Accession No. NC_000011.9).

In one example, gene expression profiling methods can be used to identify the BRAF and/or HRAS protein mutation. These include methods based on hybridization analysis of polynucleotides, methods based on sequencing of polynucleotides, and proteomics-based methods. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription polymerase chain reaction (RT-PCR) (Weis et al., Trends in Genetics 8:263-264 (1992)). Representative methods for sequencing-based gene expression analysis include Serial Analysis of Gene Expression (SAGE).

Methods for determining various mutations in BRAF, for example, V600E are known in the art. For example, a representative method for determining the presence of BRAF mutations are exemplified in U.S. Pat. No. 7,442,507 to Polsky et al, issued Oct. 28, 2008, the disclosure of which is incorporated by reference herein in its entirety. Other commercial screening protocols for BRAF mutations include the COBAS® 4800 BRAF V600 Mutation Test is an in vitro diagnostic device intended for the qualitative detection of BRAFV600E mutation in DNA extracted from formalin-fixed, paraffin-embedded human melanoma tissue. The COBAS® 4800 BRAF V600 Mutation Test is a real-time PCR test on the COBAS® 4800 system, and is intended to be used as an aid in selecting melanoma patients whose tumors carry the BRAFV600E mutation. The COBAS® 4800 BRAF V600 Mutation Test is commercially available from Roche Molecular Diagnostics.

In various embodiments, the methods to treat a BRAF kinase protein mutated cancer, and/or a HRAS protein mutated cancer provided herein can be employed by administering a therapeutically effective amount of a composition comprising a compound of Formula III, Formula III(a), Formula V, Formula V(a), Formula A, Formula A¹, Formula A², Formula A³, or a pharmaceutically acceptable salt thereof. The therapeutically effective amount of the compound of Formula III, Formula III(a), Formula V, Formula V(a), Formula A, Formula A¹, Formula A², Formula A³, or a pharmaceutically acceptable salt thereof can vary according to well known factors in the oncological arts. Such factors include, but are not limited to, the particular compound of Formula III, Formula III(a), Formula V, Formula V(a), Formula A, Formula A¹, Formula A², Formula A³, or a pharmaceutically acceptable salt thereof being administered; the severity and stage of the cancer; the presence of metastasis; the state of the subject's immune system (e.g., suppressed, compromised, stimulated); the route of administering the compound of Formula III, Formula III(a), Formula V, Formula V(a), Formula A, Formula A¹, Formula A², Formula A³, or a pharmaceutically acceptable salt thereof; the age of the patient; the general tolerance of the patient to the side effects, if any, of the composition; and the desired result (i.e., complete inhibition, or control of spreading to other tissues etc). Accordingly, it is not practical to set forth generally the amount that constitutes an effective amount of a compound of Formula III, Formula III(a), Formula V, Formula V(a), Formula A, Formula A¹, Formula A², Formula A³, or a pharmaceutically acceptable salt thereof. Those of ordinary skill in the art, however, can readily determine the appropriate amount with due consideration of such factors.

The term “therapeutically effective amount” denotes an amount of the compound of Formula III, Formula III(a), Formula V, Formula V(a), Formula A, Formula A¹, Formula A², Formula A³, or a pharmaceutically acceptable salt thereof containing composition, which is effective in achieving the desired therapeutic result, namely at least inhibiting the growth and/or spread of a cancer or cancer cells in the subject. In some embodiments, the “therapeutically effective amount” denotes an amount of the compound of Formula III, Formula III(a), Formula V, Formula V(a), Formula A, Formula A¹, Formula A², Formula A³, or a pharmaceutically acceptable salt thereof containing composition, which is effective in interfering with the autophagy capacity of at least a portion of the cancer cells within the cancer.

In some embodiments, exemplary methods of the present invention provide dosing to a subject in need thereof, a therapeutically effective amount or dose of the compound of Formula III, Formula III(a), Formula V, Formula V(a), Formula A, Formula A¹, Formula A², Formula A³, or a pharmaceutically acceptable salt thereof, ranging from about 0.01 mg per kg body weight to about 100 mg per kg body weight, per day. In some embodiments, a therapeutically effective amount or dose of the compound of Formula III, Formula III(a), Formula V, Formula V(a), Formula A, Formula A¹, Formula A², Formula A³, or a pharmaceutically acceptable salt thereof, ranging from about 1 mg per kg body weight to about 50 mg per kg body weight, per day. In some embodiments, a therapeutically effective amount or dose of the compound of Formula III, Formula III(a), Formula V, Formula V(a), Formula A, Formula A¹, Formula A², Formula A³, or a pharmaceutically acceptable salt thereof, ranges from about 10 mg per kg body weight to about 50 mg per kg body weight, per day.

In some embodiments, a therapeutically effective amount or dose of the compound of Formula III, Formula III(a), Formula V, Formula V(a), Formula A, Formula A¹, Formula A², Formula A³, or a pharmaceutically acceptable salt thereof, ranges from about 0.1 mg per kg body weight to about 75 mg per kg body weight, wherein the dosage is administered one or more times per day, or one or more times per week. The artisan, by routine type experimentation should have no substantial difficulties in determining the therapeutically effective amount in each case.

### Methods for Treating a Cancer or Cancer Metastasis Using a Combination Composition

In various embodiments, the present invention provides a method for the treatment of a cancer or a cancer metastasis in a subject, the method comprising: administering to the subject simultaneously or sequentially, a therapeutically effective amount of a combination of an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide and AZD-8055, and a compound of Formula III, Formula III(a), Formula V, Formula V(a), Formula A, Formula A¹, Formula A², Formula A³, or a pharmaceutically acceptable salt thereof.

In some embodiments, a method for the treatment of a cancer or a cancer metastasis in a subject, the method comprising: administering to the subject simultaneously or sequentially, a therapeutically effective amount of a combination of an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide and AZD-8055, and a compound of Formula A having a structure:

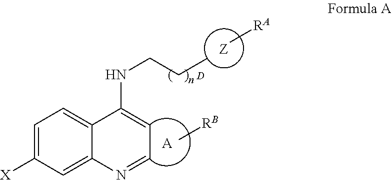

or a pharmaceutically acceptable salt thereof,
wherein:

A is an optionally substituted aryl or an optionally substituted cycloalkyl;

Z is a 3 to 7 membered heterocycloalkyl;

X is H, halogen, or —CF₃;

n^{D} is 1 to 3;

R^{A} is optionally substituted C₁₋₆ alkyl; and

R^{B} is H, optionally substituted C₁₋₆ alkyl, or optionally substituted C₁₋₆ alkoxyl.

In some embodiments, a method for the treatment of a cancer or a cancer metastasis in a subject, the method comprising: administering to the subject simultaneously or sequentially, a therapeutically effective amount of a combination of an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide and AZD-8055, and a compound of Formula A¹:

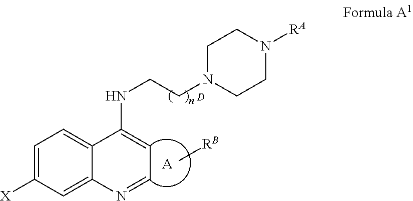

or a pharmaceutically acceptable salt thereof, wherein:

A is an optionally substituted aryl or optionally substituted cycloalkyl;

X is H, halogen, or —CF₃;

nD is 1 or 3;
RA is optionally substituted C₁₋₆ alkyl; and
RB is H, optionally substituted C₁₋₆ alkyl, or optionally substituted C₁₋₆ alkoxyl.

In some embodiments, a method for the treatment of a cancer or a cancer metastasis in a subject, the method comprising: administering to the subject simultaneously or sequentially, a therapeutically effective amount of a combination of an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide and AZD-8055, and a compound of Formula A²:

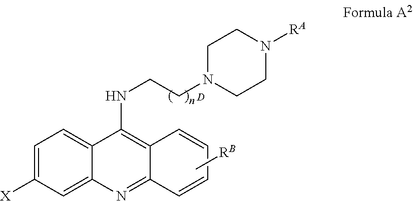

or a pharmaceutically acceptable salt thereof, wherein

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In some embodiments, a method for the treatment of a cancer or a cancer metastasis in a subject, the method comprising: administering to the subject simultaneously or sequentially, a therapeutically effective amount of a combination of an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide and AZD-8055, and a compound of Formula A³:

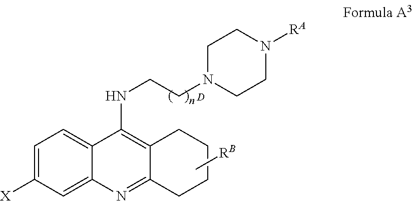

or a pharmaceutically acceptable salt thereof, wherein:

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In some embodiments, a compound of Formula III, Formula III(a), Formula V, Formula V(a), Formula A, Formula A¹, Formula A², Formula A³, or a pharmaceutically acceptable salt thereof and either N-[3-[5-(4-chlorophenyl)-1H-pyrrolo[2,3-b]pyridine-3-carbonyl]-2,4-difluorophenyl]propane-1-sulfonamide or AZD-8055, can be administered together, in a single composition, in combination, as a mixture, or a preparation, or can be administered separately in either order, sequentially in either order, or consecutively in either order. In some embodiments, if the active agents of the combination are not administered concurrently, the compound of Formula III, Formula III(a), Formula V, Formula V(a), Formula A, Formula A¹, Formula A², Formula A³, or a pharmaceutically acceptable salt thereof or the anti-cancer agent may be administered within a period of one or more minutes, hours, days, weeks, or months before or after the administration of the compound of Formula III, Formula III(a), Formula V, Formula V(a), Formula A, Formula A¹, Formula A², Formula A³, or a pharmaceutically acceptable salt thereof. When not administered together, the second active agent is typically administered within 72 hours of administering the first active agent. The actual method and order of administration of the constituents may vary according to the particular pharmaceutical formulation of the compound of Formula III, Formula III(a), Formula V, Formula V(a), Formula A, Formula A¹, Formula A², Formula A³, or a pharmaceutically acceptable salt thereof being utilized, the particular pharmaceutical formulation of the methyl (5-[2-chloro-2-methylphenyl)-1-hydroxy-3-oxo-2,3-dihydro-1H-isoindol-1-yl-]-1H-benzimidazol-2-yl)carbamate, or AZD-8055 being utilized, the particular cancer being treated, the severity of the disease state being treated, and the particular patient being treated.

In some embodiments, the exemplary methods for the treatment of various cancers includes the treatment of cancers that have a mutation in a BRAF protein kinase, and/or a mutation in HRAS protein. In some embodiments, the present methods also provide a method for the treatment of cancers harboring a BRAF mutation employing a pharmaceutical composition comprising the combination of a compound of Formula III, Formula III(a), Formula V, Formula V(a), Formula A, Formula A¹, Formula A², Formula A³, or a pharmaceutically acceptable salt thereof and methyl (5-[2-chloro-2-methylphenyl)-1-hydroxy-3-oxo-2,3-dihydro-1H-isoindol-1-yl-]-1H-benzimidazol-2-yl)carbamate, or a compound of Formula III, Formula III(a), Formula V, Formula V(a), Formula A, Formula A¹, Formula A², Formula A³, or a pharmaceutically acceptable salt thereof and AZD-8055. Such therapeutically effective combination of active agents exhibits anticancer, antitumor, and/or neoplastic efficacy, that are useful for all types of therapies for treating a variety of cancers, including BRAF mutated cancers, and/or HRAS mutated cancers, neoplasms, tumors, or metastases thereof. Exemplary cancers thus treatable using the compositions and the combinations, synergistic compositions and sensitizing compositions described herein can include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, including malignant melanoma, neuroblastoma, non-Hodgkin lymphoma, papillary thyroid carcinoma, non-small cell lung carcinoma, adenocarcinoma of lung and retinoblastoma. In some embodiments, the cancer that may be treated using the compositions, combinations and preparations disclosed herein can include: acute myeloid leukemia, melanoma, gliomas, sarcomas, histiocytic lymphoma, non-Hodgkin's lymphoma, thyroid cancer (for example, papillary thyroid carcinoma), head and neck cancer, liver cancer, colorectal cancer, pancreatic cancer, breast cancer, ovarian cancer, and lung cancer, for example, non-small cell lung carcinoma, any one of which, preferably has a BRAF protein kinase mutation, and/or a HRAS protein mutation. In some embodiments, the treated cancers, neoplasms, or tumors of the present invention is a melanoma cancer or metastasis from a melanoma cancer having a BRAF mutation, wherein the BRAF mutation is selected from V600E, V600K, V600R, V600D or combinations thereof.

In various embodiments, a typical composition, pharmaceutical composition, combination, mixture, or preparation of the constituents according to the disclosure is a compound of Formula III, Formula III(a), Formula V, Formula V(a), Formula A, Formula A¹, Formula A², Formula A³, or a pharmaceutically acceptable salt thereof, and methyl (5-[2-chloro-2-methylphenyl)-1-hydroxy-3-oxo-2,3-dihydro-1H-isoindol-1-yl-]-1H-benzimidazol-2-yl)carbamate, or a compound of Formula III, Formula III(a), Formula V, Formula V(a), Formula A, Formula A¹, Formula A², Formula A³, or a pharmaceutically acceptable salt thereof, and AZD-8055. In one embodiment, a more typical composition, pharmaceutical composition, combination, mixture, or preparation is an autophagy inhibitor selected from the group consisting of 6-Chloro-2-methoxy-N-(4-(4-methylpiperazin-1-yl)butyl)acridin-9-amine or a pharmaceutically acceptable salt thereof, or 6-chloro-N-(4-(4-methylpiperazin-1-yl)butyl)-1,2,3,4-tetrahydroacridin-9-amine or a pharmaceutically acceptable salt thereof, in combination with methyl (5-[2-chloro-2-methylphenyl)-1-hydroxy-3-oxo-2,3-dihydro-1H-isoindol-1-yl-]-1H-benzimidazol-2-yl)carbamate, or AZD-8055, prepared as a pharmaceutical composition individually or jointly, with at least one pharmaceutically acceptable excipient for administration to a subject in need thereof.

The constituents of the composition, combination, mixture, or preparation can be administered to a patient any acceptable manner that is medically acceptable, including orally, parenterally, topically, or by implantation. Oral administration includes administering the constituents of the compositions, combinations, mixtures, or preparations in the form of tablets, capsules, lozenges, suspensions, solutions, emulsions, powders, syrups, and the like. Parenteral administration of the composition, combination, mixture, or preparation can be accomplished using intravenous, subcutaneous, intramuscular, transdermally, or intratumorally routes with liquid or aerosolized formulations containing the active agent or active agents in combination and at least one pharmaceutically acceptable excipient, diluent or carrier.

In some embodiments, the combination can be administered as a single composition or formulation e.g. a tablet, pill, capsule, powder and the like, or as separate compositions, each composition providing a therapeutically effective amount of an active agent. In some embodiments, the compound of Formula III, Formula III(a), Formula V, Formula V(a), Formula A, Formula A¹, Formula A², Formula A³, or a pharmaceutically acceptable salt thereof and the anti-cancer agent are independently administered to the subject in the form of a solution, dispersion, suspension, powder, capsule, tablet, pill, time release capsule, time release tablet, and time release pill. In other embodiments, the combination or each of the compound of Formula III, Formula III(a), Formula V, Formula V(a), Formula A, Formula A¹, Formula A², Formula A³, or a pharmaceutically acceptable salt thereof and the anti-cancer agent are independently administered to the subject intravenously, intramuscularly, subcutaneously, intraperitoneally, intratumorally, orally, nasally, or combinations thereof. Preferred routes of administration can be selected based on the preferred method of formulating the combination, or each agent of the combination administered in substantially the same or in different ways.

In some embodiments, the therapeutically effective amount of each of the active agents in the combination can vary or be the same. In an exemplary embodiment, the therapeutically effective amount of the combination or each of the compound of Formula III, Formula III(a), Formula V, Formula V(a), Formula A, Formula A¹, Formula A², Formula A³, or a pharmaceutically acceptable salt thereof and the anti-cancer agent administered can range from about 0.1 mg per kg to about 100 mg per kg body weight of the subject. In other embodiments, the compound of Formula III, Formula III(a), Formula V, Formula V(a), Formula A, Formula A¹, Formula A², Formula A³, or a pharmaceutically acceptable salt thereof and the anti-cancer agent are each independently dosed to the subject in need thereof, in amounts ranging from about 0.01 mg per kg body weight to about 100 mg pre kg body weight, or, 1 mg per kg body weight to about 50 mg per kg body weight. In some embodiments, an exemplary dosage of the combination and/or each active agent of the combination can range from about 10 mg per kg body weight to about 50 mg per kg body weight of the subject. In a further embodiment, an exemplary dosage of the combination and/or each active agent of the combination can range from about 0.01 mg per kg body weight to about 25 mg per kg body weight of the subject. In still further embodiments, the compound of Formula III, Formula III(a), Formula V, Formula V(a), Formula A, Formula A¹, Formula A², Formula A³, or a pharmaceutically acceptable salt thereof and the anti-cancer agent are each administered in an amount from about 1 mg to about 1,500 mg per unit dosage form.

In each dosing scheme, an exemplary daily dose of the compound of Formula III, Formula III(a), Formula V, Formula V(a), Formula A, Formula A¹, Formula A², Formula A³, or a pharmaceutically acceptable salt thereof for the treatment of cancer or a cancer metastasis in a subject can range from 50 mg to about 1,000 mg pre day, administered in one or more doses, one to four times per day. In these described embodiments, administration of the combination comprising a compound of Formula III, Formula III(a), Formula V, Formula V(a), Formula A, Formula A¹, Formula A², Formula A³, or a pharmaceutically acceptable salt thereof and the anti-cancer agent inhibits the autophagy capacity of at least a portion of the cancer cells within the cancer.

### Synergistically Effective Combination Compositions

In various embodiments, the present invention provides pharmaceutical compositions and methods for the treatment of a cancer or a cancer metastasis in a subject, the method comprises administering to a subject in need thereof, simultaneously or sequentially, a synergistically effective therapeutic amount of a combination of a compound of Formula III, Formula III(a), Formula V, Formula V(a), Formula A, Formula A¹, Formula A², Formula A³, or a pharmaceutically acceptable salt thereof and an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide and AZD-8055. In some of the above embodiments, the methods for treatment of a cancer or a cancer metastasis comprise treating a cancer or cancer metastasis harboring a BRAF protein kinase mutation, and/or a HRAS protein mutation.

In some embodiments, the method for the treatment of a cancer or a cancer metastasis in a subject comprises administering to a subject in need thereof, simultaneously or sequentially, a synergistically effective therapeutic amount of a combination of a compound of Formula A, or a pharmaceutically acceptable salt thereof. In some of the above embodiments, the methods for treatment of a cancer or a cancer metastasis comprise treating a cancer or cancer metastasis harboring a BRAF protein kinase mutation, and/or a HRAS-protein mutation. The compound of Formula A having a structure:

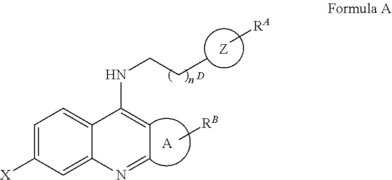

or a pharmaceutically acceptable salt thereof,
wherein:

A is optionally substituted aryl or optionally substituted cycloalkyl;

Z is a 3 to 7 membered heterocycloalkyl;

X is H, halogen, or —CF₃;

n^{D} is 1 to 3;

R^{A} is optionally substituted C₁₋₆ alkyl; and

R^{B} is H, optionally substituted C₁₋₆ alkyl, or optionally substituted C₁₋₆ alkoxyl;

and
an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide and AZD-8055.

In some embodiments, the compound of Formula A is a compound of Formula A¹:

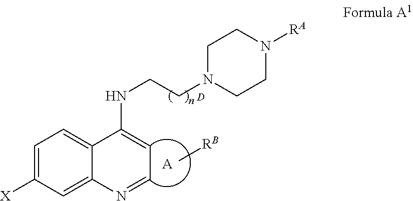

or a pharmaceutically acceptable salt thereof, wherein:
A is an optionally substituted aryl or optionally substituted cycloalkyl;
X is H, halogen, or —CF₃;
n^{D} is 1 or 3;
R^{A} is optionally substituted C₁₋₆ alkyl; and
R^{B} is H, optionally substituted C₁₋₆ alkyl, or optionally substituted C₁₋₆ alkoxyl.

In various embodiments, the methods for treatment of a cancer or a cancer metastasis harboring a BRAF protein kinase mutation, and/or a HRAS-protein mutation comprises administering a therapeutically effective amount of:

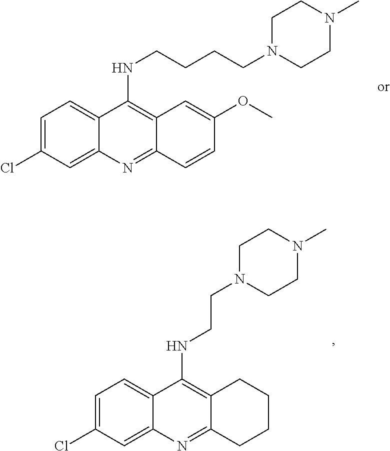 [SMILES: COc1ccc2nc3cc(Cl)ccc3c(NCCCCN3CCN(C)CC3)c2c1 ; CN1CCN(CCNc2c3c(nc4cc(Cl)ccc24)CCCC3)CC1]

or a pharmaceutically acceptable salt thereof.

As used herein, a synergistic pharmaceutical composition can comprise a synergistically effective amount of each of the active agents of the present disclosure. In some embodiments, a synergistic pharmaceutical composition can comprise a synergistically effective amount of a compound of Formula III, Formula III(a), Formula V, Formula V(a), Formula A, Formula A¹, Formula A², Formula A³, or a pharmaceutically acceptable salt thereof and a synergistically effective amount of methyl (5-[2-chloro-2-methylphenyl)-1-hydroxy-3-oxo-2,3-dihydro-1H-isoindol-1-yl-]-1H-benzimidazol-2-yl)carbamate, or a synergistically effective amount of compound of Formula III, Formula III(a), Formula V, Formula V(a), Formula A, Formula A¹, Formula A², Formula A³, or a pharmaceutically acceptable salt thereof and a synergistically effective amount of AZD-8055, and at least one pharmaceutically acceptable carrier or excipient. These synergistically effective pharmaceutical compositions of the present disclosure are useful in anticancer therapy, particularly, cancers having a determined BRAF protein kinase mutation, and/or a HRAS protein mutation, for example, a genetic mutation T1799A leading to the expression of a B-type Raf kinase, or BRAF family of serine/threonine-specific protein kinases having a mutation in the kinase domain (for example, V599E, V600E, V600K, V600R or V600D or combinations thereof).

It has been surprisingly found that the combination of a compound of Formula III, Formula III(a), Formula V, Formula V(a), Formula A, Formula A¹, Formula A², Formula A³, or a pharmaceutically acceptable salt thereof and N-[3-[5-(4-chlorophenyl)-1H-pyrrolo[2,3-b]pyridine-3-carbonyl]-2,4-difluorophenyl]propane-1-sulfonamide or a combination of a compound of Formula III, Formula III(a), Formula V, Formula V(a), Formula A, Formula A¹, Formula A², Formula A³, or a pharmaceutically acceptable salt thereof and AZD-8055, provided therapeutically synergistic antitumor activity against oncogenic BRAF tumor cells, for example BRAF mutated melanoma cancer cells. Synergies were observed in experimental treatments described herein.

It can be shown by established test models and in particular those models described herein, that the combination of the active agents of the invention results in synergistic activity compared to the effects observed with the single combination partners. The pharmacological activity of the combination of the active agents of the invention may be further demonstrated in a clinical study as well as in the procedures described herein.

In another embodiment, methods for treating a cancer using a synergistic combination of a compound of Formula III, Formula III(a), Formula V, Formula V(a), Formula A, Formula A¹, Formula A², Formula A³, or a pharmaceutically acceptable salt thereof and either N-[3-[5-(4-chlorophenyl)-1H-pyrrolo[2,3-b]pyridine-3-carbonyl]-2,4-difluorophenyl]propane-1-sulfonamide or AZD-8055, can include administering a compound of Formula III, Formula III(a), Formula V, Formula V(a), Formula A, Formula A¹, Formula A², Formula A³, or a pharmaceutically acceptable salt thereof to a subject in need thereof, orally at a synergistically therapeutic effective dose of about 1 mg/kg to about 100 mg/kg administered per oral every 1 to 14 days for 1 or more administrations. The second constituent of the combination, i.e. the anti-cancer agent, for example, either N-[3-[5-(4-chlorophenyl)-1H-pyrrolo[2,3-b]pyridine-3-carbonyl]-2,4-difluorophenyl]propane-1-sulfonamide or AZD-8055, can be administered at a synergistically therapeutic effective doses ranging from about 1 mg/kg to about 50 mg/kg or 0.1 mg per kg body weight to about 25 mg per kg body weight, dosed orally or parenterally, once, twice or three times per day, for 1 to 30 days, for 1 or more months, for 1 or more years, or until the cancer is in remission or the patient has died. Therapeutic synergy represents a therapeutic effect achieved with a tolerated regimen of a combination treatment that exceeds the optimal effect achieved at any tolerated dose of monotherapy associated with the same drugs used in the combination. In various embodiments, the synergistically effective therapeutic combination, or each of the compound of Formula III, Formula III(a), Formula V, Formula V(a), Formula A, Formula A¹, Formula A², Formula A³, or a pharmaceutically acceptable salt thereof and the anti-cancer agent in the combination, can be independently administered to the subject intravenously, intramuscularly, subcutaneously, intraperitoneally, intratumorally, orally, nasally, or combinations thereof.

The pharmaceutically acceptable carriers or excipients are well known to those having ordinary skill in the art of formulating compounds in a form of pharmaceutical compositions, combinations, mixtures, and preparations. A pharmaceutically acceptable carrier refers to one or more compatible solid or liquid filler, carrier, diluent, or encapsulating substances which are suitable for administration to mammals including humans. Pharmaceutical compositions, combinations, mixtures, and preparations suitable for parenteral administration are formulated in a sterile form which may be a sterile solution or suspension in an acceptable diluent or solvent.

The amount of active ingredients contained in the pharmaceutical composition, combination or synergistic composition may vary quite widely depending on many factors, such as the route of administration and the vehicle. In the present invention, a pharmaceutical composition may contain a synergistically effective amount of each active agent ranging from about 0.1 to about 1,000 mg, i.e. of a compound of Formula III, Formula III(a), Formula V, Formula V(a), Formula A, Formula A¹, Formula A², Formula A³, or a pharmaceutically acceptable salt thereof, and from about 0.1 to about 1,000 mg of N-[3-[5-(4-chlorophenyl)-1H-pyrrolo[2,3-b]pyridine-3-carbonyl]-2,4-difluorophenyl]propane-1-sulfonamide or from about 0.1 to about 1,000 mg of AZD-8055 all suitably formulated with at least one pharmaceutically acceptable excipient.

In some embodiments, a synergistically effective combination of a compound of Formula III, Formula III(a), Formula V, Formula V(a), Formula A, Formula A¹, Formula A², Formula A³, or a pharmaceutically acceptable salt thereof and either N-[3-[5-(4-chlorophenyl)-1H-pyrrolo[2,3-b]pyridine-3-carbonyl]-2,4-difluorophenyl]propane-1-sulfonamide or AZD-8055, can be administered on any clinically useful schedule, including, but not limited to, daily, twice weekly, weekly or every other week. Specifically, for weekly administration, typical dosages of the compound of Formula III, Formula III(a), Formula V, Formula V(a), Formula A, Formula A¹, Formula A², Formula A³, or a pharmaceutically acceptable salt thereof, might range from about 0.1 mg/kg to about 100 mg/kg body weight of the subject, or from about 1 mg per kg body weight to about 50 mg per kg body weight of the subject, adjusted as needed by standard oncological medical procedures, to accommodate any developing patient needs. In some embodiments, the compound of Formula III, Formula III(a), Formula V, Formula V(a), Formula A, Formula A¹, Formula A², Formula A³, or a pharmaceutically acceptable salt thereof and the anti-cancer agent are each independently administered in amounts ranging from about 0.1 mg per kg body weight to about 25 mg per kg body weight, or from about 10 mg per kg body weight to about 50 mg per kg body weight per dose, wherein each dose is a daily dose, or a partial daily dose. In some of these embodiments, the compound of Formula III, Formula III(a), Formula V, Formula V(a), Formula A, Formula A¹, Formula A², Formula A³, or a pharmaceutically acceptable salt thereof is either 6-Chloro-2-methoxy-N-(4-(4-methylpiperazin-1-yl)butyl)acridin-9-amine, or 6-chloro-N-(4-(4-methylpiperazin-1-yl)butyl)-1,2,3,4-tetrahydroacridin-9-amine, or pharmaceutically acceptable salts thereof.

Methods for Sensitizing a Cancer to Treatment with an Anti-Cancer Agent

In various embodiments, the present disclosure provides for methods for treating a cancer, cancer metastasis or proliferative disease. In various embodiments, an exemplary method provides a method of sensitizing cancer cells in a subject undergoing a chemotherapeutic treatment for the treatment of cancer, or a cancer metastasis, the method comprising administering a compound of Formula III, Formula III(a), Formula V, Formula V(a), Formula A, Formula A¹, Formula A², Formula A³, or a pharmaceutically acceptable salt thereof to the subject with the cancer to be treated, before or after administration of at least one chemotherapeutic agent. In various embodiments, an exemplary method provides a method of sensitizing cancer cells harboring a BRAF protein kinase mutation, and/or a HRAS protein mutation in a subject undergoing a chemotherapeutic treatment for the treatment of cancer. In some embodiments, the method comprises identifying cancer cells in the subject having a BRAF protein kinase mutation. If at least a portion of the cancer cells have a BRAF protein kinase mutation, and/or a HRAS-protein mutation, the method provides administering to the subject simultaneously or sequentially, a combination comprising a therapeutically effective amount of a compound of Formula III, Formula III(a), Formula V, Formula V(a), Formula A, Formula A¹, Formula A², Formula A³, or a pharmaceutically acceptable salt thereof, and a therapeutically effective amount of an anti-cancer agent. In various embodiments, the compound of Formula A has a structure:

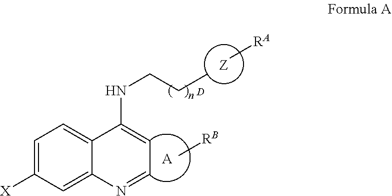

or a pharmaceutically acceptable salt thereof, wherein:

A is an optionally substituted aryl or optionally substituted cycloalkyl;

Z is a 3 to 7 membered heterocycloalkyl;

X is H, halogen, or —CF₃;

n^{D} is 1 to 3;

R^{A} is optionally substituted C₁₋₆ alkyl; and

R^{B} is H, optionally substituted C₁₋₆ alkyl, or optionally substituted C₁₋₆ alkoxyl; and wherein the anti-cancer agent is selected from the group consisting of N-[3-[5-(4-chlorophenyl)-1H-pyrrolo[2,3-b]pyridine-3-carbonyl]-2,4-difluorophenyl]propane-1-sulfonamide and AZD8055.

In one embodiment, the methods of the present invention provide treating a cancer or a cancer metastasis with an inhibitor of BRAF or mTOR activity, wherein before treatment with a BRAF or mTOR inhibitor the cancer is first sensitized to treatment with the BRAF or mTOR inhibitor, comprising providing to the cancer a pharmaceutical composition comprising a compound of Formula III, Formula III(a), Formula V, Formula V(a), Formula A, Formula A¹, Formula A², Formula A³, or a pharmaceutically acceptable salt thereof, wherein the compound of Formula III, Formula III(a), Formula V, Formula V(a), Formula A, Formula A¹, Formula A², Formula A³, or a pharmaceutically acceptable salt thereof, is provided in a therapeutically effective amount so as to sensitize the cancer cell to the later treatment with a BRAF inhibitor, or a mTOR inhibitor, or combinations of both BRAF and mTOR inhibitors; and treating the sensitized cancer cell with the BRAF or mTOR inhibitor or combination thereof.

In one embodiment, the compound of Formula III, Formula III(a), Formula V, Formula V(a), Formula A, Formula A¹, Formula A², Formula A³, or a pharmaceutically acceptable salt thereof is a compound of Formula A, or a pharmaceutically acceptable salt thereof having the structure of Formula A¹:

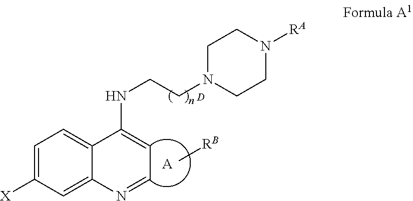

or a pharmaceutically acceptable salt thereof, wherein:

A is an optionally substituted aryl or an optionally substituted cycloalkyl;

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In one embodiment, the compound of Formula III, Formula III(a), Formula V, Formula V(a), Formula A, Formula A¹, Formula A², Formula A³, or a pharmaceutically acceptable salt thereof is a compound of Formula A²

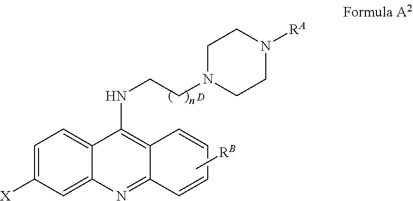

or a pharmaceutically acceptable salt thereof, wherein

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In one embodiment, the compound of Formula III, Formula III(a), Formula V, Formula V(a), Formula A, Formula A¹, Formula A², Formula A³, or a pharmaceutically acceptable salt thereof is a compound of Formula A³:

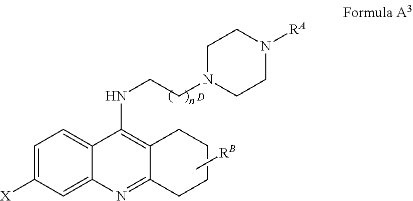

or a pharmaceutically acceptable salt thereof, wherein:

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In various embodiments, the chemotherapeutic treatment includes treatment of the subject having a cancer with a BRAF inhibitor, for example, N-[3-[5-(4-chlorophenyl)-1H-pyrrolo[2,3-b]pyridine-3-carbonyl]-2,4-difluorophenyl]propane-1-sulfonamide or an mTOR inhibitor, for example, AZD-8055. In some embodiments, the subject's cancer includes a cancer having a BRAF protein kinase mutation, and/or a HRAS-protein mutation, for example, a mutation comprising V599E, V600E, V600K, V600R or V600D or combinations thereof. In various embodiments, the cancer can include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, including malignant melanoma, neuroblastoma, non-Hodgkin lymphoma, papillary thyroid carcinoma, non-small cell lung carcinoma, adenocarcinoma of lung and retinoblastoma. In some embodiments, the cancer that may be treated using the compositions, combinations and preparations disclosed herein can include: acute myeloid leukemia, melanoma, gliomas, sarcomas, histiocytic lymphoma, non-Hodgkin's lymphoma, thyroid cancer (for example, papillary thyroid carcinoma), head and neck cancer, liver cancer, colorectal cancer, pancreatic cancer, breast cancer, ovarian cancer, and lung cancer, for example, non-small cell lung carcinoma.

The present method of treatment of a cancer or a cancer metastasis harboring a BRAF protein kinase mutation, and/or a HRAS protein mutation makes possible a potentiation of the anticancer, antitumor, and/or antineoplastic efficacy of BRAF inhibitors, such as methyl (5-[2-chloro-2-methylphenyl)-1-hydroxy-3-oxo-2,3-dihydro-1H-isoindol-1-yl-]-1H-benzimidazol-2-yl)carbamate; a V600E BRAF inhibitor; or PLX-4032 (also referred to as VEMURAFENIB®, and marketed by Roche and Plexxikon) or mTOR inhibitors, for example, AZD-8055, an ATP-competitive inhibitor of mTOR kinase activity by concurrent or sequential administration of an autophagy inhibitor compound of the present invention represented by Formula III, Formula III(a), Formula V, Formula V(a), Formula A, Formula A¹, Formula A², Formula A³, or a pharmaceutically acceptable salt thereof.

Therefore, in certain embodiments, the present invention provides combination pharmaceutical compositions comprising a therapeutically effective amount of a compound of Formula III, Formula III(a), Formula V, Formula V(a), Formula A, Formula A¹, Formula A², Formula A³, or a pharmaceutically acceptable salt thereof and a therapeutically effective amount of methyl (5-[2-chloro-2-methylphenyl)-1-hydroxy-3-oxo-2,3-dihydro-1H-isoindol-1-yl-]-1H-benzimidazol-2-yl)carbamate. In other embodiments, present invention provides combination pharmaceutical compositions comprising a therapeutically effective amount of a compound of Formula III, Formula III(a), Formula V, Formula V(a), Formula A, Formula A¹, Formula A², Formula A³, or a pharmaceutically acceptable salt thereof and a therapeutically effective amount of AZD-8055 as disclosed herein above.

The present disclosure further provides the use of a compound of Formula III, Formula III(a), Formula V, Formula V(a), Formula A, Formula A¹, Formula A², Formula A³, or a pharmaceutically acceptable salt thereof and N-[3-[5-(4-chlorophenyl)-1H-pyrrolo[2,3-b]pyridine-3-carbonyl]-2,4-difluorophenyl]propane-1-sulfonamide or a combination of a therapeutically effective amount of a compound of Formula III, Formula III(a), Formula V, Formula V(a), Formula A, Formula A¹, Formula A², Formula A³, or a pharmaceutically acceptable salt thereof and a therapeutically effective amount of AZD-8055, which can provide for an efficacious treatment at reduced doses compared to those required when each drug is used alone. In some embodiments, the combination or each of the compound of Formula III, Formula III(a), Formula V, Formula V(a), Formula A, Formula A¹, Formula A², Formula A³, or a pharmaceutically acceptable salt thereof and the anti-cancer agent can be independently administered to the subject in need thereof. In various embodiments, the pharmaceutical composition can be administered to the subject in need thereof in the form of a solution, dispersion, suspension, powder, capsule, tablet, pill, micro tablets, micro capsules, time release capsule, time release tablet, and time release pill.

In various embodiments, a pharmaceutical composition comprising the combination of agents or each of the agents independently i.e., a compound of Formula III, Formula III(a), Formula V, Formula V(a), Formula A, Formula A¹, Formula A², Formula A³, or a pharmaceutically acceptable salt thereof and the anti-cancer agent can be independently or in combination, administered to the subject intravenously, intramuscularly, subcutaneously, intraperitoneally, intratumorally, orally, nasally, or combinations thereof.

In some embodiments, the sensitizing composition comprising a compound of Formula III, Formula III(a), Formula V, Formula V(a), Formula A, Formula A¹, Formula A², Formula A³, or a pharmaceutically acceptable salt thereof, can include a compound selected from:

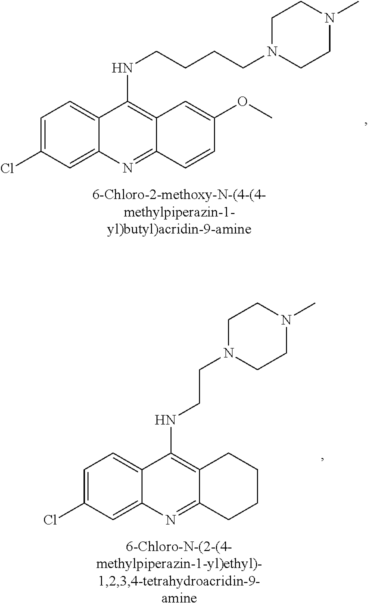 [SMILES: COc1ccc2nc3cc(Cl)ccc3c(NCCCCN3CCN(C)CC3)c2c1 ; CN1CCN(CCNc2c3c(nc4cc(Cl)ccc24)CCCC3)CC1]

or a pharmaceutically acceptable salt thereof.

In various embodiments, the combination of a compound of Formula III, Formula III(a), Formula V, Formula V(a), Formula A, Formula A¹, Formula A², Formula A³, or a pharmaceutically acceptable salt thereof and an anti-cancer agent, for example methyl (5-[2-chloro-2-methylphenyl)-1-hydroxy-3-oxo-2,3-dihydro-1H-isoindol-1-yl-]-1H-benzimidazol-2-yl)carbamateor AZD-8055 can be used to sensitize a cancer, tumor, malignancy or metastasis thereof. In some embodiments, the cancer is a cancer harboring a BRAF-kinase protein mutation, selected from V600E, V600K, V600R, V600D, or combinations thereof. In various embodiments, cancers that can be treated with the combination compositions of the present invention can include: acute myeloid leukemia, melanoma, gliomas, sarcomas, histiocytic lymphoma, non-Hodgkin's lymphoma, thyroid cancer, papillary thyroid carcinoma, head and neck cancer, liver cancer, colorectal cancer, pancreatic cancer, breast cancer, ovarian cancer, lung cancer, and non-small cell lung carcinoma. In some embodiments, BRAF protein kinase mutated and/or HRAS protein mutated cancers can include BRAF and/or HRAS mutated acute myeloid leukemia, melanoma, gliomas, sarcomas, histiocytic lymphoma, non-Hodgkin's lymphoma, thyroid cancer, papillary thyroid carcinoma, head and neck cancer, liver cancer, colorectal cancer, pancreatic cancer, breast cancer, ovarian cancer, lung cancer, and non-small cell lung carcinoma wherein any of the foregoing cancers have a BRAF mutated protein kinase, and/or a HRAS protein mutation.

In various embodiments of the present invention, methods for sensitizing cancer cells harboring a BRAF-kinase protein mutation include sensitizing melanoma cancer or a metastatic melanoma using the compositions described herein. In other embodiments of the present invention, methods for sensitizing cancer cells harboring a HRAS protein mutation include sensitizing melanoma cancer or a metastatic melanoma using the compositions described herein.

In some embodiments of the sensitization method, each patient receives an autophagy inhibitory compound of Formula III, Formula III(a), Formula V, Formula V(a), Formula A, Formula A¹, Formula A², Formula A³, or a pharmaceutically acceptable salt thereof, on a daily, or on a weekly or other clinically useful schedule, at dose levels typically used for the particular compound involved, ie. ranging from about 0.01 mg/kg to about 100 mg/kg per weight of the subject, or a daily dose ranging from about 10 mg to about 1,000 mg, administered in one or more doses, one to four times per day. These clinically useful doses can be ascertained using a carefully monitored titration dosage scheme to obtain a clinically useful range, wherein such range may depend on factors such as age, condition, sex, and extent of the disease in the patient, pharmacokinetic profile of the compound, bioavailability of the compound in the formulation used, severity of the cancer, the degree of mutation of the cancer, incidence of adverse effects etc. In some embodiments, an exemplary therapeutically effective dosing range of the compound of Formula III, Formula III(a), Formula V, Formula V(a), Formula A, Formula A¹, Formula A², Formula A³, or a pharmaceutically acceptable salt thereof, or a pharmaceutically acceptable salt thereof may be from about 0.1 mg/kg to about 100 mg/kg body weight of the subject dosed orally or parenterally, or combinations thereof. In some embodiments, an initial higher dosage of the compound of Formula III, Formula III(a), Formula V, Formula V(a), Formula A, Formula A¹, Formula A², Formula A³, or a pharmaceutically acceptable salt thereof may be administered, for example, from about 5 mg/kg to about 100 mg/kg administered orally or parenterally that may be reduced to optimal doses of about 0.1 mg/kg to about 50 mg/kg per weight of the subject. These doses may be administered to the subject at dosage levels and/or dosing frequencies that reach or approach the maximum tolerated dosed for each subject. The maximum tolerated dose for each individual subject may be determined using commonly known medical procedures. The therapeutically effective doses may be provided on a daily basis, for example, one or more doses per day, for example, 1-5 doses may be administered per day, or per week, or a regimen of similar dose levels adjusted for optimal use in the combination setting.

In view of the surprising chemotherapeutic results obtained against BRAF mutated cancers, using the combination of a compound of Formula III, Formula III(a), Formula V, Formula V(a), Formula A, Formula A¹, Formula A², Formula A³, or a pharmaceutically acceptable salt thereof, or a pharmaceutically acceptable salt thereof and either N-[3-[5-(4-chlorophenyl)-1H-pyrrolo[2,3-b]pyridine-3-carbonyl]-2,4-difluorophenyl]propane-1-sulfonamide or AZD-8055, the present invention provides a method for treating a cancer or a cancer metastasis in a subject with the combination compositions described herein.

Moreover, the use of a combination of a compound of Formula III, Formula III(a), Formula V, Formula V(a), Formula A, Formula A¹, Formula A², Formula A³, or a pharmaceutically acceptable salt thereof and N-[3-[5-(4-chlorophenyl)-1H-pyrrolo[2,3-b]pyridine-3-carbonyl]-2,4-difluorophenyl]propane-1-sulfonamide or a combination of a compound of Formula III, Formula III(a), Formula V, Formula V(a), Formula A, Formula A¹, Formula A², Formula A³, or a pharmaceutically acceptable salt thereof and AZD-8055, can provide a treatment which is safer and less toxic compared to each drug used alone. In some embodiments, the compound of Formula III, Formula III(a), Formula V, Formula V(a), Formula A, Formula A¹, Formula A², Formula A³, or a pharmaceutically acceptable salt thereof and methyl (5-[2-chloro-2-methylphenyl)-1-hydroxy-3-oxo-2,3-dihydro-1H-isoindol-1-yl-]-1H-benzimidazol-2-yl)carbamate, or a compound of Formula III, Formula III(a), Formula V, Formula V(a), Formula A, Formula A¹, Formula A², Formula A³, or a pharmaceutically acceptable salt thereof and AZD-8055 can be used simultaneously, separately or consecutively, in any order, or in a specific order that provides enhanced anti-cancer efficacy and/or a reduction in harmful side-effects.

The anticancer therapeutic effects of the chemotherapeutic or cytotoxic agents N-[3-[5-(4-chlorophenyl)-1H-pyrrolo[2,3-b]pyridine-3-carbonyl]-2,4-difluorophenyl]propane-1-sulfonamide or AZD-8055 are significantly increased by the earlier, or concurrent, or subsequent administration, of a compound of Formula III, Formula III(a), Formula V, Formula V(a), Formula A, Formula A¹, Formula A², Formula A³, or a pharmaceutically acceptable salt thereof. These enhanced anticancer therapeutic effects can be produced without an increase in toxicity, due, in part, to the synergism between the compound of Formula III, Formula III(a), Formula V, Formula V(a), Formula A, Formula A¹, Formula A², Formula A³, or a pharmaceutically acceptable salt thereof and the N-[3-[5-(4-chlorophenyl)-1H-pyrrolo[2,3-b]pyridine-3-carbonyl]-2,4-difluorophenyl]propane-1-sulfonamide or AZD-8055. The doses of the compound of Formula III, Formula III(a), Formula V, Formula V(a), Formula A, Formula A¹, Formula A², Formula A³, or a pharmaceutically acceptable salt thereof and the N-[3-[5-(4-chlorophenyl)-1H-pyrrolo[2,3-b]pyridine-3-carbonyl]-2,4-difluorophenyl]propane-1-sulfonamide or AZD-8055 can be administered as frequently as necessary. The actual method and order of administration will vary according to the particular formulation, composition, combination, mixture, or preparation, the particular cancer being treated, and the particular patient being treated.

In some embodiments, BRAF mutation harboring cancers that may be treated using the compositions, combination treatments and methods disclosed herein, include: acute myeloid leukemia, melanoma, gliomas, sarcomas, histiocytic lymphoma, non-Hodgkin's lymphoma, thyroid cancer (for example, papillary thyroid carcinoma), head and neck cancer, liver cancer, colorectal cancer, pancreatic cancer, breast cancer, ovarian cancer, and lung cancer, for example, non-small cell lung carcinoma. In some embodiments, BRAF protein kinase mutation and/or HRAS protein mutation harboring cancers that may be treated using the compositions, combination treatments and methods disclosed herein, include: non-Hodgkin lymphoma, colorectal cancer, malignant melanoma, papillary thyroid carcinoma, non-small cell lung carcinoma, and adenocarcinoma of lung.

The enhanced actions of the combination of a compound of Formula III, Formula III(a), Formula V, Formula V(a), Formula A, Formula A¹, Formula A², Formula A³, or a pharmaceutically acceptable salt thereof with a BRAF inhibitor or an mTOR inhibitor of the present disclosure are shown, by way of example, in the following standard experimental models of tumor growth, which are intended to illustrate but not to limit the present disclosure.

Treatment and Prevention of Malaria

In another group of embodiments, the compounds of the present invention, have been identified as potent anti-malarial compounds, useful in the treatment and/or prevention of malaria in a subject.

In one example, a method for the treatment and/or prevention of malaria in a subject in need of anti-malarial treatment or prevention, comprises administering to the subject in need of anti-malarial treatment and/or prevention, a therapeutically effective amount of a compound of Formula A:

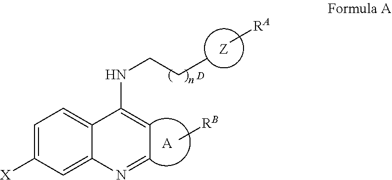

or a pharmaceutically acceptable salt thereof,
wherein:

A is an optionally substituted aryl or optionally substituted cycloalkyl;

Z is a 3 to 7 membered heterocycloalkyl;

X is H, halogen, or —CF₃;

n^{D} is 1 to 3;

R^{A} is optionally substituted C₁₋₆ alkyl; and

R^{B} is H, optionally substituted C₁₋₆ alkyl, or optionally substituted C₁₋₆ alkoxyl.

In related embodiment, the present invention provides a compound of Formula A or a pharmaceutically acceptable salt thereof, wherein the compound is a compound of Formula A¹:

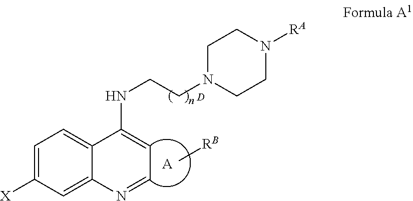

or a pharmaceutically acceptable salt thereof, wherein:

A is an optionally substituted aryl or optionally substituted cycloalkyl;

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In related embodiment, the present invention provides a compound of Formula A or a pharmaceutically acceptable salt thereof, wherein the compound is a compound of Formula A²:

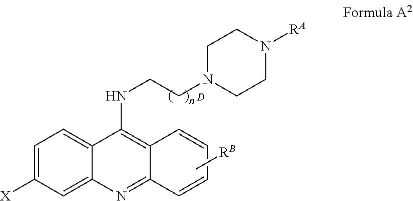

or a pharmaceutically acceptable salt thereof,

wherein:

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In related embodiment, the present invention provides a compound of Formula A or a pharmaceutically acceptable salt thereof, wherein the compound is a compound of Formula A³:

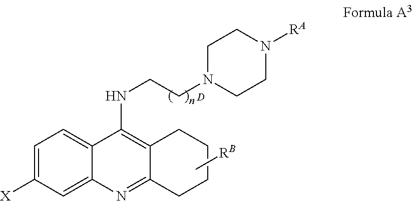

or a pharmaceutically acceptable salt thereof,
wherein:

X is H, halogen, or —CF₃;

n^{D} is 1 or 3;

R^{A} is optionally substituted C₁₋₆ alkyl; and

R^{B} is H, optionally substituted C₁₋₆ alkyl, or optionally substituted C₁₋₆ alkoxyl.

In another embodiment of the methods of the present invention, the compound of Formula A is a compound represented by the structure:

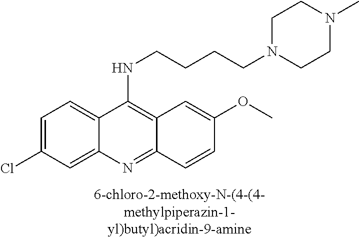 [SMILES: COc1ccc2nc3cc(Cl)ccc3c(NCCCCN3CCN(C)CC3)c2c1]

or a pharmaceutically acceptable salt thereof.

In another embodiment of the methods of the present invention, the compound of Formula A is a compound represented by the structure:

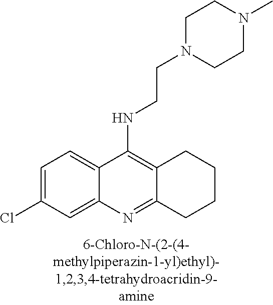 [SMILES: CN1CCN(CCNc2c3c(nc4cc(Cl)ccc24)CCCC3)CC1]

In various embodiments of the present invention, methods for treating and/or preventing malaria in a subject in need thereof, can comprise administering a therapeutic effective amount of a compound of Formula A, A¹, A², or A³, Example 7, Example 27 (Table 1) or a pharmaceutically acceptable salt thereof to the subject. In some embodiments, the compounds of the present invention can be formulated into a pharmaceutical composition in the form of a solution, dispersion, suspension, powder, capsule, tablet, pill, time release capsule, time release tablet, and time release pill, wherein the compound of the present antimalarial treatment can be prepared as a pharmaceutical composition individually or jointly, with at least one pharmaceutically acceptable excipient for administration to a subject in need thereof.

In various embodiments, the compounds of the present invention are admixed with one or more excipients, diluents or carriers, as described in greater detail above, which are known to those of skill in the art to prepare a suitable composition, or a pharmaceutical composition which may be administered to a subject in need thereof. In various embodiments, the compound of Formula A, A¹, A², or A³ or a pharmaceutically acceptable salt thereof is formulated into a pharmaceutical composition in the form of a solution, a dispersion, a suspension, a powder, a capsule, a tablet, a pill, a time release capsule, a time release tablet, or a time release pill containing one or more doses of the compound of Formula A, A¹, A², or A³ or a pharmaceutically acceptable salt thereof.

In one aspect, the invention provides pharmaceutical compositions for the treatment and/or prevention of malaria comprising compound of Formula A, A¹, A², or A³ or a pharmaceutically acceptable salt thereof according to the invention and a pharmaceutically acceptable carrier, excipient, or diluent.

In one embodiment for the treatment and/or prevention of malaria, each dose of the compound of Formula A, A¹, A², A³, Example 7, Example 27, or a pharmaceutically acceptable salt thereof administered to the subject ranges from about 0.01 mg per kg body weight to about 100 mg per kg body weight, and one or more doses are administered one or more times per day, or one or more times per week. In one embodiment, an indicated daily dosage in the larger subject, e.g. humans, is in the range from about 0.5 mg to about 1,000 mg, conveniently administered, e.g. in divided doses up to four times a day or in retard form. Suitable unit dosage forms for oral administration comprise from ca. 1 to 500 mg active ingredient.

The determination of a therapeutically effective dose of one or more compounds of the present invention can be calculated or identified using a screening method as described in the examples sections below, or can be derived through controlled clinical trials using standard pharmacological procedures approved by governing drug regulatory bodies, such as the U.S. Food and Drug Administration (FDA). A therapeutically effective dose of a compound of Formula A, A¹, A², A³, Example 7, Example 27, or a pharmaceutically acceptable salt thereof for the treatment and/or prevention of malaria in a subject in need thereof, can refer to an amount of active ingredient which shows activity against malarial parasites. One example of a therapeutically effective dose of a compound of Formula A, A¹, A², A³, Example 7, Example 27, or a pharmaceutically acceptable salt thereof, can include a dose of the compound or pharmaceutically acceptable salt thereof that results in an IC₅₀ of <10 μg/ml against P. falciparum 3D7 using malaria parasite growth inhibition assays. Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

In some embodiments, a method for the treatment and/or prevention of malaria can include administering a therapeutically effective amount of a compound of Formula A, A¹, A², A³, Example 7, Example 27, or a pharmaceutically acceptable salt thereof and one or more secondary active agents selected from: artemisinin, artemether, artesunate, arteflene, dihydroartemisinin, chlorproguanil, trimethoprim, chloroquine, quinine, mefloquine, amodiaquine, atovaquone, proguanil, lumefantrine, piperaquine, pyronaridine, halofantrine, pyrimethamine-sulfadoxine, quinacrine, pyrimethamine-dapsone, quinidine, amopyroquine, sulphonamides, primaquine, ferroquine, tafenoquine, arterolane, and pyronaridine to the subject in need thereof.

In various embodiments, the present methods for preventing or treating malaria can comprise a method for the prevention and/or treatment of malaria in a subject in need of anti-malarial prevention or treatment, the method comprising administering to the subject, a therapeutically effective amount of a compound of Formula A:

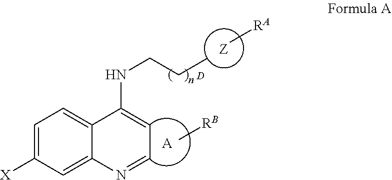

or a pharmaceutically acceptable salt thereof,
wherein:

A is an optionally substituted aryl or optionally substituted cycloalkyl;

Z is a 3 to 7 membered heterocycloalkyl;

X is H, halogen, or —CF₃;

n^{D} is 1 to 3;

R^{A} is optionally substituted C₁₋₆ alkyl; and

R^{B} is H, optionally substituted C₁₋₆ alkyl, or optionally substituted C₁₋₆ alkoxyl.

In some of these embodiments, the compound or a pharmaceutically acceptable salt thereof, can include Example 7 and/or Example 27 or a pharmaceutically acceptable salt thereof. In some of these embodiments, the malaria can be caused or affected by drug resistant Plasmodium species, including strains of any one or more of Plasmodium falciparum, Plasmodium vivax, Plasmodium malariae, or Plasmodium ovale that are resistant to any one of chloroquine, mefloquine, sulfadoxine-pyrimethamine (SP), or artemisinin. While the present invention is not bound by any particular theory, or mechanism of action, it is believed that the compounds of Formula A, A¹, A², A³, Example 7, Example 27, or a pharmaceutically acceptable salt thereof of the present invention inhibit the autophagy activity and/or capacity of the Plasmodium sp. infected cells.

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

### 6-Chloro-N-(1-ethylpiperidin-4-yl)-2-methoxyacridin-9-amine

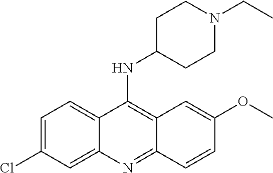 [SMILES: CCN1CCC(Nc2c3ccc(Cl)cc3nc3ccc(OC)cc23)CC1]

A mixture of 6,9-dichloro-2-methoxyacridine (100 mg, 0.36 mmol) and phenol (approximately 1.5 g) was heated to 100° C. under nitrogen atmosphere and stirred for 1 hour. 1-Ethylpiperidin-4-amine (92 mg, 0.72 mmol) was added to the mixture. The reaction was stirred at 100° C. for 5 hours, cooled to 20-25° C., and diluted with dichloromethane. The mixture was washed twice with sodium hydroxide solution (1 N) and twice with ammonium chloride solution. The phases were separated, and the organic layer was dried and concentrated. The residue was purified by Biotage column chromatography using triethylamine (5%) and methanol (5 to 15%) in dichloromethane to give the title compound; MS (Found: M+1=370).

### Example 2

### 6-Chloro-N-(2-(2-(diethylamino)ethoxy)ethyl)-2-methoxyacridin-9-amine

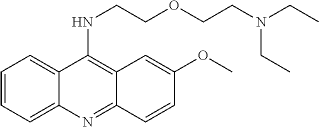 [SMILES: CCN(CC)CCOCCNc1c2ccccc2nc2ccc(OC)cc12]

Following the general procedure of Example 1 and making non-critical variations, but using 6,9-dichloro-2-methoxyacridine and commercially available 2-(2-aminoethoxy)-N,N-diethylethanamine, the title compound was obtained; MS (Found M+1=402).

### Example 3

### 6-Chloro-2-methoxy-N-(4-methoxybutyl)acridin-9-amine

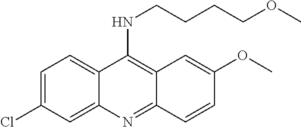 [SMILES: COCCCCNc1c2ccc(Cl)cc2nc2ccc(OC)cc12]

Following the general procedure of Example 1 and making non-critical variations, but using 6,9-dichloro-2-methoxyacridine and commercially available 4-methoxybutan-1-amine, the title compound was obtained; MS (Found M+1=345).

### Example 4

### 6-Chloro-2-methoxy-N-(4-(pyrrolidin-1-yl)butyl)acridin-9-amine

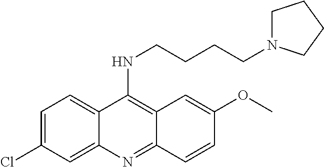 [SMILES: COc1ccc2nc3cc(Cl)ccc3c(NCCCCN3CCCC3)c2c1]

### Step 1. Synthesis of 4-(benzyloxycarbonylamino)butyl methanesulfonate

To a solution of benzyl 4-hydroxybutylcarbamate (1.1 g, 4.9 mmol) and triethylamine (1.7 mL, 9.8 mmol) in THF was added methanesulfonyl chloride (0.67 g, 5.9 mmol) at 0° C. The reaction was stirred at 20-25° C. for 6 hours and concentrated. The residue was partitioned between ethyl acetate and water. The phases were separated, and the organic layer was washed with hydrochloride solution (1 N), saturated sodium bicarbonate solution, and saline. The separated organic layer was dried and concentrated to give 4-(benzyloxycarbonylamino)butyl methanesulfonate (1.2 g).

### Step 2. Synthesis of benzyl 4-(pyrrolidin-1-yl)butylcarbamate

To a pressure vessel was added 4-(benzyloxycarbonylamino)butyl methanesulfonate (330 mg, 1.10 mmol) and pyrrolidine (234 mg, 3.3 mmol) in THF. The reaction was heated to 100° C., stirred overnight, cooled to 20-25° C., and concentrated. The crude concentrate was purified by Biotage column chromatography to give benzyl 4-(pyrrolidin-1-yl)butylcarbamate (200 mg).

### Step 3. Synthesis of 4-(pyrrolidin-1-yl)butan-1-amine

To a solution of benzyl 4-(pyrrolidin-1-yl)butylcarbamate (196 mg, 0.71 mmol) was added catalytic amount of Pd/C (5%). The reaction was stirred under a hydrogen atmosphere overnight and filtered. The filtrate was concentrated to give 4-(pyrrolidin-1-yl)butan-1-amine (83 mg)

### Step 4. Synthesis of the Title Compound

Following the general procedure of Example 1 and making non-critical variations, but using 6,9-dichloro-2-methoxyacridine and 4-(pyrrolidin-1-yl)butan-1-amine (Step 3), the title compound was obtained; MS (Found M+1=384).

### Example 5

### N¹-tert-butyl-N⁴-(6-chloro-2-methoxyacridin-9-yl)butane-1,4-diamine

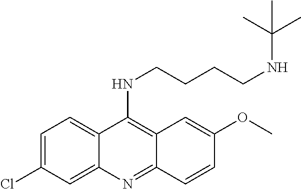 [SMILES: COc1ccc2nc3cc(Cl)ccc3c(NCCCCNC(C)(C)C)c2c1]

Following the general procedure of Example 1 and making non-critical variations, but using 6,9-dichloro-2-methoxyacridine and commercially available N¹-tert-butylbutane-1,4-diamine, the title compound was obtained; MS (Found M+1=386).

### Example 6

### N-(4-(6-Chloro-2-methoxyacridin-9-ylamino)butyl)-N-ethylmethanesulfonamide

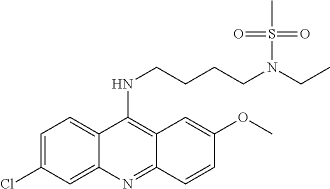 [SMILES: CCN(CCCCNc1c2ccc(Cl)cc2nc2ccc(OC)cc12)S(C)(=O)=O]

### Step 1. Synthesis of benzyl 4-(ethylamino)butylcarbamate

Following the general procedure of Example 4, Step 2, and making non-critical variations but using 4-(benzyloxycarbonylamino)butyl methanesulfonate and ethyl amine in THF, the compound of Step 1 was obtained.

### Step 2. Synthesis of benzyl 4-(N-ethylmethylsulfonamido)butylcarbamate

To a mixture of benzyl 4-(ethylamino)butylcarbamate (Step 1, 250 mg, 1.00 mmol) in dichloromethane was added pyridine (145 mg, 1.8 mmol) and then methanesulfonyl chloride (137 mg, 1.20 mmol) at 0° C. The reaction was stirred overnight and diluted with dichloromethane. The phases were separated, and the organic phase was washed with hydrochloride solution (1 N), saturated sodium bicarbonate and saline. The separated organic layer was concentrated and purified by Biotage column chromatography to give benzyl 4-(N-ethylmethylsulfonamido)butylcarbamate (231 mg).

### Step 3. Synthesis of N-(4-aminobutyl)-N-ethylmethanesulfonamide

Following the general procedure of Example 4, Step 3, but using benzyl 4-(N-ethylmethylsulfonamido)butylcarbamate (Step 2), the compound of Step 3 was obtained.

### Step 4. Synthesis of the title compound

Following the general procedure of Example 1 and making non-critical variations, but using 6,9-dichloro-2-methoxyacridine and N-(4-aminobutyl)-N-ethylmethanesulfonamide (Step 3), the title compound was obtained; MS (Found M+1=436).

### Example 7

### 6-Chloro-2-methoxy-N-(4-(4-methylpiperazin-1-yl)butyl)acridin-9-amine

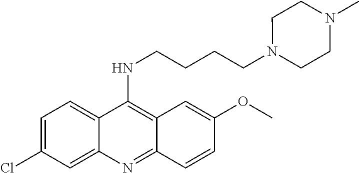 [SMILES: COc1ccc2nc3cc(Cl)ccc3c(NCCCCN3CCN(C)CC3)c2c1]

Following the general procedure of Example 1 and making non-critical variations, but using 6,9-dichloro-2-methoxyacridine and commercially available 4-(4-methylpiperazin-1-yl)butan-1-amine, the title compound was obtained, MS (Found M+1=413. ¹H NMR (CD₃OD, 300 Hz): 8.32-8.30 (d, 1H J=8.2 Hz), 8.30-7.85 (m, 1H), 7.58-7.57 (d, 1H), 7.47-7.44 (m, 1H), 7.34-7.32 (m, 1H), 4.00 (s, 3H), 3.92-3.89 (t, 2H, J=6 Hz), 2.54-2.51 (b, 4H), 2.41-2.29 (m, 6H), 2.25 (s, 3H), 1.85-1.77 (m, 2H), 1.60-1.53 (m, 2H).

### Example 8

### 3-Chloro-N-(1-ethylpiperidin-4-yl)acridin-9-amine

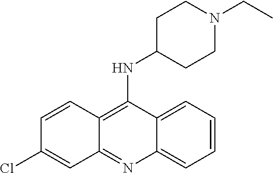 [SMILES: CCN1CCC(Nc2c3ccccc3nc3cc(Cl)ccc23)CC1]

Following the general procedure of Example 1 and making non-critical variations, but using 3,9-dichloroacridine (J. Med. Chem. 1985, 28. 940-944) and 1-ethylpiperidin-4-amine, the title compound was obtained; MS (Found M+1=340).

### Example 9

### N-(4-(6-Chloro-2-methoxyacridin-9-ylamino)butyl)-N-ethylacetamide

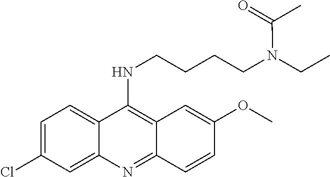 [SMILES: CCN(CCCCNc1c2ccc(Cl)cc2nc2ccc(OC)cc12)C(C)=O]

### Step 1. Synthesis of N-(4-aminobutyl)-N-ethylacetamide

Following the general procedure of Example 6, Steps 2 and 3, and making non-critical variations N-(4-aminobutyl)-N-ethylacetamide was obtained.

### Step 2. Synthesis of the title compound

Following the general procedure of Example 1, and making non-critical variations but using 6,9-dichloro-2-methoxyacridine and N-(4-aminobutyl)-N-ethylacetamide (Step 1), the title compound was obtained; MS (Found M+1=400).

### Example 10

### N¹-(6-Chloro-2-methoxyacridin-9-yl)-N⁴-(cyclopropylmethyl)-N⁴-methylbutane-1,4-diamine

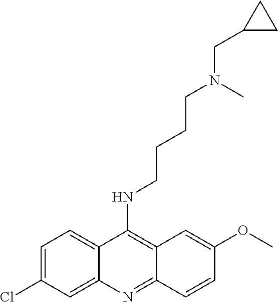 [SMILES: COc1ccc2nc3cc(Cl)ccc3c(NCCCCN(C)CC3CC3)c2c1]

### Step 1. Synthesis of N¹-(cyclopropylmethyl)-N¹-methylbutane-1,4-diamine

Following the general procedure of Example 4, Steps 2 and 3, and making non-critical variations but using 4-(benzyloxycarbonylamino)butyl methanesulfonate and commercially available 1-cyclopropyl-N-methylmethanamine, N¹-(cyclopropylmethyl)-N¹-methylbutane-1,4-diamine was obtained.

### Step 2. Synthesis of Title Compound

Following the general procedure of Example 1 and making non-critical variations but using 6,9-dichloro-2-methoxyacridine and N¹-(cyclopropylmethyl)-N¹-methylbutane-1,4-diamine (Step 1), the title compound was obtained; MS (Found M+1=398). 1H NMR (CDCl₃, 300 Hz): 8.05-7.94 (m, 3H), 7.40-7.36 (m, 1H), 7.26-7.23 (m, 2H), 3.93 (s, 3H), 3.75-3.71 (t, 2H, J=6 Hz), 2.48-2.43 (t, 2H, J=6 Hz), 2.29-2.22 (m, 5H), 1.85-1.78 (m, 2H), 1.76-1.65 (m, 2H), 0.87-0.85 (m, 1H), 0.50-0.45 (d, 2H, J=2.4 Hz), 0.09-0.05 (d, 2H, J=4.8 Hz).

### Example 11

### 6-Chloro-2-methoxy-N-(2-methoxyethyl)acridin-9-amine

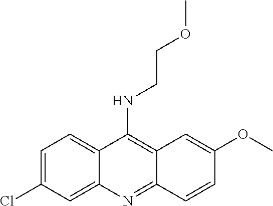 [SMILES: COCCNc1c2ccc(Cl)cc2nc2ccc(OC)cc12]

Following the general procedure of Example 1 and making non-critical variations but using 6,9-dichloro-2-methoxyacridine and 2-methoxyethanamine, the title compound was obtained; MS (Found M+1=317).

### Example 12

### N¹-(6-Chloro-2-methoxyacridin-9-yl)-N⁴-cyclopropyl-N⁴-ethylbutane-1,4-diamine

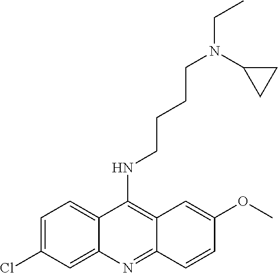 [SMILES: CCN(CCCCNc1c2ccc(Cl)cc2nc2ccc(OC)cc12)C1CC1]

### Step 1. Synthesis of benzyl 4-(cyclopropyl(ethyl)amino)butylcarbamate

Following the general procedure of Example 4, Step 2, and making non-critical variations but using 4-(benzyloxycarbonylamino)butyl methanesulfonate and commercially available N-ethylcyclopropanamine, benzyl 4-(cyclopropyl(ethyl)amino)butylcarbamate was obtained.

### Step 2. N¹-cyclopropyl-N¹-ethylbutane-1,4-diamine HCl salt

The mixture of benzyl 4-(cyclopropyl(ethyl)amino)butylcarbamate (Step 1) in HCl (6 N) was heated to reflux for 1 hour and cooled to 20-25° C. The reaction mixture was concentrated, and the residue was dried under reduced pressure to give N¹-cyclopropyl-N¹-ethylbutane-1,4-diamine HCl salt.

### Step 3. Synthesis of the Title Compound

Following the general procedure of Example 1 and making non-critical variations but using 6,9-dichloro-2-methoxyacridine, N¹-cyclopropyl-N¹-ethylbutane-1,4-diamine HCl salt (Step 2) and diisopropyethylamine (4 eq), the title compound was obtained; MS (Found M+1=398).

### Example 13

### N¹-(6-Chloro-2-methoxyacridin-9-yl)-N⁴-(cyclopropylmethyl)-N⁴-ethylbutane-1,4-diamine

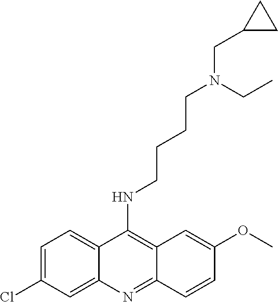 [SMILES: CCN(CCCCNc1c2ccc(Cl)cc2nc2ccc(OC)cc12)CC1CC1]

### Step 1. Synthesis of N¹-(cyclopropylmethyl)-N¹-ethylbutane-1,4-diamine

Following the general procedure of Example 4, Step 2, and making non-critical variations but using 4-(benzyloxycarbonylamino)butyl methanesulfonate and N-(cyclopropylmethyl)ethanamine, N¹-(cyclopropylmethyl)-N¹-ethylbutane-1,4-diamine was obtained

### Step 2. Synthesis of the Title Compound

Following the general procedure of Example 1 and making non-critical variations but using 6,9-dichloro-2-methoxyacridine, N¹-(cyclopropylmethyl)-N¹-ethylbutane-1,4-diamine (Step 1), the title compound is obtained; MS (Found M+1=412).

### Example 14

### N¹-(6-Chloro-2-methoxyacridin-9-yl)-N⁴,N⁴-diethyl-N¹-methylbutane-1,4-diamine

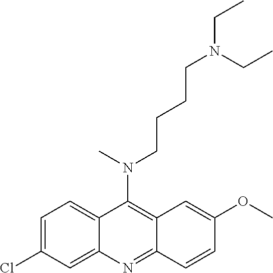 [SMILES: CCN(CC)CCCCN(C)c1c2ccc(Cl)cc2nc2ccc(OC)cc12]

Following the general procedure of Example 1 and making non-critical variations but using 6,9-dichloro-2-methoxyacridine and N¹,N¹-diethyl-N⁴-methylbutane-1,4-diamine, the title compound was obtained. MS (Found M+1=400).

### Example 15

### N-(1-Ethylpiperidin-4-yl)acridin-9-amine

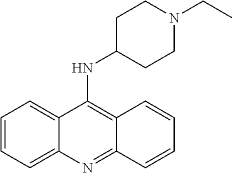 [SMILES: CCN1CCC(Nc2c3ccccc3nc3ccccc23)CC1]

Following the general procedure of Example 1 and making non-critical variations but using 9-chloroacridine and 1-ethylpiperidin-4-amine, the title compound was obtained; MS (Found M+1=306).

### Example 16

### 6-Chloro-N-(1-ethylpiperidin-4-yl)-2-fluoroacridin-9-amine

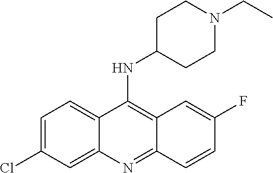 [SMILES: CCN1CCC(Nc2c3ccc(Cl)cc3nc3ccc(F)cc23)CC1]

Following the general procedure of Example 1 and making non-critical variations but using 6,9-dichloro-2-fluoroacridine (J. Med. Chem. 1985, 28. 940-944) and 1-ethylpiperidin-4-amine, the title compound was obtained; MS (Found M+1=358).

### Example 17

### 6-Chloro-2-fluoro-N-(2-(4-methylpiperazin-1-yl)ethyl)acridin-9-amine

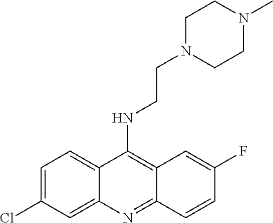 [SMILES: CN1CCN(CCNc2c3ccc(Cl)cc3nc3ccc(F)cc23)CC1]

Following the general procedure of Example 1 and making non-critical variations but using 6,9-dichloro-2-fluoroacridine and 2-(4-methylpiperazin-1-yl)ethanamine, the title compound was obtained; MS (Found M+1=373).

### Example 18

### N-(1-Ethylpiperidin-4-yl)-6-fluoro-2-methoxyacridin-9-amine

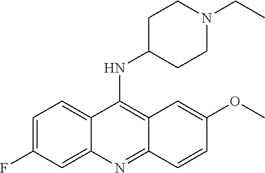 [SMILES: CCN1CCC(Nc2c3ccc(F)cc3nc3ccc(OC)cc23)CC1]

Following the general procedure of Example 1 and making non-critical variations but using 9-dichloro-6-fluoro-2-methoxyacridine and 1-ethylpiperidin-4-amine, the title compound was obtained; MS (Found M+1=354).

### Example 19

### 3-Chloro-N-(2-(4-methylpiperazin-1-yl)ethyl)acridin-9-amine

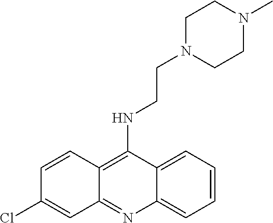 [SMILES: CN1CCN(CCNc2c3ccccc3nc3cc(Cl)ccc23)CC1]

Following the general procedure of Example 1 and making non-critical variations but using 3,9-dichloroacridine (J. Med. Chem. 1985, 28. 940-944) and 2-(4-methylpiperazin-1-yl)ethanamine, the title compound was obtained; MS (Found M+1=355).

### Example 20

### 6-Fluoro-2-methoxy-N-(2-(4-methylpiperazin-1-yl)ethyl)acridin-9-amine

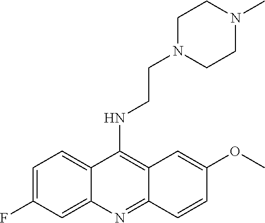 [SMILES: COc1ccc2nc3cc(F)ccc3c(NCCN3CCN(C)CC3)c2c1]

Following the general procedure of Example 1 and making non-critical variations but using 9-dichloro-6-fluoro-2-methoxyacridine and 2-(4-methylpiperazin-1-yl)ethanamine, the title compound was obtained; MS (Found M+1=369).

### Example 21

### 6-Chloro-2-methoxy-N-(2-(4-methylpiperazin-1-yl)ethyl)acridin-9-amine

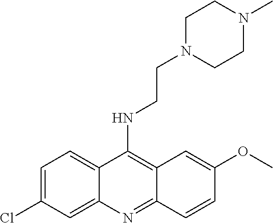 [SMILES: COc1ccc2nc3cc(Cl)ccc3c(NCCN3CCN(C)CC3)c2c1]

Following the general procedure of Example 1 and making non-critical variations but using 6,9-dichloro-2-methoxyacridine and 2-(4-methylpiperazin-1-yl)ethanamine, the title compound was obtained; MS (Found M+1=385).

### Example 22

### 6-Chloro-2-methoxy-N-(1-methylpiperidin-4-yl)acridin-9-amine

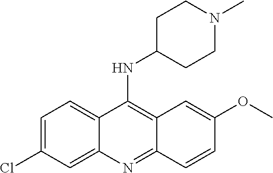 [SMILES: COc1ccc2nc3cc(Cl)ccc3c(NC3CCN(C)CC3)c2c1]

Following the general procedure of Example 1 and making non-critical variations but using 6,9-dichloro-2-methoxyacridine and 1-methylpiperidin-4-amine, the title compound was obtained; MS (Found M+1=356).

### Example 23

### 7-Chloro-2-methoxy-N-(2-(4-methylpiperazin-1-yl)ethyl)benzo[b][1,5]naphthyridin-10-amine

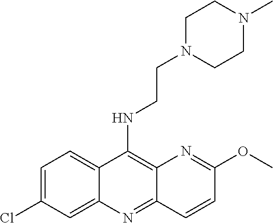 [SMILES: COc1ccc2nc3cc(Cl)ccc3c(NCCN3CCN(C)CC3)c2n1]

Following the general procedure of Example 1 and making non-critical variations but using 7,10-dichloro-2-methoxypyrido[3,2-b]quinoline and 2-(4-methylpiperazin-1-yl)ethanamine, the title compound was obtained; MS (Found M+1=386). ¹H NMR (DMSO-d6, 400 Hz): 8.43-8.41 (d, 1H, J=9.2 Hz), 8.12-8.10 (d, 1H, J=9.2 Hz), 7.87 (b, 1H), 7.2 (s, 1H), 7.2.9-7.27 (d, 1H, J=9.2 Hz) 7.25-7.24 (d, 1H, J=9.2 Hz), 4.10 (m, 2H), 4.06 (s, 3H), 2.70-2.68 (m, 2H), 2.33 (b, 8H), 2.14 (s, 3H).

### Example 24

### 7-Chloro-N-(1-ethylpiperidin-4-yl)-2-methoxybenzo[b][1,5]naphthyridin-10-amine

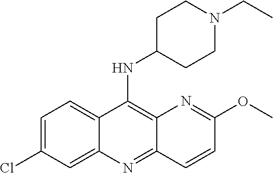 [SMILES: CCN1CCC(Nc2c3ccc(Cl)cc3nc3ccc(OC)nc23)CC1]

Following the general procedure of Example 1 and making non-critical variations but using 7,10-dichloro-2-methoxypyrido[3,2-b]quinoline and 1-ethylpiperidin-4-amine, the title compound was obtained; MS (Found M+1=371). ¹H NMR (DMSO-d6, 400 Hz): 8.43-8.42 (d, 1H, J=9.2 Hz), 8.11-8.10 (d, 1H, J=9.2 Hz), 7.84 (s, 1H), 7.37-7.35 (d, 1H, J=9.2 Hz) 7.25-7.23 (d, 1H, J=9.2 Hz), 6.95 (b, 1H), 4.98 (b, 1H), 4.00 (s, 3H), 2.85 (b, 2H), 2.30 (b, 2H), 2.02-1.99 (m 4H), 1.00-1.97 (t, 3H, J=7.2 Hz).

### Example 25

### N-(1-Ethylpiperidin-4-yl)-2-methoxyacridin-9-amine

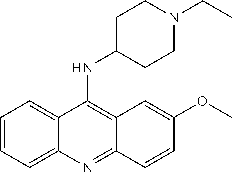 [SMILES: CCN1CCC(Nc2c3ccccc3nc3ccc(OC)cc23)CC1]

Following the general procedure of Example 1 and making non-critical variations but using 9-chloro-2-methoxyacridine and 1-ethylpiperidin-4-amine, the title compound was obtained; MS (Found M+1=336). ¹H NMR (CD₃OD, 400 Hz): 8.2.9-8.27 (d, 1H, J=8.8 Hz), 7.96-7.94 (d, 1H, J=8.8 Hz), 7.92-7.89 (d, 1H, J=9.6 Hz), 7.70-7.66 (m, 1H), 7.53 (m, 1H), 7.46-7.43 (m, 2H), 3.98 (s, 3H), 3.90-3.80 (m, 1H), 3.02-3.8 (bm, 2H), 2.46-2.41 (q, 2H, J=7.2 Hz), 2.05-2.00 (m, 4H), 1.93-1.83 (m, 2H), 1.11-1.08 (t, 3H, J=7.2 Hz).

### Example 26

### 6-Chloro-N-(1-ethylpiperidin-4-yl)-1,2,3,4-tetrahydroacridin-9-amine

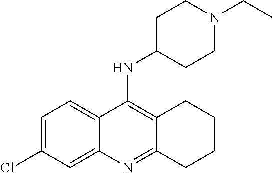 [SMILES: CCN1CCC(Nc2c3c(nc4cc(Cl)ccc24)CCCC3)CC1]

Following the general procedure of Example 1 and making non-critical variations but using 6,9-dichloro-1,2,3,4-tetrahydroacridine and 1-ethylpiperidin-4-amine, the title compound was obtained; MS (Found M+1=344).

### Example 27

### 6-Chloro-N-(2-(4-methylpiperazin-1-yl)ethyl)-1,2,3,4-tetrahydroacridin-9-amine

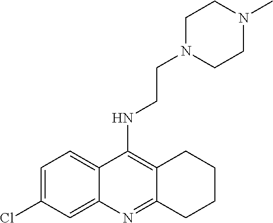 [SMILES: CN1CCN(CCNc2c3c(nc4cc(Cl)ccc24)CCCC3)CC1]

Following the general procedure of Example 1 and making non-critical variations but using 6,9-dichloro-1,2,3,4-tetrahydroacridine and 2-(4-methylpiperazin-1-yl)ethanamine, the title compound was obtained; MS (Found M+1=359). ¹H NMR (CDCl3, 300 Hz): 7.97-7.95 (m, 2H, J=9 Hz), 7.95-7.91 (d, 1H, J=9 Hz), 7.28-7.25 (m, 1H), 5.25 (b, 1H), 3.58-3.49 (m, 2H), 3.05 (m, 2H), 3.73 (m, 2H), 2.63-2.59 (m 10H), 2.39 (s, 3H), 1.94-1.90 (m, 4H).

### Example 28

### 6-Chloro-2-methoxy-N-(1-methylpyrrolidin-3-yl)acridin-9-amine

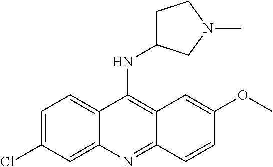 [SMILES: COc1ccc2nc3cc(Cl)ccc3c(NC3CCN(C)C3)c2c1]

Following the general procedure of Example 1 and making non-critical variations but using 6,9-dichloro-2-methoxyacridine and 1-methylpyrrolidin-3-amine, the title compound was obtained; MS (Found M+1=356)

### Example 29

### 6-Chloro-2-fluoro-N-(1-(4-methylpiperazin-1-yl)propan-2-yl)acridin-9-amine

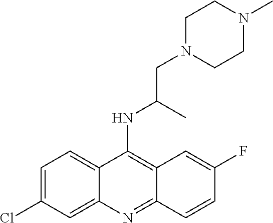 [SMILES: CC(CN1CCN(C)CC1)Nc1c2ccc(Cl)cc2nc2ccc(F)cc12]

Following the general procedure of Example 1 and making non-critical variations and starting with the appropriate starting materials, the title compound was obtained.

Examples 30-56 in Table 1 were prepared according to the above examples using appropriate starting materials. MS data is summarized for the compounds in Table 2.

TABLE 2

| Example | MS m/z (M + 1) |
|---|---|
| 30 | 334.2 |
| 31 | 352.1 |
| 32 | 368.2 |
| 33 | 364.2 |
| 34 | 443.3 |
| 35 | 382.1 |
| 36 | 386.1 |
| 37 | 403.1 |
| 38 | 368.2 |
| 39 | 385.1 |
| 40 | 369.2 |
| 41 | 403.2 |
| 42 | 399.2 |
| 43 | 372.1 |
| 44 | 358.1 |
| 45 | 384.2 |
| 46 | 370.2 |
| 47 | 412.2 |
| 48 | 426.2 |
| 49 | 442.1 |
| 50 | 442.1 |
| 51 | 428.2 |
| 52 | 426.2 |
| 53 | 462.1 |
| 54 | 384.2 |
| 55 | 385.1 |
| 56 | 384.1 |
| 64 | 400.0 |

### Biological Example 1

Tumor cell lines (H292, HCT116, A375, HCC1569, A498, N87, UACC1093, and UACC647) were cultured in RPMI 1640 supplemented with 5% fetal bovine serum and housed in a 5% CO₂ Incubator at 37° C.

For single agent IC₅₀ determination, cells were plated on a 96 well microplate and allowed 24 hours to adhere. Drugs were administered to the drug plate by the following: compound stock solutions (10 mM) were added to a drug plate where a 1:10 dilution was performed. Following the dilutions, 2 μl of test compound was transferred to the corresponding wells in the cell-containing 96-well plate with 198 μl of growth media. The compounds were tested over a range of 0.1 pM-100 μM for 72 hours. Following 72 hours of continuous exposure cell viability was determined by measuring the ATP activity using a commercially available cell viability assay kit. Luminescence intensity was used to relative drug activity compared to control wells and used to graphically determine the IC₅₀.

For combination interaction experiments, cells were plated on a 96 well microplate and allowed 24 hours to adhere. Drugs were administered to the drug plate by the following: compound stock solutions (20 mM) or combination agent stock solutions (20 mM) were added to a drug plate where a 1:10 dilution was performed. Following the dilutions, 2 μl of each VT-062 and standard agent was transferred to the corresponding wells in the cell-containing 96-well plate with 196 μl of growth media. The compound and combination agent was concurrently tested over a range of 0.1 pM-100 μM for 72 hours. Following 72 hours of continuous exposure cell viability was determined by measuring the ATP activity using a commercially available cell viability assay kit. Luminescence intensity was used to relative drug activity compared to control wells and used to graphically determine the IC₅₀ of combination.

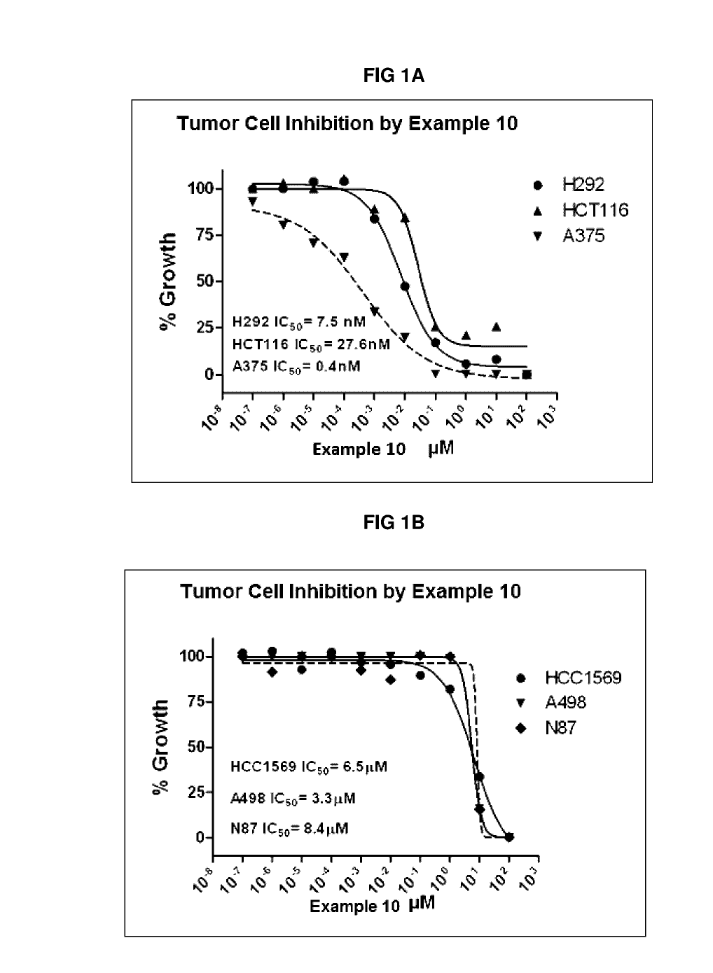

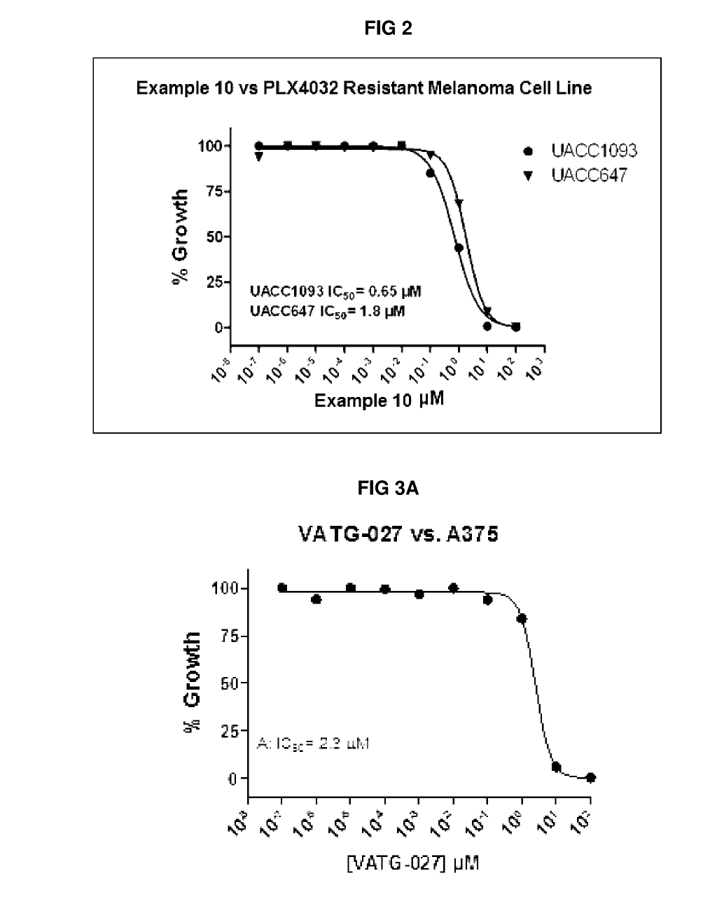

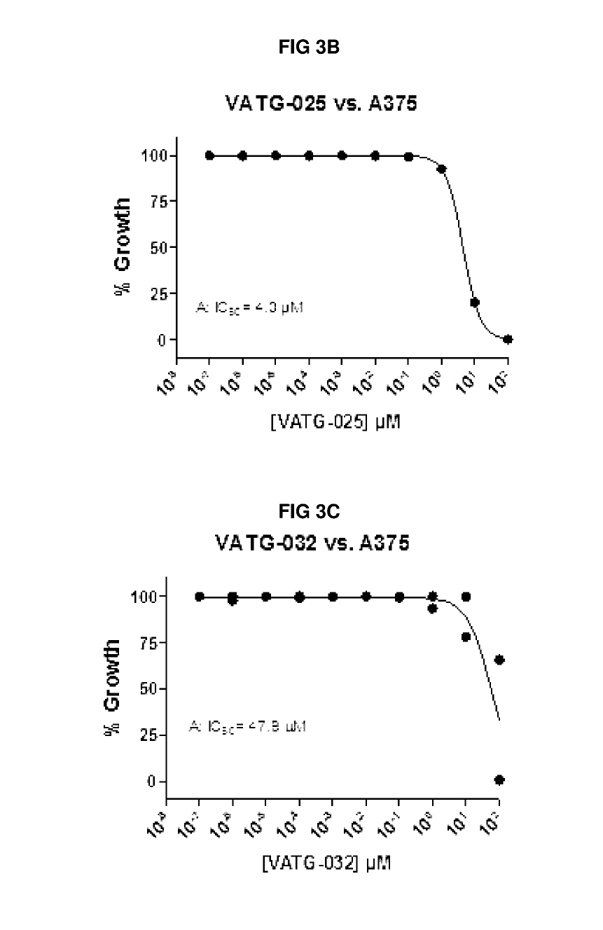

FIGS. 1A, 1B, and 1C show that Example 10 inhibited tumor cell growth by more than 75% in the cell lines tested. FIGS. 3A, 3B, and 3C show similar tumor growth inhibition for Examples 7, 26, and 27 in A375 tumor cells.

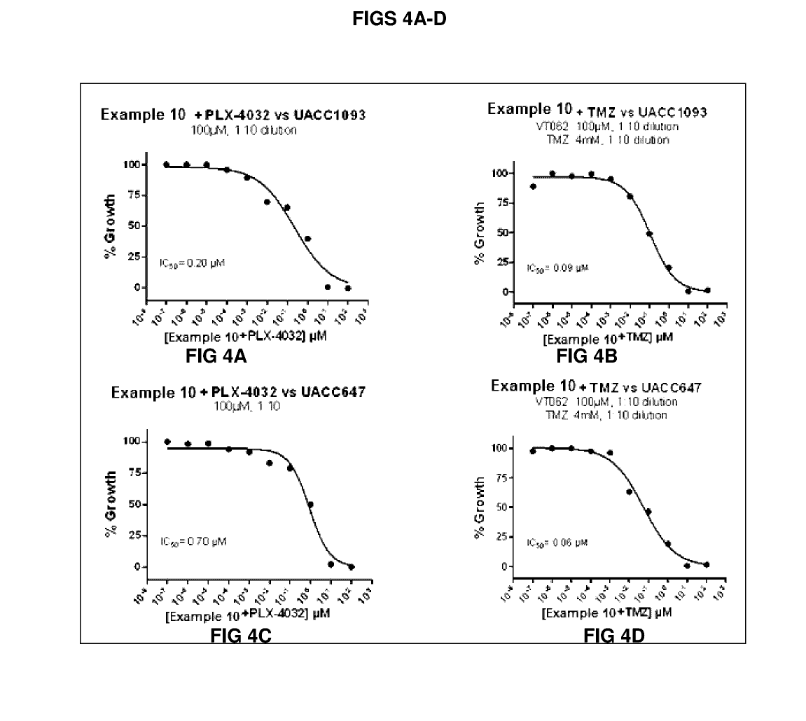

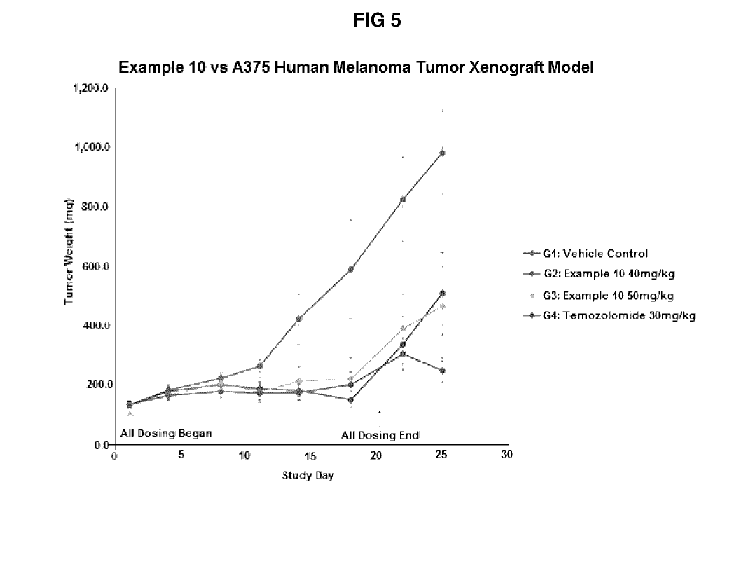

FIGS. 4A and 4C show that Example 10 in combination with PLX-4032 had a 2.6-3.25% increase in tumor growth inhibition against in combination than Example 10 alone in PLX-4032 resistant melanoma cell lines. FIGS. 4B and 4D show that Example 10 in combination with Temozolomide had a 7-30% increase in tumor growth inhibition in combination than Example 10 alone in those same cell lines.

### Biological Example 2

### Autophagy Inhibition Screen and Quantification

U2OS cells stably expressing ptfLC3 (Adgene plasmid 21074)(Kimura, et al., 2007) were seeded at 5,000 cells per well in 5A McCoy's medium (Invitrogen, Carlsbad, Calif.) with 10% fetal bovine serum (FBS, Invitrogen) in 96-well glass bottom tissue culture plates for 24 hours at 37° C. and 5% CO₂. Cells were treated with VATG compounds in a 10-point dose response for three hours, fixed with 3.7% formaldehyde, and nuclei were stained with Hoechst 33342 (Invitrogen). Cells were visualized using a 60× oil-immersion objective on a Nikon fluorescent microscope and pftLC3 fluorescence was compared with that of a DMSO vehicle control within each plate. Doses were qualitatively scored based on increased accumulation of ptfLC3 labeled punctae from zero punctae and higher.

An ED was then established for each compound. The compounds were repeated alongside chloroquine and quinacrine on U2OS cells seeded at 50,000 cells per well in 5A McCoy's with 10% FBS on number 1.5 coverglasses in 24-well tissue culture dishes. After 24 hours, the cells were treated at set doses of 0.3 uM, 1 uM, 3 uM, 10 uM, and 30 uM for three hours for confirmation. Cells were washed with 1×PBS, fixed with 3.7% formaldehyde, and nuclei were stained with Hoechst 33342 (2 ug/mL). Coverglasses were inverted onto a microscope slide using mounting gel. The microscope slides were imaged using a 60× oil-immersion objective on a Nikon Eclipse Ti fluorescent microscope and 10 images at each dose were taken for quantification. Image processing and quantification were completed with the Nikon NIS Elements software. To quantify, images were deconvoluded using a 2D blind deconvolution function with one iteration and settings of normal cell thickness and normal noise level. Regions of interest (ROI) were drawn around the edges of each cell excluding the nuclear region. Intensity thresholds were set to include all pixels equal to or greater than the intensity above the mean background fluorescence. Objects within the threshold ROIs were quantified using an automated object count function and exported to Excel (Microsoft). Although other parameters were also collected, the mean intensity of the objects was averaged between the 10 images of each dose, or approximately 35 cells. Representative images were chosen for each dose and the LUTs were set based on the mean intensity of the DMSO control. The mean intensity of each image was divided by the mean intensity of the DMSO control and the LUTs were adjusted by the percent difference to avoid viewing the background intensity.

The quantified ED₅₀ values are shown in Table 3.

TABLE 3

| Example | Autophagy Inhibition (ED50) |
|---|---|
| 1 | ***** |
| 2 | ***** |
| 3 | * |
| 4 | ***** |
| 5 | ***** |
| 6 | * |
| 7 | ***** |
| 8 | *** |
| 9 | * |
| 10 | ***** |
| 11 | * |
| 12 | *** |
| 13 | ***** |
| 14 | * |
| 15 | ***** |
| 16 | *** |
| 17 | *** |
| 18 | *** |
| 19 | *** |
| 20 | *** |
| 21 | ***** |
| 22 | *** |
| 23 | ***** |
| 24 | *** |
| 25 | ***** |
| 26 | ***** |
| 27 | *** |
| 28 | *** |

### Biological Example 3

### Human Tumor Xenograft Study

Female mice were inoculated subcutaneously in the right flank with 0.1 ml of a 50% RPMI/50% Matrigel™ (BD Biosciences, Bedford, Mass.) mixture containing a suspension of A375 Human melanoma tumor cells (approximately 5×10⁶ cells/mouse).

When tumor reached approximately 130 mg, mice were randomized into treatment groups. Body weights were recorded when the mice were randomized and were taken twice per week (on Study Days 3 and 7 for each cycle) thereafter in conjunction with tumor measurements. Treatment began on the day of randomization. Example 10 was delivered orally in a vehicle consisting of 5% DMA, 10% propylene glycol, 20% PEG 400, and 65% sterile water. Example 10 was administered daily for 21 days.

FIG. 5 shows that tumor weight in mice over the course of 25 days. Example 10 inhibited tumor growth by greater than 50% after 25 days.

### Biological Example 4

### Autophagy Inhibition Screen and Quantification

U2OS cells stably expressing ptfLC3 (Adgene plasmid 21074)(Kimura, et al., 2007) were seeded at 5,000 cells per well in 5A McCoy's medium (Invitrogen, Carlsbad, Calif.) with 10% fetal bovine serum (FBS, Invitrogen) in 96-well glass bottom tissue culture plates for 24 hours at 37° C. and 5% CO₂. Cells were treated with compounds in a 10-point dose response for three hours, fixed with 3.7% formaldehyde, and nuclei were stained with Hoechst 33342 (Invitrogen). Cells were visualized using a 60× oil-immersion objective on a Nikon fluorescent microscope and pftLC3 fluorescence was compared with that of a DMSO vehicle control within each plate. Doses were qualitatively scored based on increased accumulation of ptfLC3 labeled punctae from zero punctae and higher. An ED was established for each compound.

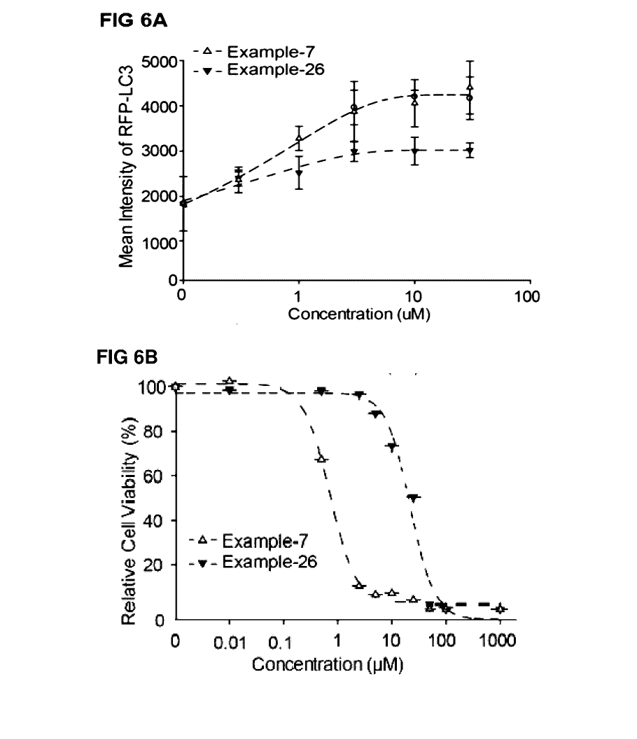

The compounds selected, Example 7 and Example 26, were repeated on U2OS cells seeded at 50,000 cells per well in 5A McCoy's with 10% FBS on number 1.5 coverglasses in 24-well tissue culture dishes. After 24 hours cells were treated at set doses of 0.3 uM, 1 uM, 3 uM, 10 uM, and 30 uM for three hours for confirmation. Cells were washed with 1×PBS, fixed with 3.7% formaldehyde, and nuclei were stained with Hoechst 33342 (2 ug/mL). Coverglasses were inverted onto a microscope slide using mounting gel. The microscope slides were imaged using a 60× oil-immersion objective on a Nikon Eclipse Ti fluorescent microscope and 10 images at each dose were taken for quantification. Image processing and quantification were completed with the Nikon NIS Elements software. To quantify, images were deconvoluded using a 2D blind deconvolution function with one iteration and settings of normal cell thickness and normal noise level. Regions of interest (ROI) were drawn around the edges of each cell excluding the nuclear region. Intensity thresholds were set to include all pixels equal to or greater than the intensity above the mean background fluorescence. Objects within the threshold ROIs were quantified using an automated object count function and exported to Excel (Microsoft). Although other parameters were also collected, the mean intensity of the objects was averaged between the 10 images of each dose, or approximately 35 cells. Representative images were chosen for each dose and the LUTs were set based on the mean intensity of the DMSO control. The mean intensity of each image was divided by the mean intensity of the DMSO control and the LUTs were adjusted by the percent difference to avoid viewing the background intensity. FIG. 6A shows the mean intensities for Example 7 and Example 26.

### Biological Example 5

### Cell Viability (LD₅₀) Screen

U2OS cells were seeded at 500 cells per well in 5A McCoy's with 10% FBS in 96-well clear bottom, black-walled tissue culture plates. After 24 hour incubation, cells were treated with compounds in triplicate with a 10-point half log dose response for 24 and 48 hours. Medium was removed with 2× CellTiter Glo (Promega) reagent mixed 1:1 with optimem (Invitrogen) was added at 100 uL per well and incubated rocking at room temperature for 15 minutes. A total of 75 uL per well was moved to a white-walled 96-well plate and read using the 96 LUM program on an EnVision plate reader 0 and exported to Excel (Microsoft) for analysis. FIG. 6B shows the relative cell viabilities of Example 7 and Example 26.

### Biological Example 6

### Demonstrating an Inhibitory Lysosomal Mechanism of Action

To determine deacidification of the lysosome, cells were incubated with LysoTracker Red, a dye that localizes to the lysosome based on the low acidity of the compartment. If the lysosome is no longer acidic, there is a loss in the amount of LysoTracker Red staining. Lysosomal inhibition was further determined by immunofluorescence of lysosome-associated membrane protein-1 (LAMP1). If the lysosome is inhibited, lysosomal turnover should decrease and an increase in the amount of LAMP1 staining would be apparent. U2OS cells were treated with Example 7 or Example 26 at 3 μM for 3 hours, supplementing LysoTracker Red for the final hour. Example 7 and Example 27 treatments all caused substantial increases in LAMP1 staining and essentially ablated LysoTracker Red staining, indicating that the compounds inhibit lysosomal turnover through deacidification.

Using image analysis software, the mean intensities of both the LAMP1 and LysoTracker Red staining were measured and represent altered intensity level between treatments on intensity plots. Individual points were measured using a line scan on the image analysis software, which measures the intensity across the path of a line at any given point. Not only does the presence of LAMP1 positive membranes increases, but the intensity in LAMP1 staining also increases with Example 7 and Example 26 treatment. The inverse relationship in intensity holds true for LysoTracker Red staining. Treatment with Example 7 and Example 26 showed less intense LysoTracker Red staining, indicating an increase in pH.

### Biological Example 7

### Inhibition Screens

U2OS cells stably expressing tfLC3 (Addgene, plasmid 21074)⁵⁸ were seeded at 5,000 cells per well in 5A McCoy's medium (Invitrogen, 16600-082) with 10% fetal bovine serum [FBS (CellGro, 35-101-CV)] in 96-well glass bottom tissue culture plates for 24 hours at 37° C. and 5% CO₂. Cells were treated with a selection of commonly used anti-malarial compounds (amodiaquine, artemisinin, chloroquine, mefloquine, primaquine, piperaquine, and quinacrine) in a 6-point dose response for three hours, fixed with 3.7% formaldehyde, and nuclei were stained with Hoechst 33342 (2 μg/mL: Invitrogen, H1399). Cells were visualized using a 60× oil-immersion objective on a Nikon Eclipse Ti fluorescent microscope. Doses were qualitatively scored for effective concentration (EC), defined as the concentration at which there was a statistically significant accumulation of tfLC3-labeled puncta over vehicle controls. Cells were later treated with compounds of Formula A (Ex. 7 and 27), in a 6-point dose response for three hours, fixed, and visualized. An EC was established for each of Example 7 and Example 27 compounds.

### Biological Example 8

### Chemical Synthesis of Autophagy Inhibitors

Anti-malarial drugs shown in Table 4 are all commercially available: amodiaquine (Chempacific, Corp. 35393), artemsinin (Sigma, 361593), chloroquine (Sigma, C6628), mefloquine (Amplachem, Inc., AA-90157), primaquine (OChem, Inc., 598P906), piperaquine (AK Scientific, H853), and quinacrine (TCI America, Q0056). VATG027 and VATG032 were synthesized as follows: Synthesis of Example 7 (VATG027): A mixture of 6,9-dichloro-2-methoxyacridine (100 mg, 0.36 mmol) and phenol (approximately 1.5 g) was heated to 100° C. under nitrogen atmosphere and stirred for 1 hour. To this mixture was added 4-(4-methylpiperazin-1-yl)butan-1-amine (123 mg, 0.72 mmol). The reaction was stirred at 100° C. for 5 hours, cooled to 20-25° C., and diluted with dichloromethane. The mixture was washed twice with sodium hydroxide solution (1 N) and twice with ammonium chloride solution. The phases were separated, and the organic layer was dried and concentrated. The residue was purified by Biotage column chromatography using triethylamine (5%) and methanol (0 to 10%) in dichloromethane to give Example 7 (92 mg, yield: 62%); MS (Found M+1=413); Synthesis of Example 27 (VATG032): Following the procedure of the synthesis of Example 7, but using 6,9-dichloro-1,2,3,4-tetrahydroacridine and 2-(4-methylpiperazin-1-yl)ethanamine as the starting material. MS (Found M+1=359).

### Biological Example 9

### Quantification of Autophagy Activity

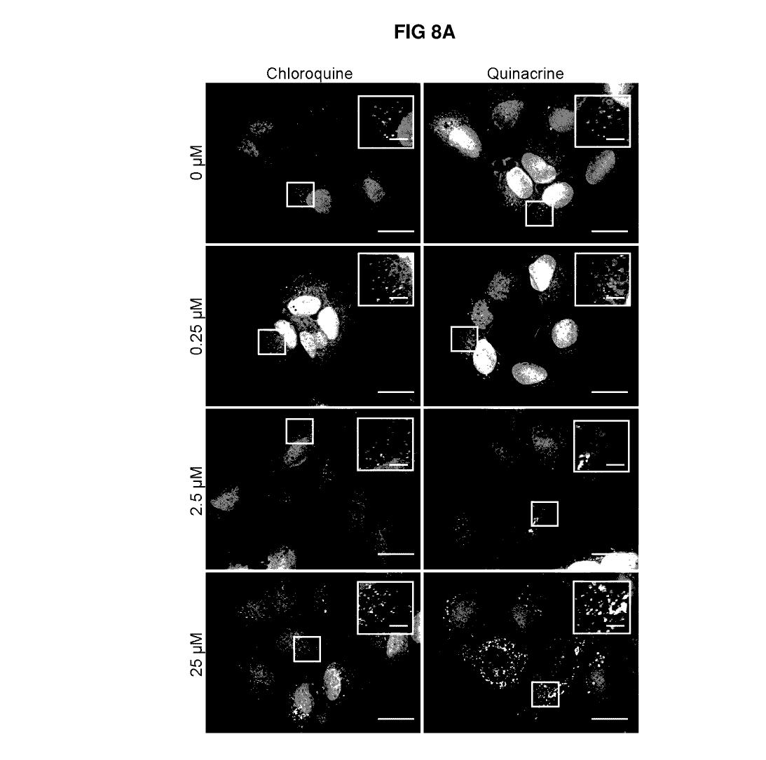

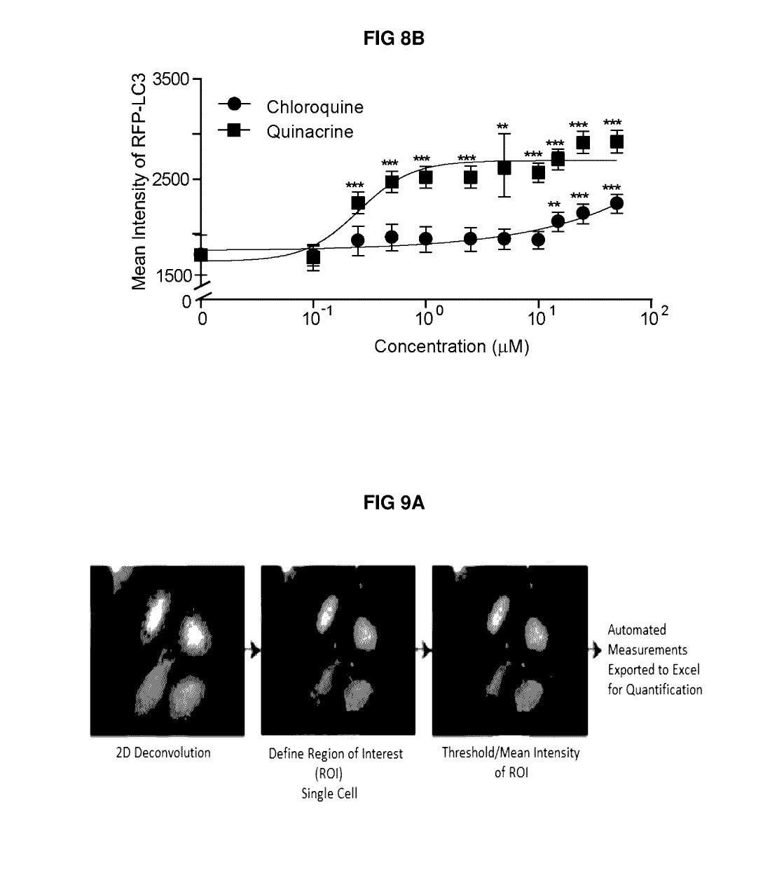

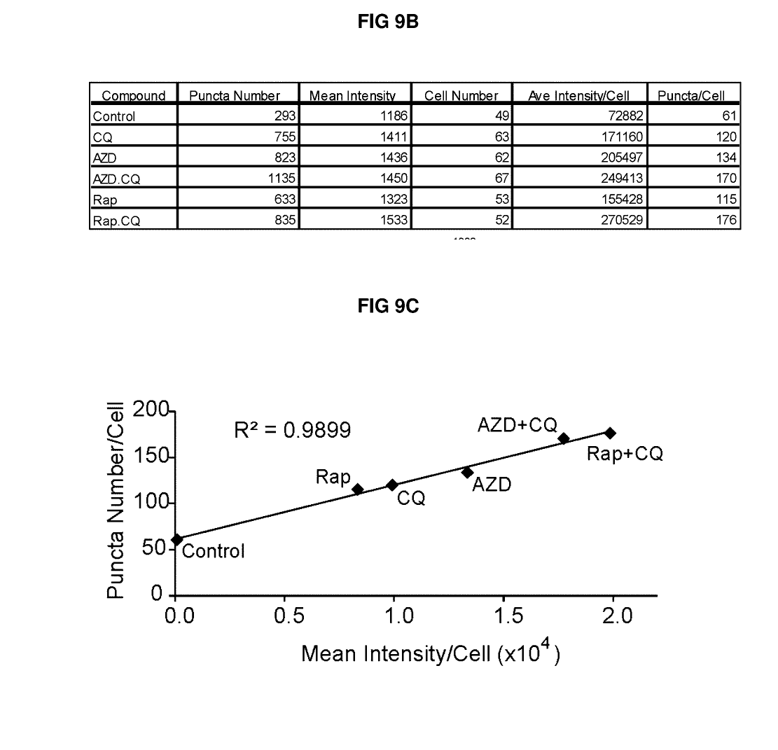

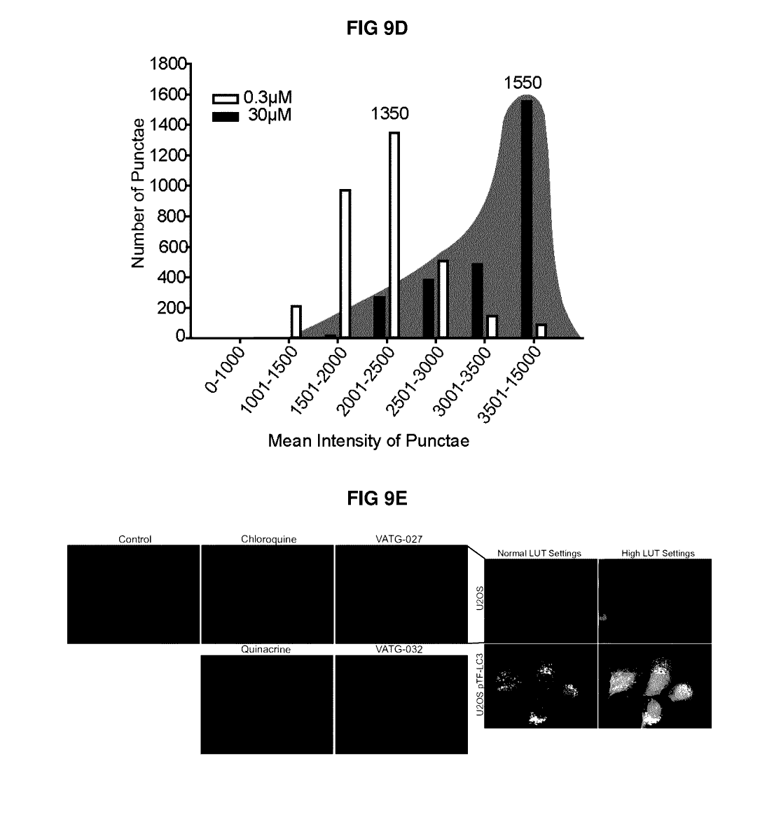

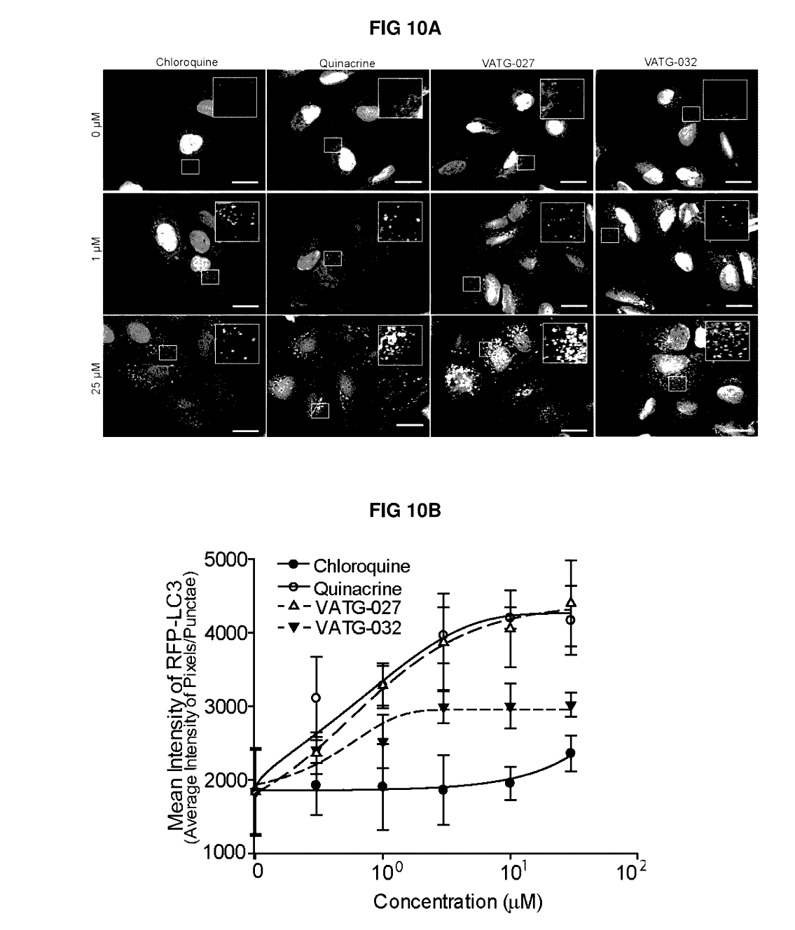

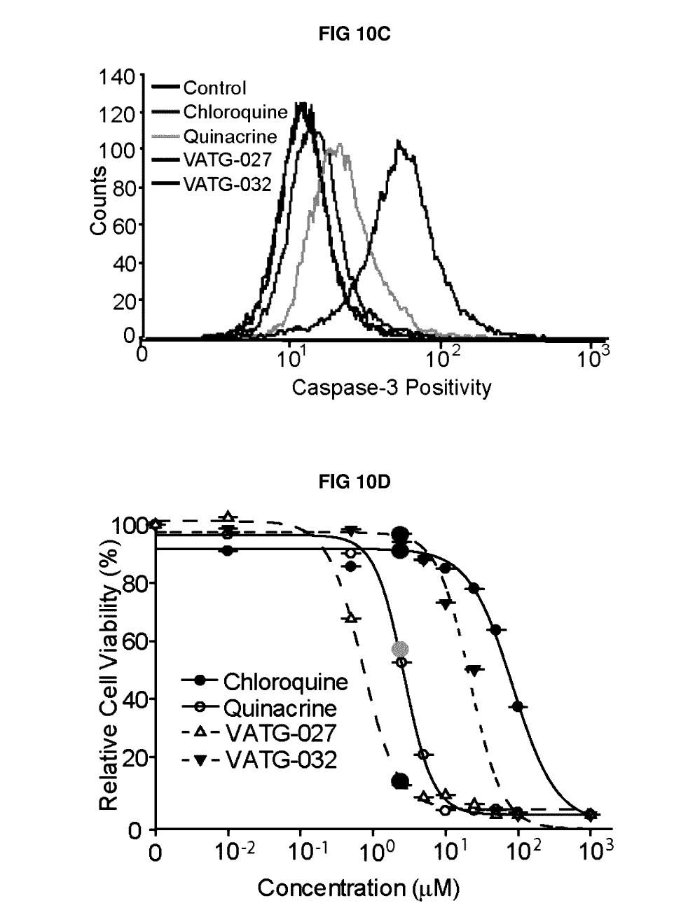

U2OS cells stably expressing tfLC3 were seeded at 50,000 cells per well in 5A McCoy's with 10% FBS on number 1.5 coverglass in 24-well tissue culture dishes. After 24 hours, cells were treated with known autophagy controls rapamycin [100 nM] (Millipore, 553210-10 mg), bafilomycin A1 [100 nM] (AG Scientific, B-1183), AZD-8055 [100 nM](Selleck Chemicals, S1555), and CQ [50 μM] as well as autophagy inhibitors (CQ, QN, Example 7, and Example 27) at doses of 0.1 μM, 0.25 μM, 0.5 μM, 1 μM, 5 μM, 15 μM, 25 μM, and 50 μM for three hours. Cells were washed with 1×PBS, fixed with 3.7% formaldehyde, and nuclei were stained with Hoechst 33342 (2 μg/mL). Coverglass was inverted onto microscope slides using mounting gel. Cells were imaged using a 60× oil-immersion objective on a Nikon Eclipse Ti fluorescent microscope and 10 images at each dose were taken for quantification. Image processing and quantification were completed with the Nikon NIS Elements software. To quantify, images were deconvolved using a 2D blind deconvolution function with one iteration and settings of normal cell thickness and normal noise level. Regions of interest (ROI) were drawn around the edges of each cell. Intensity thresholds were set to include all pixels equal to or greater than the intensity above the mean background fluorescence using the separation feature and restrictions set for puncta size (FIG. 9). Objects within the threshold for each ROI were quantified using an automated object count function and exported for analysis. Although other parameters were also collected, the mean intensity of the objects was averaged between the 10 images of each dose, or approximately 50 cells (FIGS. 9B and 9C). Representative images were chosen for each dose and the lookup table (LUT) brightness' were set based on the mean intensity of the DMSO control (FIGS. 8A and 10A). The mean intensity of each image was divided by the mean intensity of the DMSO control to control for brightness and the LUTs were adjusted by the percent difference to avoid background and for consistent visualization. All other settings (gain, exposure time, and lamp strength) were kept the same across all conditions. Puncta number was used to determine an effective dose (EC), which is the statistically significant (p-value≦0.05) increase in RFP-GFP-LC3 labeled puncta number compared to DMSO control, determined by a student t-test. Mean intensity was further chosen for quantification as it accurately represents both the increase in puncta number and area when the accumulation of autophagosomes partially fuse (FIG. 9C, 9D). Quantification of the red channel (RFP-LC3 puncta) was performed to determine the total autophagic vesicle population (both autophagosomes and autolysosomes). Auto-fluorescence of each compound was tested using wild-type U2OS cells to confirm that compound auto-fluorescence did not interfere with ptfLC3 quantification (FIG. 9E).

### Biological Example 10

### Cell Viability (IC₅₀) Screen

U2OS cells were seeded at 500 cells per well in 5A McCoy's with 10% FBS in 96-well clear bottom, black-walled tissue culture plates. All melanoma cell lines (A375, UACC-91, UACC-257, UACC-502, UACC-903, UACC-1308, UACC-1940, UACC-2534, and UACC-3291) were seeded at 5,000 cells per well in RPMI 1640 with 10% FBS in 96-well clear bottom, black-walled tissue culture plates. After 24 hour incubation, cells were treated with VATG compounds in triplicate with a 10-point half log dose response from 0.001 μM to 1000 μM for 24 and 48 hours. Medium was removed and 2× CellTiter Glo (Promega, G7571) reagent mixed 1:1 with Opti-MEM (Invitrogen, 31985062) was added at 100 μL per well and incubated at room temperature for 15 minutes while rocking. 75 μL per well was moved to a white-walled 96-well plate and luminescence quantified using the 96 LUM program on an EnVision plate reader (PerkinElmer) and exported for analysis. All triplicate data points were averaged and luminescent readings for each treatment were normalized to vehicle control for change in viability. Dose response curves were input into SigmaPlot for IC₅₀ calculations (Systat Software Inc).

### Biological Example 11

### FACS Analysis

U2OS cells were seeded in a 6 well plate in 5A McCoy's with 10% FBS at 100,000 cells per well. After 24 hours incubation, cells were treated at 1 μM, 3 μM, 10 μM, and 30 μM with CQ, QN, VATG-027, or VATG-032 for 48 hours. Media was collected and spun down discarding the supernatant to collect floating cells. Wells were treated with 250 μL 0.25% Trypsin-EDTA and cells were again collected, spun down, and supernatant discarded. Cells were then fixed in 5 mL of 70% ethanol and stored at −20° C. for 24 hours. Cells were centrifuged at 300 g for 5 min and resuspended in 1 mL 90% chilled methanol. After 30 minutes, cells were washed twice in 3 mL incubation buffer (0.5 g BSA in 100 mL 1×PBS) and resuspended in 100 μL incubation buffer for 10 minutes. Cells were then incubated with the primary cleaved caspase-3 antibody (Cell Signal Technology, 9661S) at 1:1000 in incubation buffer for one hour. Cells were washed (incubation buffer) and secondary anti-rabbit alexa 546 antibody added 1:1000 for 30 minutes. Cells were then washed and resuspended in 100 μL 1×PBS and acquired using a BD FACS Calibur.

### Biological Example 12

### Transmission Electron Microscopy

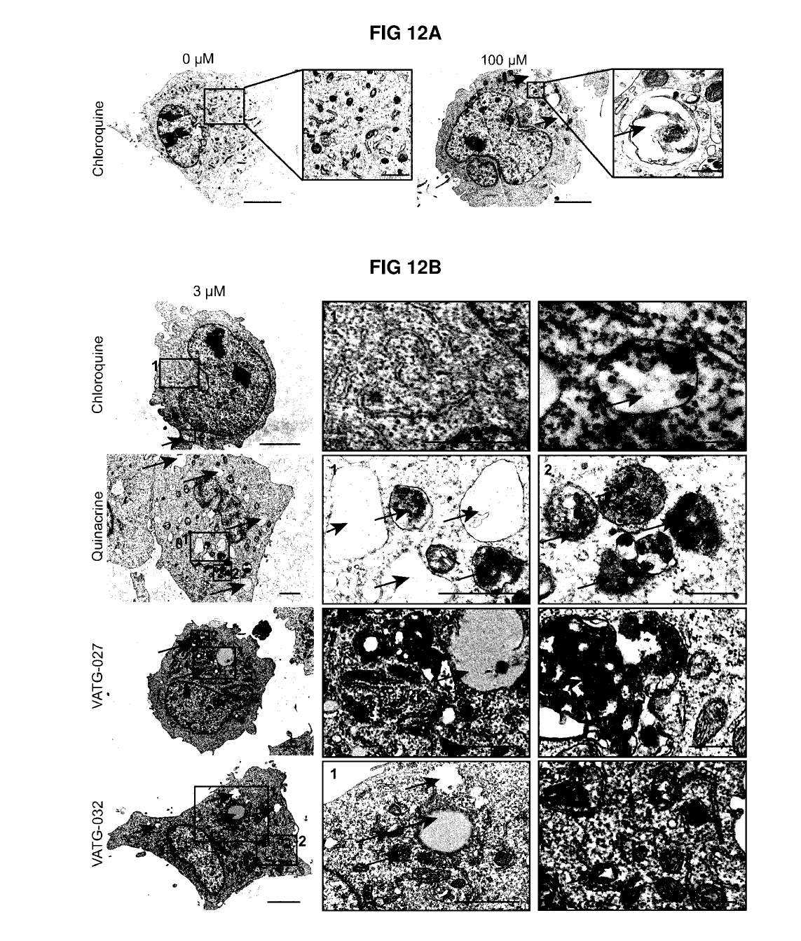

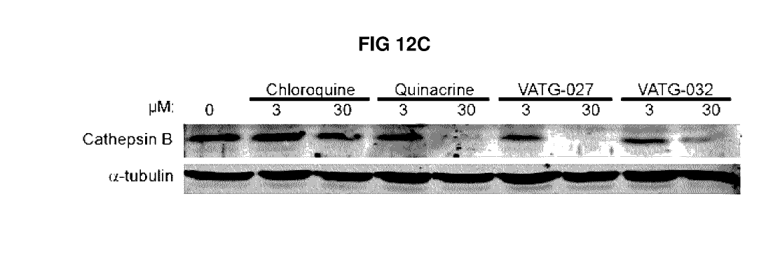

U2OS cells were seeded in 10 cm plates in 5A McCoy's with 10% FBS at 1×10⁶ cells per plate. After 24 hours incubation, cells were treated with DMSO (vehicle control), CQ (3 or 100 μM), quinicrine (3 μM), VATG-027 (3 μM), or VATG-032 (3 μM) for three hours. Following, cells were trypsinized, washed, pelleted, and resuspended in 2% glutaraldehyde fixative (Sigma, G5882). Cell pellets were embedded in 2% agarose, post-fixed in osmium tetroxide, and dehydrated with an acetone series. Cell samples were infiltrated and embedded in Poly/Bed 812 resin and polymerized at 60° C. for 24 hours. Ultrathin sections of 70 nm were generated with a Power Tome XL (Boeckeler Instruments) and placed on copper grids. Sections were examined using a JEOL 100Cx Transmission Electron Microscope at 100 kV. Lysosomal structures were identified by a single membrane structure lacking cytosolic components (FIG. 12). Transmission electron microscopy services were performed by Michigan State University Center for Advanced Microscopy (East Lansing, Mich.).

### Biological Example 13

### Lysosome Analysis by Fluorescent Microscopy

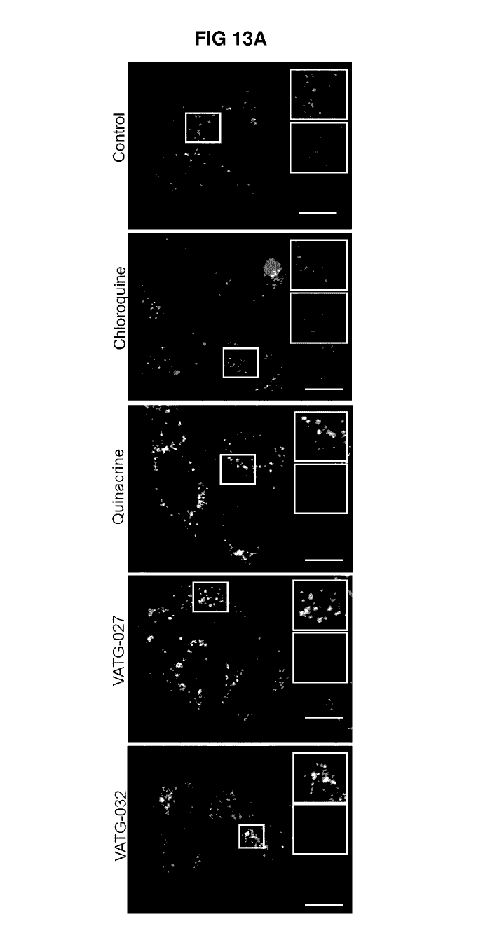

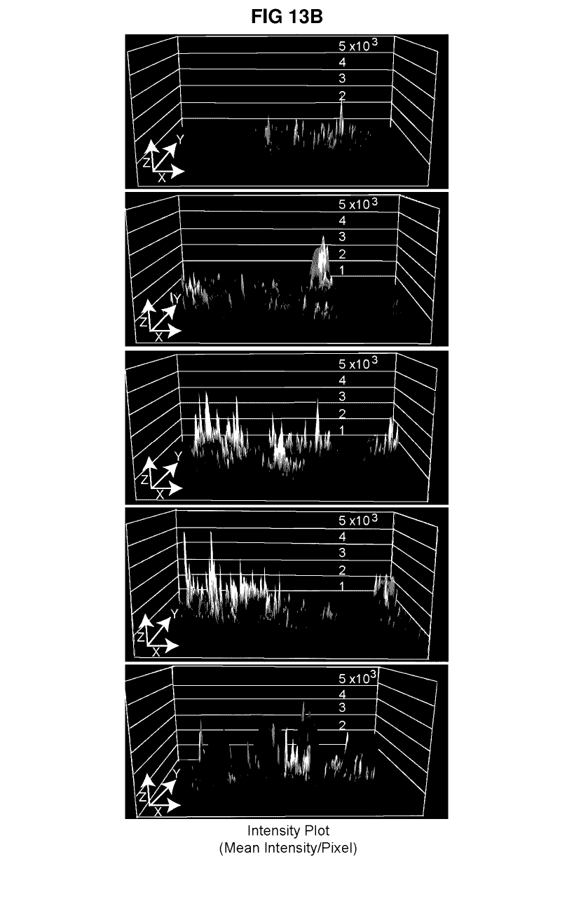

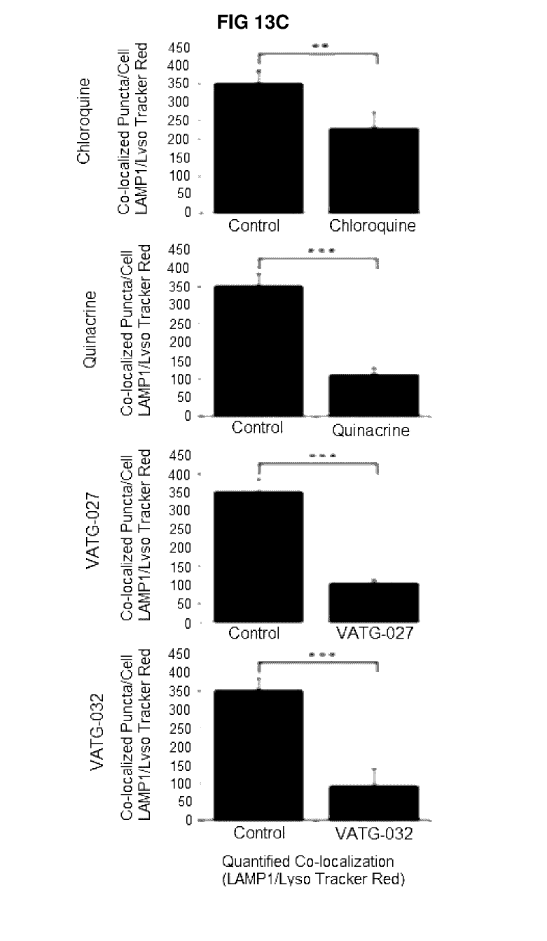

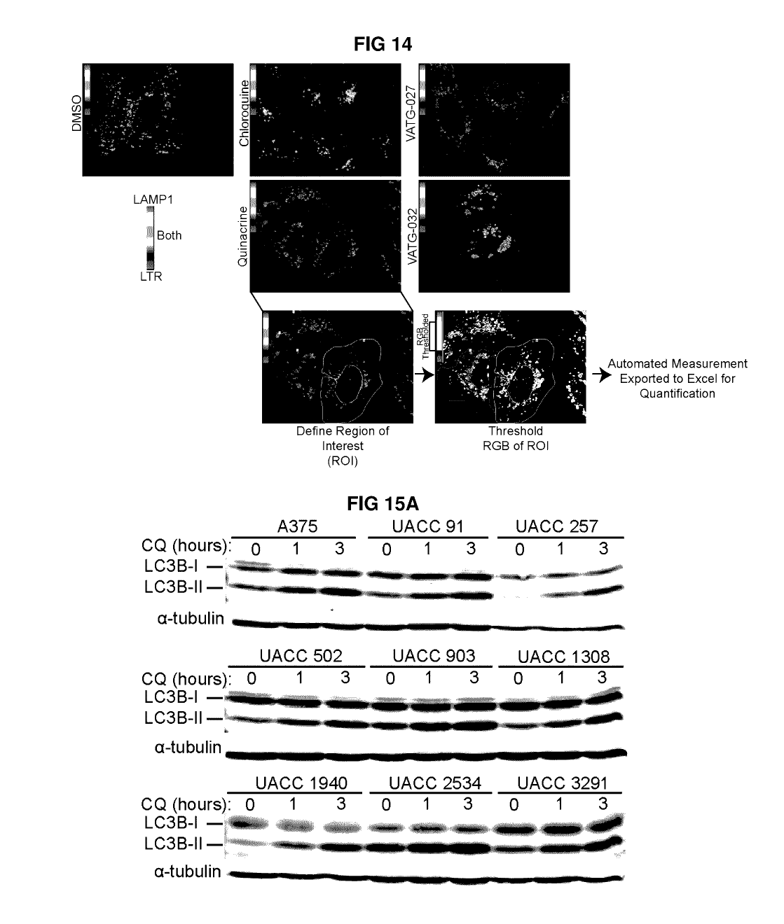

U2OS cells were seeded at 5×10⁴ cells per well in 5A McCoy's with 10% FBS on number 1.5 coverglass discs in 24-well tissue culture dishes. After 24 hours, cells were treated with 3 μM CQ, QN, VATG-027, or VATG-032 for three hours. An hour prior to fixation, media was supplemented with LysoTracker Red added at 100 nM (Invitrogen, L7528). Cells were washed with 1×PBS, fixed with 3.7% formaldehyde, permeabilized with 0.2% Triton-X 100, and blocked with 3% bovine serum albumin (BSA) in PBS. LAMP1 antibody (Santa Cruz, sc-18821) was added at 1:1000 for 16 hours at 4° C. followed by Alexa-Fluor-488-conjugated anti-mouse IgG (Invitrogen, A11008) 1:5000 for 1 hour at room temperature. Nuclei were stained with Hoechst 33342 (2 μg/mL). Coverglass discs were inverted onto a microscope slide using mounting gel. The microscope slides were imaged using a 60× oil-immersion objective on a Nikon Eclipse Ti fluorescent microscope. Intensity of the red and green channels were visualized using the intensity plot on the Nikon NIS Elements software (FIG. 13B). Co-localization was determined using the Nikon NIS Elements software by using the ratio feature which ratios the intensity of the green channel (LAMP1) over the red channel (LysoTracker Red) per pixel and displays it on a colorimetric scale (FIG. 14). The RGB threshold of only the color (green) indicating both LAMP1 and LysoTracker Red positivity was performed and data for analysis. The Mander's co-localization coefficient was produced from the Nikon NIS Elements software.

### Biological Example 14

### Western Blot Analyses

For Western blotting, U2OS cells were seeded in 10 cm plates in 5A McCoy's with 10% FBS at 1×10⁶ cells per plate. All melanoma lines were seeded in 10 cm plates in RPMI 1640 with 10% FBS at 1×10⁶ cells per plate. After 24 hours, cells were treated with CQ, QN, Example 27, or Example 7 in a dose response of 0.3 μM, 1 μM, 3 μM, 10 μM, and 30 μM for three hours. A375 were treated with PLX-4032 (Selleck Chemicals, S1267) at 10 nM, 100 nM, and 1 μM for three hours with and without CQ, QN, Example 7, and Example 27 at 3 μM and 30 μM. After treatment, cells were lysed [10 mM KPO4, 1 mM EDTA, 10 mM MgCl2, 5 mM EGTA, 50 mM bis-glycerophosphate, 0.5% NP40, 0.1% Brij35, 0.1% sodium deoxycholate, 1 mM NaVO4, 5 mM NaF, 2 mM DTT, and complete protease inhibitors (Sigma, P8340-5 mL)] and 50 μg of protein was resolved by SDS-PAGE. Proteins were transferred to PVDF membranes and probed with primary antibodies [LC3 (Sigma, L7543-200UL), anti-α-tubulin (Sigma, T6199), cathepsin B (Santa Cruz, sc-13985)] for 16 hours at 4° C. followed by a secondary antibody [HRP-linked rabbit or mouse IgG (GE Healthcare, NA934 or NA931) or Odyssey IRDye 680CW Goat anti-rabbit IgG (LI-COR, 926-32221) or IRDye 800CW Goat anti-mouse IgG (LI-COR, 926-32210)] for 1 hour at room temperature. Proteins were detected with enhanced chemiluminescence or using the Odyssey machine and quantified (FIGS. 12, 15, 16, 7 and 11).

### Biological Example 15

### Soft Agar Colony Formation Assay

In a 6-well plate, a solidified base was created using a 1:1 solution of RPMI 1640 (with 20% FBS) and 1% agarose solution. This was overlaid with A375 cells at 40,000 cells per well in RPMI 1640 (with 10% FBS) mixed 1:1 with 0.7% agarose. The soft agarose containing A375 cells was then overlaid with 0.75 mL RPMI 1640 (with 10% FBS) and incubated at 37° C. in 5% CO₂. After 24 hours, cells were treated in a dose response or at the determined LD10 with PLX-4032, CQ, QN, Example 7, or Example 27 every other day for three weeks. After three weeks of treatment, cells were fixed and stained in 1% paraformaldehyde in 1×PBS containing 0.005% crystal violet overnight. Cells were destained with multiple washes of 1×PBS to remove background staining. Plates were scanned and images quantified using Nikon NIS Elements software. To quantify, each image was sharpened and an ROI of equal size drawn around each well. Intensity thresholds were set to include all pixels equal to or greater than the intensity of the mean background fluorescence. Objects within the threshold of each ROI were quantified using an automated object count function and exported for analysis. Object number was quantified to denote total colony formation. Additivity was determined using the fractional product concept or Bliss independence model: E_{xy}=Eₓ+E_{y}−(EₓE_{y}) where E_{XY} is the additive effect of the two compounds x and y as calculated by the product of the individual effect of the two compounds, Eₓ and E_{y}. Additivity was established when the expected viability determined by the Bliss independence model was equal to the actual viability. This model was chosen since the effects of both compounds are mutually non-exclusive and follow first order Michaelis-Menten kinetics.

### Biological Example 16

### Demonstrating Autophagy Inhibition

Autophagy inhibition can be measured by fluorescent microscopy using cells expressing a tandem fluorescent (RFP-GFP) labeled LC3 sensor (tfLC3). Upon autophagosome nucleation, LC3 is localized to the autophagic membrane and the overlapping GFP and RFP fluorescence appears as yellow puncta. After the autophagosome matures, it fuses with the lysosome, forming an autolysosome. The GFP in this LC3 sensor is pH labile and becomes quenched by the acidic environment of the autolysosome. However, the RFP remains stable; therefore, autolysosomes are indicated by red puncta. Accordingly, when autophagy is inhibited at the final stage (completion), the abundance of yellow puncta is expected to increase proportionally to the level of autophagy inhibition.

For the use and convenience of the material disclosed in the present Examples, the following abbreviations are provided: CQ, chloroquine; QN, quinacrine; HCQ, hydroxychloroquine; MQ, mefloquine; LC3, light chain 3; 3-MA, 3-methlyadenine; PI3K, phosphoinositide-3-kinase; ATG, autophagy-related gene; DNR, anthracycline daunorubicin; HDAC, histone deacetylase; RFP, red fluorescent protein; GFP, green fluorescent protein; TEM, transmission electron microscope; LAMP, lysosome associated membrane protein; UACC, University of Arizona Cell Culture; MAPK, mitogen-activated protein kinase; EGFR-TKI, epidermal growth factor receptor tyrosine kinase inhibitor

To determine if other anti-malarial compounds exist which inhibit autophagy more potently than CQ, U2OS cells stably expressing tfLC3 cells were treated with CQ or six other anti-malarial agents (amodiaquine, artemesinin, mefloquine, piperaquine, primaquine, and quinacrine) for three hours. Cells were then fixed and imaged by fluorescent microscopy. From these fluorescent images the effective concentration (EC) of autophagy inhibition was determined (Table 4). The EC was determined as the concentration at which cells contained a statistically significant increase in puncta number compared to the vehicle control. To identify anti-malarial compounds that inhibited autophagy more potently than CQ, the EC of each anti-malarial was divided by the EC of CQ (EC/ECcQ) to yield a relative potency score (Table 4). Accordingly, a potency score greater than one indicated a more potent autophagy inhibitor than CQ. Two anti-malarial compounds, mefloquine (MQ) and quinacrine (QN), were found to be more potent autophagy inhibitors than CQ with relative potency scores of approximately 30 and 60, respectively.

TABLE 4
Relative autophagy inhibition for each anti-malarial compound.

| | | Effective | |
|---|---|---|---|
| | | Concentration | Potency |
| Anti-material | | (EC) | (EC/EC_{CQ}) |
| Amodiaquine | 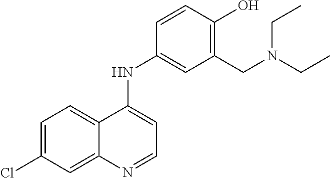 [SMILES: CCN(CC)Cc1cc(Nc2ccnc3cc(Cl)ccc23)ccc1O] | 15 μM | 1 |
| Artemisinin | 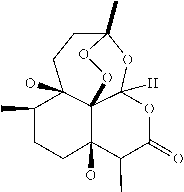 [SMILES: CC1C(=O)OC2O[C@@]3(C)CC[C@@]4(O)[C@H](C)CC[C@]1(O)[C@@]24OO3] | 15 μM | 1 |
| Chloroquine | 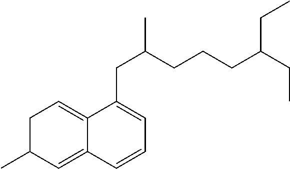 [SMILES: CCC(CC)CCCC(C)Cc1cccc2c1=CCC(C)C=2] | 15 μM | 1 |
| Mefloquine | 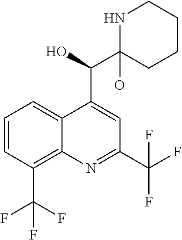 [SMILES: O[C@H](c1cc(C(F)(F)F)nc2c(C(F)(F)F)cccc12)C1(O)CCCCN1] | 0.5 μM | 30 |
| Primaquine | 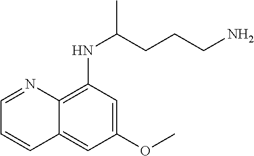 [SMILES: COc1cc(NC(C)CCCN)c2ncccc2c1] | 50 μM | 0.25 |
| Piperaquine | 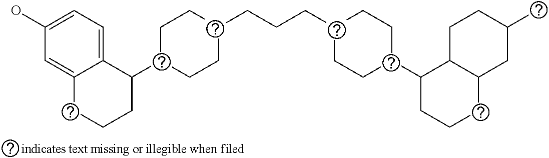 [SMILES: CC1CCC2C(CCCC2C2CCC(CCCC3CCC(C4CCCc5cc(O)ccc54)CC3)CC2)C1] | 50 μM | 0.25 |
| Quinacrine | 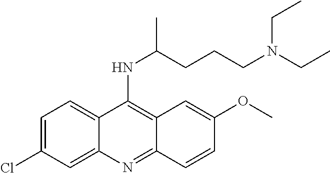 [SMILES: CCN(CC)CCCC(C)Nc1c2ccc(Cl)cc2nc2ccc(OC)cc12] | 0.25 μM | 60 |

CQ, QN, and MQ have been previously shown to inhibit autophagy, while the remaining anti-malarial agents tested (piperaquine, primaquine, amodiaquine, and artemisinin) have not, to our knowledge. Despite their reduced potency, experiments were performed to confirm that these agents function as autophagy inhibitors. Using immunoblotting for endogenous LC3, each agent was shown to induce the accumulation of LC3-II, both basally and in response to rapamycin-induced autophagy induction (FIG. 7). This data confirms that autophagy inhibition may be a common activity of many anti-malarial agents.

Next, experiments were performed to carefully characterize the autophagy inhibition of QN, the most potent autophagy inhibitor, in comparison with CQ. To this end, a ten-point dose response treatment in U2OS-tfLC3 cells was performed. While QN treatment increased the amount of LC3 puncta at nearly all doses, the same doses of CQ failed to achieve an appreciable number of puncta (FIG. 8A). In order to quantitatively confirm the results, image analysis software was used to determine the mean intensity of puncta at each dose of compound. Data gathered showed that mean intensity was a more accurate indicator of autophagy in these experiments compared to puncta number due to the large abundance of puncta that become individually indistinguishable at higher inhibitor concentrations. This autophagosome accumulation prevents accurate separation of objects, rendering puncta number less reliable. Mean intensity proved to correlate well with number of puncta, and importantly, was not negatively affected at high concentrations (FIG. 9). Treatment with QN significantly increased the mean intensity of autophagic puncta at 0.25 μM (FIG. 8B). However, CQ treatment was only able to produce a significant increase in mean intensity at 15 μM, a 60-fold higher concentration (FIG. 8B).

### Biological Example 17

### Characterization and Biological Activity of Exemplary Compounds of Formula a, Examples 7 and 27

The present inventors have surprisingly demonstrated that two classes of autophagy inhibitors may be useful therapeutically—those that potently inhibit autophagy and cause cytotoxicity as single agents; and those agents that are potent autophagy inhibitors yet relatively cytostatic, permitting use in combination therapies (as adjuvants). To develop such compounds, the anti-malarial compound QN was used as a template for rational chemical synthesis and created a series of over 60 novel small molecules. In some embodiments, changes were made to the acridine scaffold (6-chloro-2-methoxy-acridin) and R-group (N¹,N¹-diethyl-N⁴-methylpentane-1,4-diamine) of QN to form these illustrative compounds. These molecules were then screened for autophagy inhibition as well as effects on cell viability (Table 5).

TABLE 5
Relative autophagy inhibition (EC), cytotoxicity (IC₅₀), and chemical
structure of novel autophagy compounds. The EC, potency and IC₅₀, of
the compounds of Formula A, e.g., Example 7 and Example 27, are
shown in comparison to chloroquine (CQ) and quinacrine (QN).

| | Effective | | |
|---|---|---|---|
| Compound | Concentration | Potency | IC₅₀ |
| Chloroquine | 15 μM | 1 | 75 μM |
| Quinacrine | 0.25 μM | 60 | 2.5 μM |
| Example 7 | 0.1 μM | 150 | 0.7 μM |
| Example 27 | 5 μM | 3 | 27 μM |

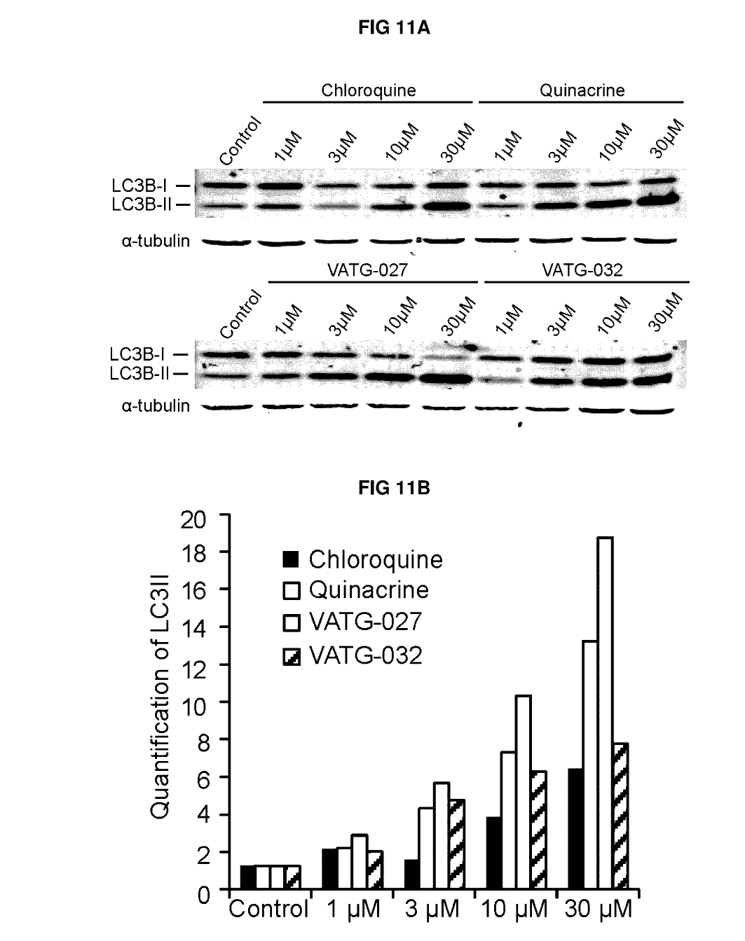

While moderate changes in autophagy inhibition and viability were seen with most chemical alterations, a few key changes had considerable impacts on cell viability (IC₅₀) and/or EC. From the most potent autophagy inhibitors, two molecules were chosen for further evaluation, each with divergent effects on cell viability (IC₅₀). While compound Example 7 (EC=5 μM), was less cytotoxic than QN with an IC₅₀ equal to 27 μM, Example 27 (EC=0.1 μM) was considerably more cytotoxic with an IC₅₀ of 0.7 μM. The autophagy inhibition and cell viability effects of Example 27 and Example 7 were carefully quantified across a concentration gradient, as described above, and compared to that of both CQ and QN (FIG. 10A-C and Table 5). Example 27 was found to be 3-fold more potent at autophagy inhibition than CQ, yet 10 times less cytotoxic than QN (Table 5). The potent autophagy inhibition coupled with low cytotoxicity makes Example 27 a candidate compound for adjuvant therapy. Example 7 was also found to be 150-fold more potent at autophagy inhibition than CQ (and 2× more potent than QN); however, it was also 3.5-fold more cytotoxic than QN. To confirm autophagy inhibition independent of a fluorescent reporter, a dose response was performed and endogenous LC3 processing was measured by Western blotting (FIG. 11A). Compounds Example 27 and Example 7 both showed increased accumulation of LC3-II, consistent with the tfLC3 observations (FIG. 10A-10C and FIG. 11A). Finally, the inventors confirmed that 24 hour treatment with each compound induced caspase 3 activation, suggesting the reduction in cell viability is at least partially the result of apoptotic cell death (FIG. 10C).

Mechanism of Action—Lysosomal Inhibition.

Without wishing to be bound by any particular mechanism or theory, it was believed that since both CQ and QN are both known to inhibit autophagy by deacidifying and preventing the lysosomal turnover of its constituents, the inventors postulated that the compounds of the present invention, for example, those of Formula III, Formula III(a), Formula V, Formula V(a), Formula A, Formula A¹, Formula A², Formula A³, or a pharmaceutically acceptable salt thereof, as exemplified by Examples 7 and 27, function by the same mechanism. To test the theory, lysosomal turnover was first evaluated by visualizing lysosomes using transmission electron microscopy (TEM). U2OS cells were initially treated with a CQ concentration above the effective dose (100 μM) for 3 hours as a positive control, and were then subsequently fixed and imaged. While few vesicles were observed in vehicle-treated cells, an accumulation of large, electron-dense and electron-lucent vesicles was found, consistent with lysosomes and endosomes, upon addition of CQ (FIG. 12A). Once this phenotype was established, cells were treated with a lower concentration (3 μM) of CQ to compare each compound at the same concentration. This dose was previously shown to inhibit autophagy for QN, Example 7, or Example 27, but not CQ (FIG. 10B). As expected, 3 μM CQ was found to be insufficient to cause a noticeable increase in either vesicle size or number compared to the vehicle control at this concentration. However, QN, Example 7, and Example 27 treatments at 3 μM, all caused an increase in both the size and number of electron-lucent and electron-dense vesicles detected (FIG. 12B). In addition, Example 7 or Example 27 treatment resulted in the appearance of several electron-opaque structures, consistent with lipid droplets (FIG. 12B).

Deacidification of lysosomes would be expected to not only prevent the maturation and turnover of the lysosomes from early to late, but also affect the functionality of lysosomal enzymes and consequently the turnover of lysosomal constituents. To determine if lysosomal activity was inhibited, lysosomal protease cathepsin B activity was measured by immunoblotting (FIG. 12C). Lysates were harvested from cells and treated with CQ, QN, Example 7, and Example 27 at both 3 μM and 30 μM for 6 hours. QN and Example 7 showed a nearly complete loss of active cathepsin B at 30 μM, while Example 27 showed a significant decrease at the same dose. In contrast, CQ showed little effect on active cathepsin B at either dose. Taken together, these results suggest that the compounds of Formula III, Formula III(a), Formula V, Formula V(a), Formula A, Formula A¹, Formula A², Formula A³, or a pharmaceutically acceptable salt thereof, for example, compounds Example 7 and 27, are considerably more potent than CQ at blocking lysosomal activity and turnover.

Next, the inventors sought to confirm the inhibition of lysosomal turnover induced by the compounds of the present invention, for example, those of Formula III, Formula III(a), Formula V, Formula V(a), Formula A, Formula A¹, Formula A², Formula A³, or a pharmaceutically acceptable salt thereof, by evaluating the abundance of endogenous lysosomal protein, LAMP1. In addition, lysosome acidity was assessed by co-staining cells with LysoTracker Red, a dye that localizes to the lysosome based on the low pH. Cells were treated with 3 μM CQ, QN, Example 7, or Example 27 for 3 hours, with LysoTracker Red supplemented for the final hour. Following, cells were fixed and stained with endogenous LAMP1 antibodies and both LAMP1 and LysoTracker Red imaged. Results indicated that CQ failed to yield an appreciable change in LAMP1 positive membranes and LysoTracker Red staining at 3 μM (FIGS. 13A and 13B). In contrast, QN, Example 7, and Example 27 treatments all caused substantial increases in LAMP1 staining and essentially eliminated LysoTracker Red staining (FIGS. 13A and 13B). To quantify this phenotype, the co-localization of LAMP1 and LysoTracker Red was measured using image analysis software. The ratio of intensity of each signal across pixels of individual vesicles was measured and displayed using a colorimetric scale, where red indicates the presence of LAMP1-only, purple indicates the presence of LysoTracker Red-only, and green indicates the presence of both (FIG. 14). In addition, Mander's colocalization coefficient (MCC) values were determined for each treatment (FIG. 13C). Not only does the presence of LAMP1-positive membranes increase, but the intensity of LAMP1 staining also increases with QN, Example 7, and Example 27 treatment (FIG. 13C). In addition, the results indicate that the inverse holds true for LysoTracker Red staining; treatment with QN, Example 7, or Example 27 decreased LysoTracker Red staining more so than CQ, suggesting a more substantial loss of lysosomal acidity at these lower concentrations. Collectively, these results suggest that these compounds of Formula A function by deacidifying lysosomes and impairing their turnover.

Determination of Autophagic Flux in BRAF Mutant Melanoma Lines

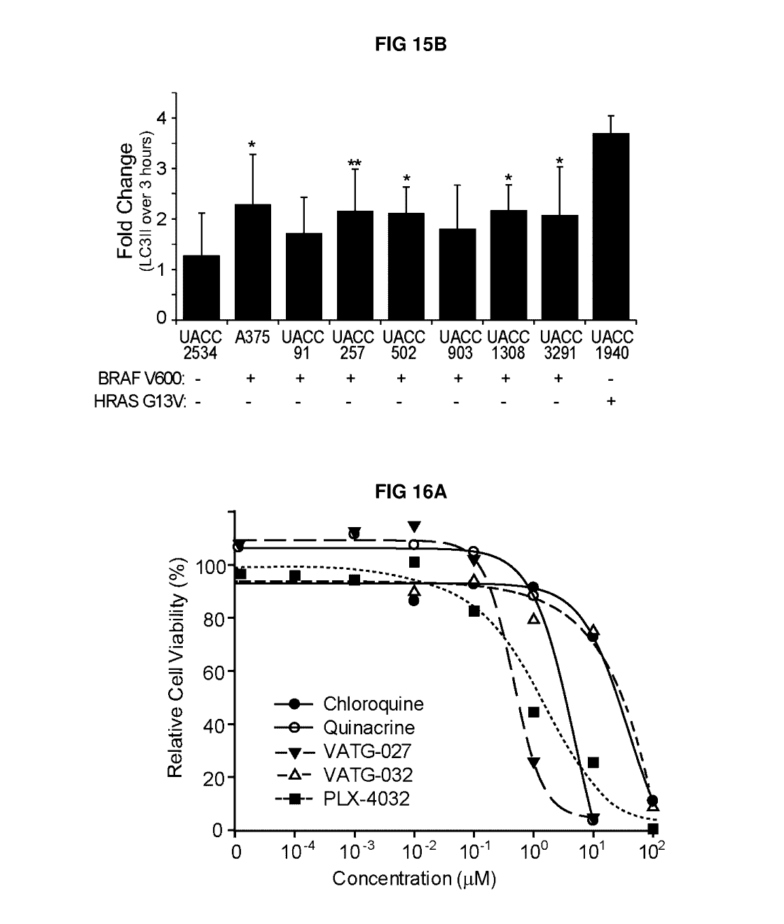

Oncogenic mutation of BRAF (V600E), a genetic driver in greater than 50% of melanomas, has been shown to increase autophagy, potentially as a cell survival mechanism. Accordingly, the potential therapeutic utility of autophagy inhibition in the A375 melanoma cell line and eight metastatic patient-derived lines (UACC-91, UACC-257, UACC-502, UACC-903, UACC-1308, UACC-1940, UACC-2534, and UACC-3291), seven of which contain the BRAF-V600E mutation determined by Sanger sequencing was evaluated. First, basal autophagic flux was determined in each cell line by measuring the accumulation of LC3-II (normalized to a loading control) in response to lysosome inhibition. Cells were treated for one or three hours with CQ and quantitative western blotting was used to measure the fold-change in LC3-II levels over time (normalized to a-tubulin to control for protein loading). Importantly, the inventors have found that cell lines expressing the BRAF-V600E mutation had a high level of autophagic flux, with greater than 2-fold accumulation of LC3-II by three hours (FIG. 15A). In addition, while one cell line expressing wild-type BRAF (UACC-1940) exhibited high autophagic flux, another wild-type BRAF cell line (UACC-2534) did not. Upon further investigation into the mutational status of the two cell lines, UACC-1940 cells contain a mutation in HRAS (G13V), which activates the MAPK pathway similar to BRAF (FIG. 15B). Together, all melanoma lines showed measurable levels of autophagic flux; however, the cell line that was not driven by either oncogenic BRAF or RAS showed the lowest level of autophagic flux.

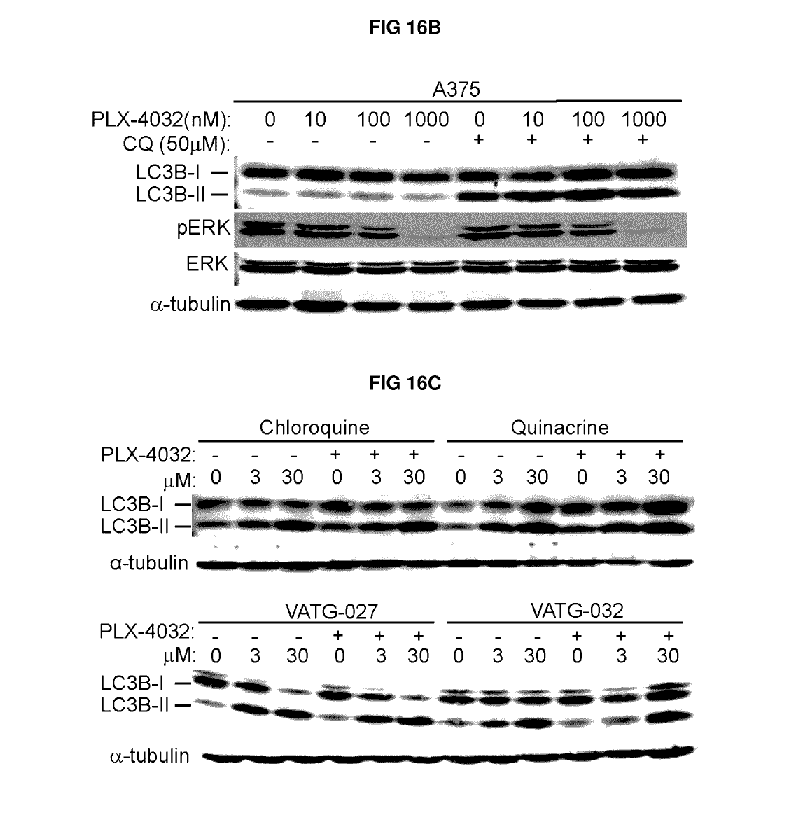

Next, the sensitivity of each melanoma cell line to CQ, QN, Example 7, and Example 27 was assessed. CQ reduced cell viability with IC₅₀ values ranging from 13 μM to 40 μM. Example 27 affected cell viability in a similar manner, yielding IC₅₀ values between 15 μM and 42 μM. Consistent with observations from U2OS cells, QN was considerably more cytotoxic than CQ, with IC₅₀ values between 1.9 μM and 3.9 μM. Example 7 treatment produced IC₅₀ values that closely matched those of QN, between 0.4 μM to 2.7 μM. Overall, the four inhibitors affected viability of the nine melanoma cell lines comparable to that observed in U2OS cells (Table 6; FIG. 16A).

TABLE 6
IC₅₀ (μm) of CQ, QN, Example 7, Example 27 and PLX-4032

| | | UACC- | UACC- | UACC- | UACC- | UACC- | UACC- | UACC- | UACC- | |
|---|---|---|---|---|---|---|---|---|---|---|
| | A375 | 91 | 257 | 502 | 903 | 1308 | 1940 | 2534 | 3291 | U20S |
| Chloroquine | 24.6 μM | 13.4 μM | 14 μM | 24.3 μM | 34.1 μM | 39.6 μM | 19.6 μM | 18 μM | 28.1 μM | 75 μM |
| Quinacrine | 2.3 μM | 2.9 μM | 3 μM | 2.2 μM | 2.7 μM | 3.9 μM | 1.9 μM | 2.2 μM | 2 μM | 2.5 μM |
| Example 7 | 0.4 μM | 0.5 μM | 2 μM | 1.8 μM | 1.5 μM | 2.1 μM | 4 μM | 2.7 μM | 0.8 μM | 0.7 μM |
| Example 27 | 24.3 μM | 22.2 μM | 25.3 μM | 42.3 μM | 27.2 μM | 14.9 μM | 21.6 μM | 26.6 μM | 25.3 μM | 27 μM |
| PLX-4032 | 1 μM | 0.6 μM | 4 μM | 4.2 μM | 69 μM | 0.6 μM | 3.3 μM | 19.3 μM | 1.5 μM | — |

### Biological Example 18

### Combination treatment of PLX-4032 and Autophagy Inhibitors

Since the melanoma cell lines are capable of undergoing autophagy and sensitive to autophagy inhibition, the inventors questioned whether autophagy inhibitors could improve the efficacy of, or sensitize melanoma cells against cytotoxic and chemotherapeutic drugs for advanced metastatic melanoma. In one example, PLX-4032 drug “vemurafenib” (marketed as ZELBORAF®) is a B-Raf enzyme inhibitor developed by Plexxikon and Genentech for the treatment of late-stage melanoma. The name “vemurafenib” comes from V600E mutated BRAF inhibition. PLX-4032, selectively targets the V600E mutant BRAF and it is unknown how this drug may affect autophagic flux. To determine the effects of the drug on autophagic flux, the present inventors performed experiments to determine whether PLX-4032 induces autophagy, as has been observed with other targeted agents. In some embodiments, the accumulation of LC3-II in response to lysosome inhibition was measured by quantitative Western blotting, as described above. A375 cells were treated with PLX-4032 (10 nM, 100 nM, and 1 μM) for three hours in the presence or absence of CQ. While PLX-4032 effectively blocked oncogenic BRAF signaling, as measured by reduced phosphorylation of the downstream effector, ERK1/2, autophagy was not significantly altered (FIG. 6B).

Furthermore, A375 cells were treated with PLX-4032 in combination with CQ, QN, Example 27, and Example 7, and again, basal autophagic flux was not significantly altered by PLX-4032 with Example 7 and Example 27 showing greater increases in LC3II accumulation as seen previously in U2OS cells (FIG. 16C). This data suggests that while mutant BRAF-V600E-expressing cell lines undergo basal autophagy, chemical inhibition of oncogenic MAPK signaling does not alter the overall flux.

Despite the lack of autophagy induction by PLX-4032, the autophagic capacity retained in cells during treatment suggests that autophagy may potentially mediate cell survival. Therefore, the inventors hypothesized that an autophagy inhibitor may be effective in a combinatorial treatment regimen including PLX-4032. To evaluate the efficacy of this combinatorial treatment, soft agar colony formation assays was performed to assess anchorage independent growth, one hallmark of cellular transformation and tumor growth.

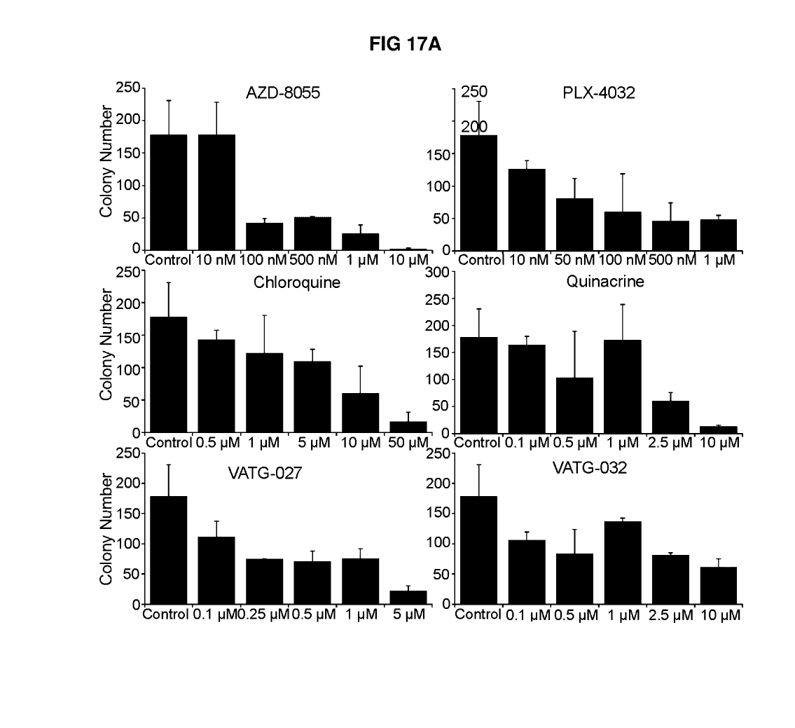

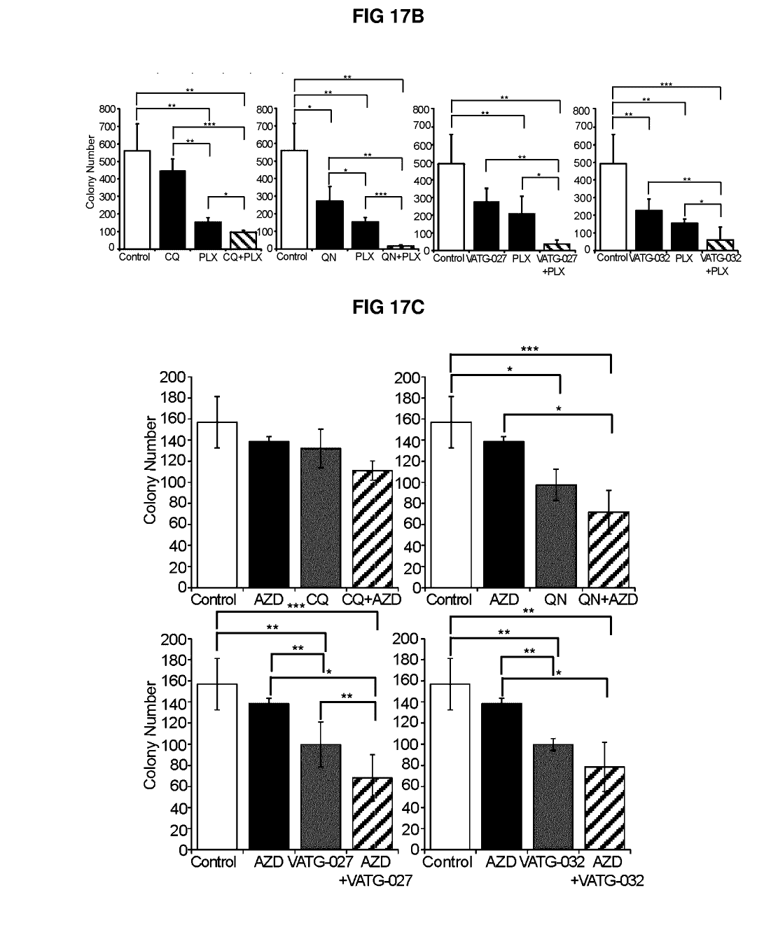

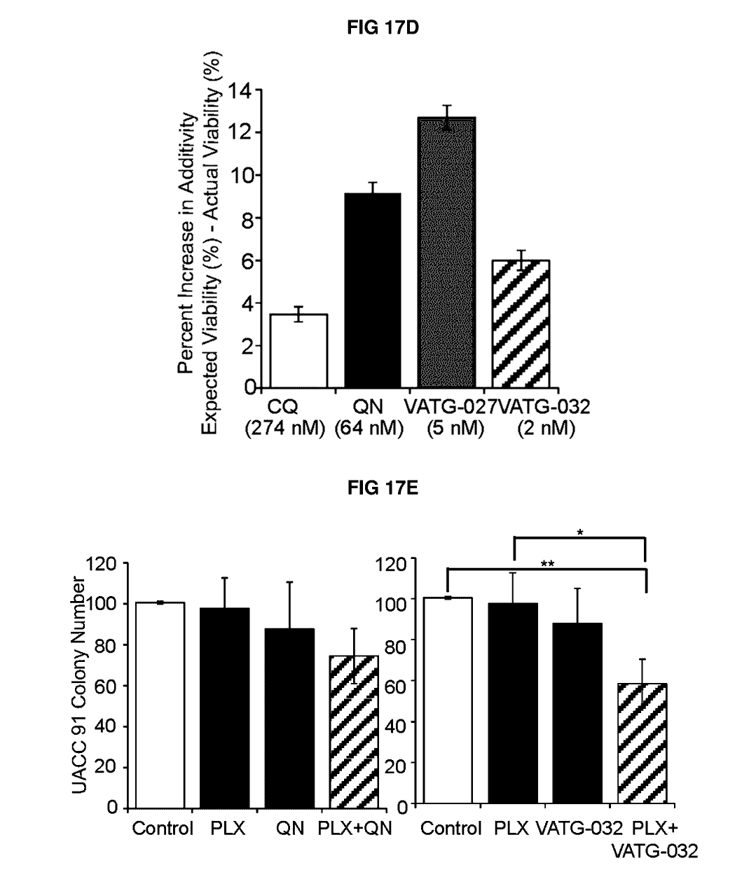

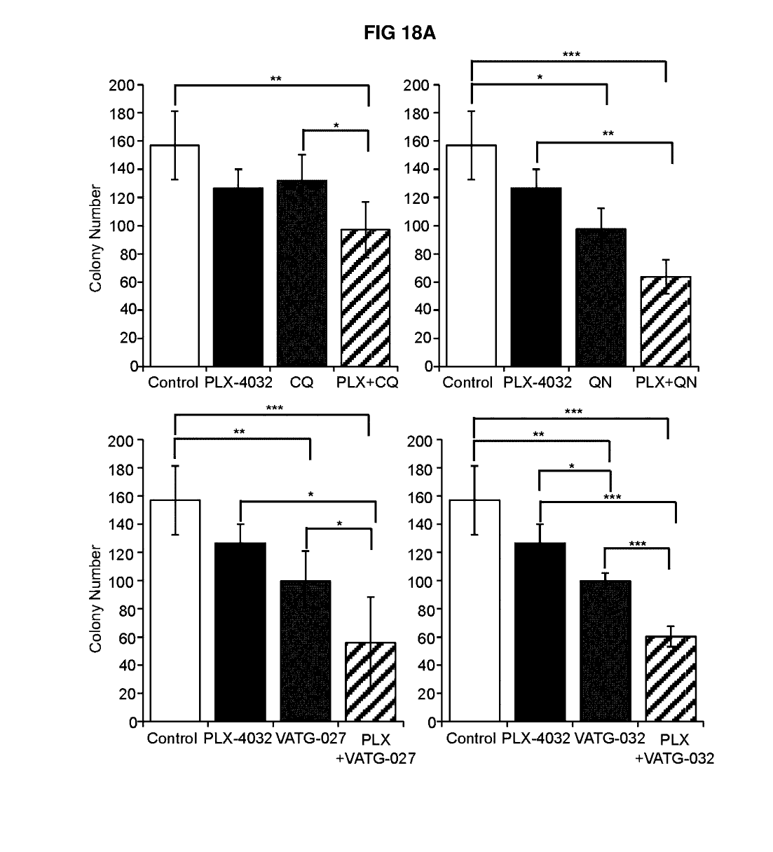

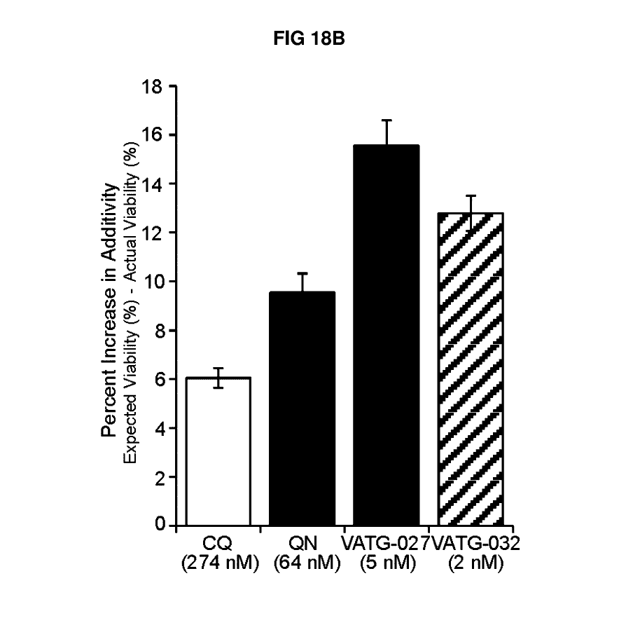

A375 cells were plated in soft agar for three weeks and treated every other day with a range of concentrations of PLX-4032, CQ, QN, Example 7, or Example 27. In addition, AZD-8055, a catalytic mTOR inhibitor, was evaluated to determine how autophagy inhibitors may affect cell sensitivity to a known autophagy stimulus (FIG. 17A). The concentration of each single agent which yielded a minimal effect (˜10%; IC₁₀) was determined. Following, cells plated in soft agar were treated with the IC₁₀ of PLX-4032 alone or in combination with the IC₁₀ of CQ, QN, Example 7, or Example 27 (as well as each autophagy inhibitors at their IC₁₀ alone) (FIG. 18A). These same combinatorial soft agar experiments were also completed with an mTOR inhibitor, AZD-8055 in place of PLX-4032 (FIG. 17C).

To determine if the effects of each combination were more than, less than, or equal to additive, predictions for additivity were made using the Bliss Independence (Table 7), as disclosed in Yan H, Zhang B, Li S, Zhao Q. A formal model for analyzing drug combination effects and its application in TNF-alpha-induced NFkappaB pathway. BMC Syst. Biol. 2010; 4:50; Chou, T C. Drug combination studies and their synergy quantification using the Chou-Talalay method. Cancer Res 2010; 70:440-6 and Hiss D C, Gabriels G A, Folb P I. Combination of tunicamycin with anticancer drugs synergistically enhances their toxicity in multidrug-resistant human ovarian cystadenocarcinoma cells. Cancer Cell Int 2007; 7:5, the disclosure of all of these references is hereby incorporated by reference in their entireties.

TABLE 7
Bliss independence model calculations of additivity for autophagy
inhibitors with PLX-4032 and AZD-8055.

| | | | | Autophagy Inhibitor = | |
|---|---|---|---|---|---|
| | | Single Compound | | PLX-4032 | |
| | | Relative | Relative | Expected | |
| | | Growth | Growth | Bliss | |
| | | Inhibition | Inhibition | Additive | Actual |
| | | Compound | Compound | Inhibition | Growth |
| Compound A | Compound B | A | B | Value | Inhibition |
| Chloroquine | PLX-4032 | 0.16 | 0.19 | 0.33 | 0.38 |
| Quinacrine | PLX-4032 | 0.38 | 0.19 | 0.50 | 0.59 |
| Example 7 | PLX-4032 | 0.37 | 0.19 | 0.49 | 0.64 |
| Example 27 | PLX-4032 | 0.37 | 0.19 | 0.49 | 0.62 |
| Chloroquine | AZD-8055 | 0.16 | 0.12 | 0.26 | 0.29 |
| Quinacrine | AZD-8055 | 0.38 | 0.12 | 0.45 | 0.54 |
| Example 7 | AZD-8055 | 0.37 | 0.12 | 0.44 | 0.57 |
| Example 27 | AZD-8055 | 0.37 | 0.12 | 0.44 | 0.50 |

E_{xy} = Eₓ = +E_{y} − (Eₓ * E_{y}), with Eₓ being compound A and E_{y} being compound B.

CQ and PLX-4032 was found to reduce colony formation by 38%, slightly greater than the predicted effect if these agents interact additively (33%) (FIG. 18A; Table 7). Similarly, combinatorial treatment of QN with PLX-4032 reduced colony formation by 59%, just greater than the expected value of 50%. Both compounds of Formula A, Example 7 and Example 27 were more significant than QN at increasing the combination effect of PLX-4032, reducing colony formation by 64% (compared to an expected value of 49%) and 62% (compared to an expected value of 49%), respectively (FIG. 18A; Table 7). Similar results were seen with the combinatorial treatment of AZD-8055 and in the BRAF V600E mutant UACC91 cell line. (FIG. 17C-17E). To provide consistency with prior experiments, PLX-4032 combinations using autophagy inhibitors at 3 μM (except Example 7, which was used at 1 μM owing to its substantial activity as a single agent) were evaluated and confirmed that combinatorial effects exceeded additivity (FIG. 17B). Taken together, these results suggest that autophagy inhibitors have utility in melanoma treatment, both as single agents and in combination with other chemotherapeutic or cytotoxic agents, for example, B-RAF enzyme inhibitors such as PLX-4032 and mTOR inhibitors, for example, AZD-8055.

In the present Biological Examples, melanoma cell models were utilized to evaluate the therapeutic potential of autophagy inhibitors. Melanoma is an aggressive cancer that has several well identified oncogenes and tumor suppressors and mutations in these genes have been shown to upregulate autophagy and survival in melanoma in several reports. Three common genes that are recurrently mutated in melanoma, as well as many other cancers, which include RAS, BRAF, and PTEN, which in turn activate PI3K/AKT/mTOR and RAS/RAF/MEK/MAPK pathways and have been shown to deregulate autophagy. In a recent report, it was shown that inhibition of both the mTOR pathway (using an mTOR inhibitor, temsirolimus) and autophagy (using HCQ) produced synergistic effects in melanoma cell death. Further detailed reports have shown that this hyperactivated MAPK pathway prevents mTORC1 nutrient sensing, specifically its inhibition due to the lack of leucine. They further assessed the role of autophagy and nutrient sensing in vivo using human melanoma xenografts, and this combination of a leucine-free diet and an autophagy inhibitor dramatically reduced tumor volume. Taken together, there is mounting evidence for the role of autophagy inhibition in melanoma tumorigenesis.

In the present Biological Examples, active basal autophagy was confirmed in a panel of nine melanoma cell lines and found that all were sensitive to autophagy inhibition. Through the use of quantitative microscopy and rational chemical synthesis, novel autophagy inhibitors were further identified with up to a 50-fold increase in inhibition compared to that of CQ. The data provided herein suggests that these compounds function to deacidify the lysosome and thus, inhibit the delivery and degradation of autophagic vesicles, similar to CQ. Importantly, the inventors have shown that autophagy inhibitors decrease cell viability, both as single agents and in combination with therapeutics, supporting the hypothesis that autophagy promotes cell survival. In addition to activity as single agents, autophagy inhibitory compounds of Formula A were found to sensitize cells to the BRAF-V600E-specific inhibitor, PLX-4032. This is consistent with evidence that many therapeutics, including targeted agents, can benefit from the addition of an autophagy inhibitor as an adjuvant.

In colon cancer cells containing a RAS mutation, it was shown that inhibition of autophagy using Bafilomycin A1 increased cell death; in addition, the combination of the chemotherapeutic 5-fluorouracil (5FU) with CQ lead to a further increase in cell death than when used alone. The effectiveness of autophagy inhibition in colon cancer is particularly exciting as 18% of colon cancers share the BRAF V600E mutation common in melanoma, suggesting the work presented here could be applicable to additional cancer types. Further, inhibition of autophagy using both CQ and mefloquine, another anti-malarial, was able to induce cell death in breast cancer lines also expressing RAS and BRAF mutations.

Many chemotherapeutic treatment strategies have also been shown to upregulate autophagy, a counterproductive effect as upregulated autophagy promotes aberrant survival. This was demonstrated in a study of lung cancer with the epidermal growth factor receptor (EGFR) tyrosine kinase inhibitors (TKIs), gefitinib and erlotinib. Treatment with these TKIs conferred a marked increase in autophagy activation and cytotoxicity was significantly enhanced upon the addition of CQ. Similarly, in a model of cervical cancer, it was found that supplementing cisplatin treatment, which induced autophagy, with CQ enhanced the cytotoxicity that was seen with cisplatin alone. Increased effectiveness of therapies by cotreatment with autophagy inhibitors demonstrates the value of targeting autophagy in future treatment strategies.

The role of autophagy in cancer is complex and context dependent; this especially includes models with mutations in BRAF. As mentioned earlier, a correlation between increased autophagy and mutant BRAF in cell lines has been reported, demonstrating the importance of autophagy inhibition. Despite this, others have suggested that while supporting high basal autophagy, mutant BRAF confers resistance to autophagy activation by mTORC1 inhibition. This discrepancy is nicely discussed in a brief review where Corazzari and Lovat emphasize that not only the tumor stage, but the type and differing mutations may account for these differing results. Our data highlights the importance in assessing cellular autophagic flux and the need for more potent autophagy inhibitors in aggressive cancers and therapeutic treatments. Furthermore, the better than additive effects that were seen with the combination of PLX-4032 and autophagy inhibitors was performed in soft colony formation assays; which may be better for observing anti-tumor effects, as opposed to acute drug toxicity in standard 2D cultures.

Despite this, CQ remains a relatively weak autophagy inhibitor requiring a substantial dose for full potency in vivo. Several studies have explored the development of CQ analogs; however, these compounds were primarily investigated for efficacy in malaria treatments and have not been explored as cancer therapeutics. Studies that have investigated the use of CQ analogs in cancer treatment have primarily focused on their ability to induce cell death as single agents. Even though cytotoxic compounds are valuable, potent autophagy inhibition alone does not necessarily elicit cytotoxic effects. The present invention provides evidence suggesting that inhibition of autophagy can be accomplished with compounds that are relatively well-tolerated by cells (i.e., Example 27). The development of such potent autophagy inhibitors provides an opportunity for use as adjuvants in treatment strategies, effectively blocking autophagy-mediated cancer cell survival without significantly increasing toxicity as a single agent. This type of compound provides an exciting outlet for sensitization of cancer cells to the latest anti-cancer therapeutics.

### Biological Example 19

### In Vitro Compound Screening of Plasmodium falciparum Asexual Blood Stage

In this example, compounds of Formula A, Formula A¹, Formula A², Formula A³ or pharmaceutically acceptable salts thereof, are tested in Plasmodium sp infected cells to determine the effectiveness of these compounds to determine their anti-malarial activity. Exemplary compounds, Example 7 and Example 27 (See Table 1) were dissolved in DMSO at 10 mg/mL and 4× compound dilutions were prepared in screening medium [RPMI 1640 with no hypoxanthine, HEPES (5.94 μg/L), NaHCO3 (2.1 μg/L), Neomycin (100 μg/mL), and Albumax II (5 μg/L)]. Human red blood cells were prepared without and with stock cultures of P. falciparum strains, NF54 (Chloroquinine (CQ) sensitive) and K1 (CQ resistant), to a parasitemia of 0.3% and hematocrit of 2.5%. Using a 96-well plate, 100 μL/well of red blood solution was added along with a 2-fold serial dilution of Example 7 and Example 27. Plates were incubated at 37° C. with 93% N2, 4% CO₂, and 3% O₂ for 48 hours, after which 50 μL of [3H]-hypoxanthine (0.5 μCi) was added to each well. After 24 hours, plates were harvested with a Betaplate™ cell harvester (Wallac, Zurich Switzerland) which transferred the red blood cells onto a glass fiber filter and washed with distilled water. Dried filters were inserted into a plastic foil with 10 mL of scintillation fluid and counted in a Betaplate™ liquid scintillation counter (Wallac, Zurich Switzerland). Results were obtained as counts per minute (cpm) per well at each concentration. The 50% inhibitory concentration (IC₅₀) value was evaluated by Logit regression analysis. Compounds were scored as no activity: IC₅₀>1000 ng/mL, low activity: IC₅₀ 50-1000 ng/mL, or good activity: IC₅₀<50 ng/mL.

Evaluation of In Vitro Cytotoxicity (LD₅₀)

L6 cells were seeded at 4×10⁴ cells per well in a 96-well microtiter plate with each well containing 100 μL of RPMI 1640 medium, supplemented with 1% L-glutamine (200 mM), and 10% fetal bovine serum. Serial drug dilutions of Example 7 and Example 27 with seven 3-fold dilution steps, covering a range from 90 to 0.12 μg/mL, were prepared. After 72 h of incubation, the plates were inspected under an inverted microscope to ensure growth of the controls and sterile conditions. Alamar Blue (10 μL, 12.5 mg of resazurin dissolved in 100 mL of double-distilled water) was then added to each well, and the plates were incubated for another 2 hours. Plates were read with a microplate fluorometer using an excitation wavelength of 536 nm and an emission wavelength of 588 nm. Data were analyzed using microplate reader software. Podophyllotoxin was used as a standard (L6 IC₅₀=0.04 μg/mL).

In Vivo Compound Efficacy in Plasmodium berghei

Mice were infected intravenously with parasitized red blood cells on day 0 (2×10⁷ parasitized erythrocytes per mL). Experimental mice were treated at 4, 24, 48, and 72 hours post-infection with an oral dose of Example 7 at 50 mg/kg and were compared to an infected control group for the % reduction in parasitaemia on day 4 (96 hours post-infection) and for mean survival (monitored up to 30 days post-infection). A compound was considered curative if the animal survived to day 30 after infection with no detectable parasites.

Results:

Examples 7 and 27 Demonstrate Antimalarial Activity In Vitro

To determine whether the compounds of Formula A, Example 7 and Example 27, displayed anti-malarial activity, the inventors investigated their effects on in vitro blood cultures of the malaria-causing parasite Plasmodium falciparum. Two-fold dilution curves of both Example 7 and Example 27 were assayed against the chloroquinine (CQ)-sensitive Plasmodium falciparum NF54 strain, and the CQ-resistant K1 strain. Compounds were scored as having no activity: IC₅₀>1000 ng/mL; low activity: IC₅₀ 50-1000 ng/mL; or good activity: IC₅₀<50 ng/mL. In the CQ-sensitive NF54 strain, Example 7 and Example 27 showed antimalarial activity with IC₅₀ values of 4 nM (good activity) and 323 nM (low activity), respectively (Table 8).

TABLE 8
Antimalarial activity (IC₅₀) against P. falciparum NF54 and K1 in vitro,
cytotoxicity (LD₅₀) in L6 cells, and their fold difference of LD₅₀/IC₅₀ (therapeutic window).

| | P. falciparum | P. falciparum | Cytotoxicity | | |
|---|---|---|---|---|---|
| | NF54 | K1 | in L6 Cells | NF54 | K1 |
| Compound | (CQ sensitive) | (CQ resistant) | (LD₅₀) | Fold | Fold |
| Chloroquine | 68 nM ± 1 nM | 196 nM ± 31 nM | — | — | — |
| Example 7 | 4 nM ± 1.3 nM | 23 nM ± 2.5 nM | 1.9 μM ± 0.01 nM | 475 | 82 |
| Example 27 | 323 nM ± 78 nM | 131 nM ± 8 nM | 26 μM ± 1 nM | 80 | 19 |

Similar results were observed with the CQ-resistant K1 strain, in which Example 7 and Example 27 showed antimalarial activity with IC₅₀ values of 23 nM (good activity) and 131 nM (low activity), respectively (Table 8).

Cytotoxicity of Example 7 and Example 27 Allow for an Therapeutic Window

Next, the inventors wanted to determine if there is a therapeutic window for Example 7 and Example 27 in the treatment of P. falciparum. A therapeutic window for P. falciparum treatment is defined as a 10-fold differential between the in vitro cytotoxicity (LD₅₀) within a mammalian cell line and the antimalarial IC₅₀ (LD₅₀/IC₅₀) as defined by the MMV. Using the rat skeletal muscle L6 mammalian cell line, dose-response curves for Example 7 and Example 27 were performed and the LD₅₀ determined. The LD₅₀ of Example 7 is approximately 1.9 μM and Example 27 is 26 μM, making the therapeutic windows for both compounds more than sufficient at >10-fold in each Plasmodium strain (Table 8). Example 7 showed the greatest therapeutic window with a fold change of 475 in the NF54 strain (CQ sensitive) and a fold change of 82 in the K1 strain (CQ resistant). However, MMV experience with successful compounds shows that those compounds with an in vitro activity against the laboratory strains of P. falciparum at <10 nM have the most success, predicating the continuation of only VATGO14 in vivo (Burrows, van Huijsduijnen et al. 2013).

Example 7 Demonstrates Antimalarial Activity Against P. berghei In Vivo

The potent anti-malarial activity of Example 7, combined with its therapeutic window in vitro, led us to explore its activity in vivo using a murine model of malaria. Mice are infected with the murine specific Plasmodium, P. berghei, after which an antimalarial treatment regimen is given. Example 7 was given at 50 mg/kg at times 4, 24, 48, and 72 hours post-infection of mice already infected with P. berghei. The reduction in parasitemia, measured as the total blood load of the P. berghei parasite, was compared to an infected control group for the percent reduction of parasite on day 4 (96 hours post-infection). Example 7 was unexpectedly shown to be effective in reducing parasitemia by 99% compared to the infected control group after 4 days, making it an effective antimalarial in these preclinical models.

### DISCUSSION

The current success of malaria elimination has relied heavily on the cooperation of multiple factors working together in concert; biological, parasitological, social, and environmental factors (Alonso, Brown et al. 2011). However, despite the large success in the reduction of malaria, antimalarial resistance remains a sizeable issue. Resistance occurs through the failure of people to complete a full-course of their antimalarial treatment allowing Plasmodium to develop mutations in genes that can efflux drugs or counteract their mechanisms of action (Foley and Tilley 1998; Jensen and Mehlhorn 2009). With growing amounts of resistance occurring, a need for new antimalarial medicines is growing.

In the present invention, the inventors characterized the antimalarial activity of two exemplary compounds; Example 7 and Example 27 (Table 1). The inventors discovered that both Example 7 and Example 27 have antimalarial effects on the malaria-causing P. falciparum strains, NF54 (chloroquine sensitive) and K1 (chloroquine resistant). Moreover, they have appropriate cytotoxicity profiles to ensure therapeutic windows in malaria treatment. Example 7 showed unexpected efficacy in the murine malaria model, P. berghei. These compounds not only fill a need for new antimalarial compounds, but have shown better autophagy inhibiting properties than chloroquine making them potentially more effective as antimalarial agents.

With the rise in chloroquine resistant strains of Plasmodium in the world, the promise of new therapeutic options for malaria, such as these, brings hope.

### Other Embodiments

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

### REFERENCES

1. Klionsky D J. Autophagy: from phenomenology to molecular understanding in less than a decade. Nat Rev Mol Cell Biol 2007; 8:931-7.
2. Kohli L, Roth K A. Autophagy: cerebral home cooking. Am J Pathol 2010; 176:1065-71.
3. Simonsen A, Tooze S A. Coordination of membrane events during autophagy by multiple class III PI3-kinase complexes. J Cell Biol 2009; 186:773-82.
4. Rosenfeldt M T, Ryan K M. The role of autophagy in tumour development and cancer therapy. Expert Rev Mol Med 2009; 11:e36.
5. White E. Deconvoluting the context-dependent role for autophagy in cancer. Nat Rev Cancer 2012; 12:401-10.
6. Mathew R, White E. Autophagy, stress, and cancer metabolism: what doesn't kill you makes you stronger. Cold Spring Harb Symp Quant Biol 2011; 76:389-96.
7. Mizushima N, Levine B, Cuervo A M, Klionsky D J. Autophagy fights disease through cellular self-digestion. Nature 2008; 451:1069-75.
8. Yang Z, Klionsky D J. Eaten alive: a history of macroautophagy. Nat Cell Biol 2010; 12:814-22.
9. Wu W K, Coffelt S B, Cho C H, Wang X J, Lee C W, Chan F K, et al. The autophagic paradox in cancer therapy. Oncogene 2012; 31:939-53.
10. Mathew R, Karantza-Wadsworth V, White E. Role of autophagy in cancer. Nat Rev Cancer 2007; 7:961-7.
11. Kondo Y, Kanzawa T, Sawaya R, Kondo S. The role of autophagy in cancer development and response to therapy. Nat Rev Cancer 2005; 5:726-34.
12. Shingu T, Fujiwara K, Bogler O, Akiyama Y, Moritake K, Shinojima N, et al. Stage-specific effect of inhibition of autophagy on chemotherapy-induced cytotoxicity. Autophagy 2009; 5:537-9.
13. Guo J Y, Chen H Y, Mathew R, Fan J, Strohecker A M, Karsli-Uzunbas G, et al. Activated Ras requires autophagy to maintain oxidative metabolism and tumorigenesis. Genes Dev 2011; 25:460-70.
14. Yang S, Wang X, Contino G, Liesa M, Sahin E, Ying H, et al. Pancreatic cancers require autophagy for tumor growth. Genes Dev 2011; 25:717-29.
15. Maddodi N, Huang W, Havighurst T, Kim K, Longley B J, Setaluri V. Induction of autophagy and inhibition of melanoma growth in vitro and in vivo by hyperactivation of oncogenic BRAF. J Invest Dermatol 2010; 130:1657-67.
16. Fan Q W, Cheng C, Hackett C, Feldman M, Houseman B T, Nicolaides T, et al. Akt and autophagy cooperate to promote survival of drug-resistant glioma. Sci Signal 2010; 3:ra81.
17. Boya P, Gonzalez-Polo R A, Casares N, Perfettini J L, Dessen P, Larochette N, et al. Inhibition of macroautophagy triggers apoptosis. Mol Cell Biol 2005; 25:1025-40.
18. Morselli E, Galluzzi L, Kepp O, Vicencio J M, Criollo A, Maiuri M C, et al. Anti- and pro-tumor functions of autophagy. Biochim Biophys Acta 2009; 1793:1524-32.
19. Liu B, Cheng Y, Liu Q, Bao J K, Yang J M. Autophagic pathways as new targets for cancer drug development. Acta Pharmacol Sin 2010; 31:1154-64.
20. Apel A, Herr I, Schwarz H, Rodemann H P, Mayer A. Blocked autophagy sensitizes resistant carcinoma cells to radiation therapy. Cancer Res 2008; 68:1485-94.
21. Foley M, Tilley L. Quinoline antimalarials: mechanisms of action and resistance and prospects for new agents. Pharmacol Ther 1998; 79:55-87.
22. Loeb R F. Activity of a new antimalarial agent, chloroquine (SN7618). JAMA 1946; 130:1069-70.
23. Solomon V R, Lee H. Chloroquine and its analogs: a new promise of an old drug for effective and safe cancer therapies. Eur J Pharmacol 2009; 625:220-33.
24. Amaravadi R K, Yu D, Lum J J, Bui T, Christophorou M A, Evan G I, et al. Autophagy inhibition enhances therapy-induced apoptosis in a Myc-induced model of lymphoma. J Clin Invest 2007; 117:326-36.
25. Ma X H, Piao S, Wang D, McAfee Q W, Nathanson K L, Lum J J, et al. Measurements of tumor cell autophagy predict invasiveness, resistance to chemotherapy, and survival in melanoma. Clin Cancer Res 2011; 17:3478-89.
26. Han W, Sun J, Feng L, Wang K, Li D, Pan Q, et al. Autophagy inhibition enhances daunorubicin-induced apoptosis in K562 cells. PLoS One 2011; 6:e28491.
27. Rao R, Balusu R, Fiskus W, Mudunuru U, Venkannagari S, Chauhan L, et al. Combination of pan-histone deacetylase inhibitor and autophagy inhibitor exerts superior efficacy against triple-negative human breast cancer cells. Mol Cancer Ther 2012; 11:973-83.
28. Li D D, Sun T, Wu X Q, Chen S P, Deng R, Jiang S, et al. The Inhibition of Autophagy Sensitises Colon Cancer Cells with Wild-Type p53 but Not Mutant p53 to Topotecan Treatment. PLoS One 2012; 7:e45058.
29. National Library of Medicine U S. Clinical trials investigating the use of chloroquine in cancer: NCT01575782, NCT00969306, NCT01446016, NCT01023477, NCT01469455, NCT01438177, NCT01727531, NCT00224978. clinicaltrials.gov: Bethesda, Md., 2013.
30. National Library of Medicine U S. Clinical trials investigating the use of hydroxychloroquine in cancer: NCT01273805, NCT00933803, NCT01006369, NCT01480154, NCT00765765, NCT00728845, NCT01266057, NCT00813423, NCT01494155, NCT01023737, NCT00786682, NCT00726596, NCT01417403, NCT00809237, NCT00909831, NCT00714181, NCT00714181, NCT01206530, NCT01026844, NCT00486603, NCT01649947, NCT00977470, NCT01506973, NCT01128296, NCT00568880, NCT01144169, NCT00962845, NCT01292408, NCT01689987, NCT01550367, NCT01396200, NCT01227135, NCT01602588, NCT01510119, NCT00031824, NCT01548768, NCT00908089, NCT01687179, NCT00405275, NCT00771056, NCT01709578. clinicaltrials.gov: Bethesda, Md., 2013.
31. Sotelo J, Briceno E, Lopez-Gonzalez M A. Adding chloroquine to conventional treatment for glioblastoma multiforme: a randomized, double-blind, placebo-controlled trial. Ann Intern Med 2006; 144:337-43.
32. Klionsky D J. Guidelines to the use and interpretation of assays for monitoring autophagy. Autophagy 2012; 8:445-544.
33. Martin K R, Barua D, Kauffman A L, Westrate L M, Posner R G, Hlavacek W S, et al. Computational model for autophagic vesicle dynamics in single cells. Autophagy 2013; 9:74-92.
34. Singh R, Kaushik S, Wang Y, Xiang Y, Novak I, Komatsu M, et al. Autophagy regulates lipid metabolism. Nature 2009; 458:1131-5.
35. Velikkakath A K, Nishimura T, Oita E, Ishihara N, Mizushima N. Mammalian Atg2 proteins are essential for autophagosome formation and important for regulation of size and distribution of lipid droplets. Mol Biol Cell 2012; 23:896-909.
36. Chen B, Tardell C, Higgins B, Packman K, Boylan J F, Niu H. BRAFV600E negatively regulates the AKT pathway in melanoma cell lines. PLoS One 2012; 7:e42598.
37. Davies H, Bignell G R, Cox C, Stephens P, Edkins S, Clegg S, et al. Mutations of the BRAF gene in human cancer. Nature 2002; 417:949-54.
38. Flaherty K T, Puzanov I, Kim K B, Ribas A, McArthur G A, Sosman J A, et al. Inhibition of mutated, activated BRAF in metastatic melanoma. N Engl J Med 2010; 363:809-19.
39. Yan H, Zhang B, Li S, Zhao Q. A formal model for analyzing drug combination effects and its application in TNF-alpha-induced NFkappaB pathway. BMC Syst Biol 2010; 4:50.
40. Chou T C. Drug combination studies and their synergy quantification using the Chou-Talalay method. Cancer Res 2010; 70:440-6.
41. Hiss D C, Gabriels G A, Folb P I. Combination of tunicamycin with anticancer drugs synergistically enhances their toxicity in multidrug-resistant human ovarian cystadenocarcinoma cells. Cancer Cell Int 2007; 7:5.
42. Flaherty K T, Hodi F S, Fisher D E. From genes to drugs: targeted strategies for melanoma. Nat Rev Cancer 2012; 12:349-61.
43. Janku F, McConkey D J, Hong D S, Kurzrock R. Autophagy as a target for anticancer therapy. Nat Rev Clin Oncol 2011; 8:528-39.
44. Corazzari MaL, Penny E. Harnessing Autophagy for Melanoma Benefit. Cell Biology: Research & Therapy 2013; 2.
45. Xie X, White E P, Mehnert J M. Coordinate autophagy and mTOR pathway inhibition enhances cell death in melanoma. PLoS One 2013; 8:e55096.
46. Sheen J H, Zoncu R, Kim D, Sabatini D M. Defective regulation of autophagy upon leucine deprivation reveals a targetable liability of human melanoma cells in vitro and in vivo. Cancer Cell 2011; 19:613-28.
47. Sasaki K, Tsuno N H, Sunami E, Tsurita G, Kawai K, Okaji Y, et al. Chloroquine potentiates the anti-cancer effect of 5-fluorouracil on colon cancer cells. BMC Cancer 2010; 10:370.
48. Wu Y C, Wu W K, Li Y, Yu L, Li Z J, Wong C C, et al. Inhibition of macroautophagy by bafilomycin A1 lowers proliferation and induces apoptosis in colon cancer cells. Biochem Biophys Res Commun 2009; 382:451-6.
49. Sharma N, Thomas S, Golden E B, Hofman F M, Chen T C, Petasis N A, et al. Inhibition of autophagy and induction of breast cancer cell death by mefloquine, an antimalarial agent. Cancer Lett 2012; 326:143-54.
50. Han W, Pan H, Chen Y, Sun J, Wang Y, Li J, et al. EGFR tyrosine kinase inhibitors activate autophagy as a cytoprotective response in human lung cancer cells. PLoS One 2011; 6:e18691.
51. Xu Y, Yu H, Qin H, Kang J, Yu C, Zhong J, et al. Inhibition of autophagy enhances cisplatin cytotoxicity through endoplasmic reticulum stress in human cervical cancer cells. Cancer Lett 2012; 314:232-43.
52. Armstrong J L, Corazzari M, Martin S, Pagliarini V, Falasca L, Hill D S, et al. Oncogenic B-RAF signaling in melanoma impairs the therapeutic advantage of autophagy inhibition. Clin Cancer Res 2011; 17:2216-26.
53. De D, Krogstad F M, Byers L D, Krogstad D J. Structure-activity relationships for antiplasmodial activity among 7-substituted 4-aminoquinolines. J Med Chem 1998; 41:4918-26.
54. Iwaniuk D P, Whetmore E D, Rosa N, Ekoue-Kovi K, Alumasa J, de Dios A C, et al. Synthesis and antimalarial activity of new chloroquine analogues carrying a multifunctional linear side chain. Bioorg Med Chem 2009; 17:6560-6.
55. Anderson T, Nkhoma S, Ecker A, Fidock D. How can we identify parasite genes that underlie antimalarial drug resistance? Pharmacogenomics 2011; 12:59-85.
56. Muregi F W, Wamakima H N, Kimani F T. Novel drug targets in malaria parasite with potential to yield antimalarial drugs with long useful therapeutic lives. Curr Pharm Des 2012; 18:3505-21.
57. Solomon V R, Hu C, Lee H. Design and synthesis of chloroquine analogs with anti-breast cancer property. Eur J Med Chem 2010; 45:3916-23.
58. Kimura S, Noda T, Yoshimori T. Dissection of the autophagosome maturation process by a novel reporter protein, tandem fluorescent-tagged LC3. Autophagy 2007; 3:452-60.

1. A method of treating a cancer, or a cancer metastasis in a subject in need thereof, the method comprising: administering to the subject, a therapeutically effective amount of a compound of Formula A: 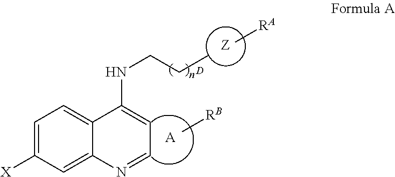
or a pharmaceutically acceptable salt thereof,
wherein:
A is an optionally substituted aryl or optionally substituted cycloalkyl;
Z is a 3 to 7 membered heterocycloalkyl;
X is H, halogen, or —CF₃;
n^{D} is 1 to 3;
R^{A} is optionally substituted C₁₋₆ alkyl; and
R^{B} is H, optionally substituted C₁₋₆ alkyl, or optionally substituted C₁₋₆ alkoxyl.

2. The method of claim 1, wherein the cancer or cancer metastasis is a cancer or cancer metastasis comprising cancer cells harboring a B-type RAF kinase (BRAF kinase) protein mutation.

3. The method of claim 1, wherein the pharmaceutical composition is administered to the subject in need thereof, in the form of a solution, dispersion, suspension, powder, capsule, tablet, pill, time release capsule, time release tablet, and time release pill.

4. The method of claim 3, wherein the pharmaceutical composition is administered to the subject intravenously, intramuscularly, subcutaneously, intraperitoneally, intratumorally, orally, or nasally.

5. The method of claim 3, wherein the pharmaceutical composition contains a therapeutically effective amount of the compound ranging from about 0.1 mg per kg body weight to about 100 mg per kg body weight.

6. The method of claim 3, wherein the pharmaceutical composition contains a therapeutically effective amount of the compound ranging from about 1 mg per kg body weight to about 50 mg per kg body weight.

7. The method of claim 3, wherein the pharmaceutical composition contains a therapeutically effective amount of the compound ranging from about 10 mg per kg body weight to about 50 mg per kg body weight.

8. The method of claim 3, wherein the pharmaceutical composition is administered to the subject at a dosage of the compound in a range of from about 0.1 mg per kg body weight to about 75 mg per kg body weight, wherein the dosage is administered one or more times per day, or one or more times per week.

9. The method of claim 3, wherein the pharmaceutical composition interferes with the autophagy capacity of at least a portion of the cancer cells within the cancer.

10. The method of claim 1, wherein the compound of Formula A or a pharmaceutically acceptable salt thereof is a compound of Formula A¹: 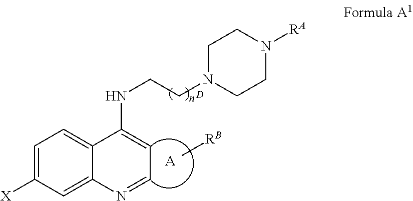
or a pharmaceutically acceptable salt thereof, wherein:
A is an optionally substituted aryl or optionally substituted cycloalkyl;
X is H, halogen, or —CF₃;
n^{D} is 1 or 3;
R^{A} is optionally substituted C₁₋₆ alkyl; and
R^{B} is H, optionally substituted C₁₋₆ alkyl, or optionally substituted C₁₋₆ alkoxyl.

11. The method of claim 1, wherein the compound of Formula A or a pharmaceutically acceptable salt thereof is a compound of Formula A²: 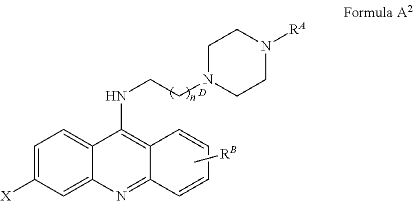
or a pharmaceutically acceptable salt thereof,
wherein:
X is H, halogen, or —CF₃;
n^{D} is 1 or 3;
R^{A} is optionally substituted C₁₋₆ alkyl; and
R^{B} is H, optionally substituted C₁₋₆ alkyl, or optionally substituted C₁₋₆ alkoxyl.

12. The method of claim 1, wherein the compound of Formula A or a pharmaceutically acceptable salt thereof is a compound of Formula A³: 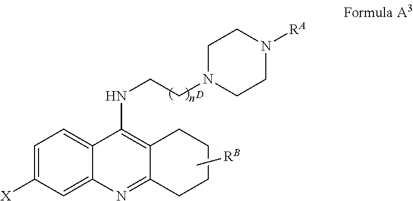
or a pharmaceutically acceptable salt thereof,
wherein:
X is H, halogen, or —CF₃;
n^{D} is 1 or 3;
R^{A} is optionally substituted C₁₋₆ alkyl; and
R^{B} is H, optionally substituted C₁₋₆ alkyl, or optionally substituted C₁₋₆ alkoxyl.

13. The method of any one of claims 10-12, wherein the pharmaceutical composition is administered to the subject in the form of a solution, dispersion, suspension, powder, capsule, tablet, pill, time release capsule, time release tablet, and time release pill.

14. The method of claim 13, wherein the pharmaceutical composition is administered to the subject intravenously, intramuscularly, subcutaneously, intraperitoneally, intratumorally, orally, or nasally.

15. The method of claim 13, wherein the pharmaceutical composition contains a therapeutically effective amount of the compound ranging from about 0.1 mg per kg body weight to about 100 mg per kg body weight.

16. The method of claim 13, wherein the pharmaceutical composition contains a therapeutically effective amount of the compound ranging from about 1 mg per kg body weight to about 50 mg per kg body weight.

17. The method of claim 13, wherein the pharmaceutical composition contains a therapeutically effective amount of the compound ranging from about 10 mg per kg body weight to about 50 mg per kg body weight.

18. The method of claim 13, wherein the pharmaceutical composition is administered to the subject at a dosage of the compound in a range of from about 0.1 mg per kg body weight to about 75 mg per kg body weight, wherein the dosage is administered one or more times per day, or one or more times per week.

19. The method of claim 13, wherein the pharmaceutical composition interferes with the autophagy capacity of at least a portion of the cancer cells within the cancer.

20. The method of any one of claims 1-19, wherein the compound is 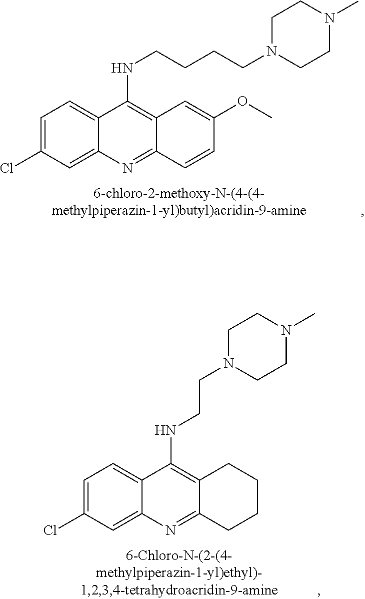 [SMILES: COc1ccc2nc3cc(Cl)ccc3c(NCCCCN3CCN(C)CC3)c2c1 ; CN1CCN(CCNc2c3c(nc4cc(Cl)ccc24)CCCC3)CC1]
or pharmaceutically acceptable salts thereof.

21. The method of any one of claims 1-20, wherein the pharmaceutical composition comprises a pharmaceutically acceptable excipient.

22. The method of any one of claims 1-21, wherein the pharmaceutical composition is administered orally.

23. The method of any one of claims 1-21, wherein the pharmaceutical composition is administered parenterally.

24. The method of claim 2, wherein the BRAF-kinase protein mutation of the cancer is selected from V600E, V600K, V600R, V600D or combinations thereof.

25. The method of claim 2, wherein the BRAF-kinase protein mutated cancer or cancer metastasis is selected from: acute myeloid leukemia, melanoma, gliomas, sarcomas, histiocytic lymphoma, non-Hodgkin's lymphoma, thyroid cancer, papillary thyroid carcinoma, head and neck cancer, liver cancer, colorectal cancer, pancreatic cancer, breast cancer, ovarian cancer, lung cancer, and non-small cell lung carcinoma.

26. The method of any one of claims 1-2, and 24-25, wherein the cancer is a melanoma cancer or a metastatic melanoma having a mutation in the cancer's BRAF-kinase protein.

27. A method of sensitizing cancer cells in a subject undergoing a chemotherapeutic treatment for the treatment of cancer, the method comprising: identifying cancer cells in the subject, and if the cancer cells are identified in said subject, administering to the subject simultaneously or sequentially, a combination comprising a therapeutically effective amount of a compound of Formula III, Formula III(a), Formula V, Formula V(a), Formula A, Formula A¹, Formula A², Formula A³, or a pharmaceutically acceptable salt thereof, and a therapeutically effective amount of an anti-cancer agent, wherein said anti-cancer agent is selected from the group consisting of N-[3-[5-(4-chlorophenyl)-1H-pyrrolo[2,3-b]pyridine-3-carbonyl]-2,4-difluorophenyl]propane-1-sulfonamide and AZD8055.

28. The method of claim 27, wherein identifying cancer cells comprises identifying cancer cells harboring a BRAF protein mutation, and if cancer cells harboring a BRAF mutation are identified in said subject, administering to the subject simultaneously or sequentially, a combination comprising a therapeutically effective amount of a compound of Formula III, Formula III(a), Formula V, Formula V(a), Formula A, Formula A¹, Formula A², Formula A³, or a pharmaceutically acceptable salt thereof, and a therapeutically effective amount of an anti-cancer agent, wherein said anti-cancer agent is selected from the group consisting of N-[3-[5-(4-chlorophenyl)-1H-pyrrolo[2,3-b]pyridine-3-carbonyl]-2,4-difluorophenyl]propane-1-sulfonamide and AZD8055.

29. The method of claim 28, wherein if the subject is identified as having cancer cells harboring a BRAF mutation, the subject is administered simultaneously or sequentially, a combination comprising a therapeutically effective amount of a compound of Formula A, Formula A¹, Formula A², Formula A³, or a pharmaceutically acceptable salt thereof, and a therapeutically effective amount of an anti-cancer agent, wherein said anti-cancer agent is selected from the group consisting of N-[3-[5-(4-chlorophenyl)-1H-pyrrolo[2,3-b]pyridine-3-carbonyl]-2,4-difluorophenyl]propane-1-sulfonamide and AZD8055.

30. The method of any one of claims 27-28, wherein the combination or each of the compound of Formula III, Formula III(a), Formula V, Formula V(a), Formula A, Formula A¹, Formula A², Formula A³, or a pharmaceutically acceptable salt thereof and the anti-cancer agent are independently administered to the subject in the form of a solution, dispersion, suspension, powder, capsule, tablet, pill, time release capsule, time release tablet, and time release pill.

31. The method of any one of claims 27-28, wherein the combination or each of the compound of Formula III, Formula III(a), Formula V, Formula V(a), Formula A, Formula A¹, Formula A², Formula A³, or a pharmaceutically acceptable salt thereof and the anti-cancer agent are independently administered to the subject intravenously, intramuscularly, subcutaneously, intraperitoneally, intratumorally, orally, nasally, or combinations thereof.

32. The method of any one of claims 27-28, wherein the combination or each of the compound of Formula III, Formula III(a), Formula V, Formula V(a), Formula A, Formula A¹, Formula A², Formula A³, or a pharmaceutically acceptable salt thereof and the anti-cancer agent are independently present in amounts ranging from about 0.01 mg per kg to about 100 mg per kg body weight of the subject.

33. The method of any one of claims 27-28, wherein the compound of Formula III, Formula III(a), Formula V, Formula V(a), Formula A, Formula A¹, Formula A², Formula A³, or a pharmaceutically acceptable salt thereof and the anti-cancer agent are each independently present in amounts ranging from about 1 mg per kg body weight to about 50 mg per kg body weight.

34. The method of any one of claims 27-28, wherein the compound of Formula III, Formula III(a), Formula V, Formula V(a), Formula A, Formula A¹, Formula A², Formula A³, or a pharmaceutically acceptable salt thereof and the anti-cancer agent are each independently present in amounts ranging from about 10 mg per kg body weight to about 50 mg per kg body weight.

35. The method of any one of claims 27-28, wherein the compound of Formula III, Formula III(a), Formula V, Formula V(a), Formula A, Formula A¹, Formula A², Formula A³, or a pharmaceutically acceptable salt thereof and the anti-cancer agent are each independently present in amounts ranging of from about 0.1 mg per kg body weight to about 25 mg per kg body weight.

36. The method of any one of claims 27-28, wherein the compound of Formula III, Formula III(a), Formula V, Formula V(a), Formula A, Formula A¹, Formula A², Formula A³, or a pharmaceutically acceptable salt thereof and the anti-cancer agent are each administered in an amount from about 1 mg to about 1,500 mg per unit dosage form.

37. The method of any one of claims 27 to 36, wherein the combination inhibits the autophagy capacity of at least a portion of the cancer cells within the cancer.

38. The method of any one of claims 27-37, wherein the compound is 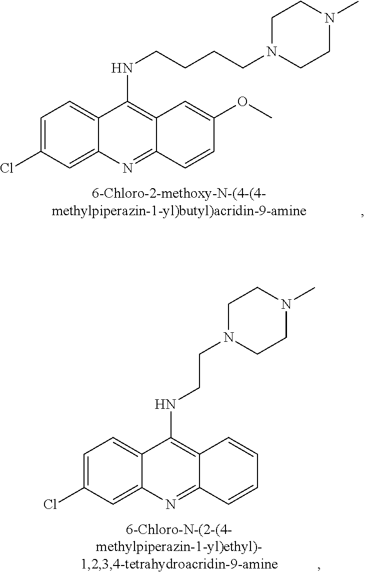 [SMILES: COc1ccc2nc3cc(Cl)ccc3c(NCCCCN3CCN(C)CC3)c2c1 ; CN1CCN(CCNc2c3ccccc3nc3cc(Cl)ccc23)CC1]
or pharmaceutically acceptable salts thereof.

39. The method of claim 27, wherein the combination comprises a pharmaceutically acceptable excipient.

40. The method of claim 27, wherein the combination is administered orally.

41. The method of claim 28, wherein the BRAF-kinase protein mutation is selected from V600E, V600K, V600R, V600D or combinations thereof.

42. The method of claim 41, wherein the cancer treated in the subject is selected from: acute myeloid leukemia, melanoma, gliomas, sarcomas, histiocytic lymphoma, non-Hodgkin's lymphoma, thyroid cancer, papillary thyroid carcinoma, head and neck cancer, liver cancer, colorectal cancer, pancreatic cancer, breast cancer, ovarian cancer, lung cancer, and non-small cell lung carcinoma.

43. The method of claim 42, wherein the cancer is a melanoma cancer or a metastatic melanoma.

44. A pharmaceutical composition for the treatment of cancer, the composition comprising a combination of a therapeutically effective amount of a compound of Formula III, Formula III(a), Formula V, Formula V(a), Formula A, Formula A¹, Formula A², Formula A³, or a pharmaceutically acceptable salt thereof, and a therapeutically effective amount of an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide and AZD8055; and
a pharmaceutically acceptable excipient.

45. The pharmaceutical composition of claim 44, wherein the compound is a compound of Formula A having a structure: 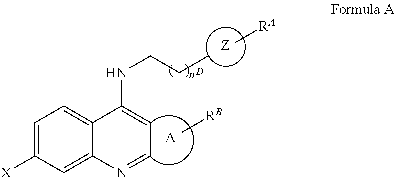
or a pharmaceutically acceptable salt thereof,
wherein:
A is an optionally substituted aryl or optionally substituted cycloalkyl ring;
Z is a 3 to 7 membered heterocycloalkyl ring;
X is H, halogen, or —CF₃;
n^{D} is 1 to 3;
R^{A} is optionally substituted C₁₋₆ alkyl; and
R^{B} is H, optionally substituted C₁₋₆ alkyl, or optionally substituted C₁₋₆ alkoxyl;

46. The pharmaceutical composition of claim 45, wherein the compound of Formula A or a pharmaceutically acceptable salt thereof is a compound of Formula A²: 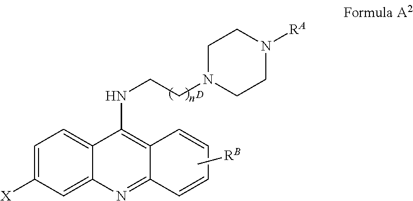
or a pharmaceutically acceptable salt thereof,
wherein:
X is H, halogen, or —CF₃;
n^{D} is 1 or 3;
R^{A} is optionally substituted C₁₋₆ alkyl; and
R^{B} is H, optionally substituted C₁₋₆ alkyl, or optionally substituted C₁₋₆ alkoxyl.

47. The pharmaceutical composition of claim 45, wherein the compound of Formula A or a pharmaceutically acceptable salt thereof is a compound of Formula A³: 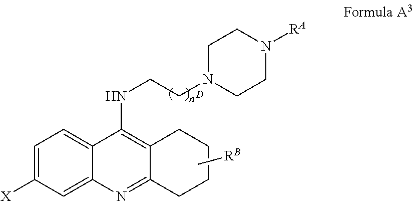
or a pharmaceutically acceptable salt thereof,
wherein:
X is H, halogen, or —CF₃;
n^{D} is 1 or 3;
R^{A} is optionally substituted C₁₋₆ alkyl; and
R^{B} is H, optionally substituted C₁₋₆ alkyl, or optionally substituted C₁₋₆ alkoxyl.

48. The pharmaceutical composition of any one of claims 45-47, wherein R^{B} is hydroxyl, amino, C₁₋₆ alkoxyl, carboxy, cyano, and halogen.

49. The pharmaceutical composition of claim 48, wherein R^{B} is C₁₋₆ alkoxyl.

50. The pharmaceutical composition of any one of claims 45-47, wherein R^{B} is H.

51. The pharmaceutical composition of any one of claims 45-50, wherein R^{A} is optionally substituted C₁₋₆ alkyl.

52. The pharmaceutical composition of any one of claim 51, wherein R^{A} is methyl, ethyl, propyl, butyl, pentyl or hexyl, unsubstituted or substituted with one to three of hydroxyl, amino, C₁₋₆ alkoxyl, carboxy, cyano, and halogen.

53. The pharmaceutical composition of any one of claims 45-52, wherein R^{A} is optionally substituted methyl.

54. The pharmaceutical composition of any one of claims 45-52, wherein R^{A} is unsubstituted methyl.

55. The pharmaceutical composition of any one of claims 45-54, wherein R^{B} is H, optionally substituted C₁₋₆ alkyl, or optionally substituted C₁₋₆ alkoxyl.

56. The pharmaceutical composition of any one of claims 45-55, wherein R^{B} is optionally substituted C₁₋₆ alkyl, or optionally substituted C₁₋₆ alkoxyl, each of which, may be unsubstituted or substituted with one to three of hydroxyl, amino, C₁₋₆ alkoxyl, carboxy, cyano, and halogen.

57. The pharmaceutical composition of any one of claims 45-56, wherein R^{B} is H or C₁₋₆ alkoxyl, selected from the group consisting of: methoxy, ethoxy, propoxy, butoxy, pentoxy and hexyloxy, each of the which may be unsubstituted or substituted with one to three of hydroxyl, amino, C₁₋₆ alkoxyl, carboxy, cyano, and halogen.

58. The pharmaceutical composition of any one of claims 45-57, wherein R^{B} is H or methoxy.

59. The pharmaceutical composition of claim 45, wherein the compound of Formula A is: 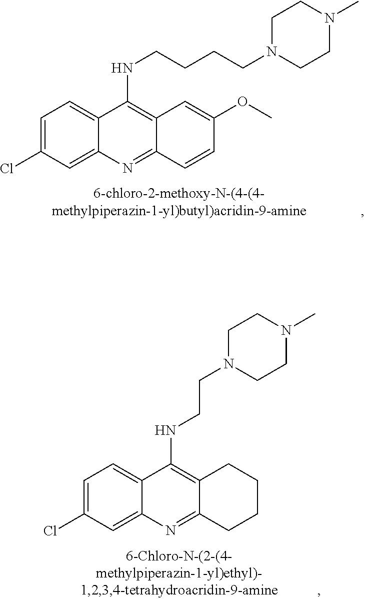 [SMILES: COc1ccc2nc3cc(Cl)ccc3c(NCCCCN3CCN(C)CC3)c2c1 ; CN1CCN(CCNc2c3c(nc4cc(Cl)ccc24)CCCC3)CC1]
or a pharmaceutically acceptable salt thereof.

60. The pharmaceutical composition of any one of claims 44-59, wherein the pharmaceutical composition contains a therapeutically effective amount of the compound of Formula III, Formula III(a), Formula V, Formula V(a), Formula A, Formula A¹, Formula A², Formula A³, or a pharmaceutically acceptable salt thereof, ranging from about 0.1 mg per kg body weight to about 100 mg per kg body weight.

61. The pharmaceutical composition of any one of claims 44-60, wherein the pharmaceutical composition contains a therapeutically effective amount of the compound of Formula III, Formula III(a), Formula V, Formula V(a), Formula A, Formula A¹, Formula A², Formula A³, or a pharmaceutically acceptable salt thereof, ranging from about 1 mg per kg body weight to about 50 mg per kg body weight.

62. The pharmaceutical composition of any one of claims 44-61, wherein the pharmaceutical composition contains a therapeutically effective amount of the compound of Formula III, Formula III(a), Formula V, Formula V(a), Formula A, Formula A¹, Formula A², Formula A³, or a pharmaceutically acceptable salt thereof, ranging from about 10 mg per kg body weight to about 50 mg per kg body weight.

63. The pharmaceutical composition of any one of claims 44-62, wherein the pharmaceutical composition contains a therapeutically effective amount of the anti-cancer agent ranging from about 0.1 mg per kg body weight to about 100 mg per kg body weight.

64. The pharmaceutical composition of any one of claims 44-63, wherein the pharmaceutical composition contains a therapeutically effective amount of the anti-cancer agent ranging from about 0.1 mg per kg body weight to about 50 mg per kg body weight.

65. The pharmaceutical composition of any one of claims 44-64, wherein the pharmaceutical composition contains a therapeutically effective amount of the anti-cancer agent ranging from about 1 mg per kg body weight to about 25 mg per kg body weight.

66. The pharmaceutical composition of any one of claims 44-65, wherein the pharmaceutical composition contains a therapeutically effective amount of the anti-cancer agent ranging from about 1 mg to about 1,500 mg.

67. The pharmaceutical composition of any one of claims 44-66, wherein the pharmaceutical composition produces a synergistic therapeutic effect as compared to sole administration of the compound of Formula A or a pharmaceutically acceptable salt thereof, or the anti-cancer agent.

68. A method for the treatment of a cancer or a cancer metastasis in a subject, the method comprising: administering to the subject simultaneously or sequentially, a therapeutically effective amount of a combination of an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide and AZD-8055; and a compound of Formula III, Formula III(a), Formula V, Formula V(a), Formula A, Formula A¹, Formula A², Formula A³, or a pharmaceutically acceptable salt thereof.

69. A method for the treatment of a cancer or a cancer metastasis in a subject, the method comprising: administering to the subject simultaneously or sequentially, a therapeutically effective amount of a combination of an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide and AZD-8055;
and a compound of Formula A having a structure: 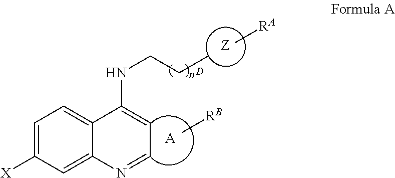
or a pharmaceutically acceptable salt thereof,
wherein:
A is an optionally substituted aryl or optionally substituted cycloalkyl;
Z is a 3 to 7 membered heterocycloalkyl;
X is H, halogen, or —CF₃;
n^{D} is 1 to 3;
R^{A} is optionally substituted C₁₋₆ alkyl; and
R^{B} is H, optionally substituted C₁₋₆ alkyl, or optionally substituted C₁₋₆ alkoxyl;
wherein the compound of Formula A or a pharmaceutically acceptable salt thereof sensitizes the cancer or cancer metastasis to the effects of the anti-cancer agent.

70. The method of claim 68 or 69, wherein the combination or each of the compound of Formula III, Formula III(a), Formula V, Formula V(a), Formula A, Formula A¹, Formula A², Formula A³, or a pharmaceutically acceptable salt thereof and the anti-cancer agent are independently administered to the subject in the form of a solution, dispersion, suspension, powder, capsule, tablet, pill, time release capsule, time release tablet, and time release pill.

71. The method of claim 70, wherein the combination or each of the compound of Formula III, Formula III(a), Formula V, Formula V(a), Formula A, Formula A¹, Formula A², Formula A³, or a pharmaceutically acceptable salt thereof and the anti-cancer agent are independently administered to the subject intravenously, intramuscularly, subcutaneously, intraperitoneally, intratumorally, orally, nasally, or combinations thereof.

72. The method of claim 68, wherein the combination or each of the compound of Formula III, Formula III(a), Formula V, Formula V(a), Formula A, Formula A¹, Formula A², Formula A³, or a pharmaceutically acceptable salt thereof and the anti-cancer agent are independently present in the combination in amounts ranging from about 0.1 mg per kg to about 100 mg per kg body weight of the subject.

73. The method of claim 72, wherein the compound of Formula III, Formula III(a), Formula V, Formula V(a), Formula A, Formula A¹, Formula A², Formula A³, or a pharmaceutically acceptable salt thereof and the anti-cancer agent are each independently present in the combination in amounts ranging from about 1 mg per kg body weight to about 50 mg per kg body weight.

74. The method of claim 72, wherein the compound of Formula III, Formula III(a), Formula V, Formula V(a), Formula A, Formula A¹, Formula A², Formula A³, or a pharmaceutically acceptable salt thereof and the anti-cancer agent are each independently present in the combination in amounts ranging from about 10 mg per kg body weight to about 50 mg per kg body weight.

75. The method of claim 72, wherein the compound of Formula III, Formula III(a), Formula V, Formula V(a), Formula A, Formula A¹, Formula A², Formula A³, or a pharmaceutically acceptable salt thereof and the anti-cancer agent are each independently present in the combination in amounts ranging of from about 0.1 mg per kg body weight to about 25 mg per kg body weight.

76. The method of claim 68, wherein the compound of Formula III, Formula III(a), Formula V, Formula V(a), Formula A, Formula A¹, Formula A², Formula A³, or a pharmaceutically acceptable salt thereof and the anti-cancer agent are each administered in an amount from about 1 mg to about 1000 mg per unit dosage form.

77. The method of any one of claims 68 to 76, wherein the combination inhibits the autophagy capacity of at least a portion of the cancer cells within the cancer.

78. The method of any one of claims 68-77, wherein the compound is 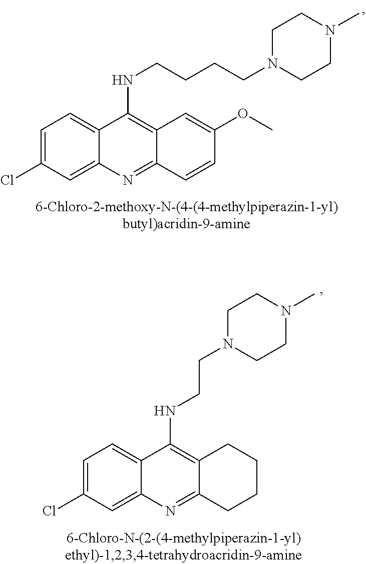 [SMILES: COc1ccc2nc3cc(Cl)ccc3c(NCCCCN3CCN(C)CC3)c2c1 ; CN1CCN(CCNc2c3c(nc4cc(Cl)ccc24)CCCC3)CC1]
or pharmaceutically acceptable salts thereof.

79. The method of claim 68, wherein the combination comprises a pharmaceutically acceptable excipient.

80. The method of claim 68, wherein the combination is administered orally.

81. The method of claim 68, wherein the cancer or the cancer metastasis harbors a BRAF-kinase protein mutation selected from V600E, V600K, V600R, V600D or combinations thereof.

82. The method of claim 81, wherein the cancer or the cancer metastasis harboring the BRAF-kinase protein mutation is selected from: acute myeloid leukemia, melanoma, gliomas, sarcomas, histiocytic lymphoma, non-Hodgkin's lymphoma, thyroid cancer, papillary thyroid carcinoma, head and neck cancer, liver cancer, colorectal cancer, pancreatic cancer, breast cancer, ovarian cancer, lung cancer, and non-small cell lung carcinoma.

83. The method of claim 82, wherein the cancer or the cancer metastasis harboring the BRAF-kinase protein mutation is a melanoma cancer or a metastatic melanoma.

84. A method for treating a cancer or a cancer metastasis in a subject, the method comprising administering to said subject, simultaneously or sequentially, a synergistically effective therapeutic amount of a combination of a compound of Formula III, Formula III(a), Formula V, Formula V(a), Formula A, Formula A¹, Formula A², Formula A³, or a pharmaceutically acceptable salt thereof, and an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide and AZD-8055.

85. The method of claim 84, wherein the combination comprises a compound of Formula A having a structure: 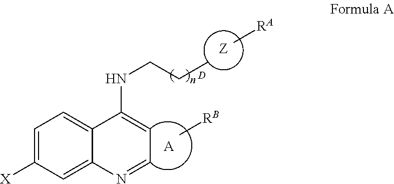
or a pharmaceutically acceptable salt thereof,
wherein:
A is an optionally substituted aryl or optionally substituted cycloalkyl;
Z is a 3 to 7 membered heterocycloalkyl;
X is H, halogen, or —CF₃;
n^{D} is 1 to 3;
R^{A} is optionally substituted C₁₋₆ alkyl; and
R^{B} is H, optionally substituted C₁₋₆ alkyl, or optionally substituted C₁₋₆ alkoxyl;
and an anti-cancer agent selected from the group consisting of N-[3-[5-(4-chlorophenyl)-1H-pyrrolo[2,3-b]pyridine-3-carbonyl]-2,4-difluorophenyl]propane-1-sulfonamide and AZD-8055.

86. The method of claim 84 or 85, wherein the combination or each of the compound of Formula III, Formula III(a), Formula V, Formula V(a), Formula A, Formula A¹, Formula A², Formula A³, or a pharmaceutically acceptable salt thereof and the anti-cancer agent are independently administered to the subject in the form of a solution, dispersion, suspension, powder, capsule, tablet, pill, time release capsule, time release tablet, and time release pill.

87. The method of claim 84 or 85, wherein the combination or each of the compound of Formula III, Formula III(a), Formula V, Formula V(a), Formula A, Formula A¹, Formula A², Formula A³, or a pharmaceutically acceptable salt thereof and the anti-cancer agent are independently administered to the subject intravenously, intramuscularly, subcutaneously, intraperitoneally, intratumorally, orally, nasally, or combinations thereof.

88. The method of claim 84 or 85, wherein the combination or each of the compound of Formula III, Formula III(a), Formula V, Formula V(a), Formula A, Formula A¹, Formula A², Formula A³, or a pharmaceutically acceptable salt thereof and the anti-cancer agent are independently present in amounts ranging from about 0.01 mg per kg to about 100 mg per kg body weight of the subject.

89. The method of claim 88, wherein the compound of Formula III, Formula III(a), Formula V, Formula V(a), Formula A, Formula A¹, Formula A², Formula A³, or a pharmaceutically acceptable salt thereof and the anti-cancer agent are each independently present in amounts ranging from about 1 mg per kg body weight to about 50 mg per kg body weight.

90. The method of claim 88, wherein the compound of Formula A or a pharmaceutically acceptable salt thereof and the anti-cancer agent are each independently present in amounts ranging from about 10 mg per kg body weight to about 50 mg per kg body weight.

91. The method of claim 88, wherein the compound of Formula A or a pharmaceutically acceptable salt thereof and the anti-cancer agent are each independently present in amounts ranging of from about 0.1 mg per kg body weight to about 25 mg per kg body weight.

92. The method of any one of claims 84-91, wherein the combination inhibits the autophagy capacity of at least a portion of the cancer cells within the cancer or cancer metastasis.

93. The method of any one of claims 84-92, wherein the compound is 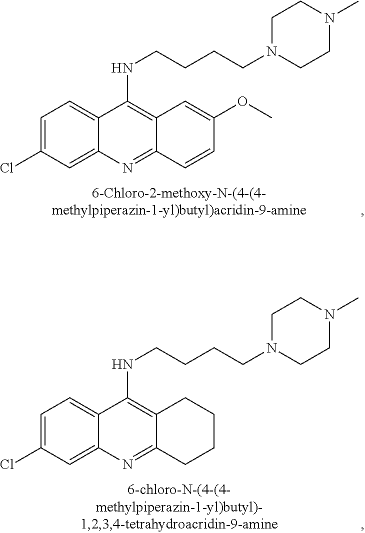 [SMILES: COc1ccc2nc3cc(Cl)ccc3c(NCCCCN3CCN(C)CC3)c2c1 ; CN1CCN(CCCCNc2c3c(nc4cc(Cl)ccc24)CCCC3)CC1]
or pharmaceutically acceptable salts thereof.

94. The method of claim 84 or 85, wherein the combination comprises a pharmaceutically acceptable excipient.

95. The method of claim 84 or 85, wherein the combination is administered orally.

96. The method of any one of claims 84-95, wherein the cancer or the cancer metastasis harbors a BRAF-kinase protein mutation selected from V599E, V600E, V600K, V600R, V600D or combinations thereof.

97. The method of claim 96, wherein the cancer or the cancer metastasis harboring the BRAF-kinase protein mutation is selected from: acute myeloid leukemia, melanoma, gliomas, sarcomas, histiocytic lymphoma, non-Hodgkin's lymphoma, thyroid cancer, papillary thyroid carcinoma, head and neck cancer, liver cancer, colorectal cancer, pancreatic cancer, breast cancer, ovarian cancer, lung cancer, and non-small cell lung carcinoma.

98. The method of any one of claim 97, wherein the cancer or the cancer metastasis harboring the BRAF-kinase protein mutation is a melanoma cancer or a metastatic melanoma.

99. The method of any one of claims 84-95, wherein the cancer or the cancer metastasis harbors a HRAS protein mutation.

100. The method of claim 1, wherein the cancer or cancer metastasis is a cancer or cancer metastasis comprising cancer cells harboring a HRAS protein mutation.

101. The method of claim 100, wherein the HRAS protein mutation of the cancer is the mutation G13V.

102. The method of any one of claims 100-101, wherein the HRAS protein mutated cancer is selected from: acute myeloid leukemia, melanoma, gliomas, sarcomas, histiocytic lymphoma, non-Hodgkin's lymphoma, thyroid cancer, papillary thyroid carcinoma, head and neck cancer, liver cancer, colorectal cancer, pancreatic cancer, breast cancer, ovarian cancer, lung cancer, and non-small cell lung carcinoma.

103. The method of any one of claims 100-102, wherein the cancer is a melanoma cancer or a metastatic melanoma having a mutation in the cancer's HRAS protein.

104. The method of claim 27, wherein identifying cancer cells comprises identifying cancer cells harboring a HRAS protein mutation, and if cancer cells harboring said HRAS protein mutation are identified in said subject, administering to the subject simultaneously or sequentially, a combination comprising a therapeutically effective amount of a compound of Formula III, Formula III(a), Formula V, Formula V(a), Formula A, Formula A¹, Formula A², Formula A³, or a pharmaceutically acceptable salt thereof, and a therapeutically effective amount of an anti-cancer agent, wherein said anti-cancer agent is selected from the group consisting of N-[3-[5-(4-chlorophenyl)-1H-pyrrolo[2,3-b]pyridine-3-carbonyl]-2,4-difluorophenyl]propane-1-sulfonamide and AZD8055.

105. The method of claim 104, wherein if the subject is identified as having cancer cells harboring said HRAS protein mutation, the subject is administered simultaneously or sequentially, a combination comprising a therapeutically effective amount of a compound of Formula A, Formula A¹, Formula A², Formula A³, or a pharmaceutically acceptable salt thereof, and a therapeutically effective amount of an anti-cancer agent, wherein said anti-cancer agent is selected from the group consisting of N-[3-[5-(4-chlorophenyl)-1H-pyrrolo[2,3-b]pyridine-3-carbonyl]-2,4-difluorophenyl]propane-1-sulfonamide and AZD8055.

106. The method of claim 104, wherein the HRAS protein mutation is G13V.

107. The method of any one of claim 105 or 106, wherein the cancer treated in the subject is selected from: acute myeloid leukemia, melanoma, gliomas, sarcomas, histiocytic lymphoma, non-Hodgkin's lymphoma, thyroid cancer, papillary thyroid carcinoma, head and neck cancer, liver cancer, colorectal cancer, pancreatic cancer, breast cancer, ovarian cancer, lung cancer, and non-small cell lung carcinoma.

108. The method of claim 107, wherein the cancer is a melanoma cancer or a metastatic melanoma.

109. A method for the prevention or treatment of malaria in a subject in need of anti-malarial prevention or treatment, the method comprising: administering to the subject, a therapeutically effective amount of a compound of Formula A: 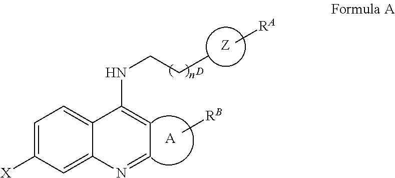
or a pharmaceutically acceptable salt thereof,
wherein:
A is an optionally substituted aryl or optionally substituted cycloalkyl;
Z is a 3 to 7 membered heterocycloalkyl;
X is H, halogen, or —CF₃;
n^{D} is 1 to 3;
R^{A} is optionally substituted C₁₋₆ alkyl; and
R^{B} is H, optionally substituted C₁₋₆ alkyl, or optionally substituted C₁₋₆ alkoxyl.

110. The method of claim 109, wherein the compound of Formula A or a pharmaceutically acceptable salt thereof is formulated into a pharmaceutical composition in the form of a solution, dispersion, suspension, powder, capsule, tablet, pill, time release capsule, time release tablet, and time release pill containing one or more doses of the compound of Formula A or a pharmaceutically acceptable salt thereof.

111. The method of claim 110, wherein the pharmaceutical composition is administered to the subject intravenously, intramuscularly, subcutaneously, intraperitoneally, orally, or nasally.

112. The method of claim 110, wherein the pharmaceutical composition contains a therapeutically effective amount of the compound of Formula A, or a pharmaceutically acceptable salt thereof ranging from about 0.01 mg per kg body weight to about 100 mg per kg body weight.

113. The method of claim 110, wherein the pharmaceutical composition contains a therapeutically effective amount of the compound of Formula A, or a pharmaceutically acceptable salt thereof ranging from about 1 mg per kg body weight to about 50 mg per kg body weight.

114. The method of claim 110, wherein the pharmaceutical composition contains a therapeutically effective amount of the compound of Formula A, or a pharmaceutically acceptable salt thereof ranging from about 10 mg per kg body weight to about 50 mg per kg body weight.

115. The method of claim 110, wherein each dose of the compound of Formula A, or a pharmaceutically acceptable salt thereof administered to the subject ranges from about 0.01 mg per kg body weight to about 100 mg per kg body weight, and one or more doses are administered one or more times per day, or one or more times per week.

116. The method of claim 110, wherein the pharmaceutical composition when administered to the subject interferes with the autophagy capacity of at least a portion of Plasmodium sp. infected cells.

117. The method of claim 109, wherein the compound of Formula A or a pharmaceutically acceptable salt thereof is a compound of Formula A¹: 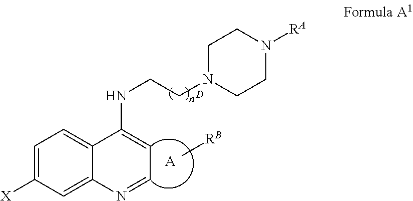
or a pharmaceutically acceptable salt thereof, wherein:
A is an optionally substituted aryl or optionally substituted cycloalkyl;
X is H, halogen, or —CF₃;
n^{D} is 1 or 3;
R^{A} is optionally substituted C₁₋₆ alkyl; and
R^{B} is H, optionally substituted C₁₋₆ alkyl, or optionally substituted C₁₋₆ alkoxyl.

118. The method of claim 109, wherein the compound of Formula A or a pharmaceutically acceptable salt thereof is a compound of Formula A²: 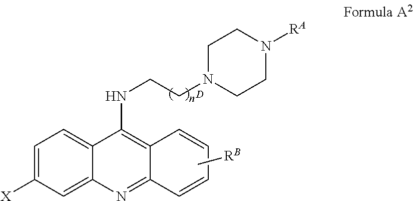
or a pharmaceutically acceptable salt thereof,
wherein:
X is H, halogen, or —CF₃;
n^{D} is 1 or 3;
R^{A} is optionally substituted C₁₋₆ alkyl; and
R^{B} is H, optionally substituted C₁₋₆ alkyl, or optionally substituted C₁₋₆ alkoxyl.

119. The method of claim 109, wherein the compound of Formula A or a pharmaceutically acceptable salt thereof is a compound of Formula A³: 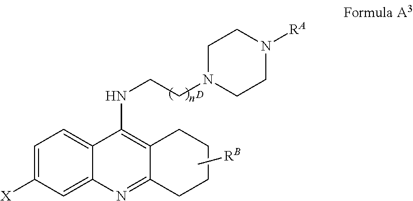
or a pharmaceutically acceptable salt thereof,
wherein:
X is H, halogen, or —CF₃;
n^{D} is 1 or 3;
R^{A} is optionally substituted C₁₋₆ alkyl; and
R^{B} is H, optionally substituted C₁₋₆ alkyl, or optionally substituted C₁₋₆ alkoxyl.

120. The method of any one of claims 117-119, wherein the compound of Formula A¹, A², or A³ or a pharmaceutically acceptable salt thereof is formulated into a pharmaceutical composition in the form of a solution, a dispersion, a suspension, a powder, a capsule, a tablet, a pill, a time release capsule, a time release tablet, or a time release pill containing one or more doses of the compound of Formula A¹, A², or A³ or a pharmaceutically acceptable salt thereof.

121. The method of claim 120, wherein the pharmaceutical composition is administered to the subject intravenously, intramuscularly, subcutaneously, intraperitoneally, orally, or nasally.

122. The method of claim 120, wherein the pharmaceutical composition contains a therapeutically effective dose amount of the compound of Formula A¹, A², or A³ or a pharmaceutically acceptable salt thereof, ranging from about 0.01 mg per kg body weight to about 100 mg per kg body weight.

123. The method of claim 120, wherein the pharmaceutical composition contains a therapeutically effective dose amount of the compound of Formula A¹, A², or A³ or a pharmaceutically acceptable salt thereof ranging from about 1 mg per kg body weight to about 50 mg per kg body weight.

124. The method of claim 120, wherein the pharmaceutical composition contains a therapeutically effective dose amount of the compound of Formula A¹, A², or A³ or a pharmaceutically acceptable salt thereof ranging from about 10 mg per kg body weight to about 50 mg per kg body weight.

125. The method of claim 120, wherein each dose of the compound of Formula A¹, A², or A³, or a pharmaceutically acceptable salt thereof administered to the subject ranges from about 0.01 mg per kg body weight to about 100 mg per kg body weight, and one or more doses are administered one or more times per day, or one or more times per week.

126. The method of claim 120, wherein the pharmaceutical composition when administered to the subject interferes with the autophagy capacity of at least a portion of Plasmodium sp. infected cells.

127. The method of any one of claims 109-126, wherein the compound is 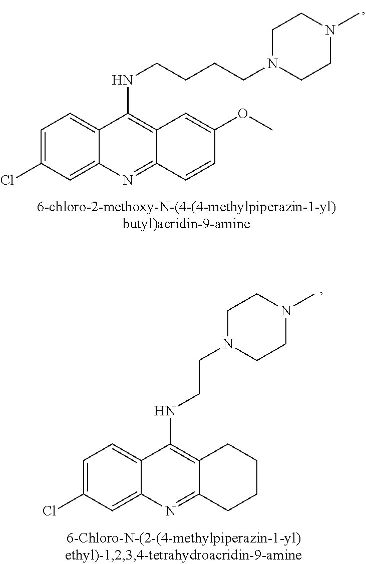 [SMILES: COc1ccc2nc3cc(Cl)ccc3c(NCCCCN3CCN(C)CC3)c2c1 ; CN1CCN(CCNc2c3c(nc4cc(Cl)ccc24)CCCC3)CC1]
or a pharmaceutically acceptable salt thereof.

128. The method of any one of claims 109-127, wherein the pharmaceutical composition comprises a pharmaceutically acceptable excipient.

129. The method of any one of claims 109-127, wherein the pharmaceutical composition is administered orally.

130. The method of any one of claims 109-127, wherein the pharmaceutical composition is administered parenterally.

131. The method of any one of claims 109-130, wherein the subject is infected with a Plasmodium sp. selected from the group consisting of Plasmodium falciparum, Plasmodium vivax, Plasmodium malariae, or Plasmodium ovale.

132. The method of claim 131, wherein the Plasmodium sp is chloroquine, mefloquine, sulfadoxine-pyrimethamine (SP), or artemisinin resistant.
